(19) United States
(12) Patent Application Publication
(10) Pub. No.: US 2025/0188190 A1
(43) Pub. Date: Jun. 12, 2025

(54) IN UTERO TREATMENT OF A FETUS HAVING GENETIC DISEASE/NEUROMUSCULAR DISEASE

(71) Applicant: argenx BV, Ghent (BE)

(72) Inventors: Roeland Vanhauwaert, Zwijnaarde (Ghent) (BE); Karen Silence, Zwijnaarde (Ghent) (BE); Steven J. Burden, Sharon, CT (US); Julien Oury, Brooklyn, NY (US)

(21) Appl. No.: 18/946,047

(22) Filed: Nov. 13, 2024

Related U.S. Application Data
(63) Continuation of application No. PCT/EP2023/063007, filed on May 15, 2023.
(60) Provisional application No. 63/364,689, filed on May 13, 2022.

(51) Int. Cl. C07K 16/40; A61K 9/00; A61K 39/00; A61P 25/28

(52) U.S. Cl. CPC C07K 16/40; A61K 9/0019; A61P 25/28; A61K 2039/505; A61K 2039/54; C07K 2317/565; C07K 2317/71; C07K 2317/75

(57) ABSTRACT

The present invention relates to an antibody-based molecule for use in the treatment of a disease or condition resulting from a genetic defect in a fetus wherein the antibody is administered to the mother of said fetus.

### CROSS REFERENCE TO RELATED APPLICATIONS

This application is a Continuation of International Patent Application No. PCT/EP2023/063007, filed May 15, 2023, which claims the benefit of and priority to U.S. Provisional Patent Application No. 63/364,689, filed May 13, 2022, the contents of each of which are incorporated herein by reference in their entirety.

### SUBMISSION OF SEQUENCE LISTING XML

The content of the following Sequence Listing XML file is incorporated herein by reference in its entirety: a computer readable form (CRF) of the Sequence Listing (file name: 404373_T2205_214218_ST26; date created: Nov. 12, 2024; and size: 378,615 bytes).

### FIELD OF THE INVENTION

The present invention relates to an antibody-based molecule for use in the treatment of a disease or condition resulting from a genetic defect in a fetus wherein the antibody-based molecule is administered to the mother of said fetus.

### BACKGROUND

Many neuromuscular disorders are hallmarked by impaired neuromuscular junctions (NMJs) and caused by genetic defects. Due to the importance of muscle specific receptor tyrosine kinase (MuSK) signalling for establishing and maintaining synapses, it is tempting to speculate that stimulating MuSK might have therapeutic potential for these disorders. Following this hypothesis, it was shown that activation of MuSK kinase activity by agonist antibodies restored neuromuscular synapse formation and prevented early lethality and late-onset disease in a mouse model of Congenital Myasthenia (CM) carrying a mutation in Docking Protein 7 (Dok7). This therapeutic strategy, which avoids the complex requirements for gene therapy (Arimura, S. et al. Neuromuscular disease. DOK7 gene therapy benefits mouse models of diseases characterized by defects in the neuromuscular junction. Science 345, 1505-1508 (2014)), might be beneficial for humans with DOK7 CM.

Moreover, this strategy has the potential for widespread use to treat genetic disorders in humans for which the disease mechanism is understood and suitable targets have been identified. Some of them would require a treatment in utero and the present invention is aimed at overcoming this issue.

### SUMMARY OF THE INVENTION

Provided herein is an antibody-based molecule for use in the treatment of a disease or condition resulting from a genetic defect in a fetus wherein the antibody-based molecule is administered to the mother of said fetus. In some embodiments, the disease or condition is a neuromuscular disease.

In another aspect, provided herein is an antibody-based molecule for use in the treatment of a fetus having a neuromuscular disease, wherein said disease is not associated with the presence of a genetic defect.

In some embodiments, the antibody-based molecule transfers via the placenta from the mother to the fetus. In some embodiments, the fetus is characterized by abnormal development, abnormal growth, expected prenatal death and/or expected early postnatal death, absent the administration of the antibody-based molecule. In some embodiments, the fetus is further characterized by abnormal neuromuscular junction formation, abnormal synapse development, and/or deficient motor function. In some embodiments, the antibody-based molecule is administered to the mother intraperitoneally, intravenously, subcutaneously in utero or intramuscularly. In some embodiments, the mother is administered as early as possible after the fetus is diagnosed with the genetic defect, or the mother is administered shortly prior to planned or expected conception to enable the fetus to be treated as early as possible by said antibody-based molecule.

In some embodiments, the antibody-based molecule is administered to the mother prior to the birth of the fetus (or newborn) and is administered to the fetus (or newborn) after birth. In some preferred embodiments, the mother is administered a loading dose or loading doses of the antibody-based molecule followed by a maintenance dose or maintenance doses administered to the newborn. In some embodiments, the maintenance dose is administered to the newborn when the disease occurs or relapses. In some embodiments, the combination of a loading dose or loading doses with a maintenance dose or maintenance doses reverses, delays and/or prevents the development of the disease or condition relative to a fetus who is not administered with said combination, wherein the fetus or newborn is assessed by metabolic parameters comprising O₂ consumption, CO₂ production, energy expenditure, total activity, water intake, food intake, and/or body weight.

In some embodiments, said treatment results in the reversion, delay and/or prevention of developmental abnormalities, fetal weight gain, postnatal survival, prolonged postnatal survival, improved postnatal development and/or postnatal weight gain, relative to a fetus whose mother is not administered with the antibody-based molecule or a fetus who is not administered with the combination, wherein the treated fetus is assessed by size estimation, growth curve, physical movement, heart rate monitoring, prenatal survival and/or postnatal survival. In some embodiments, said treatment further results in a rescue of abnormal synaptic development, abnormal synapse maturation and/or deficient motor function relative to a fetus whose mother is not administered with the antibody-based molecule or a fetus who is not administered with the combination, wherein the treated fetus is assessed by synapses counting, synapse size estimation, and/or acetylcholine receptor (AChR) density estimation at the synapse. In some embodiments, the antibody-based molecule is an anti-MuSK antibody or antigen binding fragment thereof, and the known target is MuSK. In some embodiment, the antibody-based molecule is administered at the time at which NMJ are about to be formed.

In some embodiments, the antibody-based molecule binds the MuSK Frizzled (Fz)-like domain sequence of SEQ ID NO: 129, is an agonist MuSK antibody and/or has reduced or eliminated effector function.

In some embodiments, the antibody-based molecule comprises a heavy chain variable domain (VH) and a light chain variable domain (VL) and/or a CDR, wherein:
the VH or VL has an amino acid sequence that is at least 80% identical or similar to the VH or VL as identified in table 3, and
the CDR has an amino acid sequence that is at least 80% identical or similar to the CDR as identified in table 1 or 2.

In some embodiments, the anti-MuSK antibody or antigen binding fragment thereof comprises a variable heavy chain domain (VH) and a variable light chain domain (VL):
wherein the VH comprises:
   a CDR-H1 amino acid sequence which comprises SEQ ID NO: 147 or has 1, 2, 3, 4 or 5 amino acid alterations relative to SEQ ID NO: 147,
   a CDR-H2 amino acid sequence which comprises SEQ ID NO: 153 or has 1, 2, 3, 4 or 5 amino acid alterations relative to SEQ ID NO: 153,
   a CDR-H3 amino acid sequence which comprises SEQ ID NO: 156 or has 1, 2, 3, 4 or 5 amino acid alterations relative to SEQ ID NO: 156, and
wherein the VL comprises:
   a CDR-L1 amino acid sequence which comprises SEQ ID NO: 159 or has 1, 2, 3, 4 or 5 amino acid alterations relative to SEQ ID NO: 159,
   a CDR-L2 amino acid sequence which comprises SEQ ID NO: 172 or has 1, 2, 3, 4 or 5 amino acid alterations relative to SEQ ID NO: 172,
   a CDR-L3 amino acid sequence which comprises SEQ ID NO: 195 or has 1, 2, 3, 4 or 5 amino acid alterations relative to SEQ ID NO: 195.

In some embodiments, the anti-MuSK antibody or antigen binding fragment thereof comprises:
a heavy chain variable domain (VH) comprising an amino acid sequence that is at least 80% identical or similar to SEQ ID NO: 234, and
a light chain variable domain (VL) comprising an amino acid sequence that is at least 80% identical or similar to SEQ ID NO: 235.

In some embodiments, the anti-MuSK antibody or antigen binding fragment thereof comprises:
1) a heavy chain variable domain (VH) comprising an amino acid sequence that is at least 80% identical or similar to SEQ ID NO: 234, and a light chain variable domain (VL) comprising an amino acid sequence that is at least 80% identical or similar to SEQ ID NO: 235, and
2) wherein the VH comprises:
   a CDR-H1 amino acid sequence which comprises SEQ ID NO: 147 or has 1, 2, 3, 4 or 5 amino acid alterations relative to SEQ ID NO: 147,
   a CDR-H2 amino acid sequence which comprises SEQ ID NO: 153 or has 1, 2, 3, 4 or 5 amino acid alterations relative to SEQ ID NO: 153,
   a CDR-H3 amino acid sequence which comprises SEQ ID NO: 156 or has 1, 2, 3, 4 or 5 amino acid alterations relative to SEQ ID NO: 156, and
3) wherein the VL comprises:
   a CDR-L1 amino acid sequence which comprises SEQ ID NO: 159 or has 1, 2, 3, 4 or 5 amino acid alterations relative to SEQ ID NO: 159,
   a CDR-L2 amino acid sequence which comprises SEQ ID NO: 172 or has 1, 2, 3, 4 or 5 amino acid alterations relative to SEQ ID NO: 172,
   a CDR-L3 amino acid sequence which comprises SEQ ID NO: 195 or has 1, 2, 3, 4 or 5 amino acid alterations relative to SEQ ID NO: 195.

In some embodiments, the anti-MuSK antibody or antigen binding fragment thereof comprises:
a) a full length heavy chain comprising SEQ ID NO: 268 and
b) a full length light chain comprising SEQ ID NO: 269, and
c) wherein the full length heavy chain comprises L234A and L235A mutations numbered according the EU numbering system.

In some embodiments, the anti-MuSK antibody or antigen binding fragment thereof comprises:
a full length heavy chain comprising SEQ ID NO: 270 and
a full length light chain comprising SEQ ID NO: 271, and
wherein the full length heavy chain comprises L234A and L235A mutations numbered according the EU numbering system.

In some embodiments, the anti-MuSK antibody or antigen binding fragment thereof comprises a heavy chain variable domain (VH) and a light chain variable domain (VL),
where the heavy chain variable domain comprises:
   a CDR-H1 amino acid sequence comprising SEQ ID NO: 147 or having 1, 2, 3, 4 or 5 amino acid alterations relative to SEQ ID NO: 147,
   a CDR-H2 amino acid sequence comprising SEQ ID NO: 150 or having 1, 2, 3, 4 or 5 amino acid alterations relative to SEQ ID NO: 150, and
   a CDR-H3 amino acid sequence comprising SEQ ID NO: 156 or having 1, 2, 3, 4 or 5 amino acid alterations relative to SEQ ID NO:156 and
where the light chain variable domain comprises:
   a CDR-L1 amino acid sequence comprising SEQ ID NO: 159 or having 1, 2, 3, 4 or 5 amino acid alterations relative to SEQ ID NO: 159,
   a CDR-L2 amino acid sequence comprising SEQ ID NO: 172 or having 1, 2, 3, 4 or 5 amino acid alterations relative to SEQ ID NO: 172, and
   a CDR-L3 amino acid sequence comprising SEQ ID NO: 183 or having 1, 2, 3, 4 or 5 amino acid alterations relative to SEQ ID NO:183.

In some embodiments, the anti-MuSK antibody or antigen binding fragment thereof comprises:
a heavy chain variable domain (VH) comprising an amino acid sequence that is at least 80% identical or similar to SEQ ID NO: 202, and
a light chain variable domain (VL) comprising an amino acid sequence that is at least 80% identical or similar to SEQ ID NO: 203.

In some embodiments, the anti-MuSK antibody or antigen binding fragment thereof comprises:
1) a heavy chain variable domain (VH) comprising an amino acid sequence that is at least 80% identical or similar to SEQ ID NO: 202, and a light chain variable domain (VL) comprising an amino acid sequence that is at least 80% identical or similar to SEQ ID NO: 203, and
2) where the heavy chain variable domain comprises:
   a CDR-H1 amino acid sequence comprising SEQ ID NO: 147 or having 1, 2, 3, 4 or 5 amino acid alterations relative to SEQ ID NO: 147,
   a CDR-H2 amino acid sequence comprising SEQ ID NO: 150 or having 1, 2, 3, 4 or 5 amino acid alterations relative to SEQ ID NO: 150, and
   a CDR-H3 amino acid sequence comprising SEQ ID NO: 156 or having 1, 2, 3, 4 or 5 amino acid alterations relative to SEQ ID NO:156 and
3) where the light chain variable domain comprises:
   a CDR-L1 amino acid sequence comprising SEQ ID NO: 159 or having 1, 2, 3, 4 or 5 amino acid alterations relative to SEQ ID NO: 159,
   a CDR-L2 amino acid sequence comprising SEQ ID NO: 172 or having 1, 2, 3, 4 or 5 amino acid alterations relative to SEQ ID NO: 172, and
   a CDR-L3 amino acid sequence comprising SEQ ID NO: 183 or having 1, 2, 3, 4 or 5 amino acid alterations relative to SEQ ID NO:183.

In some embodiments, the anti-MuSK antibody or antigen binding fragment thereof comprises:
a) a full length heavy chain comprising SEQ ID NO: 282 and
b) a full length light chain comprising SEQ ID NO: 283, and
c) wherein the full length heavy chain comprises L234A and L235A mutations numbered according the EU numbering system.

In a second aspect, there is provided a polynucleotide for use in the treatment of a disease or condition resulting from a genetic defect in a fetus wherein the polynucleotide is administered to the mother of said fetus, said polynucleotide comprising a nucleotide sequence which encodes the antibody-based molecule of any preceding embodiments, or a VH, VL or CDR thereof.

In another aspect, there is provided an expression vector for use in the treatment of a disease or condition resulting from a genetic defect in a fetus wherein the expression vector is administered to the mother of said fetus, comprising the polynucleotide of the second aspect of the invention, preferably operably linked to a regulatory region which allows expression of the antibody-based molecule of any preceding embodiments, or VH, VL or CDR thereof in a host cell or cell-free expression system.

In another aspect, there is provided a host cell or cell-free expression system for use in the treatment of a disease or condition resulting from a genetic defect in a fetus wherein the host cell or cell-free expression system is administered to the mother of said fetus, containing the expression vector of precedent third aspect of this invention.

In another aspect, there is provided a composition for use in the treatment of a disease or condition resulting from a genetic defect in a fetus wherein the composition is administered to the mother of said fetus, comprising an antibody-based molecule as defined in any one of precedent embodiments, a polynucleotide as defined in the second aspect of this invention, an expression vector as defined in the third aspect of this invention, or a host cell or cell-free expression system as defined in the fourth aspect of this invention. In some embodiments, the composition is a pharmaceutical composition comprising at least one pharmaceutically acceptable carrier or excipient.

In some embodiments, the polynucleotide, the expression vector or the host cell or cell-free expression system is administered to the fetus (or newborn) after birth.

In another aspect, there is provided a method for treatment of a disease or condition resulting from a genetic defect in a fetus wherein an antibody-based molecule, a polynucleotide, an expression vector, a host cell or cell-free expression system, or a composition is administered to the mother of said fetus. In some embodiments, the antibody-based molecule, the polynucleotide, the expression vector or the host cell or cell-free expression system, or the composition is administered to the fetus (or newborn) after birth.

### LEGEND TO THE DRAWINGS

FIG. 1: Dok7 1124_1129dup (Dok7 CM) mice in a CBA-C57BL/6 background, injected with ARGX-119, are fertile. Dok7 CM male and female mice were chronically injected with ARGX-119 at P4 (postnatal day 4) (20 mg/kg), P18 (postnatal day 18) (10 mg/kg) and P38 (postnatal day 18) (10 mg/kg). Once they reached sexual maturity, male and female Dok7 CM mice were mated. The female mouse became pregnant and delivered eight pups.

FIG. 2: ARGX-119 is transferred from Dok7CM pregnant mothers, allowing the mutant Dok7 CM offspring to survive postnatally. Dok7 CM female mouse in a CBA-C57BL/6 background, chronically injected with ARGX-119, gave birth to eight Dok7 CM pups. Five of eight Dok7 CM progeny, who were not themselves injected with ARGX-119, were rescued from early lethality, and gained weight over the first few weeks after birth. Three of eight Dok7 CM progeny never gained weight and died within a week after birth.

FIG. 3: Dok7 CM offspring in a mixed CBA-C57BL/6 background, not injected with ARGX-119, but born from Dok7 CM female mice, injected with ARGX-119, survive up to two months. Five of eight Dok7 CM progeny, who were not themselves injected with ARGX-119, survived two months before being sacrificed. Three of eight Dok7 CM progeny died within eight days after birth.

FIGS. 4A and 4B: Progeny from Dok7^{CM/+} CBA-C57BL/6 female, injected with ARGX-119, or Isotype control mAb, during pregnancy were recovered postnatally at the expected Mendelian ratio. Dok7^{CM/+} male and female mice in a mixed CBA-C57BL/6 genetic background were mated. After fertilization, female mice were injected twice at E5 (day 5 embryo) and E15 (day 15 embryo) with ARGX-119 ((1), 20 mg/kg), or isotype control mAb (or motavizumab) ((2), 20 mg/kg). χ² analysis of the progeny shows that the occurrence of genotypes is unlikely to occur by chance, indicating that homozygous Dok7^{CM/CM} mice born from Dok7^{CM/+} mice injected with ARGX-119 (n=10) or isotype control mAb (or motavizumab) (n=4) survive postnatally. χ² analysis test is a statistical test used to compare observed results with expected results. The purpose of this test is to determine if a difference between observed data and expected data is due to chance, or if it is due to a relationship between the variables being studied. A p-value higher than 0.05 (>0.05) is not statistically significant and indicates strong evidence for the null hypothesis. FIGS. 4A-4B indicate that there is no significant difference between the two groups of 1) the observed progeny numbers of each genotype (WT, DOK7^{CM/+}, DOK7^{CM/CM}) and 2) the expected progeny numbers of each genotype (WT, DOK7^{CM/+, DOK}7^{CM/CM}), born from Dok7^{CM/+} mice. FIG. 4C: Strategy to test mode of transfer of ARGX-119 to progeny from Dok7^{CM/+} female mice in a mixed CBA-C57BL/6 background who were injected with ARGX-119 during pregnancy. At birth, newborn pups from ARGX-119-injected mother are transferred to a Dok7^{CM/+} foster female mouse that was injected with motavizumab (1), and newborn pups from motavizumab-injected mother are transferred to a Dok7^{CM/+} foster female mouse that was injected with ARGX-119 (2). Injections had no fatal consequences on the delivery. X²=Chi-square, n=5 experiments.

FIG. 5A: ARGX-119 is transferred from placenta of Dok7^{CM/+} pregnant mothers, allowing the mutant Dok7 CM offspring to survive postnatally, and allowing the mutant Dok7 CM offspring to gain weight postnatally. Dok7^{CM/+} (CBA-C57BL/6) female mice were injected at E5 and E15 with ARGX-119 (20 mg/kg), or isotype control mAb (or motavizumab) (20 mg/kg). Newborn pups from ARGX-119-injected mother were transferred to a Dok7^{CM/+} (CBA-C57BL/6) foster female mouse that had received isotype control mAb (or motavizumab) (1). In addition, pups from isotype control mAb (or motavizumab)-injected mother were transferred to a Dok7^{CM/+} foster female mouse that had been treated with ARGX-119 (2). In neither case, were the progeny themselves injected with ARGX-119, or isotype control mAb (or motavizumab). Dok7 CM progeny, who were born from an ARGX-119-injected Dok7^{CM/+} female mice in a mixed CBA-C57BL/6 background and fostered after birth with an isotype control mAb (motavizumab)-injected female, are rescued from early lethality, and gained weight overtime. Unlikely, Dok7 CM progeny, who were born from isotype control mAb (motavizumab)-injected Dok7^{CM/+} female in a mixed CBA-C57BL/6 background and fostered with an ARGX-119-injected Dok7^{CM/+}, are not rescued from early lethality and never gained weight, proving ARGX-119 is transferred from blood and not milk of Dok7^{CM/+} female mice. Plots show the values for each individual mouse over a period of 60 days. Note: Three mice were sacrificed around P60 for histology analysis. FIG. 5B: ARGX-119 is transferred from placenta of Dok7 cm pregnant mothers in a mixed CBA-C57BL/6 background, allowing the mutant Dok7 CM offspring to gain weight postnatally. Dok7 CM progeny, who were born from ARGX-119-injected Dok7^{CM/+} female mice in a mixed CBA-C57BL/6 background and fostered after birth with motavizumab, gained weight overtime. Unlikely, Dok7 CM progeny, who were born from motavizumab Dok7^{CM/+} mice in a mixed CBA-C57BL/6 background and fostered with an ARGX-119-injected Dok7^{CM/+}, never gained weight. Plots show the values for each individual mouse over a period of 60 days.

FIG. 6: ARGX-119, transferred from placenta of Dok7^{CM/+} pregnant mothers, allows Dok7 CM offspring to survive at least two months. 10 of 10 Dok7 CM progeny, who were born from an ARGX-119-injected Dok7^{CM/+} female mouse during pregnancy and fostered after birth with an isotype control mAb (or motavizumab)-injected female, survived at least two months, and up to six months (1). All 5 Dok7 CM progeny, who were born from an isotype control mAb (or motavizumab)-injected Dok7^{CM/+} female mouse and fostered with a Dok7^{CM/+} female mouse who had been injected with ARGX-119 during pregnancy, died during the first two weeks after birth (2). Note: Three mice were sacrificed around P60 for histology analysis.

FIG. 7: ARGX-119 restores synapse development. ARGX-119 is transferred from placenta of Dok7^{CM/+} pregnant mothers, allowing the mutant Dok7 CM offspring to restore synapse development. Diaphragm muscles from P60 Dok7^{+/CM} and Dok7 mutant mice who were born from an ARGX-119-injected Dok7^{CM/+} female mouse during pregnancy and fostered after birth with an isotype control mAb (or motavizumab)-injected female were stained with Alexa 488-anti-BGT to label AChRs (red) and antibodies against Beta-3-Tubulin and Synapsin to label motor axons and nerve terminals (green). Scale bars, 100 μm.

FIG. 8: ARGX-119 restores synapse development. Diaphragm muscles from P60 Dok7^{+/CM} and Dok7 mutant mice who were born from an ARGX-119-injected Dok7^{CM/+} female mouse during pregnancy and fostered after birth with an isotype control mAb (or motavizumab)-injected female were stained with Alexa 488-anti-BGT to label AChRs (red) and antibodies against Beta-3-Tubulin and Synapsin to label motor axons and nerve terminals (green). Scale bars, 10 μm. In Dok7 CM progeny, who were born from ARGX-119-injected Dok7^{CM/+} female mice in a mixed CBA-C57BL/6 background and fostered after birth with motavizumab, synapses matured from a simple, plaque-like shape to a complex, pretzel-like shape, characteristic of mature murine neuromuscular synapses. Scale bar, 10 μm.

FIG. 9: ARGX-119 restores synapse development. Scatter plots show the number of synapses, synaptic size, density of synaptic AChRs, colocalization of Synapsin over AChR, denervation, and fragmentation of synapses from n=3 P60 Dok7^{+/CM} ice and n=3 P60 Dok7 mutant mice who were born from an ARGX-119-injected Dok7^{CM/+} female mouse during pregnancy and fostered after birth with an isotype control mAb (or motavizumab)-injected female. Dok7 CM progeny, who were born from ARGX-119-injected Dok7^{CM/+} female mice in a mixed CBA-C57BL/6 background and fostered after birth with motavizumab, the number of synapses, synaptic size and density of synaptic AChRs were restored to 67%, 45%, and 40%, respectively, of normal levels. The mean in percentage±SEM values from 3 mice (>50 synapses per mouse) normalized to non-injected wildtype mice average are shown. Two-sided Student's t-test (ns, not significant, p, *<0.005, ***<0.0005).

FIGS. 10A and 10B: Progeny from Dok7^{CM/+} C57BL/6 female in a full inbred CBA-C57BL/6 background, injected with ARGX-119 during pregnancy were recovered postnatally at the expected Mendelian ratio. FIG. 10C: After fertilization, Dok7^{CM/+} female mice in a C57BL/6 genetic background were injected twice at E5 and E15 with ARGX-119 (20 mg/kg) or isotype control mAb (or motavizumab) (20 mg/kg). χ² analysis of the progeny shows that the occurrence of genotypes is unlikely to occur by chance, indicating that homozygous Dok7^{CM/CM} mice born from Dok7^{CM/+} mice injected with ARGX-119 survive postnatally. Injection with ARGX-119 had no fatal consequences on the delivery. Dok7 CM C57BL/6 mice born from Dok7^{CM/+} female injected with isotype control mAb (or motavizumab) die at birth, like untreated Dok7 CM C57BL/6 mice. X²=Chi-square, n=3 experiments.

FIG. 11A: Dok7CM Mice in a full inbred C57BL/6 background are rescued from neonatal lethality by injecting their pregnant mothers with ARGX-119. After fertilization, Dok7^{CM/+} (C57BL/6) female mice were injected at E5 and E15 with ARGX-119 (20 mg/kg). The progeny not injected themselves with ARGX-119 are rescued from early lethality and gained weight overtime. FIG. 11B: Dok7 CM Progeny from Dok7^{CM/+} C57BL/6 female, injected with ARGX-119 during pregnancy, gain weight. Dok7 CM progeny not injected themselves with ARGX-119 but born from Dok7^{CM/+} C57BL/6 female, injected with ARGX-119 during pregnancy, gained weight overtime. Plots show the values for each individual mouse over a period of 60 days.

FIG. 12: Dok7 CM Mice in a full inbred C57BL/6 background can survive nearly two months by Injecting their pregnant mothers with ARGX-119. 6 of 6 Dok7 CM C57BL/6 progeny, who were born from an ARGX-119-injected Dok7 CM C57BL/6 female mouse during pregnancy, survived nearly two months postnatally. Dok7 CM C57BL/6 mice born from Dok7^{CM/+} female injected with isotype control motavizumab die at birth.

FIG. 13: ARGX-119 restores synapse development in Dok7 CM Progeny born from Dok7^{CM/+} C57BL/6 female in a full inbred background, injected with ARGX-119 during pregnancy. Diaphragm muscles from P60 Dok7^{+/CM} and Dok7 mutant mice who were born from an ARGX-119-injected Dok7 CM C57BL/6 female mouse during pregnancy were stained with Alexa 488-anti-BGT to label AChRs (red) and antibodies against Beta-3-Tubulin and Synapsin to label motor axons and nerve terminals (green). Scale bars, 100 μm.

FIG. 14: ARGX-119 restores synapse development in Dok7 CM Progeny born from Dok7^{CM/+} C57BL/6 female in a full inbred background, injected with ARGX-119 during pregnancy. Diaphragm muscles from P60 Dok7^{+/CM} and Dok7 mutant mice who were born from an ARGX-119-injected Dok7 CM C57BL/6 female mouse during pregnancy were stained with Alexa 488-anti-BGT to label AChRs (red) and antibodies against Beta-3-Tubulin and Synapsin to label motor axons and nerve terminals (green). Scale bars, 10 μm. In Dok7 CM progeny, who were born from ARGX-119-injected Dok7^{CM/+} female mice in a full inbred CBA-C57BL/6 background, synapses matured from a simple, plaque-like shape to a complex, pretzel-like shape, characteristic of mature murine neuromuscular synapses. Scale bar, 10 μm.

FIG. 15: ARGX-119 restores synapse development in Dok7 CM Progeny born from Dok7^{CM/+} C57BL/6 female in a full inbred background, injected with ARGX-119 during pregnancy. Scatter plots showthe number of synapses, synaptic size, density of synaptic AChRs, colocalization of Synapsin over AChR, denervation, and fragmentation of synapses from n=3 P60 Dok7^{+/CM} mice and n=3 P60 Dok7 mutant mice who were born from an ARGX-119-injected Dok7 CM C57BL/6 female mouse during pregnancy. In Dok7 CM progeny, who were born from ARGX-119-injected Dok7^{CM/+} female mice in a full inbred CBA-C57BL/6 background, the number of synapses, synaptic size and density of synaptic AChRs were restored to 46%, 59%, and 49%, respectively, of normal levels. The mean in percentage t SEM values from 3 mice (>50 synapses per mouse) normalized to non-injected wildtype mice average are shown. Two-sided Student's t-test (p, *<0.05)

FIG. 16A: MuSK agonist antibodies does not rescue Rapsyn^{-/-} mutant mice from neonatal lethality. Strategy to rescue Rapsyn^{-/-} mutant mice in a full inbred C57BL/6 background from neonatal lethality. After fertilization, Rapsyn^{+/-} female mice in a C57BL/6 genetic background were injected twice at E5 and E15 with ARGX-119 (20 mg/kg) or isotype control motavizumab (20 mg/kg). Their progeny was not injected with ARGX-119 or motavizumab. FIG. 16B: Progeny from Rapsyn^{+/-} C57BL/6 female, injected with ARGX-119 during pregnancy were not recovered postnatally at the expected Mendelian ratio. χ² analysis of the progeny shows that the occurrence of genotypes is unlikely to occur by chance, indicating that Rapsyn^{-/-} mice in a C57BL/6 genetic background born from Rapsyn^{+/-} mice injected with ARGX-119 don't survive postnatally (n=3 experiments).

FIG. 17A: MuSK agonist antibodies rescue Agrin^{ΔZ/ΔZ} mutant mice from neonatal lethality. A. Strategy to rescue Agrin^{ΔZ/ΔZ} mutant mice in a full inbred C57BL/6 background from neonatal lethality. After fertilization, Agrin^{ΔZ/+} female mice in a C57BL/6 genetic background were injected twice at E13 with MuSK agonist antibodies 3B2 (20 mg/kg) or isotype control motavizumab (20 mg/kg). Their progeny was not injected with 3B2 or motavizumab. FIG. 17B: Progeny from Agrin^{ΔZ/+} C57BL/6 female, injected with 3B2 during pregnancy were recovered postnatally at the expected Mendelian ratio. χ2 analysis of the progeny shows that the occurrence of genotypes is unlikely to occur by chance, indicating that Agrin^{ΔZ/ΔZ} mice in a C57BL/6 genetic background born from Agrin^{ΔZ/+} mice injected with 3B2 survive postnatally. Injection with 3B2 had no fatal consequences on the delivery (n=14 experiments). Agrin^{ΔZ/ΔZ} C57BL/6 mice born from Agrin^{ΔZ/+} female injected with isotype control motavizumab die at birth, like untreated Agrin^{ΔZ/ΔZ} mice C57BL/6 mice (n=3 experiments).

FIG. 18: Agrin^{ΔZ/ΔZ} mice in a C57BL/6 background are partially rescued by injecting their pregnant mothers with 3B2. Agrirn® C57BL/6 progeny, who were born from an 3B2-injected Agrin^{ΔZ/+} C57BL/6 female mouse during pregnancy, can survive on average nearly 40 days, and up to 55 days. Agrin^{ΔZ/ΔZ} C57BL/6 mice born from Agrin^{ΔZ/+} female injected with isotype control motavizumab die at birth. Plot shows the percentage of mice surviving over 60 days.

FIG. 19A: Postnatal injection of MuSK agonist antibodies 3B2 shows a moderate effect on survival or weight gain of Agrin^{ΔZ/ΔZ} mice. Agrin^{ΔZ/ΔZ} C57BL/6 progeny, who were born from 3B2-injected Agrin^{ΔZ/+} 57BL/6 female mice during pregnancy ultimately shows disease relapse around P30. Red dots indicate natural death or sacrifice at disease end point (˜20% weight loss); orange dots indicate the end of experiment. FIG. 19B: Agrin^{ΔZ/ΔZ} C57BL/6 progeny, who were born from an 3B2-injected Agrin^{ΔZ/+} C57BL/6 female mouse during pregnancy ultimately shows disease relapse around P30. Whey they lost weight (˜10% weight loss), they were re-injected with 3B2 (10 mg/kg), and monitored. Plots show the values for each individual mouse overtime. Red dots indicate natural death or sacrifice at disease end point (˜20% weight loss).

FIGS. 20A-20G: Agrin^{ΔZ/ΔZ} mice display respiratory, energy expenditure, activity, water intake, food intake, and body weight deficits compared to wildtype mice when disease relapses. P40 Agrin^{ΔZ/ΔZ} C57BL/6 mice, who were born from 3B2-injected Agrin^{ΔZ/+} C57BL/6 female mice during pregnancy, were placed in metabolic chambers and monitored 24 hours a day for 4 days. O₂ consumption (FIG. 20A), CO₂ production (FIG. 20B), energy expenditure (FIG. 20C), total activity (FIG. 20D), water intake (FIG. 20E), food intake (FIG. 20F), and body weight measurements (FIG. 20G), were taken. The scatter plots show the values for 10 control (wildtype or Agrin^{ΔZ/+}) mice and 10 Agrin^{ΔZ/ΔZ} mice and the mean in percentage normalized to wildtype mice average±SEM values. Two-sided Student's t-test (p, *<0.05, p, *<0.005, p, **<0.0005, ****<0.00005).

FIG. 21: Agrin^{ΔZ/ΔZ} mice display motor deficits when disease relapses. Agrin^{ΔZ/ΔZ} mice in a C57BL/6 background, who were born from 3B2-injected Agrin^{ΔZ/+} C57BL/6 female mice during pregnancy, display motor deficits at disease onset (10% weight loss) as assessed by the latency to fall from a rotating rotarod compared to non-injected wildtype mice. Plots show individual data points and mean in percentage normalized to non-injected mice average±SEM. Two-sided Student's t-test (p, *<0.05).

FIGS. 22A-22B: 3B2 partially restores synapse development in Agrin^{ΔZ/ΔZ} Progeny born from Agrin^{ΔZ/+} C57BL/6 female, injected with 3B2 during pregnancy. Upper panel, diaphragm muscles from P10 and P40 mice were stained with Alexa 488-a-BGT to label AChRs (red) and antibodies to β-III Tubulin/Synapsin to label motor axons/nerve terminals (green). Scale bar, 10 μm. FIGS. 22C-22H display scatterplots which show number of synapses (FIG. 22C), the synaptic size (FIG. 22D), AChR density at the synapse (FIG. 22E), colocalization of synapse and AChR (FIG. 22F), the percent of denervated synapses (FIG. 22G), and fragmented synapses (FIG. 22H).

### DETAILED DESCRIPTION OF THE INVENTION

### General Definitions

The following terms or definitions are provided solely to aid in the understanding of the invention. Unless specifically defined herein, all terms used herein have the same meaning as they would to one skilled in the art of the present invention. Practitioners are particularly directed to Sambrook et al., Molecular Cloning: A Laboratory Manual, 2nd ed., Cold Spring Harbor Press, Plainsview, New York (1989); and Ausubel et al., Current Protocols in Molecular Biology (Supplement 47), John Wiley & Sons, New York (1999), for definitions and terms of the art. The definitions provided herein should not be construed to have a scope less than understood by a person of ordinary skill in the art.

Unless indicated otherwise, all methods, steps, techniques and manipulations that are not specifically described in detail can be performed and have been performed in a manner known per se, as will be clear to the skilled person. Reference is for example again made to the standard handbooks, to the general background art referred to above and to the further references cited therein.

As used herein, the singular forms “a”, “an”, and “the” include both singular and plural referents unless the context clearly dictates otherwise.

The terms “comprising”, “comprises” and “comprised of” as used herein are synonymous with ‘including’, ‘includes’ or ‘containing’, ‘contains’, and are inclusive or open-ended and do not exclude additional, non-recited members, compounds, products, elements or method steps. The expression “essentially consists of” used in the context of a product or a composition (“a product essentially consisting of” or “a composition essentially consisting of”) means that additional molecules may be present but that such molecule does not change/after the characteristic/activity/functionality of said product or composition. For example, a composition may essentially consist of an antibody or an antibody fragment if the composition as such would exhibit similar characteristic/activity/functionality as one of the antibodies or as the one of the antibody fragments.

The recitation of numerical ranges by endpoints includes all numbers and fractions subsumed within the respective ranges, as well as the recited endpoints.

The term “about” as used herein when referring to a measurable value such as a parameter, an amount, a temporal duration, and the like, is meant to encompass variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” refers is itself also specifically, and preferably, disclosed. The terms ‘disorder’ and ‘disease’ are used herein interchangeably.

As used herein, amino acid residues will be indicated either by their full name or according to the standard three-letter or one-letter amino acid code. As used herein, the terms “polypeptide” or “protein” are used interchangeably, and refer to a polymeric form of amino acids of any length, which can include coded and non-coded amino acids, chemically or biochemically modified or derivatized amino acids, and polypeptides having modified peptide backbones. A“peptide” is also a polymer of amino acids with a length which is usually of up to 50 amino acids. A polypeptide or peptide is represented by an amino acid sequence.

As used herein, the terms “nucleic acid molecule”, “polynucleotide”, “polynucleic acid”, “nucleic acid” are used interchangeably and refer to polymeric form of nucleotides of any length, either deoxyribonucleotides or ribonucleotides, or analogs thereof. A nucleic acid molecule is represented by a nucleic acid sequence, which is primarily characterized by its base sequence. Polynucleotides may have any three-dimensional structure, and may perform any function, known or unknown. Non-limiting examples of polynucleotides include a gene, a gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, control regions, isolated RNA of any sequence, nucleic acid probes, and primers. The nucleic acid molecule may be linear or circular.

As used herein, the term “homology” denotes at least secondary structural identity or similarity between two macromolecules, particularly between two polypeptides or polynucleotides, from same or different taxons, wherein said similarity is due to shared ancestry. Hence, the term ‘homologues’ denotes so-related macromolecules having said secondary and optionally tertiary structural similarity. For comparing two or more nucleotide sequences, the ‘(percentage of) sequence identity’ between a first nucleotide sequence and a second nucleotide sequence may be calculated using methods known by the person skilled in the art, e.g. by dividing the number of nucleotides in the first nucleotide sequence that are identical to the nucleotides at the corresponding positions in the second nucleotide sequence by the total number of nucleotides in the first nucleotide sequence and multiplying by 100% or by using a known computer algorithm for sequence alignment such as NCBI Blast. In determining the degree of sequence similarity between two amino acid sequences, the skilled person may take into account so-called ‘conservative’ amino acid substitutions, which can generally be described as amino acid substitutions in which an amino acid residue is replaced with another amino acid residue of similar chemical structure and which has little or essentially no influence on the function, activity or other biological properties of the polypeptide. Possible conservative amino acid substitutions have been exemplified herein. Amino acid sequences and nucleic acid sequences are said to be ‘exactly the same’ if they have 100% sequence identity over their entire length.

Throughout this application, each time one refers to a specific amino acid sequence SEQ ID NO (take SEQ ID NO: Y as example), one may replace it by: a polypeptide comprising an amino acid sequence that has at least 80% sequence identity or similarity with amino acid sequence SEQ ID NO: Y. Throughout this application, the wording “a sequence is at least X % identical with another sequence” may be replaced by “a sequence has at least X % sequence identity with another sequence”.

Each amino acid sequence described herein by virtue of its identity percentage (at least 80%) with a given amino acid sequence respectively has in a further preferred embodiment an identity of at least 80%, 81%, 82%, 83%, 84%, 85%, 86%, 87%, 88%, 89%, 90%, 91%, 92%, 93%, 94%, 95%, 96%, 97%, 98%, 99% or more identity with the given amino acid sequence respectively. In a preferred embodiment, sequence identity is determined by comparing the whole length of the sequences as identified herein. Each amino acid sequence described herein by virtue of its similarity percentage (at least 80%) with a given amino acid sequence respectively has in a further preferred embodiment a similarity of at least 80%, 81%, 82%, 83%, 84%, 85%, 86%, 87%, 88%, 89%, 90%, 91%, 92%, 93%, 94%, 95%, 96%, 97%, 98%, 99% qwerty or more similarity with the given amino acid sequence respectively. In a preferred embodiment, sequence similarity is determined by comparing the whole length of the sequences as identified herein. Unless otherwise indicated herein, identity or similarity with a given SEQ ID NO means identity or similarity based on the full length of said sequence (i.e. over its whole length or as a whole). “Sequence identity” is herein defined as a relationship between two or more amino acid (polypeptide or protein) sequences or two or more nucleic acid (polynucleotide) sequences, as determined by comparing the sequences. The identity between two amino acid sequences is preferably defined by assessing their identity within a whole SEQ ID NO as identified herein or part thereof. Part thereof may mean at least 50% of the length of the SEQ ID NO, or at least 60%, or at least 70%, or at least 80%, or at least 90%.

In the art, “identity” also means the degree of sequence relatedness between amino acid sequences, as the case may be, as determined by the match between strings of such sequences. “Similarity” between two amino acid sequences is determined by comparing the amino acid sequence and its conserved amino acid substitutes of one polypeptide to the sequence of a second polypeptide. “Identity” and “similarity” can be readily calculated by known methods, including but not limited to those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heine, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48:1073 (1988).

Preferred methods to determine identity are designed to give the largest match between the sequences tested. Methods to determine identity and similarity are codified in publicly available computer programs. Preferred computer program methods to determine identity and similarity between two sequences include e.g. the GCG program package (Devereux, J., et al., Nucleic Acids Research 12 (1): 387 (1984)), BestFit, FASTA, BLASTN, and BLASTP (Altschul, S. F. et al., J. Mol. Biol. 215:403-410 (1990)), EMBOSS Needle (Madeira, F., et al., Nucleic Acids Research 47(W1): W636-W641 (2019)). The BLAST program is publicly available from NCBI and other sources (BLAST Manual, Altschul, S., et al., NCBI NLM NIH Bethesda, MD 20894; Altschul, S., et al., J. Mol. Biol. 215:403-410 (1990)). The EMBOSS program is publicly available from EMBL-EBI. The well-known Smith Waterman algorithm may also be used to determine identity. The EMBOSS Needle program is the preferred program used.

Preferred parameters for polypeptide sequence comparison include the following: Algorithm: Needleman and Wunsch, J. Mol. Biol. 48 (3):443-453 (1970); Comparison matrix: BLOSUM62 from Henikoff and Henikoff, Proc. Natl. Acad. Sci. USA. 89:10915-10919 (1992); Gap Open Penalty: 10; and Gap Extend Penalty: 0.5. A program useful with these parameters is publicly available as the EMBOSS Needle program from EMBL-EBI. The aforementioned parameters are the default parameters for a Global Pairwise Sequence alignment of proteins (along with no penalty for end gaps).

Preferred parameters for nucleic acid comparison include the following: Algorithm: Needleman and Wunsch, J. Mol. Biol. 48:443-453 (1970); Comparison matrix: DNA full; Gap Open Penalty: 10; Gap Extend Penalty: 0.5. A program useful with these parameters is publicly available as the EMBOSS Needle program from EMBL-EBI. The aforementioned parameters are the default parameters for a Global Pairwise Sequence alignment of nucleotide sequences (along with no penalty for end gaps).

Also provided herein are embodiments wherein any embodiment described herein may be combined with any one or more other embodiments, provided the combination is not mutually exclusive.

### An Antibody-Based Molecule for Use in the Treatment of a Fetus.

The present invention is based on the surprising discovery that a fetus with a known genetic defect could benefit from treatment by an antibody-based molecule as early as possible, thereby being rescued from syndromes associated to the genetic defects, such as early (postnatal) death, and/or abnormal growth.

In a first aspect of this invention, there is provided an antibody-based molecule for use in the treatment of a disease or condition resulting from a genetic defect in a fetus wherein the antibody-based molecule is administered to the mother of said fetus.

All antibodies, antibody-based molecules or antigen-binding fragments thereof defined herein are encompassed as such in the present invention. The antibodies, antibody-based molecules or antigen-binding fragments thereof are for use in the treatment of a disease or condition resulting from a genetic defect in a fetus wherein the antibody-based molecule is administered to the mother of said fetus.

Antibody-based molecules include, without limitation antibodies, full antibodies, epitope binding fragments of whole antibodies, antigen binding fragments of whole antibodies and antibody derivatives. Therefore in an embodiment, the expression “antibody-based molecule” may be replaced by antibody.

An epitope binding fragment of an antibody can be obtained through the actual fragmenting of a parental antibody (for example, a Fab or (Fab)2 fragment). Alternatively, the epitope binding fragment is an amino acid sequence that comprises a portion of the amino acid sequence of such parental antibody. As used herein, a molecule is said to be a “derivative” of an antibody (or relevant portion thereof) if it is obtained through the actual chemical modification of a parent antibody or portion thereof, or if it comprises an amino acid sequence that is substantially similar to the amino acid sequence of such parental antibody or relevant portion thereof (for example, differing by up to 30%, up to 20%, up to 10%, up to 5%, up to 4%, up to 3%, up to 2%, or up to 1% from such parental molecule or such relevant portion thereof, e.g., a chain or a variable domain thereof, or by up to 30 amino acid residues, up to 20 amino acid residues, up to 10, 9, 8, 7, 6, 5, 4, 3, 2, or 1 amino acid residues from such parental molecule or relevant portion thereof), e.g., a chain or a variable domain thereof.

In an embodiment, an antibody-based molecule of the present invention is an intact immunoglobulin or a molecule having an epitope-binding fragment thereof or having an antigen binding fragment thereof. As used herein, the terms “fragment”, “region”, “portion”, and “domain” are generally intended to be synonymous, unless the context of their use indicates otherwise. Naturally occurring antibodies typically comprise a tetramer, which is usually composed of at least two heavy (H) chains and at least two light (L) chains. Each heavy chain is comprised of a heavy chain variable domain (VH) and a heavy chain constant region (CH), usually comprised of three domains (CH1, CH2 and CH3 domains). Heavy chains can be of any isotype, including IgG (IgG1, IgG2, IgG3 and IgG4 subtypes), IgA (IgA1 and IgA2 subtypes), IgM and IgE. Each light chain is comprised of a light chain variable domain (VL) and a light chain constant region (CL). Light chains include kappa chains and lambda chains. The VLs and VHs are typically responsible for antigen recognition, while the heavy and light chain constant regions may mediate the binding of the immunoglobulin to host tissues or factors, including various cells of the immune system (e.g., effector cells) and the first component (Clq) of the classical complement system. The VH and VL can be further subdivided into regions of hypervariability, termed “complementarity determining regions,” or “CDRs,” that are interspersed with regions of more conserved sequence, termed “framework regions” (FR). Each VH and VL is composed of three CDRs and four FRs arranged from amino-terminus to carboxy-terminus in the following order: FR1-CDR1-FR2-CDR2-FR3-CDR3-FR4. The variable domains of the heavy and light chains contain a binding region that interacts with an antigen. Of particular relevance are antibodies and their epitope-binding fragments that have been “isolated” so as to exist in a physical milieu distinct from that in which it may occur in nature or that have been modified so as to differ from a naturally-occurring antibody in amino acid sequence.

In the context of this application, a variable heavy chain domain (VH) may be called a heavy chain variable region or a variable heavy chain, and a variable light chain domain (VL) may be called a light chain variable region or a variable light chain.

Fragments of antibodies (including Fab and (Fab)2 fragments) that exhibit epitope-binding ability can be obtained, for example, by protease cleavage of intact antibodies. Single domain antibody fragments possess only one variable domain (e.g., VL or VH). Examples of the epitope-binding fragments encompassed within the present invention include (i) Fab′ or Fab fragments, which are monovalent fragments containing the VL, VH, CL and CH1; (ii) F(ab′)2 fragments, which are bivalent fragments comprising two Fab fragments linked by a disulfide bridge at the hinge region; (iii) Fd fragments consisting essentially of the VH and CH1; (iv) Fv fragments consisting essentially of a VL and VH, (v) dAb fragments (Ward et al. “Binding Activities Of A Repertoire Of Single Immunoglobulin Variable Domains Secreted From Escherichia coli,” Nature 341:544-546 (1989), which is hereby incorporated by reference in its entirety), which consist essentially of a VH or VL and also called domain antibodies (Holt et al. “Domain Antibodies: Proteins For Therapy,” Trends Biotechnol. 21(11):484-490 (2003), which is hereby incorporated by reference in its entirety); (vi) nanobodies (Revets et al. “Nanobodies As Novel Agents For Cancer Therapy,” Expert Opin. Biol. Ther. 5(1):111-124 (2005), which is hereby incorporated by reference in its entirety), and (vii) isolated complementarity determining regions (CDRs). An epitope-binding fragment may contain 1, 2, 3, 4, 5 or all 6 of the CDR of such antibody. In an embodiment, a fragment (or region or portion or domain) of an antibody comprises, essentially consists of, or consists of 30 to 100 amino acids or 50 to 150 amino acids or 70 to 200 amino acids. In an embodiment, the length of a fragment (or region or portion or domain) of an antibody is at least 40%, 50%, 60%, 70%, 80%, 90% or 95% of the length of the antibody (full length antibody). In an embodiment, a fragment is an epitope binding fragment or a functional fragment of said antibody meaning it is expected it will elicit an activity of the antibody at least to some extent “At least to some extent” may mean at least 20%, 30%, 40%, 50%, 60%, 70%, 80%, 90%, 95%, 100%, 150%, 200% or more. In an embodiment, the fragment of the antibody or the antibody should elicit a detectable activity of the antibody. An activity of the antibody has been earlier defined herein.

Such antibody fragments may be obtained using conventional techniques known to those of skill in the art. For example, F(ab′)2 fragments may be generated by treating a full-length antibody with pepsin. The resulting F(ab′)2 fragment may be treated to reduce disulfide bridges to produce Fab′ fragments. Fab fragments may be obtained by treating an IgG antibody with papain and Fab′ fragments may be obtained with pepsin digestion of IgG antibody. A Fab′ fragment may be obtained by treating an F(ab)2 fragment with a reducing agent, such as dithiothreitol. Antibody fragments may also be generated by expression of nucleic acids encoding such fragments in recombinant cells (see e.g., Evans et al. “Rapid Expression Of An Anti-Human C5 Chimeric Fab Utilizing A Vector That Replicates In COS And 293 Cells,” J. Immunol. Meth. 184:123-38 (1995), which is hereby incorporated by reference in its entirety). For example, a chimeric gene encoding a portion of a F(ab′)2 fragment could include DNA sequences encoding the CH1 domain and hinge region of the heavy chain, followed by a translational stop codon to yield such a truncated antibody fragment molecule. Suitable fragments capable of binding to a desired epitope may be readily screened for utility in the same manner as an intact antibody. Preferably, suitable fragments are fused with an IgG Fc domain.

Antibody derivatives include those molecules that contain at least one epitope-binding domain of an antibody, and are typically formed using recombinant techniques. One exemplary antibody derivative includes a single chain Fv (scFv). A scFv is formed from the two domains of the Fv fragment, the VL and the VH, which may be encoded by separate genes. Such gene sequences or their encoding cDNA are joined, using recombinant methods, by a flexible linker (typically of about 10, 12, 15 or more amino acid residues) that enables them to be made as a single protein chain in which the VL and VH associate to form monovalent epitope-binding molecules (see e.g., Bird et al. “Single-Chain Antigen-Binding Proteins,” Science 242:423-426 (1988); and Huston et al. “Protein Engineering Of Antibody Binding Sites: Recovery Of Specific Activity In An Anti-Digoxin Single-Chain Fv Analogue Produced In Escherichia coli,” Proc. Natl. Acad. Sci. (U.S.A.) 85:5879-5883 (1988), which are hereby incorporated by reference in their entirety). Alternatively, by employing a flexible linker that is not too short (e.g., not less than about 9 residues) to enable the VL and VH of different single polypeptide chains to associate together, one can form a bispecific antibody, having binding specificity for two different epitopes. In another embodiment, the antibody derivative is a divalent or bivalent single-chain variable fragment, engineered by linking two scFvs together either in tandem (i.e., tandem scFv), or such that they dimerize to form a diabody (Holliger et al. “‘Diabodies’: Small Bivalent And Bispecific Antibody Fragments,” Proc. Natl. Acad. Sci. (U.S.A.) 90(14), 6444-8 (1993), which is hereby incorporated by reference in its entirety). In yet another embodiment, the antibody is a triabody, i.e., a trivalent single chain variable fragment, engineered by linking three scFvs together, either in tandem or in a trimer formation to form a triabody. In another embodiment, the antibody is a tetrabody of four single chain variable fragments. In another embodiment, the antibody is a “linear antibody” which is an antibody comprising a pair of tandem Fd segments (VH-CH1-VH-CH1) that form a pair of antigen binding regions (see Zapata et al. Protein Eng. 8(10):1057-1062 (1995), which is hereby incorporated by reference in its entirety). In another embodiment, the antibody derivative is a minibody, consisting of the single-chain Fv regions coupled to the CH3 (i.e., scFv-CH3).

These and other useful antibody fragments and derivatives in the context of the present invention are discussed further herein. It also should be understood that the term antibody-based molecule, unless specified otherwise, also includes antibody-like polypeptides, such as chimeric antibodies and humanized antibodies, antigen binding fragments and antibody fragments retaining the ability to specifically bind to the antigen (epitope-binding fragments, antigen binding fragments or functional fragments) provided by any known technique, such as enzymatic cleavage, peptide synthesis, and recombinant techniques.

An antibody as generated herein may be of any isotype. As used herein, “isotype” refers to the immunoglobulin class (for instance IgG1, IgG2, IgG3, IgG4, IgD, IgA, IgE, or IgM) that is encoded by heavy chain constant region genes. The choice of isotype typically will be guided by the desired effector functions, and/or FcRn interaction, such as antibody-dependent cellular cytotoxicity (ADCC) induction. Exemplary isotypes are IgG1, IgG2, IgG3, and IgG4. Particularly useful isotypes of the MuSK antibodies disclosed herein include IgG1 and IgG2.

Either of the human light chain constant regions, kappa or lambda, may be used. If desired, the class of a MuSK antibody of the present invention may be switched by known methods. For example, an antibody of the present invention that was originally IgM may be class switched to an IgG antibody of the present invention. Further, class switching techniques may be used to convert one IgG subclass to another, for instance from IgG1 to IgG2. Thus, the effector function of the antibodies of the present invention may be changed by isotype switching to, e.g., an IgG1, IgG2, IgG3, IgG4, IgD, IgA, IgE, or IgM antibody for various therapeutic uses.

In an embodiment, one, two, or more amino acid substitutions are introduced into an IgG constant region Fc region to alter the effector function(s) of the antibody-based molecule. For example, one or more amino acids selected from amino acid residues 234, 235, 236, 237, 238, 239, 243, 265, 267, 268, 292, 297, 300, 318, 320, 322, 327, 328, 329, 330, 331, 332, and 396, numbered according to the EU numbering system (https://www.imgt.org/IMGTScientificChart/Numbering/Hu_GHGnber.html#notes, and Edelman, G. M. et al., Proc. Natl. Acad. USA, 63, 78-85 (1969). PMID: 5257969), can be replaced with a different amino acid residue such that the antibody-based molecule has an altered affinity for an effector ligand but retains the antigen-binding ability. In an embodiment, the amino acid 234 or 235 has been replaced. In another embodiment, the amino acids 234 and 235 have been replaced. In this context, a preferred amino acid sequence of a human IgG constant Fc region comprises SEQ ID NO:266 or 267. In this context, for example, the amino acids 234 and 235 numbered according to the EU numbering system correspond to amino acids 7 and 8 in SEQ ID NO:266 and 267 (i.e. a human IgG constant Fc region of an antibody-based molecule disclosed herein), or the amino acids 234 and 235 numbered according the EU numbering system correspond to amino acids 238 and 239 in SEQ ID NO:268 and 270 (i.e. a human full length heavy chain of an antibody-based molecule disclosed herein). The positions typically differ, because variable regions vary in length, which introduces a “delta” between the numberings. In the case depicted above, that delta is 4. Accordingly, the same holds for other amino acid positions identified above (i.e. 236, 237, 238, 239, 243, 265, 267, 268, 292, 297, 300, 318, 320, 322, 327, 328, 329, 330, 331, 332, and 396) numbered according to the EU numbering system when identifying the corresponding positions in SEQ ID NO: 266 or 267 or 268 or 270. Within the application as filed, one can either refer to the position of an amino acid using the EU numbering system or using the actual position in a given Fc region (for example SEQ ID NO: 266 or 267) or in a full length heavy chain (for example SEQ ID NO: 268 or 270).

Accordingly, in an embodiment, 1, 2, 3, 4, 5, 6, 7, 8, 9, 10, 11, 12, 13, 14, 15, 16 or 17 amino acid substitutions are introduced into SEQ ID NO: 266 or 267. In an embodiment, 1, 2, 3, 4 amino acid substitutions are introduced into SEQ ID NO:266 or 267. In an embodiment, 1 or 2 amino acid substitutions are introduced into SEQ ID NO:266 or 267.

Accordingly, in an embodiment, 1, 2, 3, 4, 5, 6, 7, 8, 9, 10, 11, 12, 13, 14, 15, 16 or 17 amino acid substitutions are introduced into SEQ ID NO: 266 or 267 and said substitutions are introduced at amino acid positions selected from amino acid 234, 235, 236, 237, 238, 239, 243, 265, 267, 268, 292, 297, 300, 318, 320, 322, 327, 328, 329, 330, 331, 332, and 396 numbered according the EU numbering system of said sequence. In an embodiment, 1 or 2 amino acid substitutions are introduced into SEQ ID NO:266 or 267. In an embodiment, the amino acid 234 or 235 numbered according to the EU numbering system of SEQ ID NO: 266 or 267 has been replaced. In another embodiment, the amino acids 234 and 235 numbered according to the EU numbering system of SEQ ID NO: 266 or 267 have been replaced.

The effector ligand to which affinity is altered can be, for example, an Fc receptor or the C1 component of complement. This approach is described in further detail in U.S. Pat. Nos. 5,624,821 and 5,648,260, each of which is herein incorporated by reference in its entirety. In an embodiment, one or more amino acid substitutions may be introduced into the Fc region of the antibody-based molecule described herein to remove potential glycosylation sites on the Fc region, which may reduce Fc receptor binding (see, e.g., Shields R L et al., (2001) J Biol Chem 276: 6591-604, which is herein incorporated by reference in its entirety). In an embodiment, the binding to an effector ligand is reduced of at least 10%, 20%, 30%, 40%, 50%, 60%, 70%, 80%, 90%, or is no longer detectable compared to the binding to the same ligand by the antibody not having any amino acid substitutions into its human IgG constant Fc region.

In a first embodiment, one or more of the following mutations have been introduced into the constant region of the antibody-based molecule described herein (all numbered according to the EU numbering system): an N297A substitution; an N297Q substitution; an L234A substitution; an L234D substitution; an L234E substitution; an L234G substitution; an L234H substitution; an L234F substitution; an L234K substitution; an L234Q substitution; an L234R substitution; an L234S substitution; an L234T substitution; an L235A substitution; an L235D substitution; an L235E substitution; an L235F substitution; an L235G substitution; an L235V substitution; an L235H substitution; an L2351 substitution; an L235K substitution; an L235R substitution; an L235S substitution; L235T substitution; an L235Q substitution; an L237A substitution; an S239D substitution; an E233P substitution; an L234V substitution; a C236 deletion; a G236E substitution; a G236R substitution; a G236K substitution; a G237A substitution; a P238A substitution; an F243L substitution; a D265A substitution; an S267E substitution; an H268A substitution; an R292P substitution; a Y300L substitution; a K322A substitution; a K322Q substitution; an A327Q substitution; an L328F substitution; an L328R substitution; a P329A substitution; a P329G substitution; an A330L substitution; an A330S substitution; a P331S substitution; an 1332E substitution; or a P396L substitution.

In a second embodiment, one or more of the following mutations have been introduced into the constant region of the antibody-based molecule described herein (all numbered according to the EU numbering system): an L234A and/or an L235A substitution; an L234A and an L235A substitution; an L234A, an L235A and a P329G substitution; an L234A, an L235A and a G236K substitution; an 1234A, an L235A and a G236E substitution; an 1234A, an L235A and a G236R substitution; an L234A and a G236R substitution; an L234A, L235S and a G236R substitution; an L234A, L235T and a G236R substitution; an L234D, L235H and a G236R substitution; an L234D, L235K and a G236R substitution; an L234D and a G236R substitution; an L234D, L235Q and a G236R substitution; an L234D, L235S and a G236R substitution; an L234E, L235D and a G236R substitution; an L234E, L235H and a G236R substitution; an L234E, L2351 and a G236R substitution; an 1234G, L235H and a G236R substitution; an 1234G, L235Q and a G236R substitution; an 1234G, L235S and a G236R substitution; an L234H, L2351 and a G236R substitution; an L234H, L235S and a G236R substitution; an L234K, L235Q and a G236R substitution; an L234K, L235R and a G236R substitution; an L234K, L235S and a G236R substitution; an L234K, L235T and a G236R substitution; an L234K, L235V and a G236R substitution; an L234Q, L235A and a G236R substitution; an L234Q, L235D and a G236R substitution; an L234Q, L235H and a G236R substitution; an L234Q and a G236R substitution; an L234Q, L235Q and a G236R substitution; an L234Q, L235R and a G236R substitution; an L234Q, L235S and a G236R substitution; an L234Q, L235T and a G236R substitution; an L234Q, L235V and a G236R substitution; an L234R, L235D and a G236R substitution; an L234R, L235E and a G236R substitution; an L234R, L235H and a G236R substitution; an L234R, L2351 and a G236R substitution; an L234R, L235K and a G236R substitution; an L234R and a G236R substitution; an L234R, L2350 and a G236R substitution; an L234R, L235R and a G236R substitution; an L234R, L235T and a G236R substitution; an L234S, L235E and a G236R substitution; an L234S, L235G and a G236R substitution; an L234S, L235H and a G236R substitution; an L234S, L2351 and a G236R substitution; an L234S and a G236R substitution; an L234S, L235R and a G236R substitution; L234S, L235T and a G236R substitution; L234S, L235V and a G236R substitution; an L234T, L235A and a G236R substitution; an L234T, L235D and a G236R, an L234T, L235H and a G236R substitution; an L234T, L2351 and a G236R substitution; an L234T, L235K and a G236R substitution; an L234T, L2350 and a G236R substitution; an L234T, L235R and a G236R substitution; an L234T, L235S and a G236R substitution; an L234T, L235T and a G236R substitution; an L234T, L235V and a G236R substitution; a G236R and an L328R substitution; an L234A, an L235A, a G237A, a P238S, an H268A, an A330S and a P331S substitution; an E233P, an L234V, an L235A, a G326 deletion, an A327G, an A330S and a P331S substitution; an L235A and a G236R substitution; an L235S and a G236R substitution.

In a third embodiment, one or more of the following mutations have been introduced into the Fc region SEQ ID NO: 266 or SEQ ID NO: 267 of the antibody-based molecule described herein (all numbered according to the EU numbering system): an N297A substitution; an N297Q substitution; an L234A substitution; an L234D substitution; an L234E substitution; an L234G substitution; an L234H substitution; an L234F substitution; an L234K substitution; an L234Q substitution; an L234R substitution; an L234S substitution; an L234T substitution; an L235A substitution; an L235D substitution; an L235E substitution; an L235F substitution; an L235G substitution; an L235V substitution; an L235H substitution; an L2351 substitution; an L235K substitution; an L235R substitution; an L235S substitution; L235T substitution; an L235Q substitution; an L237A substitution; an S239D substitution; an E233P substitution; an L234V substitution; a C236 deletion; a G236E substitution; a G236R substitution; a G236K substitution; a G237A substitution; a P238A substitution; an F243L substitution; a D265A substitution; an S267E substitution; an H268A substitution; an R292P substitution; a Y300L substitution; a K322A substitution; a K322Q substitution; an A327Q substitution; an L328F substitution; an L328R substitution; a P329A substitution; a P329G substitution; an A330L substitution; an A330S substitution; a P331S substitution; an 1332E substitution; or a P396L substitution.

In a fourth embodiment, one or more of the following mutations have been introduced into the Fc region SEQ ID NO: 266 or SEQ ID NO: 267 of the antibody-based molecule described herein (all numbered according to the EU numbering system): an L234A and/or an L235A substitution; an L234A and an L235A substitution; an L234A, an L235A and a P329G substitution; an L234A, an L235A and a G236K substitution; an L234A, an L235A and a G236E substitution; an L234A, an L235A and a G236R substitution; an L234A and a G236R substitution; an L234A, L235S and a G236R substitution; an L234A, L235T and a G236R substitution; an L234D, L235H and a G236R substitution; an L234D, L235K and a G236R substitution; an L234D and a G236R substitution; an L234D, L235Q and a G236R substitution; an L234D, L235S and a G236R substitution; an L234E, L235D and a G236R substitution; an L234E, L235H and a G236R substitution; an L234E, L2351 and a G236R substitution; an 1234G, L235H and a G236R substitution; an L234G, L2350 and a G236R substitution; an L234G, L235S and a G236R substitution; an L234H, L2351 and a G236R substitution; an L234H, L235S and a G236R substitution; an L234K, L2350 and a G236R substitution; an L234K, L235R and a G236R substitution; an L234K, L235S and a G236R substitution; an L234K, L235T and a G236R substitution; an L234K, L235V and a G236R substitution; an L234Q, L235A and a G236R substitution; an L234Q, L235D and a G236R substitution; an L234Q, L235H and a G236R substitution; an L234Q and a G236R substitution; an L234Q, L235Q and a G236R substitution; an L234Q, L235R and a G236R substitution; an L234Q, L235S and a G236R substitution; an L234Q, L235T and a G236R substitution; an L234Q, L235V and a G236R substitution; an L234R, L235D and a G236R substitution; an L234R, L235E and a G236R substitution; an L234R, L235H and a G236R substitution; an L234R, L2351 and a G236R substitution; an L234R, L235K and a G236R substitution; an L234R and a G236R substitution; an L234R, L235Q and a G236R substitution; an L234R, L235R and a G236R substitution; an L234R, L235T and a G236R substitution; an L234S, L235E and a G236R substitution; an L234S, L235G and a G236R substitution; an L234S, L235H and a G236R substitution; an L234S, L2351 and a G236R substitution; an L234S and a G236R substitution; an L234S, L235R and a G236R substitution; L234S, L235T and a G236R substitution; L234S, L235V and a G236R substitution; an L234T, L235A and a G236R substitution; an L234T, L235D and a G236R, an L234T, L235H and a G236R substitution; an L234T, L2351 and a G236R substitution; an L234T, L235K and a G236R substitution; an L234T, L235Q and a G236R substitution; an L234T, L235R and a G236R substitution; an L234T, L235S and a G236R substitution; an L234T, L235T and a G236R substitution; an L234T, L235V and a G236R substitution; a G236R and an L328R substitution; an L234A, an L235A, a G237A, a P238S, an H268A, an A330S and a P331S substitution; an E233P, an L234V, an L235A, a G326 deletion, an A327G, an A330S and a P331S substitution; an L235A and a G236R substitution; an L235S and a G236R substitution.

In an embodiment, one or more of the following mutations are introduced into the Fc region SEQ ID NO: 266 or SEQ ID NO: 267 of the antibody-based molecule described herein: an L234A and/or an L235A substitution (numbered according to the EU numbering system). In an embodiment, the following mutations are introduced into the Fc region SEQ ID NO: 266 or SEQ ID NO: 267 of the antibody-based molecule described herein: an L234A and an L235A substitutions numbered according to the EU numbering system. This embodiment results in an antibody-based molecule with a heavy chain represented by SEQ ID NO:268 or 270.

Such an antibody with altered, diminished even abolished effector function is attractive in the context of the invention.

In an embodiment, the antibody-based molecules of the present invention are “humanized,” particularly if they are to be employed for therapeutic purposes. The term “humanized” refers to a chimeric molecule, generally prepared using recombinant techniques, having an antigen-binding site derived from an immunoglobulin from a non-human species and a remaining immunoglobulin structure based upon the structure and/or sequence of a human immunoglobulin. The antigen-binding site may comprise either complete non-human antibody variable domains fused to human constant domains, or only the complementarity determining regions (CDRs) of such variable domains grafted to appropriate human framework regions of human variable domains. The framework residues of such humanized molecules may be wild-type (e.g., fully human) or they may be modified to contain one or more amino acid substitutions not found in the human antibody whose sequence has served as the basis for humanization. Humanization lessens or eliminates the likelihood that a constant region of the molecule will act as an immunogen in human individuals, but the possibility of an immune response to the foreign variable region remains (LoBuglio, A. F. et al. “Mouse/Human Chimeric Monoclonal Antibody In Man: Kinetics And Immune Response,” Proc. Natl. Acad. Sci. USA 86:4220-4224 (1989), which is hereby incorporated by reference in its entirety). Another approach focuses not only on providing human-derived constant regions, but modifying the variable regions so as to reshape them as closely as possible to human form. The variable regions of both heavy and light chains contain three complementarity-determining regions (CDRs) which vary in response to the antigens in question and determine binding capability. The CDRs are flanked by four framework regions (FRs) which are relatively conserved in a given species and which putatively provide a scaffolding for the CDRs. When non-human antibodies are prepared with respect to a particular antigen, the variable regions can be “reshaped” or “humanized” by grafting CDRs derived from non-human antibody onto the FRs present in the human antibody to be modified. Suitable methods for humanizing the non-human antibody described herein are known in the art see e.g., Sato, K. et al., Cancer Res 53:851-856 (1993); Riechmann, L et al., “Reshaping Human Antibodies for Therapy,” Nature 332:323-327 (1988); Verhoeyen, M. et al., “Reshaping Human Antibodies: Grafting An Antilysozyme Activity,” Science 239:1534-1536 (1988); Kettleborough, C. A. et al., “Humanization Of A Mouse Monoclonal Antibody By CDR-Grafting: The Importance Of Framework Residues On Loop Conformation,” Protein Engineering 4:773-3783 (1991); Maeda, H. et al., “Construction Of Reshaped Human Antibodies With HIV-Neutralizing Activity,” Human Antibodies Hybridoma 2:124-134 (1991); Gorman, S. D. et al., “Reshaping A Therapeutic CD4 Antibody,” Proc. Natl. Acad. Sci. USA 88:4181-4185 (1991); Tempest, P. R. et al., “Reshaping A Human Monoclonal Antibody To Inhibit Human Respiratory Syncytial Virus Infection In Vivo,” Bio/Technology 9:266-271 (1991); Co, M. S. et al., “Humanized Antibodies For Antiviral Therapy,” Proc. Natl. Acad. Sci. USA 88:2869-2873 (1991); Carter, P. et al., “Humanization Of An Anti-p185her2 Antibody For Human Cancer Therapy,” Proc. Natl. Acad. Sci. USA 89:4285-4289 (1992); and Co, M. S. et al., “Chimeric And Humanized Antibodies With Specificity For The CD33 Antigen,” J. Immunol. 148:1149-1154 (1992), which are hereby incorporated by reference in their entirety. In some embodiments, humanized MuSK antibodies of the present invention preserve all CDR sequences (for example, a humanized antibody containing all six CDRs from the llama or mouse antibody). In other embodiments, humanized MuSK antibodies of the present invention have one or more CDRs (one, two, three, four, five, six) which are altered with respect to the original antibody. Methods of humanizing an antibody are well-known in the art and suitable for humanizing the antibodies disclosed herein (see, e.g., U.S. Pat. No. 5,225,539 to Winter; U.S. Pat. Nos. 5,530,101 and 5,585,089 to Queen and Selick; U.S. Pat. No. 5,859,205 to Robert et al.; U.S. Pat. No. 6,407,213 to Carter; and U.S. Pat. No. 6,881,557 to Foote, which are hereby incorporated by reference in their entirety).

In some antibodies only part of a CDR, namely the subset of CDR residues required for binding termed the “specificity determining residues” (“SDRs”), are needed to retain binding of the antibody. CDR residues not contacting antigen and not in the SDRs can be identified based on previous studies from regions of Kabat CDRs lying outside Chothia hypervariable loops (see, Kabat et al., SEQUENCES OF PROTEINS OF IMMUNOLOGICAL INTEREST, National Institutes of Health Publication No. 91-3242 (1991); Chothia, C. et al., “Canonical Structures For The Hypervariable Regions Of Immunoglobulins,” J. Mol. Biol. 196:901-917 (1987), which are hereby incorporated by reference in their entirety), by molecular modelling and/or empirically, or as described in Gonzales, N. R. et al., “SDR Grafting Of A Murine Antibody Using Multiple Human Germline Templates To Minimize Its Immunogenicity,” Mol. Immunol. 41:863-872 (2004), which is hereby incorporated by reference in its entirety. In such humanized antibodies, at positions in which one or more donor CDR residues is absent or in which an entire donor CDR is omitted, the amino acid residue occupying the position can be an amino acid residue occupying the corresponding position (by Kabat numbering) in the acceptor antibody sequence. The number of such substitutions of acceptor for donor amino acids in the CDRs to include reflects a balance of competing considerations. Such substitutions are potentially advantageous in decreasing the number of non-human amino acids in a humanized antibody and consequently decreasing potential immunogenicity. However, substitutions can also cause changes of affinity, and significant reductions in affinity are preferably avoided. Substitutions may also cause changes of activity. Such substitutions causing a significant reduction in activity are also preferably avoided. In this context, the antibody or antibody fragment should still exhibit a detectable activity of the antibody as earlier defined herein or an activity of the antibody at least to some extent. Positions for substitution within CDRs and amino acids to substitute can also be selected empirically.

Phage display technology can alternatively be used to increase (or decrease) CDR affinity of the antibody-based molecules of the present invention. This technology, referred to as affinity maturation, employs mutagenesis or “CDR walking” and re-selection using the target antigen or an antigenic fragment thereof to identify antibodies having CDRs that bind with higher (or lower) affinity to the antigen when compared with the initial or parental antibody (see, e.g. Glaser et al., “Antibody Engineering By Codon-Based Mutagenesis In A Filamentous Phage Vector System,” J. Immunology 149:3903-3913 (1992), which is hereby incorporated by reference in its entirety). Mutagenizing entire codons rather than single nucleotides results in a semi-randomized repertoire of amino acid mutations. Libraries can be constructed consisting of a pool of variant clones each of which differs by a single amino acid alteration in a single CDR from another member of such library and which contain variants potentially representing each possible amino acid substitution for each CDR residue. Mutants with increased (or decreased) binding affinity for the antigen can be screened by contacting the immobilized mutants with labelled antigen. Any screening method known in the art can be used to identify variant antibody-based binding molecules with increased or decreased affinity to the antigen (e.g., ELISA) (See Wu, H. et al., “Stepwise In Vitro Affinity Maturation Of Vitaxin, An Alphav Beta3-Specific Humanized mAb,” Proc. Natl. Acad. Sci. USA 95:6037-6042 (1998); Yelton et al., “Affinity Maturation Of The BR96 Anti-Carcinoma Antibody By Codon-Based Mutagenesis,” J. Immunology 155:1994 (1995), which are hereby incorporated by reference in their entirety). CDR walking, which randomizes the light chain may be used (see, Schier, R. et al., “Isolation Of Picomolar Affinity Anti-c-erbB-2 Single-Chain Fv By Molecular Evolution Of The Complementarity Determining Regions In The Center Of The Antibody Binding Site,” J. Mol. Biol. 263:551-567 (1996), which is hereby incorporated by reference in its entirety).

Methods for affinity maturation of antibody-based molecules are described herein and disclosed for example, in Krause, J. C. et al., “An Insertion Mutation That Distorts Antibody Binding Site Architecture Enhances Function of a Human Antibody,” MBio. 2(1): e00345-10 (2011); Kuan, C. T. et al., “Affinity-Matured Anti-Glycoprotein NMB Recombinant Immunotoxins Targeting Malignant Gliomas And Melanomas,” Int. J. Cancer 10.1002/ijc.25645 (2010); Hackel, B. J. et al., “Stability And CDR Composition Biases Enrich Binder Functionality Landscapes,” J. Mol. Biol. 401(1):84-96 (2010); Montgomery, D. L. et al., “Affinity Maturation And Characterization Of A Human Monoclonal Antibody Against HIV-1 gp41,” MAbs 1(5):462-474 (2009); Gustchina, E. et al., “Affinity Maturation By Targeted Diversification Of The CDR-H2 Loop Of A Monoclonal Fab Derived From A Synthetic Naïve Human Antibody Library And Directed Against The Internal Trimeric Coiled-Coil Of Gp41 Yields A Set Of Fabs With Improved HIV-1 Neutralization Potency And Breadth,” Virology 393(1):112-119 (2009); Finlay, W. J. et al., “Affinity Maturation Of A Humanized Rat Antibody For Anti-RAGE Therapy: Comprehensive Mutagenesis Reveals A High Level Of Mutational Plasticity Both Inside And Outside The Complementarity-Determining Regions,” J. Mol. Biol. 388(3):541-558 (2009); Bostrom, J. et al., “Improving Antibody Binding Affinity And Specificity For Therapeutic Development,” Methods Mol. Biol. 525:353-376 (2009); Steidl, S. et al., “In Vitro Affinity Maturation Of Human GM-CSF Antibodies By Targeted CDR-Diversification,” Mol. Immunol. 46(1):135-144 (2008); and Barderas, R. et al., “Affinity Maturation Of Antibodies Assisted By In Silico Modeling,” Proc. Natl. Acad. Sci. USA 105(26):9029-9034 (2008), which are hereby incorporated by reference in their entirety.

In the context of this application, an amino acid alteration (or change or modification) may be an amino acid substitution; addition, deletion or chemical modification.

### In Utero Treatment

In an embodiment, the invention provides an antibody-based molecule for use in the treatment of a disease of condition resulting from a genetic defect in a fetus wherein the antibody-based molecule is administered to the mother of said fetus, wherein said antibody-based molecule transfers via placenta from the mother to the fetus.

In this context, the term “mother” may mean the carrier of the fetus during the pregnancy, also may be called “the birth mother”. The term “mother” may include the true biological mother of the fetus, a traditional surrogate who gets artificially inseminated, carries and delivers the new-born, or a gestational surrogate (no genetic ties to the child) whose uterus is placed with the fertilized embryo, who then carries the baby until birth.

Placenta is the materno-fetal interface, delivering nutrients and oxygen to the fetus and acting as a selective barrier between mother and the fetus. Factors that affect molecule transfer across the placenta include placental surface area, placental thickness, pH of maternal and fetal blood, placental metabolism, uteroplacental blood flow, presence of placental drug transporters, molecular weight of drug, lipid solubility of the drug, pKa, protein binding (e.g. antibody binding to a receptor), and concentration gradient across the placenta. In an embodiment, the mechanism of placental transfer is simple diffusion, facilitated diffusion using a carrier, active transport using ATG or pinocytosis. In an embodiment, the placental transfer of molecules is via diffusion. In an embodiment, placental transfer of the antibody-based molecules decrease with increasing molecular weight. In a preferred embodiment, the weight of the antibody-based molecules is smaller than 170 kDa, 160 kDa, 150 kDa, 140 kDa, 130 kDa, 120 kDa, 120 kDa, 110 kDa, 100 kDa, 90 kDa, 80 kDa, 70 kDa, 60 kDa, or 50 kDa. In an embodiment, the placenta transfer of the antibody-based molecules is mediated by a receptor located at the placenta. In an embodiment, the antibody-based molecules bind to a receptor (e.g. FcRn receptor) located at the placenta, which mediates the transplacental transfer of said antibody-based molecules.

In an embodiment, an antibody-based molecule for use in the treatment of a disease or condition resulting from a genetic defect in a fetus wherein the antibody-based molecule is administered to the mother of said fetus, wherein the fetus is a carrier of genetic defect with a known target for treatment by said antibody-based molecule. In the current therapeutic practice for pregnant patients, e.g. administration of monoclonal antibodies or IVIg to a pregnant patient, actually, transfer of the therapeutic from the mother to the fetus is undesired because of potential risks of fetal exposure. It is thus a surprising aspect of the invention that the antibody-based molecule is administered to the mother of the fetus (the fetus in this case being the patient to be treated, e.g. for a neuromuscular disease) during the pregnancy, for example during first-trimester, second-trimester, third trimester or in combination of first-, second-, and/or third trimesters. In a preferred embodiment, the antibody-based molecule is administered to the mother of the fetus during the pregnancy during the first-trimester or second-trimester. In a more preferred embodiment, the antibody-based molecule is administered to the mother of the fetus during the pregnancy as soon as possible after conception. The early administration of the antibody-based molecule aims to increase chances that the synapses in the neuromuscular junctions (NMJs) of the fetus are properly formed (and as such avoid defects at the neuromuscular junctions of the fetus).

A disease or condition resulting from a genetic defect is caused in whole or in part by a change in the DNA sequence away from the normal sequence. Genetic defect can be caused by a mutation in one gene (monogenic disorder), by mutations in multiple genes (multifactorial inheritance disorder), by a combination of gene mutations and environmental factors, or by damage to chromosomes (changes in the number or structure of entire chromosomes, the structures that carry genes). Types of genetic mutation include base substitutions, deletions and insertions. In an embodiment, the genetic defect is caused by a mutation in one of the genes from the group consisting of CHRNA1, CHRNB1, CHRND, CHRNE, CHRNG, RAPSN, DOK7, AGRIN, LRP4, MUSK, PLEC, SCN4A, COLQ, COL13A1, AGRN, CHAT, SLC5A7, SLC18A3, SNAP25, VAMP1, SYT2, PREPL, MYO9A, SLC25A1, ALG2, ALG14, DPAGT1, GFPT1, GMPPB, LAMA5, LAMB2, MUNC13-1, PREP1, SYB1 and/or genes that encodes subunits of acetylcholine receptors (AChRs). In an embodiment, the genetic defect is caused by a mutation in one of the genes from the group consisting of CHRNA1, CHRNB1, CHRND, CHRNE, CHRNG, DOK7, AGRIN, LRP4, MUSK, PLEC, SCN4A, COLQ, COL13A1, AGRN, CHAT, SLC5A7, SLC18A3, SNAP25, VAMP1, SYT2, PREPL, MYO9A, SLC25A1, ALG2, ALG14, DPAGT1, GFPT1, GMPPB, LAMA5, LAMB2, MUNC13-1, PREP1, SYB1, SMN1, SMN2 and/or genes that encodes subunits of acetylcholine receptors (AChRs). In an embodiment, the genetic defect is caused by a mutation in DOK7, AGRIN, LRP4 and/or MUSK, and the known target for treatment is MuSK. In this context, the antibody-based molecule of the present invention specifically acts on MuSK, thereby restoring disrupted acetylcholine signalling pathway or other signalling pathways important for neuromuscular junction formation, and the disrupted signalling pathway is linked to or due to blocked, deactivated, attenuated or disrupted MuSK signalling pathway in a cell, preferably a muscle cell.

In an embodiment, the genetic defect is a DOK7 mutation. In an embodiment, the DOK7 mutation is one of the mutations selected from the group consisting of c.7G>A, c.48C>T, IVS1+25_39del15, c.91C>A, c.101-124_141del; 176_206delinsAG, c.230C>T, c.325G>T, c.414C>T, c.415G>C, c.437deC, c.473G>A, c.481G>A, c.496G>A, c.512G>A, c.513C>T, IVS5-37_11de127, c.539G>C, c.548_551delTCCT, c.596delT, c.601C>T, c.1124_1127dupTGCC (or DOK7^{1124_1127 dup}), c.1138delG, c.1143delC, c.1143_1144insC, c.1185C>G, c.1263_1264insC, c.1296_1311del16, c.1139_1342dupCTGG, c.1378C>T, c.1378dupC, c.1387_1388insC, c.1487G>T, c.1504_1505insTA, c.1511_1513delCTT. In an embodiment, the DOK7 mutation is DOK7^{1124_1127 dup} and the known target for treatment is MuSK. In an embodiment, an anti-MuSK antibody or the antigen-binding fragment thereof rescues a disease or condition resulting from DOK7 deficiency.

In an embodiment, the genetic defect is a AGRIN mutation. In an embodiment, the AGRIN mutation is one of the mutations selected from p.R1671Q, p.R1698P, p.L1664P mutations, and amino acid deletion or deletions in AGRIN, and the known target for said treatment is MuSK. In an embodiment, an anti-MuSK antibody or the antigen-binding fragment thereof rescues a disease or condition resulting from AGRIN deficiency or mutation.

In an embodiment, the invention provides an antibody-based molecule for use in the treatment of a disease or condition resulting from a genetic defect in a fetus wherein the antibody-based molecule is administered to the mother of said fetus, wherein the genetic defect is detected by genetic screening tests of one or both of the parents and/or the fetus, and/or genetic diagnostic tests of the fetus, and/or fetal imaging.

In an embodiment, the genetic screening tests of one or both of the parents are genetic testing on the blood or tissue sample of the parents. In an embodiment, the genetic diagnostic test of the fetus is performed using the blood of the mother. In an embodiment, the genetic diagnostic tests is performed using the blood or tissue of the fetus. In an embodiment, the genetic diagnostic tests of the fetus are invasive tests (e.g. amniocentesis, chorionic villus sampling) and/or non-invasive tests (e.g. noninvasive prenatal testing (NIPT)). In a preferred embodiment, the genetic diagnostic test of the fetus is a NIPT test. In an embodiment, the fetal imaging is ultrasound examination of the fetus.

In an embodiment, the genetic screening test of one or both of the parents is performed before or during pregnancy. In a preferred embodiment, the genetic screening test of one or both of the parents is performed before pregnancy. In an embodiment, the genetic diagnostic testing of the fetus is performed during the pregnancy, for example during first-trimester, second-trimester, third trimester or in combination of first-, second-, and/or third trimesters. In a preferred embodiment, the genetic diagnostic testing of the fetus is performed during the first-trimester or second-trimester. In a more preferred embodiment, the genetic diagnostic testing of the fetus is performed as soon as possible after conception.

In an embodiment, the genetic screening tests of one or both of the parents provides information about whether one of the parents is a carrier of a certain genetic defect or a gene for certain inherited disorders. In an embodiment, the genetic screening tests provides predictive information about whether their fetus may have certain genetic defects or disorders. In an embodiment, the genetic defect of the fetus is detected by genetic screening tests of one or both of the parents. In an embodiment, the genetic defect of the fetus is detected by genetic diagnostic tests of the fetus. In an embodiment, the genetic defect of the fetus is detected by fetal imaging of the fetus. In an embodiment, the genetic defect of the fetus is detected by combination of genetic screening tests of one or both of the parents, genetic diagnostic screening of the fetus, and/or fetal imaging. In an embodiment, one of the parents of the fetus is a carrier of the genetic defect. In an embodiment, both parents are the carrier of the genetic defect In an embodiment, both parents are silent for the disease or condition resulting from a genetic defect and the fetus contains said genetic defect. In an embodiment, one or both of the parents have the disease or condition resulting from the genetic defect, and the fetus has the disease or condition resulting from the genetic defect.

In an embodiment, diagnosis of genetic defects includes at least one of the results selected from the group consisting of a positive screening for a genetic defect of one of the parents, positive genetic diagnostic testing for a genetic defect of the fetus, and an abnormal development or growth observed from fetal imaging. Diagnosis may be assessed by a physician or veterinarian. In an embodiment, genetic screening tests of the pregnant mother and ultrasound exams identifies aneuploidy for the fetus, defects of the brain and spine, defects of the abdomen, heart, bone, muscle, facial features, and/or growth of the fetus.

In an embodiment, the invention provides an antibody-based molecule for use in the treatment of a disease or condition resulting from a genetic defect in a fetus wherein the antibody-based molecule is administered to the mother of said fetus, wherein the antibody-based molecule binds to the known target in said fetus. A known target for antibody-based molecule are molecules presenting an antigen or epitope for binding of an antibody-based molecule, which are known to be a target for treatment. In some embodiments, the antibody-based molecule is an antibody. In an embodiment, the antibody-based molecule is anti-MuSK agonists or antigen-binding fragment thereof, and the known target in the fetus is MuSK.

In an embodiment, the invention provides an antibody-based molecule for use in the treatment of a disease or condition resulting from a genetic defect in a fetus wherein the antibody-based molecule is administered to the mother of said fetus, wherein the fetus is characterized by abnormal development, abnormal growth, prenatal death and/or early postnatal death, absent the administration of the antibody-based molecule. Development abnormalities may be assessed by fetal measurements (e.g. fetal imaging or ultrasound) including the crown-rump length (CRL), biparietal diameter (BPD), femur length (FL), head circumference (HC), occipitofrontal diameter (OFD), abdominal circumference (AC), and humerus length (HL), as well as calculation of the estimated fetal weight (EFW). An antibody-based molecule may reverse, delay or prevent the developmental abnormalities of the treated fetus. The fetal subject may be considered to have development abnormalities or abnormal growth, when at least one of such fetal measurements deviates at least 10%, 20%, 30%, 40%, 50%, 60%, 70%, 80%, 90% from a normal fetus at the same gestational age. The development status of a treated fetus may be assessed using assays known to the skilled person. In this context, the subject may be an animal. The fetal subject may be considered to have prenatal death and/or early postnatal death, when the fetus die during gestation or within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31 days or within 1, 2, 3, 4, 5, 6, 7, 8, 9, 10, 11, 12 months, or within 1, 2, 3, 4, 5 years after birth. In an embodiment, the fetus is further characterized by abnormal neuromuscular junction formation, abnormal synapse development, and/or deficient motor function. The abnormal neuromuscular junction formation, abnormal synapse development, and/or deficient motor function may be assessed using assays (for example immunostaining, imaging techniques etc.) known to the skilled person, for example by assessing the number of synapses, the synaptic size or area, AChR density at the synapse, colocalization of synapse and AChR, the amount of denervated synapses, and/or fragmented synapses. The fetal subject may be considered to have abnormal neuromuscular junction formation, abnormal synapse development, and/or deficient motor function, when at least one of such synapse measurements deviates at least 30%, 40%, 50%, 60%, 70%, 80%, 90% from a normal fetus at the same gestational age.

In an embodiment, the invention provides an antibody-based molecule for use in the treatment of a disease or condition resulting from a genetic defect in a fetus wherein the antibody-based molecule is administered to the mother of said fetus, wherein the disease or condition is a neuromuscular disease. In an embodiment, a neuromuscular disease is characterized by an impaired neuromuscular transmission and/or an NMJ denervation. In an embodiment, an impaired neuromuscular transmission or impaired NMJ denervation may be characterized by a deficient MuSK signaling: deficient MuSK phosphorylation, deficient MuSK signaling and/or deficient acetylcholine receptor clustering.

In an embodiment, the fetus is treated for an impaired neuromuscular transmission and/or impaired NMJ. In a preferred embodiment, the fetus is treated for an impaired neuromuscular transmission wherein MuSK antibodies or antigen binding fragment thereof is administered to the mother of said fetus. The activation of MuSK is expected to transduce signal at the NMJ of the fetus and therefore to restore at least to some extent an impaired neuromuscular transmission. The MuSK agonist antibody and related aspects of the invention (i.e. polynucleotide, expression vector, host cell, cell-free expression system as earlier defined herein) are said to restore at least to some extent an impaired neuromuscular transmission when they are able to elicit an agonistic MuSK activity. In the context of the application, an agonistic MuSK activity may be replaced by the triggering of a MuSK-induced signal in a muscle cell at the NMJ. A MuSK-induced signal may be at least one of the induction of MuSK dimerization, the induction of MuSK tyrosine phosphorylation, the induction of AChR clustering at the NMJ (or the clustering is assessed in vitro in myotubes AChR patches), the increase of the number of fully innervated NMJ, the decrease of the number of fully denervated NMJ, an improvement of the reliability of synapse release, a restoration of synaps in the presence of axons, a prevention of altered synapse formation, a stabilization of formed synapses, a prevention/stabilization or even a reduction/decrease of motor neuron death, an expected postnatal survival, prolonged postnatal survival, or extension of the lifespan of a treated fetus. The first muscle fibres that appear are known as primary fibres (about embryonic day (E) 11-14 in the mouse limbs), around which secondary fibres form at the time when innervation begins to be established (about E14-16) (see Ontell & Kozeka, 1984, Kim and Burden, 2008. Nat. Neurosci. 1:19-27; Yang et al. 2001. Neuron 30:399-410.) In an embodiment, the MuSK agonist antibody or related aspects of the invention (i.e. polynucleotide, expression vector, host cell, cell-free expression system as earlier defined herein) is administered to the mother when the NMJ of the fetus is about to form. In a preferred embodiment, the fetus is treated for the first time before birth, preferably at E10, E11, E12, E13, E14, E15, E16, E17, E18, E19 or E20. In an embodiment, the impaired NMJ or the impaired formation of the NMJ of the treated fetus is prevented, diminished, reversed, delayed or postponed. Within this embodiment, said MuSK agonist antibody or related aspects of the invention defined herein are able to restore at least to some extent an impaired neuromuscular transmission when they are able to elicit an agonistic MuSK activity. The restoration of at least to some extent the impaired neuromuscular transmission has been earlier defined herein.

In an embodiment, there is provided an antibody-based molecule for use in the treatment of a disease or condition resulting from a genetic defect in a fetus wherein the antibody-based molecule is administered to the mother of said fetus, wherein the genetic disease is neuromuscular disease selected from the group consisting of congenital myasthenia (CM), spinal muscular atrophy (SMA), myasthenia gravis (MG), Lambert-Eaton myasthenic syndrome 30 (LEMS), poliomyelitis, post-poliomyelitis, Kennedy syndrome, hereditary spastic paraplegia, multifocal neuropathy, progressive bulbar palsy, progressive muscular atrophy. In an embodiment, the fetus is treated in combination with genetic treatment for the known genetic target, wherein the treatment of the fetus by MuSK antibodies or antigen binding fragment thereof prepares said fetus to be in a better condition for gene therapy. In an embodiment, the fetus is treated in combination with genetic therapy for the known genetic target. In an embodiment, the genetic therapy may be performed using antisense oligonucleotide, a gene replacement therapy, or small molecule to increase the target gene which comprises a defect to cause the genetic disease.

In an embodiment, the neuromuscular disease is congenital myasthenia (CM). In an embodiment, the neuromuscular disease is congenital myasthenia (CM), and the CM is caused by at least one of the genetic defects in CHRNA1, CHRNB1, CHRND, CHRNE, CHRNG, RAPSN, DOK7, AGRIN, LRP4, MUSK, PLEC, SCN4A, COLQ, COL13A1, AGRN, CHAT, SLC5A7, SLC18A3, SNAP25, VAMP1, SYT2, PREPL, MYO9A, SLC25A1, ALG2, ALG14, DPAGT1, GFPT1, GMPPB, LAMAS, LAMB2, MUNC13-1, PREP1, SYB1 and/or genes that encodes subunits of acetylcholine receptors (AChRs). In an embodiment, the neuromuscular disease is congenital myasthenia (CM) and the genetic defect is MUSK mutation. In an embodiment, the neuromuscular disease is congenital myasthenia (CM) and the genetic defect is DOK7 mutation. In an embodiment, the fetus is treated in combination with genetic treatment aiming at correcting the mutated DOK7 mutation.

In an embodiment, the neuromuscular disease is spinal muscular atrophy (SMA). In preferred embodiment, the neuromuscular disease is spinal muscular atrophy (SMA) and the genetic defect is in the SMN1 gene. In a preferred embodiment, the fetus is treated with an antibody-based molecule against MuSK in combination with gene therapy, wherein the gene therapy is to correct the genetic defect in the SMN1 gene. In a more preferred embodiment, the fetus is treated with said antibody-based molecule as soon as possible following a diagnosis of a genetic defect in the SMN1 gent in the fetus. Subsequently gene therapy to correct the genetic defect in the SMN1 gene may be applied pre- or post-natal. In an even more preferred embodiment, an antisense oligonucleotide is used for the gene therapy.

In an embodiment, there is provided an antibody-based molecule for use in the treatment of a neuromuscular disease in a fetus wherein the antibody-based molecule is administered to the mother of said fetus, wherein the neuromuscular disease is caused by other factors except for genetic defects. In an embodiment, the neuromuscular disease is caused by inflammation, infection, radiation or chemical toxicity.

In an embodiment, the invention provides an antibody-based molecule for use in the treatment of a disease or condition resulting from a genetic defect in a fetus wherein the antibody-based molecule is administered to the mother of said fetus, wherein the antibody-based molecule is administered to the mother intraperitonially (IP), intravenously, subcutaneously, in utero or intramuscularly.

Intraperitonially (IP), Intramuscular injection (for example, into the arm or leg muscles) and intravenous infusion are preferred methods of administration of the antibody-based molecules of the present invention. In some methods, such molecules are administered as a sustained release composition or device, such as a Medipad™ device (Elan Pharm. Technologies, Dublin, Ireland). In some methods, the antibodies disclosed herein are injected directly into a particular tissue, for example umbilical vein injection.

In one embodiment, the invention provides an antibody-based molecule of the present invention is administered parenterally. The phrases “parenteral administration” and “administered parenterally” as used herein denote modes of administration other than enteral and topical administration, usually by injection, and include epidermal, intravenous, intramuscular, intraarterial, intracardiac, intradermal, intraperitoneal, intratendinous, subcutaneous, subcuticular, in utero and infusion. In one embodiment that antibody-based molecule is administered by intraperitoneal, intravenous or subcutaneous injection.

In an embodiment, the antibody-based molecule is administered to the mother of the fetus in an amount therapeutically effective for the treatment of said fetus. The term “therapeutically effective amount” is intended to mean the quantity or dose of an antibody-based molecule that is sufficient to produce a therapeutic effect, for example, the quantity or dose of antibody-based molecule required to elicit an antigen binding activity. In an embodiment, such antibody-based molecule is able to eradicate or at least alleviate the symptoms associated with a disease or condition resulting from a genetic defect in a fetus. An appropriate amount or dose can be determined by a physician, as appropriate.

Effective doses of the provided therapeutic molecules of the present invention for the treatment of the above-described conditions may vary depending upon many different factors, including means of administration, target site, physiological state of the mother and/or fetus, other medications administered. Treatment dosages are typically titrated to optimize their safety and efficacy. On any given day that a dosage is given, the dosage of the antibody-based molecules (e.g. MuSK antibody or antigen binding fragment thereof) as described herein may range from about 0.0001 to about 100 mg/kg, and more usually from about 0.01 to about 20 mg/kg, of the mothers body weight For example, dosages can be 0.5 mg/kg, 1 mg/kg, or 10 mg/kg of the mother's body weight or within the range of 0.5-10 mg/kg of the mother's body weight. Exemplary dosages thus include: from about 0.1 to about 10 mg/kg of the mother's body weight, from about 0.1 to about 5 mg/kg of the mother's body weight, from about 0.1 to about 2 mg/kg of the mother's body weight, from about 0.1 to about 1 mg/kg of the mother's body weight, for instance about 0.1 mg/kg of the mother's body weight, about 0.2 mg/kg of the mother's body weight, about 0.5 mg/kg of the mother's body weight, about 1 mg/kg of the mother's body weight, about 1.5 mg/kg of the mother's body weight, about 2 mg/kg of the mother's body weight, about 5 mg/kg of the mother's body weight, 10 mg/kg of the mother's body weight or about 20 mg/kg of the mother's body weight. In general, the dosing may be selected to obtain a serum titer of the therapeutic molecule in the mother that is known or expected to be effective in the treatment of an adult. Placental transfer of the therapeutic molecule will produce a similar serum titer in the fetus.

A physician or veterinarian having ordinary skill in the art may readily determine and prescribe the effective amount of the pharmaceutical composition required. For example, the physician or veterinarian could start doses of antibody-based molecule in the pharmaceutical composition at levels lower than that required in order to achieve the desired therapeutic effect and gradually increase the dosage until the desired effect is achieved. In general, a suitable daily dose of a composition of the present invention will be that amount of the compound which is the lowest dose effective to produce a therapeutic effect. Such an effective dose will generally depend upon the factors described above. Administration may e.g. be intravenous, intramuscular, intraperitoneal, in utero or subcutaneous, and for instance administered proximal to the site of the target. If desired, the effective daily dose of a pharmaceutical composition may be administered as two, three, four, five, six or more sub-doses administered separately at appropriate intervals throughout the day, optionally, in unit dosage forms. While it is possible the antibody-based molecule of the present invention to be administered alone, it is preferable to administer the antibody-based molecule as a pharmaceutical composition as described.

For therapeutic purposes, the antibody-based molecules (e.g. MuSK antibody-based molecules) of the present invention are usually administered on multiple occasions. Intervals between single dosages (e.g., a bolus or infusion) can be daily, weekly, or monthly. In some methods, dosage is adjusted to achieve a plasma concentration of 1 ng/mL to 1000 μg/mL, preferably 1-1000 μg/mL, more preferably 25-300 μg/mL. Alternatively, the therapeutic molecules of the present invention can be administered as a sustained release formulation, in which case less frequent administration is required. Dosage and frequency vary depending on the half-life of the antibody in the subject to be treated. In general, human antibodies show the longest half-life, followed by humanized antibodies, chimeric antibodies, and non-human antibodies. scFv molecules generally have short serum half-lives.

In an embodiment, the invention provides for an antibody-based molecule for use according to any of preceding claims, wherein the mother is administered as early as possible after the fetus is diagnosed with the genetic defect, or the mother is administered shortly prior to planned or expected conception to enable the fetus to be treated as early as possible by said antibody-based molecule. In an embodiment, the antibody-based molecule for use in the treatment of a disease or condition resulting from a genetic defect in a fetus, wherein the mother is administered prior to the conception with the aim of that the expected fetus is treated by the antibody-based molecule as early as possible. In an embodiment, the mother or the father is diagnosed with a genetic defect that may cause a disease or condition in their future fetus resulting from said genetic defect inherited from one of the parents. In a preferred embodiment, the mother is administered with the antibody-based molecule within 1, 2, 3, 4, 5, 6, 7, 8, 9, 10, 11, 12, 13, or 14 days prior to an expected ovulation during the period of actively seeking to be pregnant In an embodiment, the antibody-based molecule for use in the treatment of a disease or condition resulting from a genetic defect in a fetus, wherein the mother is administered during pregnancy. In an embodiment, the antibody-based molecule is administered to the mother of said fetus within 1, 2, 3, 4, 5, 6 or 7 days, or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 weeks following conception. In an embodiment, the antibody-based molecule is administered to the mother of said fetus immediately or within 1, 2, 3, 4, 5, 6 or 7 days, or 1 or 2 weeks following a positive diagnosis of the genetic defect from a genetic screening test of one of the parents or a positive genetic diagnostic test of the fetus or a diagnostic result from fetal imaging, or the combination of said tests. “Immediately” in this context may mean within 1, 2, 3, 4, 5, 6, 7, 8, 9, 10, 11, 12, 13, 14, 15, 16, 17, 18, 19, 20, 21, 22, 23, 24 hours. In another embodiment, said fetus is treated for 1, or 2, or 3, 4 or 5 times.

Pregnancy may mean 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 weeks following conception and before the birth of the fetus. Surprisingly, attractive results (prolonged post-natal survival, body weight gain) were obtained when the antibody-based molecule (anti-MuSK antibody) was administered to the mother of the fetus two times during the pregnancy with neuromuscular disease caused by DOK7 or AGRIN mutation.

For therapeutic purpose, the antibody-based molecule is usually administered several times. Intervals between single dosage can be weekly, or monthly. In some embodiments, the antibody-based molecule of the present invention is administered to the mother at least 1, at least 2, at least 3, at least 4, at least 5 times over the course of pregnancy. Depending on the development of the fetus, the skilled person may decide to modify the frequency and/or dose of administration of the antibody-based molecule.

In an embodiment, the antibody-based molecule is administered to the mother prior to the birth of the fetus (or newborn), and is administered to the fetus (or newborn) after birth.

In a preferred embodiment, the mother is administered a loading dose or loading doses of the antibody-based molecule followed by a maintenance dose or maintenance doses administered to the newborn having the genetic defect. In this context, a loading dose or loading doses may mean a dose or doses of antibody-based molecule or pharmaceutical composition comprising said antibody-based molecule administered to the mother prior to the birth of the fetus or newborn for the treatment of the disease or condition resulting from a genetic defect. A maintenance dose or maintenance doses may mean a dose or doses of antibody-based molecule or pharmaceutical composition comprising said antibody-based molecule administered to the fetus or newborn postnatally for the treatment of the disease or condition resulting from a genetic defect. In some embodiments, the fetus after birth (or the resulting newborn) is administered with a maintenance dosage when said disease or condition resulting from said genetic defect occurs or relapses in said newborn relative to a healthy newborn without said disease or condition at the same age.

In some embodiments, one, two or three loading doses are administered to the mother, wherein the loading doses are separated by 1, 2, 3, 4, 5, 6 or 7 days, or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 weeks, e.g., on day E5, day E15, and day E30. In some instances, the maintenance doses are administered to the newborn having disease or condition resulting from a genetic defect every 4, 5, 6, 7, 8, 9, 10 weeks from the beginning of 4, 5, 6, 7, 8, 9, 10 weeks after birth (e.g., for 1 month, 2 months, three months, four months, five months, six months, seven months, eight months, nine months, ten, eleven, twelve months).

In some embodiments, the combination of a loading dose or loading doses with a maintenance dose or maintenance doses reverses, delays and/or prevents the development of said disease or condition resulting from the genetic defect. The delay may be of 1, 2, 3, 4, 5, 6 or 7 days, or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, or 42 weeks or 1, 2, 3, 4, 5, 6, 7, 8, 9, 10, 11, 12, month or at least 1, 2, 3, 4 years. In this context, the development of said disease or condition resulting from the genetic defect may be assessed by the function in respiratory, energy expenditure, activity, water intake, food intake, motor deficit, and/or body weight compared to a control or healthy subject. Such functions may be assessed by measuring the corresponding metabolic parameters (for example O₂ consumption, CO₂ production, energy expenditure, total activity, water intake, food intake, latency to fall, and/or body weight measurements) known to a skilled person. In this context, the subject may be an animal.

In an embodiment, the fetus is 1, 2, 3, 4, 5, 6 or 7 days, or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, or 42 weeks old when an antibody-based molecule or a loading dose is administered to the mother, and/or 1, 2, 3, or 4 weeks, or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 90, 91, 92, 93, 94, 95, 96, 97, 98, 99, or 100 years old when an antibody-based molecule or a maintenance dose is administered to the postnatal fetus or newborn, for the treatment of a disease or condition resulting from a genetic defect.

In an embodiment, the invention provides an antibody-based molecule for use in the treatment of a disease or condition resulting from a genetic defect in a fetus wherein the antibody-based molecule is administered to the mother of said fetus, wherein the antibody-based molecule is an anti-MuSK antibody or antigen binding fragment thereof, and the known target is MuSK, wherein the antibody-based molecule is administrated at the time at which NMJs are about to be formed. In an embodiment, the MuSK is represented by an amino acid sequence that comprises the amino acid sequence of SEQ ID NO: 129 or by a sequence that has at least 80% identity or similarity to SEQ ID NO: 129. In a preferred embodiment, the anti-MuSK antibody or antigen binding fragment thereof binds to an epitope within the Frizzled (Fz)-like domain of MuSK, wherein the Fz-like domain of MuSK comprises the amino acid sequence of SEQ ID NO: 130.

In an embodiment, the invention provides an antibody-based molecule for use in the treatment of a disease or condition resulting from a genetic defect in a fetus, wherein the antibody-based molecule is administered to the mother of the fetus, wherein said treatment results in reversed, delayed and/or prevented developmental abnormalities, fetal weight gain, postnatal survival, prolonged postnatal survival, improved postnatal development and/or postnatal weight gain, relative to a fetus whose mother is not administered with the antibody-based molecule, wherein the treated fetus is assessed by size estimation, growth curve, physical movement, heart rate monitoring, prenatal survival and/or postnatal survival.

In an embodiment, the invention provides an antibody-based molecule for use in the treatment of a disease or condition resulting from a genetic defect in a fetus, wherein the antibody-based molecule is administered to the mother of the fetus, wherein said treatment results in the reversion, delay and/or prevention of abnormal synaptic development, abnormal synapse maturation, abnormal synapse generation, abnormal synapse regeneration and/or deficient motor function, wherein the treated fetus is assessed by characterization of neuromuscular synapse, synapses counting, synapse size estimation, and/or acetylcholine receptor (AChR) density estimation at the synapse. In this context, synaptic size may mean the size of the acetylcholine receptor (AChR) rich area, assessed by AChR density estimation for example. Synapses generation or synapses regeneration or synapses maturation may be characterized from the change of its shape, from example from a plaque-like shape to a complex, pretzel-like shape, using imaging techniques known to a skilled person. The synaptic development, abnormal synapse maturation and/or deficient motor function may be considered to be reversed, delayed and/or prevented, if the value of such assessment has been improved at least 10%, 20%, 30%, 40%, 50% or 60% in an experiment administering the antibody-based molecule of the invention to the mother of the fetus by comparison with the same experimental setting without administering the mother during pregnancy or in an experiment administering the antibody-based molecule of the invention to the mother of the fetus and to the fetus after birth by comparison with the same experimental setting without administering the fetus after birth. In this context, the synaptic development, abnormal synapse maturation, abnormal synapse generation, abnormal synapse regeneration and/or deficient motor function may be considered to be reversed, delayed or prevented, if such assessment may represent at least 30%, 40%, 50%, 60%, 70% of the corresponding measurements in a control subject (for example a healthy subject at the same or similar age). In an embodiment, the number of synapses can be restored to at least 40%, 45%, 50%, 55%, 60%, 65% or 70% of normal levels in a healthy subject. In an embodiment, the synaptic size can be restored to at least 40%, 45%, 50%, 55%, 60%, 65% or 70% of normal levels in a healthy subject. In an embodiment, the density of synaptic AChRs can be restored to at least 40%, 45%, 50%, 55%, 60%, 65% or 70% of normal levels in a healthy subject.

In an embodiment, the invention provides an antibody-based molecule for use in the treatment of a disease or condition resulting from a genetic defect in a fetus, wherein the antibody-based molecule is administered to the mother of the fetus, wherein said treatment results in a moderate function in respiratory, energy expenditure, activity, water intake, food intake, and/or body weight compared to a control or healthy subject Such functions may be assessed by measuring the corresponding metabolic parameters (for example 02 consumption, CO₂ production, energy expenditure, total activity, water intake, food intake, and/or body weight measurements) known to a skilled person. In this context, the subject may be an animal. In an embodiment, the treated fetus maintains a function in respiratory, energy expenditure, activity, water intake, food intake, and/or body weight at a level of at least 10%, 15%, 20%, 25%, 30%, 35%, 40%, 45%, 50%, 55%, 60%, 65%, 70%, 75%, 80% of the level of a control. Such levels may be maintained or gradually decrease until the disease or condition relapses. The decrease of the levels of any of said functions or body weight may be rescued by a maintenance dose or maintenance dosages.

The term “treatment” or “treating” as used herein means ameliorating, slowing or reversing the progress or severity of a disease or disorder, or ameliorating, slowing or reversing one or more symptoms or side effects of such disease or disorder. For purposes of this invention, “treatment” or “treating” further means an approach for obtaining beneficial or desired clinical results, where “beneficial or desired clinical results” include, without limitation, alleviation of a symptom, diminishment of the extent of a disorder or disease, stabilized (i.e., not worsening) disease or disorder state, delay, prevention or slowing of the progression a disease or disorder state, amelioration or palliation of a disease or disorder state, and remission of a disease or disorder, whether partial or total, detectable or undetectable.

The reversed, delayed and/or prevented development abnormalities may be assessed by fetal measurements (e.g. fetal imaging or ultrasound) including the crown-rump length (CRL), biparietal diameter (BPD), femur length (FL), head circumference (HC), occipitofrontal diameter (OFD), abdominal circumference (AC), and humerus length (HL), as well as calculation of the estimated fetal weight (EFW). An antibody-based molecule may reverse, delay and/or prevent the developmental abnormalities of the treated subject The development of a treated subject may have been considered to have been improved when at least one of the such fetal measurements may have been improved at least 10%, 20%, 30%, 40%, 50%, 60%, 70%, 80%, 90%, or 100% in an experiment administering the antibody-based molecule of the invention to the mother of the fetus by comparison with the same experimental setting without administering the mother during pregnancy. The development status of a treated fetus may be assessed using assays known to the skilled person. In this context, the subject may be an animal.

An antibody-based molecule may correct the abnormal weight gain of the treated fetus. The postnatal body weight after treatment is at least 50%, 60%, 70%, 80%, 90% or 100% identical compared to the body weight of a normal new-born of the same age. The weight gain of a treated fetus may be considered to have been improved when such weight gain is improved by at least 50%, 60%, 70%, 80%, 90%, or 100% in an experiment administering the antibody-based molecule of the invention to the mother of the fetus by comparison with the same experimental setting without administering the mother during pregnancy. The weight gain of a treated fetus may be assessed using assays known to the skilled person. In this context, the subject may be an animal.

The expected postnatal survival rate is at least 50%, 60%, 70%, 80%, 90% or 100% compared to a fetus whose mother is not administered with an antibody-based molecule of this invention. An antibody-based molecule may induce an increased survival rate of the treated fetus. The survival rate of treated fetus is expected to be at least 50%, 60%, 70%, 80%, 90%, or 100% compared to the non-treated fetus. The experimental part discloses some exemplary methods. In this context, the subject may be an animal.

An antibody-based molecule may induce a prolonged postnatal survival of the treated fetus. In this context, the “treated fetus” means the fetus is treated via administering the mother of said antibody-based molecule and/or via administering the fetus (or newborn) after birth. The postnatal survival rate of treated fetus may have been considered to have been increased when the lifespan of the treated fetus extends at least 1, 2, 3, 4, 5, 6, 7, 8, 9, 10, 11, 12 weeks or at least 1, 2, 3, 4, 5, 6, 7, 8, 9, 10, 11 or 12 months or at least 1, 2, 3 or more years compared to the expected lifespan of a subject having the same disease or condition, whose mother has not been administered with an antibody-based molecule of the invention. In this context, the subject may be an animal.

Each of the therapeutic effects characterized herein could be seen as a stabilization of the disorder. In an embodiment, the use of an antibody-based exhibits a therapeutic effect on the treated fetus defined herein. In an embodiment, the use of an antibody-based molecule exhibits a therapeutic effect on the treated fetus defined herein wherein the antibody-based is administered to the mother of said fetus. Accordingly, i an embodiment, the use of an antibody-based molecule exhibits a therapeutic effect on the fetus having a disease or condition resulting from a genetic defect. In a preferred embodiment, the use of an antibody-based molecule exhibits a therapeutic effect on the fetus having a disease or condition resulting from a genetic defect, wherein the disease or condition is a neuromuscular disease.

In another embodiment, the use of an antibody-based molecule exhibits a therapeutic effect on the fetus having a neuromuscular disease, wherein said disease is not associated with the presence of a genetic defect Preferably, this disease is caused by factors such as inflammation, infection, radiation or chemical toxicity.

### Anti-MuSK Antibody or Antigen-Binding Fragment Thereof

In an embodiment, the invention provides an antibody-based molecule for use in the treatment of a disease or condition resulting from a genetic defect in a fetus wherein the antibody-based molecule is administered to the mother of said fetus, is an anti-MuSK antibody or antigen binding fragment thereof, and the known target is MuSK.

The term “anti-MuSK antibody” may be replaced by the term “MuSK antibody”. Any anti-MuSK antibody-based molecule including anti-MuSK antibodies, epitope-binding domains thereof, antigen binding fragments thereof and antibody derivatives that is capable of binding muscle-specific tyrosine protein kinase (MuSK) is encompassed within the present invention. In an embodiment, such anti-MuSK antibody is also able to activate the signalling and/or phosphorylation of MuSK. The invention provides the insight that such antibody-based molecules are useful for the treatment of conditions where a subject is in need of increased MuSK signalling or MuSK phosphorylation, such as neuromuscular disease or conditions. Therefore in a first aspect, there is provided an anti-MuSK antibody or antigen-binding fragment thereof for use in the treatment of a neuromuscular disorder in a human subject.

MuSK is a receptor tyrosine kinase that is expressed in skeletal muscle and has a crucial, master role in forming and maintaining neuromuscular synapses (Burden et al., “The Role of MuSK in Synapse Formation and Neuromuscular Disease,” Cold Spring Harb. Perspect. Biol. 5:a009167 (2013), which is hereby incorporated by reference in its entirety). MuSK is a single pass, 120 kDa transmembrane protein, composed of an extracellular region containing three Ig-like domains and a Frizzled (Fz)-like domain, and an intracellular region containing a juxtamembrane region, a kinase domain and a short cytoplasmic tail (Jennings et al., “Muscle-Specific trk-Related Receptor with a Kringle Domain Defines a Distinct Class of Receptor Tyrosine Kinases,” Proc. Natl. Acad. Sci. USA 90:2895-2899 (1993) and Valenzuela et al., “Receptor Tyrosine Kinase Specific for the Skeletal Muscle Lineage: Expression in Embryonicmuscle, at the Neuromuscular Junction, and After Injury,” Neuron 15: 573-584 (1995), which are hereby incorporated by reference in their entirety). MuSK phosphorylation is stimulated by agrin, a signal provided by motor neurons. Once activated, MuSK stimulates pathways that (1) cluster and anchor AChRs and additional muscle proteins critical for synaptic transmission, (2) enhance transcription of genes encoding synaptic proteins in muscle ‘synaptic nuclei’ and (3) promote the production of retrograde signals that promote presynaptic differentiation and attachment of motor nerve terminals to muscle. In the absence of MuSK, neuromuscular synapses fail to form (Burden et al., “The Role of MuSK in Synapse Formation and Neuromuscular Disease,” Cold Spring Harb. Perspect. Biol. 5:a009167 (2013), which is hereby incorporated by reference in its entirety). In addition to its role during synapse formation, MuSK is also required to maintain adult synapses, as inhibition of MuSK expression in adult muscle leads to profound defects in presynaptic and postsynaptic differentiation (Kong et al., “Inhibition of Synapse Assembly in Mammalian Muscle in vivo by RNA Interference,” EMBO Rep 5:183-188 (2004) and Hesser et al., “Synapse Disassembly and Formation of New Synapses in Postnatal Muscle Upon Conditional Inactivation of MuSK,” Mol. Cell. Neurosci. 31:470-480 (2006), which are hereby incorporated by reference in their entirety). Consistent with these findings in mice, mutations that impair MuSK kinase activity or inhibit signalling steps downstream from MuSK cause myasthenia (CM), characterized by structurally and functionally defective synapses, leading to muscle weakness and fatigue (Beeson et al., “Dok-7 Mutations Underlie a Neuromuscular Junction Synaptopathy,” Science 313:1975-1978 (2006); Muller et al., “Phenotypical Spectrum of DOK7 Mutations in Congenital Myasthenic Syndromes,” Brain 130:1497-1506 (2007); and Selcen et al., “A Compensatory Subpopulation of Motor Neurons in a Mouse Model of Amyotrophic Lateral Sclerosis,” J. Comp. Neurol. 490:209-219 (2008), which are hereby incorporated by reference in their entirely).

The amino acid sequence of human MuSK has the amino acid sequence of SEQ ID NO: 129 below.

(SEQ ID NO: 129)
MRELVNIPLVHILTLVAFSGTEKLPKAPVITTPLETVDALVEEVA
TFMCAVESYPQPEISWTRNKILIKLFDTRYSIRENGQLLTILSVE
DSDDGIYCCTANNGVGGAVESCGALQVKMKPKITRPPINVKIIEG
LKAVLPCTTMGNPKPSVSWIKGDSPLRENSRIAVLESGSLRIHNV
QKEDAGQYRCVAKNSLGTAYSKVVKLEVEVFARILRAPESHNVTF
GSFVTLHCTATGIPVPTITWIENGNAVSSGSIQESVKDRVIDSRL
QLFITKPGLYTCIATNKHGEKFSTAKAAATISIAEWSKPQKDNKG
YCAQYRGEVCNAVLAKDALVFLNTSYADPEEAQELLVHTAWNELK
VVSPVCRPAAEALLCNHIFQECSPGVVPTPIPICREYCLAVKELF
CAKEWLVMEEKTHRGLYRSEMHLLSVPECSKLPSMHWDPTACARL
PHLDYNKENLKTFPPMTSSKPSVDIPNLPSSSSSSFSVSPTYSMT
VIISIMSSFAIFVLLTITTLYCCRRRKQWKNKKRESAAVTLTTLP
SELLLDRLHPNPMYQRMPLLLNPKLLSLEYPRNNIEYVRDIGEGA
FGRVFQARAPGLLPYEPFTMVAVKMLKEEASADMQADFQREAALM
AEFDNPNIVKLLGVCAVGKPMCLLFEYMAYGDLNEFLRSMSPHTV
CSLSHSDLSMRAQVSSPGPPPLSCAEQLCIARQVAAGMAYLSERK
FVHRDLATRNCLVGENMVVKIADFGLSRNIYSADYYKANENDAIP
IRWMPPESIFYNRYTTESDVWAYGVVLWEIFSYGLQPYYGMAHEE
VIYYVRDGNILSCPENCPVELYNLMRLCWSKLPADRPSFTSIHRI
LERMCERAEGTVSV

In accordance with the present invention, the MuSK antibody-based molecules described herein bind to an epitope within the Frizzled (Fz)-like domain of the MuSK protein. The Fz-like domain of MuSK has the amino acid sequence of SEQ ID NO: 130 as shown below:

(SEQ ID NO: 130)
DNKGYCAQYRGEVCNAVLAKDALVFLNTSYADPEEAQELLVHTAW
NELKVVSPVCRPAAEALLCNHIFQECSPGVVPTPIPICREYCLAV
KELFCAKEWLVMEEKTHRGLYRSEMHLLSVPECSKLPSMHWDPTA
CARL

The term “epitope” as used herein refers to an antigenic determinant capable of being bound to an antibody. Epitopes usually comprise surface groupings of molecules such as amino acids or sugar side chains and usually have specific three-dimensional structural characteristics, as well as specific charge characteristics. Conformational and non-conformational epitopes are distinguished in that the binding to the former, but not the latter, is lost in the presence of denaturing solvents. An epitope may comprise amino acid residues directly involved in the binding (also called the immunodominant component of the epitope) and other amino acid residues, which are not directly involved in the binding, such as amino acid residues that are effectively blocked by the specific antigen-binding peptide (in other words, the amino acid residue is within the footprint of the specific antigen-binding peptide). An epitope typically includes at least 3, and more usually, at least 5, 6, 7, 8, 9, 10, 11, 12, 13, 14, 15, 16, 17, 18, 19, 20 or more amino acids in a unique spatial conformation.

In an embodiment, the MuSK antibody or antigen binding fragment, for use according to the invention binds the MuSK Frizzled (Fz) like domain. In an embodiment, the MuSK antibody or antigen binding fragment immunospecifically bind an epitope within the MuSK Fz-like domain sequence of SEQ ID NO: 130 more frequently, more rapidly, with greater duration and/or with greater affinity or avidity than an alternative epitope. In an embodiment, the MuSK antibody-based molecules described herein bind immunospecifically to any 2, 3, 4, 5, 6, 7, 8, 9, 10 or more amino acid residues of SEQ ID NO: 130. The term “affinity”, “specific binding”, “binding”, “immunospecific binding”, “binding activity” or “specific binding activity”, as used herein, refers to the degree to which an antibody or an antibody fragment as defined herein binds to an epitope within the MuSK-Fz-like domain sequence of SEQ ID NO:130.

In an embodiment, the MuSK antibody-based molecules as disclosed herein bind to the MuSK Fz-like domain with an affinity corresponding to a KD of about 10⁻⁷ M or less. For example, the MuSK antibody-based molecules disclosed herein bind to the MuSK Fz-like domain with an affinity corresponding to a KD of about 10⁻⁸ M, of about 10⁹ M, of about 10⁻¹⁰ M, of about 10¹¹ M, of about 10⁻¹² M or less when determined by, for instance, surface plasmon resonance (SPR) technology in a Biacore 3000 instrument (preferably using the antibody as the ligand and MuSK as the analyte). The MuSK antibody-based molecules as disclosed herein bind to the MuSK Fz-like domain with an affinity corresponding to a KD that is at least ten-fold lower, such as at least 100-fold lower, for instance at least 1,000-fold lower, such as at least 10,000-fold lower, for instance at least 100,000-fold lower than its affinity for binding to a non-specific antigen (e.g., bovine serum albumin, casein, etc.). The amount with which the affinity is lower is dependent on the KD of the antibody, so that when the KD of the antibody is very low (that is, the antibody is highly specific), then the amount with which the affinity for the antigen is lower than the affinity for a non-specific antigen may be at least 10,000 fold. The term “kd” (sec −1 or 1/s), as used herein, refers to the dissociation rate constant of a particular antibody-antigen interaction. The value is also referred to as the koff value. The term “ka” (M−1×sec−1 or 1/Ms), as used herein, refers to the association rate constant of a particular antibody-antigen interaction. The term “KD” (M), as used herein, refers to the dissociation equilibrium constant of a particular antibody-antigen interaction and is obtained by dividing the kd by the ka. The term “KA” (M−1 or 1/M), as used herein, refers to the association equilibrium constant of a particular antibody-antigen interaction and is obtained by dividing the ka by the kd.

In an embodiment, the MuSK antibody-based molecules described herein have a pH-dependent binding affinity for MuSK that allows for antibody recycling to enhance antigen binding. For example, in an embodiment, the association rate constant or dissociation rate constant may differ under acidic vs. neutral vs. basic pH conditions. In one embodiment, the MuSK antibody-based molecules described herein have a higher dissociation rate constant under acidic pH conditions, e.g., pH of <7.0, compared to neutral pH conditions, e.g., pH of ˜7.0-7.9. In some embodiments, the MuSK antibody-based molecules described herein have a 2-fold to 3-fold higher dissociation rate constant (i.e., decreased binding affinity) at an acidic pH (e.g., pH ˜5.5) as compared to a neutral pH. (pH ˜7.4). In an embodiment, the MuSK antibody-based molecules bind the MuSK Fz-like domain with a higher affinity at neutral pH conditions than at acidic pH conditions. In other words, in an embodiment, the MuSK antibody-based molecule binds the MuSK Fz-like domain with a higher dissociation rate at acidic pH conditions than under neutral pH conditions. Neutral pH conditions may be defined as being a pH comprised from 7.0 to 7.9. Acidic pH conditions may be defined as being a pH being less than 7.0. Higher may mean at least 10%, 20%, 30%, 40%, 50%, 60%, 70%, 80%, 90%, 100%, 150%, 200%, 250%, 300% higher. Antibodies having this pH dependent dissociation characteristic dissociate from the antigen after binding and activation but before lysosomal degradation. Once dissociated, the antibody is transported via the neonatal Fc receptor back into circulation and is released to bind more antigen.

In some embodiments, binding of the MuSK antibodies of the present invention to their respective epitopes within the Fz-like domain activates MuSK signalling. In particular, when the MuSK antibodies of the present invention bind their respective epitope of the MuSK Fz-like domain, this binding induces MuSK phosphorylation and activation. The MuSK antibodies of the present invention induce MuSK phosphorylation by about 50% to about 100% relative to MuSK phosphorylation induced by agrin activation (as measured, e.g., in a C2C12 phosphorylation assay). In an embodiment, the MuSK antibodies of the present invention induce about 55%, 60%, 65%, 70%, 75%, 80%, 85%, 90%, 95% or 100% 95% MuSK phosphorylation (relative to MuSK phosphorylation induced by agrin activation). In an embodiment, the MuSK antibody-based molecules of the present invention induce about 90% to about 100% MuSK phosphorylation (relative to MuSK phosphorylation induced by agrin activation), upon MuSK binding. Phosphorylation of MuSK may be assessed using techniques known to the skilled person such as western blotting or a C2C12 myotube phosphorylation assay. Such antibodies activating MuSK signalling (i.e. induction of the dimerization of MuSK, induction of the tyrosine phosphorylation of MuSK) are agonist antibodies.

In some embodiments, the MuSK antibodies of the present invention, i.e., MuSK antibodies that bind to the Fz-domain of MuSK, do not interfere (i.e., do not block, impede, inhibit, or reduce) with natural ligand binding and stimulation of MuSK. In some embodiments, the MuSK antibodies co-stimulate MuSK activation with its natural ligand, i.e., agrin, to produce an additive effect of activation, e.g, MuSK phosphorylation. Thus, in some embodiments, the MuSK antibodies of the present invention potentiate natural MuSK activation, i.e., phosphorylation, induced by natural ligand binding. Such MuSK antibodies are agonist antibodies. In some embodiments, the antibodies of the invention, in combination with the natural ligand, activate MuSK (i.e., MuSK phosphorylation) to >100% of endogenous activation levels such as at least 110%, 130%, 150%, 200% of endogenous activation levels.

Accordingly, in an embodiment, activities of the MuSK antibody-based molecules of the invention include: (i) binding to an epitope of human muscle-specific tyrosine-protein kinase (MuSK), said epitope present in the MuSK Frizzled (Fz)-like domain sequence of SEQ ID NO: 130, wherein said antibody-based molecule induces MuSK phosphorylation upon binding to its epitope, and/or (ii) binding to the MuSK Fz-like domain does not block, impede, or inhibit natural or endogenous MuSK ligand induced phosphorylation, and may potentiate said natural or endogenous MuSK ligand induced phosphorylation, and (iii) binding to the MuSK Fz-like domain occurs with a higher affinity at neutral pH conditions than at acidic pH conditions. All these features have been further defined herein.

In an embodiment, an anti-MuSK antibody or antigen binding fragment thereof is provided which:
binds the MuSK Frizzled (Fz)-like domain sequence of SEQ ID NO: 129,
is an agonist MuSK antibody and/or
has reduced or eliminated effector function.

This MuSK antibody or antigen-binding fragment thereof is preferably for use in the treatment of a disease or condition resulting from a genetic defect in a fetus wherein the antibody-based molecule is administered to the mother of said fetus.

Reduced or eliminated effector function may be obtained as earlier described herein by introducing mutation in the human IgG constant Fc region. Preferably, at least 1, 2, 3, 4, 5, 6, 7, 8, 9, 10, 11, 12, 13, 14, 15, 16 or 17 amino acid substitutions are introduced into said Fc region. Preferably, at least 1, 2, 3, 4, amino acid substitutions are introduced into said Fc region.

Said Fc region may comprise SEQ ID NO: 266 or 267 and said substitutions are introduced at amino acid positions selected from amino acid 234, 235, 236, 237, 238, 239, 243, 265, 267, 268, 292, 297, 300, 318, 320, 322, 327, 328, 329, 330, 331, 332, and 396 numbered according the EU numbering system of said sequence.

In an embodiment, said Fc region may comprise SEQ ID NO: 266 or 267 and said substitutions are introduced at amino acid positions selected from amino acid 234 or 235 (numbered according to the EU numbering system) of said sequence.

In an embodiment, said Fc region may comprise SEQ ID NO: 266 or 267 and said substitutions are introduced at amino acid positions selected from amino acid 234 and 235 (numbered according to the EU numbering system) of said sequence.

In an embodiment, one or more of the following mutations (all numbered according to the EU numbering system) are introduced into the human IgG constant Fc region SEQ ID NO: 266 or SEQ ID NO: 267 of the antibody-based molecule described herein: an N297A substitution; an N297Q substitution; an L234A substitution; an L234D substitution; an L234E substitution; an L234G substitution; an L234H substitution; an L234F substitution; an L234K substitution; an L234Q substitution; an L234R substitution; an L234S substitution; an L234T substitution; an L235A substitution; an L235D substitution; an L235E substitution; an L235F substitution; an L235G substitution; an L235V substitution; an L235H substitution; an L2351 substitution; an L235K substitution; an L235R substitution; an L235S substitution; L235T substitution; an L235Q substitution; an L237A substitution; an S239D substitution; an E233P substitution; an L234V substitution; a C236 deletion; a G236E substitution; a G236R substitution; a G236K substitution; a G237A substitution; a P238A substitution; an F243L substitution; a D265A substitution; an S267E substitution; an H268A substitution; an R292P substitution; a Y300L substitution; a K322A substitution; a K322Q substitution; an A327Q substitution; an L328F substitution; an L328R substitution; a P329A substitution; a P329G substitution; an A330L substitution; an A330S substitution; a P331S substitution; an 1332E substitution; or a P396L substitution.

In one embodiment, each of the combinations of mutations described earlier in the fourth embodiment of this application in the human IgG constant Fc region of the antibody-based molecule described herein may be made.

In a preferred embodiment, L234A or L235A numbered according to the EU numbering system substitution is introduced into the human IgG constant Fc region of the antibody-based molecule described herein. In a more preferred embodiment, L234A and L235A numbered according to the EU numbering system substitutions are introduced into the human IgG constant Fc region of the antibody-based molecule described herein. This embodiment results in an antibody-based molecule with a heavy chain represented by SEQ ID NO:268 or 270.

In an even more preferred embodiment, said anti-MuSK antibody or antigen binding fragment thereof, comprises:
a) a heavy chain variable domain (VH) comprising an amino acid sequence that is at least 80% identical or similar to SEQ ID NO: 234 and
b) a light chain variable domain (VL) comprising an amino acid sequence that is at least 80% identical or similar to SEQ ID NO: 235.

Within this context, the identity or similarity is of at least 81%, 82%, 83%, 84%, 85%, 86%, 87%, 88%, 89%, 90%, 91%, 92%, 93%, 94%, 95%, 96%, 97%, 98%, 99% or 100%.

Preferred anti-MuSK antibody or antigen binding fragment thereof, comprises:
a) A full length heavy chain comprising SEQ ID NO: 268 and
b) A full length light chain comprising SEQ ID NO: 269.

Preferred anti-MuSK antibody or antigen binding fragment thereof, comprises:
c) A full length heavy chain comprising SEQ ID NO: 270 and
d) A full length light chain comprising SEQ ID NO: 271.

In an embodiment, the MuSK-antibody based molecule as described herein comprises the amino acid sequence of any one, any two, any three, any four, any five, or any six CDRs as provided in Tables 1 and 2 herein.

In one embodiment, the antibody-based molecule that binds to human muscle-specific tyrosine-protein kinase (MuSK) comprises a heavy chain variable domain, where the heavy chain variable domain comprises:
(i) a complementarity-determining region 1 (CDR-H1) comprising an amino acid sequence of any one of SEQ ID NOs: 1-16, 135, 136, 147-149 or a modified amino acid sequence of any one of SEQ ID NOs: 1-16, 135, 136, or 147-149 said modified sequence having 1, 2, 3, 4 or 5 amino acid alterations relative to SEQ ID NOs: 1-16, 135, 136 or 147-149;
(ii) a complementarity-determining region 2 (CDR-H2) comprising an amino acid sequence of any one of SEQ ID NOs: 17-32, 137, 138, 150-155 or a modified amino acid sequence of any one of SEQ ID NOs: 17-32, 137, 138, or 150-155 said modified sequences having 1, 2, 3, 4 or 5 amino acid alterations relative to SEQ ID NOs: 17-32, 137, 138, or 150-155; and
(iii) a complementarity-determining region 3 (CDR-H3) comprising an amino acid sequence of any one of SEQ ID NOs: 33-48, 139, 140, 156-158, 240-251, or a modified amino acid sequence of any one of SEQ ID NO: 33-48, 139, 140, 156-158, or 240-251, said modified sequence having 1, 2, 3, 4 or 5 amino acid alterations relative to SEQ ID NOs: 33-48, 139, 140, 156-158, or 240-251.

In an embodiment, the antibody-based molecule that binds to human muscle-specific tyrosine-protein kinase (MuSK) comprises: (i) a heavy chain variable domain comprising the CDR-H1 of SEQ ID NO: 1 or having 1, 2, 3, 4 or 5 amino acid alterations relative to SEQ ID NO: 1, the CDR-H2 of SEQ ID NO: 17 or having 1, 2, 3, 4 or 5 amino acid alterations relative to SEQ ID NO:17, and the CDR-H3 of SEQ ID NO: 33 or having 1, 2, 3, 4 or 5 amino acid alterations relative to SEQ ID NO:33; (ii) a heavy chain variable domain comprising the CDR-H1 of SEQ ID NO: 2 or having 1, 2, 3, 4 or 5 amino acid alterations relative to SEQ ID NO:2, the CDR-H2 of SEQ ID NO: 18 or having 1, 2, 3, 4 or 5 amino acid alterations relative to SEQ ID NO:18, and the CDR-H3 of SEQ ID NO: 34 or having 1, 2, 3, 4 or 5 amino acid alterations relative to SEQ ID NO:34; (iii) a heavy chain variable domain comprising the CDR-H1 of SEQ ID NO: 3 or having 1, 2, 3, 4 or 5 amino acid alterations relative to SEQ ID NO:3, the CDR-H2 of SEQ ID NO: 19 or having 1, 2, 3, 4 or 5 amino acid alterations relative to SEQ ID NO:19, and the CDR-H3 of SEQ ID NO: 35 or having 1, 2, 3, 4 or 5 amino acid alterations relative to SEQ ID NO:35; (iv) a heavy chain variable domain comprising the CDR-H1 of SEQ ID NO: 4 or having 1, 2, 3, 4 or 5 amino acid alterations relative to SEQ ID NO:4, the CDR-H2 of SEQ ID NO: 20 or having 1, 2, 3, 4 or 5 amino acid alterations relative to SEQ ID NO:20, and the CDR-H3 of SEQ ID NO: 36 or having 1, 2, 3, 4 or 5 amino acid alterations relative to SEQ ID NO:36; (v) a heavy chain variable domain comprising the CDR-H1 of SEQ ID NO: 5 or having 1, 2, 3, 4 or 5 amino acid alterations relative to SEQ ID NO:5, the CDR-H2 of SEQ ID NO: 21 or having 1, 2, 3, 4 or 5 amino acid alterations relative to SEQ ID NO:21, and the CDR-H3 of SEQ ID NO: 37 or having 1, 2, 3, 4 or 5 amino acid alterations relative to SEQ ID NO:37; (vi) a heavy chain variable domain comprising the CDR-H1 of SEQ ID NO: 6 or having 1, 2, 3, 4 or 5 amino acid alterations relative to SEQ ID NO:6, the CDR-H2 of SEQ ID NO: 22 or having 1, 2, 3, 4 or 5 amino acid alterations relative to SEQ ID NO:22, and the CDR-H3 of SEQ ID NO: 38 or having 1, 2, 3, 4 or 5 amino acid alterations relative to SEQ ID NO:38; (vii) a heavy chain variable domain comprising the CDR-H1 of SEQ ID NO: 7 or having 1, 2, 3, 4 or 5 amino acid alterations relative to SEQ ID NO:7, the CDR-H2 of SEQ ID NO: 23 or having 1, 2, 3, 4 or 5 amino acid alterations relative to SEQ ID NO:23, and the CDR-H3 of SEQ ID NO: 39 or having 1, 2, 3, 4 or 5 amino acid alterations relative to SEQ ID NO:39; (viii) a heavy chain variable domain comprising the CDR-H1 of SEQ ID NO: 8 or having 1, 2, 3, 4 or 5 amino acid alterations relative to SEQ ID NO:8, the CDR-H2 of SEQ ID NO: 24 or having 1, 2, 3, 4 or 5 amino acid alterations relative to SEQ ID NO:24, and the CDR-H3 of SEQ ID NO: 40 or having 1, 2, 3, 4 or 5 amino acid alterations relative to SEQ ID NO:40; (ix) a heavy chain variable domain comprising the CDR-H1 of SEQ ID NO: 9 or having 1, 2, 3, 4 or 5 amino acid alterations relative to SEQ ID NO:9, the CDR-H2 of SEQ ID NO: 25 or having 1, 2, 3, 4 or 5 amino acid alterations relative to SEQ ID NO:25, and the CDR-H3 of SEQ ID NO: 41 or having 1, 2, 3, 4 or 5 amino acid alterations relative to SEQ ID NO:41; (x) a heavy chain variable domain comprising the CDR-H1 of SEQ ID NO: 10 or having 1, 2, 3, 4 or 5 amino acid alterations relative to SEQ ID NO:10, the CDR-H2 of SEQ ID NO: 26 or having 1, 2, 3, 4 or 5 amino acid alterations relative to SEQ ID NO:26, and the CDR-H3 of SEQ ID NO: 42 or having 1, 2, 3, 4 or 5 amino acid alterations relative to SEQ ID NO:42; (xi) a heavy chain variable domain comprising the CDR-H1 of SEQ ID NO: 11 or having 1, 2, 3, 4 or 5 amino acid alterations relative to SEQ ID NO:11, the CDR-H2 of SEQ ID NO: 27 or having 1, 2, 3, 4 or 5 amino acid alterations relative to SEQ ID NO:27, and the CDR-H3 of SEQ ID NO: 43 or having 1, 2, 3, 4 or 5 amino acid alterations relative to SEQ ID NO:43; (xii) a heavy chain variable domain comprising the CDR-H1 of SEQ ID NO: 12 or having 1, 2, 3, 4 or 5 amino acid alterations relative to SEQ ID NO:12, the CDR-H2 of SEQ ID NO: 28 or having 1, 2, 3, 4 or 5 amino acid alterations relative to SEQ ID NO:28, and the CDR-H3 of SEQ ID NO: 44 or having 1, 2, 3, 4 or 5 amino acid alterations relative to SEQ ID NO:44; (xiii) a heavy chain variable domain comprising the CDR-H1 of SEQ ID NO: 13 or having 1, 2, 3, 4 or 5 amino acid alterations relative to SEQ ID NO:13, the CDR-H2 of SEQ ID NO: 29 or having 1, 2, 3, 4 or 5 amino acid alterations relative to SEQ ID NO:29, and the CDR-H3 of SEQ ID NO: 45 or having 1, 2, 3, 4 or 5 amino acid alterations relative to SEQ ID NO:45; (xiv) a heavy chain variable domain comprising the CDR-H1 of SEQ ID NO: 14 or having 1, 2, 3, 4 or 5 amino acid alterations relative to SEQ ID NO:14, the CDR-H2 of SEQ ID NO: 30 or having 1, 2, 3, 4 or 5 amino acid alterations relative to SEQ ID NO:30, and the CDR-H3 of SEQ ID NO: 46 or having 1, 2, 3, 4 or 5 amino acid alterations relative to SEQ ID NO:46; (xv) a heavy chain variable domain comprising the CDR-H1 of SEQ ID NO: 15 or having 1, 2, 3, 4 or 5 amino acid alterations relative to SEQ ID NO:15, the CDR-H2 of SEQ ID NO: 31 or having 1, 2, 3, 4 or 5 amino acid alterations relative to SEQ ID NO:31, and the CDR-H3 of SEQ ID NO: 47 or having 1, 2, 3, 4 or 5 amino acid alterations relative to SEQ ID NO:47; (xvi) a heavy chain variable domain comprising the CDR-H1 of SEQ ID NO: 16 or having 1, 2, 3, 4 or 5 amino acid alterations relative to SEQ ID NO:16, the CDR-H2 of SEQ ID NO: 32 or having 1, 2, 3, 4 or 5 amino acid alterations relative to SEQ ID NO:32, and the CDR-H3 of SEQ ID NO: 48 or having 1, 2, 3, 4 or 5 amino acid alterations relative to SEQ ID NO:48; (xvii) a heavy chain variable domain comprising the CDR-H1 of SEQ ID NO: 135 or having 1, 2, 3, 4 or 5 amino acid alterations relative to SEQ ID NO:135, the CDR-H2 of SEQ ID NO: 137 or having 1, 2, 3, 4 or 5 amino acid alterations relative to SEQ ID NO:137, and the CDR-H3 of SEQ ID NO: 139 or having 1, 2, 3, 4 or 5 amino acid alterations relative to SEQ ID NO:139; and (xviii) a heavy chain variable domain comprising the CDR-H1 of SEQ ID NO: 136 or having 1, 2, 3, 4 or 5 amino acid alterations relative to SEQ ID NO:136, the CDR-H2 of SEQ ID NO: 138 or having 1, 2, 3, 4 or 5 amino acid alterations relative to SEQ ID NO:138, and the CDR-H3 of SEQ ID NO: 140 or having 1, 2, 3, 4 or 5 amino acid alterations relative to SEQ ID NO:140. The sequences of the heavy chain CDRs are provided in Table 1.

In an embodiment, the antibody-based molecule that binds to human muscle-specific tyrosine-protein kinase (MuSK) comprises: (ii.a) a heavy chain variable domain comprising the CDR-H1 of SEQ ID NO: 2 or having 1, 2, 3, 4 or 5 amino acid alterations relative to SEQ ID NO:2, the CDR-H2 of SEQ ID NO: 18 or having 1, 2, 3, 4 or 5 amino acid alterations relative to SEQ ID NO:18, and the CDR-H3 of SEQ ID NO: 240 (X2m1) or having 1, 2, 3, 4 or 5 amino acid alterations relative to SEQ ID NO:240; (ii.b) a heavy chain variable domain comprising the CDR-H1 of SEQ ID NO: 2 or having 1, 2, 3, 4 or 5 amino acid alterations relative to SEQ ID NO:2, the CDR-H2 of SEQ ID NO: 18 or having 1, 2, 3, 4 or 5 amino acid alterations relative to SEQ ID NO:18, and the CDR-H3 of SEQ ID NO: 241 (X2m2) or having 1, 2, 3, 4 or 5 amino acid alterations relative to SEQ ID NO:241; (ii.c) a heavy chain variable domain comprising the CDR-H1 of SEQ ID NO: 2 or having 1, 2, 3, 4 or 5 amino acid alterations relative to SEQ ID NO:2, the CDR-H2 of SEQ ID NO: 18 or having 1, 2, 3, 4 or 5 amino acid alterations relative to SEQ ID NO:18, and the CDR-H3 of SEQ ID NO: 242 (X2m3) or having 1, 2, 3, 4 or 5 amino acid alterations relative to SEQ ID NO:242; (ii.d) a heavy chain variable domain comprising the CDR-H1 of SEQ ID NO: 2 or having 1, 2, 3, 4 or 5 amino acid alterations relative to SEQ ID NO:2, the CDR-H2 of SEQ ID NO: 18 or having 1, 2, 3, 4 or 5 amino acid alterations relative to SEQ ID NO:18, and the CDR-H3 of SEQ ID NO: 243 (X2m4) or having 1, 2, 3, 4 or 5 amino acid alterations relative to SEQ ID NO:243; (ii.e) a heavy chain variable domain comprising the CDR-H1 of SEQ ID NO: 2 or having 1, 2, 3, 4 or 5 amino acid alterations relative to SEQ ID NO:2, the CDR-H2 of SEQ ID NO: 18 or having 1, 2, 3, 4 or 5 amino acid alterations relative to SEQ ID NO:18, and the CDR-H3 of SEQ ID NO: 244 (X2m5) or having 1, 2, 3, 4 or 5 amino acid alterations relative to SEQ ID NO:244; (ii.f) a heavy chain variable domain comprising the CDR-H1 of SEQ ID NO: 2 or having 1, 2, 3, 4 or 5 amino acid alterations relative to SEQ ID NO:2, the CDR-H2 of SEQ ID NO: 18 or having 1, 2, 3, 4 or 5 amino acid alterations relative to SEQ ID NO:18, and the CDR-H3 of SEQ ID NO: 245 (X2m6) or having 1, 2, 3, 4 or 5 amino acid alterations relative to SEQ ID NO:245; (ii.g) a heavy chain variable domain comprising the CDR-H1 of SEQ ID NO: 2 or having 1, 2, 3, 4 or 5 amino acid alterations relative to SEQ ID NO:2, the CDR-H2 of SEQ ID NO: 18 or having 1, 2, 3, 4 or 5 amino acid alterations relative to SEQ ID NO:18, and the CDR-H3 of SEQ ID NO: 246 (X2m7) or having 1, 2, 3, 4 or 5 amino acid alterations relative to SEQ ID NO:246; (ii.h) a heavy chain variable domain comprising the CDR-H1 of SEQ ID NO: 2 or having 1, 2, 3, 4 or 5 amino acid alterations relative to SEQ ID NO:2, the CDR-H2 of SEQ ID NO: 18 or having 1, 2, 3, 4 or 5 amino acid alterations relative to SEQ ID NO:18, and the CDR-H3 of SEQ ID NO: 247 (X2m8) or having 1, 2, 3, 4 or 5 amino acid alterations relative to SEQ ID NO:247.

In an embodiment, the antibody-based molecule that binds to human muscle-specific tyrosine-protein kinase (MuSK) comprises: (xvii.a) a heavy chain variable domain comprising the CDR-H1 of SEQ ID NO: 135 or having 1, 2, 3, 4 or 5 amino acid alterations relative to SEQ ID NO:135, the CDR-H2 of SEQ ID NO: 137 or having 1, 2, 3, 4 or 5 amino acid alterations relative to SEQ ID NO:137, and the CDR-H3 of SEQ ID NO: 248 (X17m1) or having 1, 2, 3, 4 or 5 amino acid alterations relative to SEQ ID NO:248; (xvii.b) a heavy chain variable domain comprising the CDR-H1 of SEQ ID NO: 135 or having 1, 2, 3, 4 or 5 amino acid alterations relative to SEQ ID NO:135, the CDR-H2 of SEQ ID NO: 137 or having 1, 2, 3, 4 or 5 amino acid alterations relative to SEQ ID NO:137, and the CDR-H3 of SEQ ID NO: 249 (X17m2) or having 1, 2, 3, 4 or 5 amino acid alterations relative to SEQ ID NO:249; (xvii.c) a heavy chain variable domain comprising the CDR-H1 of SEQ ID NO: 135 or having 1, 2, 3, 4 or 5 amino acid alterations relative to SEQ ID NO:135, the CDR-H2 of SEQ ID NO: 137 or having 1, 2, 3, 4 or 5 amino acid alterations relative to SEQ ID NO:137, and the CDR-H3 of SEQ ID NO: 250 (X17m3) or having 1, 2, 3, 4 or 5 amino acid alterations relative to SEQ ID NO:250; (xvii.d) a heavy chain variable domain comprising the CDR-H1 of SEQ ID NO: 135 or having 1, 2, 3, 4 or 5 amino acid alterations relative to SEQ ID NO:135, the CDR-H2 of SEQ ID NO: 137 or having 1, 2, 3, 4 or 5 amino acid alterations relative to SEQ ID NO:137, and the CDR-H3 of SEQ ID NO: 251 (X17m6) or having 1, 2, 3, 4 or 5 amino acid alterations relative to SEQ ID NO:251.

In an embodiment, the antibody-based molecule that binds to human muscle-specific tyrosine-protein kinase (MuSK) comprises a heavy chain variable domain, where the heavy chain variable domain comprises: (xix) a heavy chain variable domain comprising the CDR-H1 of SEQ ID NO: 147 or having 1, 2, 3, 4 or 5 amino acid alterations relative to SEQ ID NO:147, the CDR-H2 of SEQ ID NO: 150 or having 1, 2, 3, 4 or 5 amino acid alterations relative to SEQ ID NO:150, and the CDR-H3 of SEQ ID NO: 156 or having 1, 2, 3, 4 or 5 amino acid alterations relative to SEQ ID NO:156; (xx) a heavy chain variable domain comprising the CDR-H1 of SEQ ID NO: 148 or having 1, 2, 3, 4 or 5 amino acid alterations relative to SEQ ID NO:148, the CDR-H2 of SEQ ID NO: 151 or having 1, 2, 3, 4 or 5 amino acid alterations relative to SEQ ID NO:151 and the CDR-H3 of SEQ ID NO: 157 or having 1, 2, 3, 4 or 5 amino acid alterations relative to SEQ ID NO:157; (xxi) a heavy chain variable domain comprising the CDR-H1 of SEQ ID NO: 149 or having 1, 2, 3, 4 or 5 amino acid alterations relative to SEQ ID NO:149, the CDR-H2 of SEQ ID NO: 152 or having 1, 2, 3, 4 or 5 amino acid alterations relative to SEQ ID NO:152, and the CDR-H3 of SEQ ID NO: 158 or having 1, 2, 3, 4 or 5 amino acid alterations relative to SEQ ID NO:158.

In an embodiment, the antibody-based molecule that binds to human muscle-specific tyrosine-protein kinase (MuSK) comprises a heavy chain variable domain, where the heavy chain variable domain comprises (xxii) a heavy chain variable domain comprising the CDR-H1 of SEQ ID NO: 147 or having 1, 2, 3, 4 or 5 amino acid alterations relative to SEQ ID NO:147, the CDR-H2 of SEQ ID NO: 153 or having 1, 2, 3, 4 or 5 amino acid alterations relative to SEQ ID NO:153, and the CDR-H3 of SEQ ID NO:156 (3B2g1m1/3B2g2m1) or having 1, 2, 3, 4 or 5 amino acid alterations relative to SEQ ID NO:156; (xxiii) a heavy chain variable domain comprising the CDR-H1 of SEQ ID NO: 147 or having 1, 2, 3, 4 or 5 amino acid alterations relative to SEQ ID NO:147, the CDR-H2 of SEQ ID NO: 154 or having 1, 2, 3, 4 or 5 amino acid alterations relative to SEQ ID NO:154, and the CDR-H3 of SEQ ID NO: 156 (3B2g1m2/3B2g2m2) or having 1, 2, 3, 4 or 5 amino acid alterations relative to SEQ ID NO:156; (xxiv) a heavy chain variable domain comprising the CDR-H1 of SEQ ID NO: 147 or having 1, 2, 3, 4 or 5 amino acid alterations relative to SEQ ID NO:147, the CDR-H2 of SEQ ID NO: 155 or having 1, 2, 3, 4 or 5 amino acid alterations relative to SEQ ID NO:155, and the CDR-H3 of SEQ ID NO: 156 (3B2g1m4/3B2g2m4) or having 1, 2, 3, 4 or 5 amino acid alterations relative to SEQ ID NO:156. The sequences of the heavy chain CDRs are provided in Table 1.

In an embodiment, the antibody-based molecule that binds to human muscle-specific tyrosine-protein kinase (MuSK) comprises: (i) a heavy chain variable domain comprising the CDR-H1 of SEQ ID NO: 1, the CDR-H2 of SEQ ID NO: 17, and the CDR-H3 of SEQ ID NO: 33; (ii) a heavy chain variable domain comprising the CDR-H1 of SEQ ID NO: 2, the CDR-H2 of SEQ ID NO: 18, and the CDR-H3 of SEQ ID NO: 34; (iii) a heavy chain variable domain comprising the CDR-H1 of SEQ ID NO: 3, the CDR-H2 of SEQ ID NO: 19, and the CDR-H3 of SEQ ID NO: 35; (iv) a heavy chain variable domain comprising the CDR-H1 of SEQ ID NO: 4, the CDR-H2 of SEQ ID NO: 20, and the CDR-H3 of SEQ ID NO: 36; (v) a heavy chain variable domain comprising the CDR-H1 of SEQ ID NO: 5, the CDR-H2 of SEQ ID NO: 21, and the CDR-H3 of SEQ ID NO: 37; (vi) a heavy chain variable domain comprising the CDR-H1 of SEQ ID NO: 6, the CDR-H2 of SEQ ID NO: 22, and the CDR-H3 of SEQ ID NO: 38; (vii) a heavy chain variable domain comprising the CDR-H1 of SEQ ID NO: 7, the CDR-H2 of SEQ ID NO: 23, and the CDR-H3 of SEQ ID NO: 39; (viii) a heavy chain variable domain comprising the CDR-H1 of SEQ ID NO: 8, the CDR-H2 of SEQ ID NO: 24, and the CDR-H3 of SEQ ID NO: 40; (ix) a heavy chain variable domain comprising the CDR-H1 of SEQ ID NO: 9, the CDR-H2 of SEQ ID NO: 25, and the CDR-H3 of SEQ ID NO: 41; (x) a heavy chain variable domain comprising the CDR-H1 of SEQ ID NO: 10, the CDR-H2 of SEQ ID NO: 26, and the CDR-H3 of SEQ ID NO: 42; (xi) a heavy chain variable domain comprising the CDR-H1 of SEQ ID NO: 11, the CDR-H2 of SEQ ID NO: 27, and the CDR-H3 of SEQ ID NO: 43; (xii) a heavy chain variable domain comprising the CDR-H1 of SEQ ID NO: 12, the CDR-H2 of SEQ ID NO: 28, and the CDR-H3 of SEQ ID NO: 44; (xiii) a heavy chain variable domain comprising the CDR-H1 of SEQ ID NO: 13, the CDR-H2 of SEQ ID NO: 29, and the CDR-H3 of SEQ ID NO: 45; (xiv) a heavy chain variable domain comprising the CDR-H1 of SEQ ID NO: 14, the CDR-H2 of SEQ ID NO: 30, and the CDR-H3 of SEQ ID NO: 46; (xv) a heavy chain variable domain comprising the CDR-H1 of SEQ ID NO: 15, the CDR-H2 of SEQ ID NO: 31, and the CDR-H3 of SEQ ID NO: 47; (xvi) a heavy chain variable domain comprising the CDR-H1 of SEQ ID NO: 16, the CDR-H2 of SEQ ID NO: 32, and the CDR-H3 of SEQ ID NO: 48; (xvii) a heavy chain variable domain comprising the CDR-H1 of SEQ ID NO: 135, the CDR-H2 of SEQ ID NO: 137, and the CDR-H3 of SEQ ID NO: 139; and (xviii) a heavy chain variable domain comprising the CDR-H1 of SEQ ID NO: 136, the CDR-H2 of SEQ ID NO: 138, and the CDR-H3 of SEQ ID NO: 140. The sequences of the heavy chain CDR sequences are provided in Table 1 below.

In an embodiment, the antibody-based molecule that binds to human muscle-specific tyrosine-protein kinase (MuSK) comprises: (ii.a) a heavy chain variable domain comprising the CDR-H1 of SEQ ID NO: 2, the CDR-H2 of SEQ ID NO: 18, and the CDR-H3 of SEQ ID NO: 240 (X2m1); (ii.b) a heavy chain variable domain comprising the CDR-H1 of SEQ ID NO: 2, the CDR-H2 of SEQ ID NO: 18, and the CDR-H3 of SEQ ID NO: 241 (X2m2); (ii.c) a heavy chain variable domain comprising the CDR-H1 of SEQ ID NO: 2, the CDR-H2 of SEQ ID NO: 18, and the CDR-H3 of SEQ ID NO: 242 (X2m3); (ii.d) a heavy chain variable domain comprising the CDR-H1 of SEQ ID NO: 2, the CDR-H2 of SEQ ID NO: 18, and the CDR-H3 of SEQ ID NO: 243 (X2m4); (ii.e) a heavy chain variable domain comprising the CDR-H1 of SEQ ID NO: 2, the CDR-H2 of SEQ ID NO: 18, and the CDR-H3 of SEQ ID NO: 244 (X2m5); (ii.f) a heavy chain variable domain comprising the CDR-H1 of SEQ ID NO: 2, the CDR-H2 of SEQ ID NO: 18, and the CDR-H3 of SEQ ID NO: 245 (X2m6); (ii.g) a heavy chain variable domain comprising the CDR-H1 of SEQ ID NO: 2, the CDR-H2 of SEQ ID NO: 18, and the CDR-H3 of SEQ ID NO: 246 (X2m7); (ii.h) a heavy chain variable domain comprising the CDR-H1 of SEQ ID NO: 2, the CDR-H2 of SEQ ID NO: 18, and the CDR-H3 of SEQ ID NO: 247 (X2m8).

In an embodiment, the antibody-based molecule that binds to human muscle-specific tyrosine-protein kinase (MuSK) comprises: (xvii.a) a heavy chain variable domain comprising the CDR-H1 of SEQ ID NO: 135, the CDR-H2 of SEQ ID NO: 137, and the CDR-H3 of SEQ ID NO: 248 (X17m1); (xvii.b) a heavy chain variable domain comprising the CDR-H1 of SEQ ID NO: 135, the CDR-H2 of SEQ ID NO: 137, and the CDR-H3 of SEQ ID NO: 249 (X17m2); (xvii.c) a heavy chain variable domain comprising the CDR-H1 of SEQ ID NO: 135, the CDR-H2 of SEQ ID NO: 137, and the CDR-H3 of SEQ ID NO: 250 (X17m3); (xvii.d) a heavy chain variable domain comprising the CDR-H1 of SEQ ID NO: 135, the CDR-H2 of SEQ ID NO: 137, and the CDR-H3 of SEQ ID NO: 251 (X17m6).

In an embodiment, the antibody-based molecule that binds to human muscle-specific tyrosine-protein kinase (MuSK) comprises a heavy chain variable domain, where the heavy chain variable domain comprises: (xix) a heavy chain variable domain comprising the CDR-H1 of SEQ ID NO: 147, the CDR-H2 of SEQ ID NO: 150, and the CDR-H3 of SEQ ID NO: 156; (xx) a heavy chain variable domain comprising the CDR-H1 of SEQ ID NO: 148, the CDR-H2 of SEQ ID NO: 151, and the CDR-H3 of SEQ ID NO: 157; (xxi) a heavy chain variable domain comprising the CDR-H1 of SEQ ID NO: 149, the CDR-H2 of SEQ ID NO: 152, and the CDR-H3 of SEQ ID NO: 158;

In an embodiment, the antibody-based molecule that binds to human muscle-specific tyrosine-protein kinase (MuSK) comprises a heavy chain variable domain, where the heavy chain variable domain comprises (xxii) a heavy chain variable domain comprising the CDR-H1 of SEQ ID NO: 147, the CDR-H2 of SEQ ID NO: 153, and the CDR-H3 of SEQ ID NO:156 (3B2g1m1/3B2g2m1); (xxiii) a heavy chain variable domain comprising the CDR-H1 of SEQ ID NO: 147, the CDR-H2 of SEQ ID NO: 154, and the CDR-H3 of SEQ ID NO: 156 (3B2g1m2/3B2g2m2); (xxiv) a heavy chain variable domain comprising the CDR-H1 of SEQ ID NO: 147, the CDR-H2 of SEQ ID NO: 155, and the CDR-H3 of SEQ ID NO: 156 (3B2g1m4/3B2g2m4). The sequences of the heavy chain CDRs are provided in Table 1 below.

In an embodiment, the antibody-based molecule that binds to human muscle-specific tyrosine-protein kinase (MuSK) comprises a heavy chain variable domain, where the heavy chain variable domain comprises the CDR-H1 of SEQ ID NO: 147, CDR-H2 of SEQ ID NO: 153 or a CDR-H2 amino acid sequence having at least 0, 1, 2, 3, 4, or 5 alterations relative to SEQ ID NO: 153, and the CDR-H3 of SEQ ID NO:156 (3B2g2m1). In the context of this application, “3B2g2m1” is the same as “ARGX-119”. In an embodiment, the CDR-H2 amino acid sequence has at least 0, 1, 2, 3, 4, or 5 alterations relative to SEQ ID NO: 153. In accordance with this embodiment, the CDR-H2 amino acid sequence has at least 0, 1, 2, 3, 4, or 5 alterations relative to SEQ ID NO: 153, wherein said alterations are present at residues 1, 2, 6, 7, 8, 9, 10, 11, 12, 13, 14, 15, 16, 17, or any combination thereof.

In an embodiment, the antibody-based molecule that binds to human muscle-specific tyrosine-protein kinase (MuSK) comprises a VH, where the VH comprises:
a CDR-H1 amino acid sequence comprising SEQ ID NO: 147 or having 1, 2, 3, 4 or 5 amino acid alterations relative to SEQ ID NO: 147,
a CDR-H2 amino acid sequence comprising SEQ ID NO: 153 or having 1, 2, 3, 4 or 5 amino acid alterations relative to SEQ ID NO: 153, and
a CDR-H3 amino acid sequence comprising SEQ ID NO:156 or having 1, 2, 3, 4 or 5 amino acid alterations relative to SEQ ID NO:156 (3B2g2m1).

In an embodiment, the CDR-H2 of the antibody comprises a proline (P) at position 3, a tryptophan (W) at position 4, and a serine (S) or asparagine (N) at position 5. In a preferred embodiment, the CDR-H2 of the antibody comprises a proline (P) at position 3, a tryptophan (W) at position 4, and a serine (S) at position 5.

In an embodiment, the antibody-based molecule that binds to human muscle-specific tyrosine-protein kinase (MuSK) comprises a heavy chain variable domain, where the heavy chain variable domain comprises:
a CR-H1 amino acid sequence comprising or consisting of SEQ ID NO: 147,
a CDR-H2 amino acid sequence comprising or consisting of SEQ ID NO: 153, and
a CDR-H3 amino acid sequence comprising or consisting of SEQ ID NO:156 (3832g2m1).

The sequences of the heavy chain CDRs are provided in Table 2 below.

TABLE 1
VH CDR Sequences

| | HCDR1 | | HCDR2 | | HCDR3 | |
|---|---|---|---|---|---|---|
| | | SEQ | | SEQ | | SEQ |
| mAb/Fab | | ID | | ID | | ID |
| name | Sequence | NO: | Sequence | NO: | Sequence | NO: |
| X1 | SSSIH | 1 | SISSSSGSTSYADSVKG | 17 | KYWSQYYWAHYYGGLDY | 33 |
| X2 | SSSIH | 2 | SISSSYGSTSYADSVKG | 18 | SEGDRYVSGYMGMDY | 34 |
| X2m1 | SSSIH | 2 | SISSSYGSTSYADSVKG | 18 | SEGDRYVSGYFGFDY | 240 |
| X2m2 | SSSIH | 2 | SISSSYGSTSYADSVKG | 18 | SEGDRYVSGYFGLDY | 241 |
| X2m3 | SSSIH | 2 | SISSSYGSTSYADSVKG | 18 | SEGDRYVSGYSGFDY | 242 |
| X2m4 | SSSIH | 2 | SISSSYGSTSYADSVKG | 18 | SEGDRYVSGYSGLDY | 243 |
| X2m5 | SSSIH | 2 | SISSSYGSTSYADSVKG | 18 | SEGDRYVSGYFGMDY | 244 |
| X2m6 | SSSIH | 2 | SISSSYGSTSYADSVKG | 18 | SEGDRYVSGYSGMDY | 245 |
| X2m7 | SSSIH | 2 | SISSSYGSTSYADSVKG | 18 | SEGDRYVSGYMGFDY | 246 |
| X2m8 | SSSIH | 2 | SISSSYGSTSYADSVKG | 18 | SEGDRYVSGYMGLDY | 247 |
| X3 | SSSIH | 3 | SISSSSGYTYYADSVKG | 19 | SWYEMWMSGYFGFDY | 35 |
| X4 | SSSIH | 4 | SISSSSGSTYYADSVKG | 20 | GEHDYYVFGYLGMDY | 36 |
| X5 | SSSIH | 5 | SISSSSGSTSYADSVKG | 21 | SYTMFYYGGWYGSGYFGM | 37 |
| | | | | | DY | |
| X6 | SSSIH | 6 | SISSYSGYTYYADSVKG | 22 | TYGSYYVSSYTGMDY | 38 |
| X7 | SSSIH | 7 | SISSSYSSTYYADSVKG | 23 | LAGLYHYPGYLGLDY | 39 |
| X8 | SSSIH | 8 | SISSSSGSTSYADSVKG | 24 | SWSYHPWYYHVGWYTGLD | 40 |
| | | | | | Y | |
| X9 | SSSIH | 9 | SIYSSSGSTYYADSVKG | 25 | SGGEFYITSYYGMDY | 41 |
| X10 | SSSIH | 10 | SISSSYSSTSYADSVKG | 26 | KYYRWRHNKYQGFDY | 42 |
| X11 | SSSIH | 11 | SISSYSGSTYYADSVKG | 27 | SWGSYYVSGFVGFDY | 43 |
| X12 | SSSIH | 12 | YISPSSGYTSYADSVKG | 28 | QYWWPQWWITQYFGMDY | 44 |
| X13 | SSSIH | 13 | SISSSSGSTSYADSVKG | 29 | SSEHWYTIGYYGIDY | 45 |
| X14 | SSSIH | 14 | SISSSSGYTYYADSVKG | 30 | GSHHWFLWIYSGLDY | 46 |
| X15 | SSSIH | 15 | SISSSYGSTSYADSVKG | 31 | SEGDRYVSGYMGMDY | 47 |
| X16 | SSSIH | 16 | SIYSSYGYTSYADSVKG | 32 | NWGYYMYWGWYYALDY | 48 |
| X17 | YSSIH | 135 | SIYSSSGSTYYADSVKG | 137 | GDHGYYVFGYLGMDY | 139 |
| X17m1 | YSSIH | 135 | SIYSSSGSTYYADSVKG | 137 | GDHGYYVSGYLGMDY | 248 |
| X17m2 | YSSIH | 135 | SIYSSSGSTYYADSVKG | 137 | GDHGYYVYGYLGMDY | 249 |
| X17m3 | YSSIH | 135 | SIYSSSGSTYYADSVKG | 137 | GDHGYYVSGYLGFDY | 250 |
| X17m6 | YSSIH | 135 | SIYSSSGSTYYADSVKG | 137 | GEHGYYVSGYLGFDY | 251 |
| X18 | SSSIH | 136 | SISSSSGYTSYADSVKG | 138 | KYSKRAYPDYYWRGLDY | 140 |
| 14D10 | DYGMS | 147 | AIPWNGGSTYYKESVKG | 150 | RSGRIAFGALDA | 156 |
| 7G4 | DYGMS | 147 | AIPWNGGSTYYKESVKG | 150 | RSGRIAFGALDA | 156 |
| 3C4 | DYGMS | 147 | AIPWNGGSTYYKESVKG | 150 | RSGRIAFGALDA | 156 |
| 3B2 | DYGMS | 147 | AIPWNGGSTYYKESVKG | 150 | RSGRIAFGALDA | 156 |
| 3G3 | DYGMS | 147 | AIPWNGGSTYYKESVKG | 150 | RSGRIAFGALDA | 156 |
| 31G2 | DYGMS | 147 | AIPWNGGSTYYKESVKG | 150 | RSGRIAFGALDA | 156 |
| 31B7 | DYGMS | 147 | AIPWNGGSTYYKESVKG | 150 | RSGRIAFGALDA | 156 |
| 17H10 | ARYYS | 148 | VIAYDGSTYYSPSLKS | 151 | GSSRVAAAFDS | 157 |
| | WS | | | | | |
| 23B6 | ARYYS | 148 | VIAYDGSTYYSPSLKS | 151 | GSSRVAAAFDS | 157 |
| | WS | | | | | |
| 30E1 | ARYYS | 148 | VIAYDGSTYYSPSLKS | 151 | GSSRVAAAFDS | 157 |
| | WS | | | | | |
| 30A11 | ARYYS | 148 | VIAYDGSTYYSPSLKS | 151 | GSSRVAAAFDS | 157 |
| | WS | | | | | |
| 16F11 | LYYMN | 149 | VIDTHSIAYYADSVKG | 152 | GRTALVR | 158 |
| 4C11 | LYYMN | 149 | VIDTHSIAYYADSVKG | 152 | GRTALVR | 158 |
| 7A12 | LYYMN | 149 | VIDTHSIAYYADSVKG | 152 | GRTALVR | 158 |
| 7G12 | LYYMN | 149 | VIDTHSIAYYADSVKG | 152 | GRTALVR | 158 |
| 7B8 | LYYMN | 149 | VIDTHSIAYYADSVKG | 152 | GRTALVR | 158 |
| 3B2g1m1 | DYGMS | 147 | AIPWSGGSTYYKESVKG | 153 | RSGRIAFGALDA | 156 |
| 3B2g1m2 | DYGMS | 147 | AIPGSGGSTYYKESVKG | 154 | RSGRIAFGALDA | 156 |
| 3B2g1m4 | DYGMS | 147 | AIPWQGGSTYYKESVKG | 155 | RSGRIAFGALDA | 156 |
| 3B2g2m1 | DYGMS | 147 | AIPWSGGSTYYKESVKG | 153 | RSGRIAFGALDA | 156 |
| 3B2g2m2 | DYGMS | 147 | AIPGSGGSTYYKESVKG | 154 | RSGRIAFGALDA | 156 |
| 3B2g2m4 | DYGMS | 147 | AIPWQGGSTYYKESVKG | 155 | RSGRIAFGALDA | 156 |

In some embodiments, the MuSK antibody-based molecules as disclosed herein further comprise a light chain variable domain. The light chain variable domain comprises:
(i) a complementarity-determining region 1 (CDR-L1) having an amino acid sequence of any one of SEQ ID NOs: 49-64, 141, 142, 159-169, or a modified amino acid sequence of any one of SEQ ID NO: 49-64, 141, 142, or 159-169, said modified sequence having 1, 2, 3, 4 or 5 amino acid alterations relative to any one of SEQ ID NO: 49-64, 141, 142, or 159-169;
(ii) a complementarity-determining region 2 (CDR-L2) having an amino acid sequence of any one of SEQ ID NOs: 65-80, 143, 144, 170-179, or a modified amino acid sequence of any one of SEQ ID NO: 65-80, 143, 144 or 170-179, said modified sequence having 1, 2, 3, 4 or 5 amino acid alterations relative to any one of SEQ ID NO: 65-80, 143, 144 or 170-179; and
(iii) a complementarity-determining region 3 (CDR-L3) having an amino acid sequence of any one of SEQ ID NOs: 81-96, 145, 146, 180-195, or a modified amino acid sequence of any one of SEQ ID NO: 81-96, 145, 146, or 180-195, said modified sequence having 1, 2, 3, 4 or 5 amino acid alterations relative to any one of SEQ ID NO: 81-96, 145, 146 or 180-195.

In an embodiment, the light chain variable domain of the MuSK antibody based molecule disclosed herein comprises (i) a light chain variable domain comprising the CDR-L1 of SEQ ID NO: 49 or having 1, 2, 3, 4 or 5 amino acid alterations relative to SEQ ID NO:49, the CDR-L2 of SEQ ID NO: 65 or having 1, 2, 3, 4 or 5 amino acid alterations relative to SEQ ID NO:65, and the CDR-L3 of SEQ ID NO: 81 or having 1, 2, 3, 4 or 5 amino acid alterations relative to SEQ ID NO:81; (ii) a light chain variable domain comprising the CDR-L1 of SEQ ID NO: 50 or having 1, 2, 3, 4 or 5 amino acid alterations relative to SEQ ID NO:50, the CDR-L2 of SEQ ID NO: 66 or having 1, 2, 3, 4 or 5 amino acid alterations relative to SEQ ID NO:66, and the CDR-L3 of SEQ ID NO: 82 or having 1, 2, 3, 4 or 5 amino acid alterations relative to SEQ ID NO:82; (iii) a light chain variable domain comprising the CDR-L1 of SEQ ID NO: 51 or having 1, 2, 3, 4 or 5 amino acid alterations relative to SEQ ID NO:51, the CDR-L2 of SEQ ID NO: 67 or having 1, 2, 3, 4 or 5 amino acid alterations relative to SEQ ID NO:67, and the CDR-L3 of SEQ ID NO: 83 or having 1, 2, 3, 4 or 5 amino acid alterations relative to SEQ ID NO:83; (iv) a light chain variable domain comprising the CDR-L1 of SEQ ID NO: 52 or having 1, 2, 3, 4 or 5 amino acid alterations relative to SEQ ID NO:52, the CDR-L2 of SEQ ID NO: 68 or having 1, 2, 3, 4 or 5 amino acid alterations relative to SEQ ID NO:68, and the CDR-L3 of SEQ ID NO: 84 or having 1, 2, 3, 4 or 5 amino acid alterations relative to SEQ ID NO:84; (v) a light chain variable domain comprising the CDR-L1 of SEQ ID NO: 53 or having 1, 2, 3, 4 or 5 amino acid alterations relative to SEQ ID NO:53, the CDR-L2 of SEQ ID NO: 69 or having 1, 2, 3, 4 or 5 amino acid alterations relative to SEQ ID NO:69, and the CDR-L3 of SEQ ID NO: 85 or having 1, 2, 3, 4 or 5 amino acid alterations relative to SEQ ID NO:85; (vi) a light chain variable domain comprising the CDR-L1 of SEQ ID NO: 54 or having 1, 2, 3, 4 or 5 amino acid alterations relative to SEQ ID NO:54, the CDR-L2 of SEQ ID NO: 70 or having 1, 2, 3, 4 or 5 amino acid alterations relative to SEQ ID NO:70, and the CDR-L3 of SEQ ID NO: 86 or having 1, 2, 3, 4 or 5 amino acid alterations relative to SEQ ID NO:86; (vii) a light chain variable domain comprising the CDR-L1 of SEQ ID NO: 55 or having 1, 2, 3, 4 or 5 amino acid alterations relative to SEQ ID NO:55, the CDR-L2 of SEQ ID NO: 71 or having 1, 2, 3, 4 or 5 amino acid alterations relative to SEQ ID NO:71, and the CDR-L3 of SEQ ID NO: 87 or having 1, 2, 3, 4 or 5 amino acid alterations relative to SEQ ID NO:87; (viii) a light chain variable domain comprising the CDR-L1 of SEQ ID NO: 56 or having 1, 2, 3, 4 or 5 amino acid alterations relative to SEQ ID NO:56, the CDR-L2 of SEQ ID NO: 72 or having 1, 2, 3, 4 or 5 amino acid alterations relative to SEQ ID NO:72, and the CDR-L3 of SEQ ID NO: 88 or having 1, 2, 3, 4 or 5 amino acid alterations relative to SEQ ID NO:88; (ix) a light chain variable domain comprising the CDR-L1 of SEQ ID NO: 57 or having 1, 2, 3, 4 or 5 amino acid alterations relative to SEQ ID NO:57, the CDR-L2 of SEQ ID NO: 73 or having 1, 2, 3, 4 or 5 amino acid alterations relative to SEQ ID NO:73, and the CDR-L3 of SEQ ID NO: 89 or having 1, 2, 3, 4 or 5 amino acid alterations relative to SEQ ID NO:89; (x) a light chain variable domain comprising the CDR-L1 of SEQ ID NO: 58 or having 1, 2, 3, 4 or 5 amino acid alterations relative to SEQ ID NO:58, the CDR-L2 of SEQ ID NO: 74 or having 1, 2, 3, 4 or 5 amino acid alterations relative to SEQ ID NO:74, and the CDR-L3 of SEQ ID NO: 90 or having 1, 2, 3, 4 or 5 amino acid alterations relative to SEQ ID NO:90; (xi) a light chain variable domain comprising the CDR-L1 of SEQ ID NO: 59 or having 1, 2, 3, 4 or 5 amino acid alterations relative to SEQ ID NO:59, the CDR-L2 of SEQ ID NO: 75 or having 1, 2, 3, 4 or 5 amino acid alterations relative to SEQ ID NO:75, and the CDR-L3 of SEQ ID NO: 91 or having 1, 2, 3, 4 or 5 amino acid alterations relative to SEQ ID NO:91; (xii) a light chain variable domain comprising the CDR-L1 of SEQ ID NO: 60 or having 1, 2, 3, 4 or 5 amino acid alterations relative to SEQ ID NO:60, the CDR-L2 of SEQ ID NO: 76 or having 1, 2, 3, 4 or 5 amino acid alterations relative to SEQ ID NO:76, and the CDR-L3 of SEQ ID NO: 92 or having 1, 2, 3, 4 or 5 amino acid alterations relative to SEQ ID NO:92; (xiii) a light chain variable domain comprising the CDR-L1 of SEQ ID NO: 61 or having 1, 2, 3, 4 or 5 amino acid alterations relative to SEQ ID NO:61, the CDR-L2 of SEQ ID NO: 77 or having 1, 2, 3, 4 or 5 amino acid alterations relative to SEQ ID NO:77, and the CDR-L3 of SEQ ID NO: 93 or having 1, 2, 3, 4 or 5 amino acid alterations relative to SEQ ID NO:93; (xiv) a light chain variable domain comprising the CDR-L1 of SEQ ID NO: 62 or having 1, 2, 3, 4 or 5 amino acid alterations relative to SEQ ID NO:62, the CDR-L2 of SEQ ID NO: 78 or having 1, 2, 3, 4 or 5 amino acid alterations relative to SEQ ID NO:78, and the CDR-L3 of SEQ ID NO: 94 or having 1, 2, 3, 4 or 5 amino acid alterations relative to SEQ ID NO:94; (xv) a light chain variable domain comprising the CDR-L1 of SEQ ID NO: 63 or having 1, 2, 3, 4 or 5 amino acid alterations relative to SEQ ID NO:63, the CDR-L2 of SEQ ID NO: 79 or having 1, 2, 3, 4 or 5 amino acid alterations relative to SEQ ID NO:79, and the CDR-L3 of SEQ ID NO: 95 or having 1, 2, 3, 4 or 5 amino acid alterations relative to SEQ ID NO:95; (xvi) a light chain variable domain comprising the CDR-L1 of SEQ ID NO: 64 or having 1, 2, 3, 4 or 5 amino acid alterations relative to SEQ ID NO:64, the CDR-L2 of SEQ ID NO: 80 or having 1, 2, 3, 4 or 5 amino acid alterations relative to SEQ ID NO:80, and the CDR-L3 of SEQ ID NO: 96 or having 1, 2, 3, 4 or 5 amino acid alterations relative to SEQ ID NO:96; (xvii) a light chain variable domain comprising the CDR-L1 of SEQ ID NO: 141 or having 1, 2, 3, 4 or 5 amino acid alterations relative to SEQ ID NO:141, the CDR-L2 of SEQ ID NO: 143 or having 1, 2, 3, 4 or 5 amino acid alterations relative to SEQ ID NO:143, and the CDR-L3 of SEQ ID NO: 145 or having 1, 2, 3, 4 or 5 amino acid alterations relative to SEQ ID NO:145; (xviii) a light chain variable domain comprising the CDR-L1 of SEQ ID NO: 142 or having 1, 2, 3, 4 or 5 amino acid alterations relative to SEQ ID NO:142, the CDR-L2 of SEQ ID NO: 144 or having 1, 2, 3, 4 or 5 amino acid alterations relative to SEQ ID NO:144, and the CDR-L3 of SEQ ID NO: 146 or having 1, 2, 3, 4 or 5 amino acid alterations relative to SEQ ID NO:146. The sequences of the light chain CDRs are provided in Table 2 below.

In an embodiment, the light chain variable domain of the MuSK antibody based molecule disclosed herein comprises (xix) a light chain variable domain comprising the CDR-L1 of SEQ ID NO: 159 or having 1, 2, 3, 4 or 5 amino acid alterations relative to SEQ ID NO:159, the CDR-L2 of SEQ ID NO: 170 or having 1, 2, 3, 4 or 5 amino acid alterations relative to SEQ ID NO:170, and the CDR-L3 of SEQ ID NO: 180 or having 1, 2, 3, 4 or 5 amino acid alterations relative to SEQ ID NO:180; (xx) a light chain variable domain comprising the CDR-L1 of SEQ ID NO: 159 or having 1, 2, 3, 4 or 5 amino acid alterations relative to SEQ ID NO:159, the CDR-L2 of SEQ ID NO: 171 or having 1, 2, 3, 4 or 5 amino acid alterations relative to SEQ ID NO:171, and the CDR-L3 of SEQ ID NO: 181 or having 1, 2, 3, 4 or 5 amino acid alterations relative to SEQ ID NO:181; (xxi) a light chain variable domain comprising the CDR-L1 of SEQ ID NO: 160 or having 1, 2, 3, 4 or 5 amino acid alterations relative to SEQ ID NO:160, the CDR-L2 of SEQ ID NO: 172 or having 1, 2, 3, 4 or 5 amino acid alterations relative to SEQ ID NO:172, and the CDR-L3 of SEQ ID NO: 182 or having 1, 2, 3, 4 or 5 amino acid alterations relative to SEQ ID NO:182; (xxii) a light chain variable domain comprising the CDR-L1 of SEQ ID NO: 159 or having 1, 2, 3, 4 or 5 amino acid alterations relative to SEQ ID NO:159, the CDR-L2 of SEQ ID NO: 172 or having 1, 2, 3, 4 or 5 amino acid alterations relative to SEQ ID NO:172, and the CDR-L3 of SEQ ID NO: 183 or having 1, 2, 3, 4 or 5 amino acid alterations relative to SEQ ID NO:183; (xxiii) a light chain variable domain comprising the CDR-L1 of SEQ ID NO: 159 or having 1, 2, 3, 4 or 5 amino acid alterations relative to SEQ ID NO:159, the CDR-L2 of SEQ ID NO: 171 or having 1, 2, 3, 4 or 5 amino acid alterations relative to SEQ ID NO:171, and the CDR-L3 of SEQ ID NO: 184 or having 1, 2, 3, 4 or 5 amino acid alterations relative to SEQ ID NO:184; (xxiv) a light chain variable domain comprising the CDR-L1 of SEQ ID NO: 159 or having 1, 2, 3, 4 or 5 amino acid alterations relative to SEQ ID NO:159, the CDR-L2 of SEQ ID NO: 173 or having 1, 2, 3, 4 or 5 amino acid alterations relative to SEQ ID NO:173, and the CDR-L3 of SEQ ID NO: 185 or having 1, 2, 3, 4 or 5 amino acid alterations relative to SEQ ID NO:185; (xxv) a light chain variable domain comprising the CDR-L1 of SEQ ID NO: 159 or having 1, 2, 3, 4 or 5 amino acid alterations relative to SEQ ID NO:159, the CDR-L2 of SEQ ID NO: 173 or having 1, 2, 3, 4 or 5 amino acid alterations relative to SEQ ID NO:173, and the CDR-L3 of SEQ ID NO: 186 or having 1, 2, 3, 4 or 5 amino acid alterations relative to SEQ ID NO:186; (xxvi) a light chain variable domain comprising the CDR-L1 of SEQ ID NO: 161 or having 1, 2, 3, 4 or 5 amino acid alterations relative to SEQ ID NO:161, the CDR-L2 of SEQ ID NO: 174 or having 1, 2, 3, 4 or 5 amino acid alterations relative to SEQ ID NO:174, and the CDR-L3 of SEQ ID NO: 187 or having 1, 2, 3, 4 or 5 amino acid alterations relative to SEQ ID NO:187; (xxvii) a light chain variable domain comprising the CDR-L1 of SEQ ID NO: 162 or having 1, 2, 3, 4 or 5 amino acid alterations relative to SEQ ID NO:162, the CDR-L2 of SEQ ID NO: 174 or having 1, 2, 3, 4 or 5 amino acid alterations relative to SEQ ID NO:174, and the CDR-L3 of SEQ ID NO: 188 or having 1, 2, 3, 4 or 5 amino acid alterations relative to SEQ ID NO:188; (xxviii) a light chain variable domain comprising the CDR-L1 of SEQ ID NO: 163 or having 1, 2, 3, 4 or 5 amino acid alterations relative to SEQ ID NO:163, the CDR-L2 of SEQ ID NO: 174 or having 1, 2, 3, 4 or 5 amino acid alterations relative to SEQ ID NO:174, and the CDR-L3 of SEQ ID NO: 188 or having 1, 2, 3, 4 or 5 amino acid alterations relative to SEQ ID NO:188; (xxix) a light chain variable domain comprising the CDR-L1 of SEQ ID NO: 164 or having 1, 2, 3, 4 or 5 amino acid alterations relative to SEQ ID NO:164, the CDR-L2 of SEQ ID NO: 174 or having 1, 2, 3, 4 or 5 amino acid alterations relative to SEQ ID NO:174, and the CDR-L3 of SEQ ID NO: 189 or having 1, 2, 3, 4 or 5 amino acid alterations relative to SEQ ID NO:189; ( )=) a light chain variable domain comprising the CDR-L1 of SEQ ID NO: 165 or having 1, 2, 3, 4 or 5 amino acid alterations relative to SEQ ID NO:165, the CDR-L2 of SEQ ID NO: 175 or having 1, 2, 3, 4 or 5 amino acid alterations relative to SEQ ID NO:175, and the CDR-L3 of SEQ ID NO: 190 or having 1, 2, 3, 4 or 5 amino acid alterations relative to SEQ ID NO:190; (xxxi) a light chain variable domain comprising the CDR-L1 of SEQ ID NO: 166 or having 1, 2, 3, 4 or 5 amino acid alterations relative to SEQ ID NO:166, the CDR-L2 of SEQ ID NO: 176 or having 1, 2, 3, 4 or 5 amino acid alterations relative to SEQ ID NO:176, and the CDR-L3 of SEQ ID NO: 191 or having 1, 2, 3, 4 or 5 amino acid alterations relative to SEQ ID NO:191; (xxxi) a light chain variable domain comprising the CDR-L1 of SEQ ID NO: 167 or having 1, 2, 3, 4 or 5 amino acid alterations relative to SEQ ID NO:167, the CDR-L2 of SEQ ID NO: 177 or having 1, 2, 3, 4 or 5 amino acid alterations relative to SEQ ID NO:177, and the CDR-L3 of SEQ ID NO: 192 or having 1, 2, 3, 4 or 5 amino acid alterations relative to SEQ ID NO:192; (xxii) a light chain variable domain comprising the CDR-L1 of SEQ ID NO: 168 or having 1, 2, 3, 4 or 5 amino acid alterations relative to SEQ ID NO:168, the CDR-L2 of SEQ ID NO: 178 or having 1, 2, 3, 4 or 5 amino acid alterations relative to SEQ ID NO:178, and the CDR-L3 of SEQ ID NO: 193 or having 1, 2, 3, 4 or 5 amino acid alterations relative to SEQ ID NO:193; (xxiii) a light chain variable domain comprising the CDR-L1 of SEQ ID NO: 169 or having 1, 2, 3, 4 or 5 amino acid alterations relative to SEQ ID NO:169, the CDR-L2 of SEQ ID NO: 179 or having 1, 2, 3, 4 or 5 amino acid alterations relative to SEQ ID NO:179, and the CDR-L3 of SEQ ID NO: 194 or having 1, 2, 3, 4 or 5 amino acid alterations relative to SEQ ID NO:194.

In an embodiment, the light chain variable domain of the MuSK antibody based molecule disclosed herein comprises (i) a light chain variable domain comprising the CDR-L1 of SEQ ID NO: 49, the CDR-L2 of SEQ ID NO: 65, and the CDR-L3 of SEQ ID NO: 81; (ii) a light chain variable domain comprising the CDR-L1 of SEQ ID NO: 50, the CDR-L2 of SEQ ID NO: 66, and the CDR-L3 of SEQ ID NO: 82; (iii) a light chain variable domain comprising the CDR-L1 of SEQ ID NO: 51, the CDR-L2 of SEQ ID NO: 67, and the CDR-L3 of SEQ ID NO: 83; (iv) a light chain variable domain comprising the CDR-L1 of SEQ ID NO: 52, the CDR-L2 of SEQ ID NO: 68, and the CDR-L3 of SEQ ID NO: 84; (v) a light chain variable domain comprising the CDR-L1 of SEQ ID NO: 53, the CDR-L2 of SEQ ID NO: 69, and the CDR-L3 of SEQ ID NO: 85; (vi) a light chain variable domain comprising the CDR-L1 of SEQ ID NO: 54, the CDR-L2 of SEQ ID NO: 70, and the CDR-L3 of SEQ ID NO: 86; (vii) a light chain variable domain comprising the CDR-L1 of SEQ ID NO: 55, the CDR-L2 of SEQ ID NO: 71, and the CDR-L3 of SEQ ID NO: 87; (viii) a light chain variable domain comprising the CDR-L1 of SEQ ID NO: 56, the CDR-L2 of SEQ ID NO: 72, and the CDR-L3 of SEQ ID NO: 88; (ix) a light chain variable domain comprising the CDR-L1 of SEQ ID NO: 57, the CDR-L2 of SEQ ID NO: 73, and the CDR-L3 of SEQ ID NO: 89; (x) a light chain variable domain comprising the CDR-L1 of SEQ ID NO: 58, the CDR-L2 of SEQ ID NO: 74, and the CDR-L3 of SEQ ID NO: 90; (xi) a light chain variable domain comprising the CDR-L1 of SEQ ID NO: 59, the CDR-L2 of SEQ ID NO: 75, and the CDR-L3 of SEQ ID NO: 91; (xii) a light chain variable domain comprising the CDR-L1 of SEQ ID NO: 60, the CDR-L2 of SEQ ID NO: 76, and the CDR-L3 of SEQ ID NO: 92; (xiii) a light chain variable domain comprising the CDR-L1 of SEQ ID NO: 61, the CDR-L2 of SEQ ID NO: 77, and the CDR-L3 of SEQ ID NO: 93; (xiv) a light chain variable domain comprising the CDR-L1 of SEQ ID NO: 62, the CDR-L2 of SEQ ID NO: 78, and the CDR-L3 of SEQ ID NO: 94; (xv) a light chain variable domain comprising the CDR-L1 of SEQ ID NO: 63, the CDR-L2 of SEQ ID NO: 79, and the CDR-L3 of SEQ ID NO: 95; (xvi) a light chain variable domain comprising the CDR-L1 of SEQ ID NO: 64, the CDR-L2 of SEQ ID NO: 80, and the CDR-L3 of SEQ ID NO: 96; (xvii) a light chain variable domain comprising the CDR-L1 of SEQ ID NO: 141, the CDR-L2 of SEQ ID NO: 143, and the CDR-L3 of SEQ ID NO: 145; (xviii) a light chain variable domain comprising the CDR-L1 of SEQ ID NO: 142, the CDR-L2 of SEQ ID NO: 144, and the CDR-L3 of SEQ ID NO: 146. The sequences of the light chain CDRs are provided in Table 2 below.

In an embodiment, the light chain variable domain of the MuSK antibody based molecule disclosed herein comprises (xix) a light chain variable domain comprising the CDR-L1 of SEQ ID NO: 159, the CDR-L2 of SEQ ID NO: 170, and the CDR-L3 of SEQ ID NO: 180; (xx) a light chain variable domain comprising the CDR-L1 of SEQ ID NO: 159, the CDR-L2 of SEQ ID NO: 171, and the CDR-L3 of SEQ ID NO: 181; (xxi) a light chain variable domain comprising the CDR-L1 of SEQ ID NO: 160, the CDR-L2 of SEQ ID NO: 172, and the CDR-L3 of SEQ ID NO: 182; (xxii) a light chain variable domain comprising the CDR-L1 of SEQ ID NO: 159, the CDR-L2 of SEQ ID NO: 172, and the CDR-L3 of SEQ ID NO: 183; (xxiii) a light chain variable domain comprising the CDR-L1 of SEQ ID NO: 159, the CDR-L2 of SEQ ID NO: 171, and the CDR-L3 of SEQ ID NO: 184; (xxiv) a light chain variable domain comprising the CDR-L1 of SEQ ID NO: 159, the CDR-L2 of SEQ ID NO: 173, and the CDR-L3 of SEQ ID NO: 185; (xxv) a light chain variable domain comprising the CDR-L1 of SEQ ID NO: 159, the CDR-L2 of SEQ ID NO: 173, and the CDR-L3 of SEQ ID NO: 186; (xxvi) a light chain variable domain comprising the CDR-L1 of SEQ ID NO: 161, the CDR-L2 of SEQ ID NO: 174, and the CDR-L3 of SEQ ID NO: 187; (xxvii) a light chain variable domain comprising the CDR-L1 of SEQ ID NO: 162, the CDR-L2 of SEQ ID NO: 174, and the CDR-L3 of SEQ ID NO: 188; (xxviii) a light chain variable domain comprising the CDR-L1 of SEQ ID NO: 163, the CDR-L2 of SEQ ID NO: 174, and the CDR-L3 of SEQ ID NO: 188; (xxix) a light chain variable domain comprising the CDR-L1 of SEQ ID NO: 164, the CDR-L2 of SEQ ID NO: 174, and the CDR-L3 of SEQ ID NO: 189; (xxx) a light chain variable domain comprising the CDR-L1 of SEQ ID NO: 165, the CDR-L2 of SEQ ID NO: 175, and the CDR-L3 of SEQ ID NO: 190; (xxxi) a light chain variable domain comprising the CDR-L1 of SEQ ID NO: 166, the CDR-L2 of SEQ ID NO: 176, and the CDR-L3 of SEQ ID NO: 191; (xxxi) a light chain variable domain comprising the CDR-L1 of SEQ ID NO: 167, the CDR-L2 of SEQ ID NO: 177, and the CDR-L3 of SEQ ID NO: 192; (xxxii) a light chain variable domain comprising the CDR-L1 of SEQ ID NO: 168, the CDR-L2 of SEQ ID NO: 178, and the CDR-L3 of SEQ ID NO: 193; (xxxiii) a light chain variable domain comprising the CDR-L1 of SEQ ID NO: 169, the CDR-L2 of SEQ ID NO: 179, and the CDR-L3 of SEQ ID NO: 194.

In an embodiment, the light chain variable domain of the MuSK antibody based molecule disclosed herein comprises the CDR-L1 of SEQ ID NO: 159, the CDR-L2 of SEQ ID NO: 172, and the CDR-L3 of SEQ ID NO: 195 or a CDR-L3 having 1, 2, 3, 4 or 5 amino acid alterations relative to the amino acid sequence of SEQ ID NO: 195, wherein said alteration is present at residue 1, 2, 6, 7, 8, 9, 10, 11, 12, 13, 14, 15, 16, 17, or any combination thereof.

In an embodiment, the antibody-based molecule that binds to human muscle-specific tyrosine-protein kinase (MuSK) comprises a light chain variable domain, where the light chain variable domain comprises:
a CDR-L1 amino acid sequence comprising SEQ ID NO:159 or having 1, 2, 3, 4 or 5 amino acid alternations relative to SEQ ID NO: 159,
a CDR-L2 amino acid sequence comprising SEQ ID NO: 172 or having 1, 2, 3, 4 or 5 amino acid alternations relative to SEQ ID NO: 172, and
a CDR-L3 amino acid sequence comprising SEQ ID NO: 195 or having 1, 2, 3, 4 or 5 amino acid alternations relative SEQ ID NO:195 (3B2g2m1).

In an embodiment, the antibody-based molecule that binds to human muscle-specific tyrosine-protein kinase (MuSK) comprises a light chain variable domain, where the light chain variable domain comprises:
a CDR-L1 amino acid sequence comprising or consisting of SEQ ID NO: 159,
a CDR-L2 amino acid sequence comprising or consisting of SEQ ID NO: 172, and
a CDR-L3 amino acid sequence comprising or consisting of SEQ ID NO:195 (3B2g2m1).

The sequences of the light chain CDRs are provided in Table 2 below.

TABLE 2
VL CDR Sequences

| | LCDR1 | | LCDR2 | | LCDR3 | |
|---|---|---|---|---|---|---|
| mAb/ | | SEQ | | SEQ | | SEQ |
| Fab | | ID | | ID | | ID |
| name | Sequence | NO: | Sequence | NO: | Sequence | NO: |
| X1 | RASQSVSSAVA | 49 | SASSLYS | 65 | QQSSSSLIT | 81 |
| X2 | RASQSVSSAVA | 50 | SASSLYS | 66 | QQSGVWLIT | 82 |
| X3 | RASQSVSSAVA | 51 | SASSLYS | 67 | QQSSSSLIT | 83 |
| X4 | RASQSVSSAVA | 52 | SASSLYS | 68 | QQSYKPGALIT | 84 |
| X5 | RASQSVSSAVA | 53 | SASSLYS | 69 | QQSSSSLIT | 85 |
| X6 | RASQSVSSAVA | 54 | SASSLYS | 70 | QQSSSSLIT | 86 |
| X7 | RASQSVSSAVA | 55 | SASSLYS | 71 | QQSSRSSLLT | 87 |
| X8 | RASQSVSSAVA | 56 | SASSLYS | 72 | QQSSSSLIT | 88 |
| X9 | RASQSVSSAVA | 57 | SASSLYS | 73 | QQSSSSLIT | 89 |
| X10 | RASQSVSSAVA | 58 | SASSLYS | 74 | QQSLWYPVT | 90 |
| X11 | RASQSVSSAVA | 59 | SASSLYS | 75 | QQNSYYLIT | 91 |
| X12 | RASQSVSSAVA | 60 | SASSLYS | 76 | QQSSSSLIT | 92 |
| X13 | RASQSVSSAVA | 61 | SASSLYS | 77 | QQSYGSFSLIT | 93 |
| X14 | RASQSVSSAVA | 62 | SASSLYS | 78 | QQGSYHLIT | 94 |
| X15 | RASQSVSSAVA | 63 | SASSLYS | 79 | QQSGVWLIT | 95 |
| X16 | RASQSVSSAVA | 64 | SASSLYS | 80 | QQWSSAQALIT | 96 |
| X17 | RASQSVSSAVA | 141 | SASSLYS | 143 | QQSYKPGALIT | 145 |
| X18 | RASQSVSSAVA | 142 | SASSLYS | 144 | QQSYWWPIT | 146 |
| 14D10 | GLSSGSVTSSNYPD | 159 | TTNSRHS | 170 | ALYMGGGSNVYV | 180 |
| 7G4 | GLSSGSVTSSNYPD | 159 | STNSRHS | 171 | ALYMGRGSNKDYV | 181 |
| 3C4 | GLSSGSVTASNYPD | 160 | STDSRHS | 172 | ALYMYSDSKLYV | 182 |
| 3B2 | GLSSGSVTSSNYPD | 159 | STDSRHS | 172 | GLYMYSGSKNYV | 183 |
| 3G3 | GLSSGSVTSSNYPD | 159 | STNSRHS | 171 | ALYMGSDIRNYV | 184 |
| 31G2 | GLSSGSVTSSNYPD | 159 | STNSRLS | 173 | ALYMGSGSRNYV | 185 |
| 31B7 | GLSSGSVTSSNYPD | 159 | STNSRLS | 173 | ALYMGSESRNYV | 186 |
| 17H10 | GGNRIGGKSVQ | 161 | ADSRRPS | 174 | HVWGSTASAD | 187 |
| 23B6 | GGDNIGSKNAQ | 162 | ADSRRPS | 174 | HVWDSSTNAW | 188 |
| 30E1 | GGDNIGSKNTQ | 163 | ADSRRPS | 174 | HVWDSSTNAW | 188 |
| 30A11 | GGDNIASKNVQ | 164 | ADSRRPS | 174 | QVWDSSTNVAV | 189 |
| 16F11 | KSSQSVVFGSNQKSY | 165 | YASTQES | 175 | QQAYSAPT | 190 |
| | LN | | | | | |
| 4C11 | RSSQSVLYSSNQKNY | 166 | WASARES | 176 | QQSYKPPYG | 191 |
| | LN | | | | | |
| 7A12 | ESSQSVLYNQKNYLN | 167 | WASTRQS | 177 | QQAYNAPLT | 192 |
| 7G12 | KSSQRVQLGSNQKSY | 168 | YASTQQS | 178 | QQGYSAPFT | 193 |
| | LN | | | | | |
| 7B8 | KSSQSVLYNQKNYLA | 169 | WASTRES | 179 | QQGYSVPYT | 194 |
| 3B2g1m1 | GLSSGSVTSSNYPD | 159 | STDSRHS | 172 | GLYMYSGSKNYV | 183 |
| 3B2g1m2 | GLSSGSVTSSNYPD | 159 | STDSRHS | 172 | GLYMYSGSKNYV | 183 |
| 3B2g1m4 | GLSSGSVTSSNYPD | 159 | STDSRHS | 172 | GLYMYSGSKNYV | 183 |
| 3B2g2m1 | GLSSGSVTSSNYPD | 159 | STDSRHS | 172 | GLYSYSGSKNYV | 195 |
| 3B2g2m2 | GLSSGSVTSSNYPD | 159 | STDSRHS | 172 | GLYSYSGSKNYV | 195 |
| 3B2g2m4 | GLSSGSVTSSNYPD | 159 | STDSRHS | 172 | GLYSYSGSKNYV | 195 |

Suitable amino acid modifications to the heavy chain CDR sequences and/or the light chain CDR sequences of the MuSK antibody-based molecule disclosed herein include, for example, conservative substitutions or functionally equivalent amino acid residue substitutions that result in variant CDR sequences having similar or enhanced binding characteristics to those of the CDR sequences disclosed herein as described above. Encompassed by the present invention are CDRs of Tables 1 and 2 containing 0, 1, 2, 3, 4, 5, or more amino acid alterations (depending on the length of the CDR) that maintain or enhance MuSK binding of the antibody. Suitable amino acid modifications to the heavy chain CDR sequences of Table 1 and/or the light chain CDR sequences of Tables 1 and 2 include, for example, conservative substitutions or functionally equivalent amino acid residue substitutions that result in variant CDR sequences having similar or enhanced binding characteristics to those of the CDR sequences of Table 1 and Table 2. Conservative substitutions are those that take place within a family of amino acids that are related in their side chains. Genetically encoded amino acids can be divided into four families: (1) acidic (aspartate, glutamate); (2) basic (lysine, arginine, histidine); (3) nonpolar (alanine, valine, leucine, isoleucine, proline, phenylalanine, methionine, tryptophan); and (4) uncharged polar (glycine, asparagine, glutamine, cysteine, serine, threonine, tyrosine). Phenylalanine, tryptophan, and tyrosine are sometimes classified jointly as aromatic amino acids. Alternatively, the amino acid repertoire can be grouped as (1) acidic (aspartate, glutamate); (2) basic (lysine, arginine histidine), (3) aliphatic (glycine, alanine, valine, leucine, isoleucine, serine, threonine), with serine and threonine optionally grouped separately as aliphatic-hydroxyl; (4) aromatic (phenylalanine, tyrosine, tryptophan); (5) amide (asparagine, glutamine); and (6) sulfur-containing (cysteine and methionine) (Stryer (ed.), Biochemistry, 2nd ed, WH Freeman and Co., 1981, which is hereby incorporated by reference in its entirety). Non-conservative substitutions can also be made to the heavy chain CDR sequences of Table 1 and the light chain CDR sequences of Table 2. Non-conservative substitutions involve substituting one or more amino acid residues of the CDR with one or more amino acid residues from a different class of amino acids to improve or enhance the binding properties of CDR. The amino acid sequences of the heavy chain variable domain CDRs of Table 1 and/or the light chain variable domain CDRs of Table 2 may further comprise one or more internal neutral amino acid insertions or deletions that maintain or enhance MuSK binding.

In an embodiment, the MuSK antibody-based molecule comprises:
(i) a heavy chain variable domain comprising the CDR-H1 of SEQ ID NO: 1, the CDR-H2 of SEQ ID NO: 17, and the CDR-H3 of SEQ ID NO: 33, and a light chain variable domain comprising the CDR-L1 of SEQ ID NO: 49, the CDR-L2 of SEQ ID NO: 65, and the CDR-L3 of SEQ ID NO: 81;
(ii) a heavy chain variable domain comprising the CDR-H1 of SEQ ID NO: 2, the CDR-H2 of SEQ ID NO: 18, and the CDR-H3 of SEQ ID NO: 34, and a light chain variable domain comprising the CDR-L1 of SEQ ID NO: 50, the CDR-L2 of SEQ ID NO: 66, and the CDR-L3 of SEQ ID NO: 82;
(iii) a heavy chain variable domain comprising the CDR-H1 of SEQ ID NO: 3, the CDR-H2 of SEQ ID NO: 19, and the CDR-H3 of SEQ ID NO: 35, and a light chain variable domain comprising the CDR-L1 of SEQ ID NO: 51, the CDR-L2 of SEQ ID NO: 67, and the CDR-L3 of SEQ ID NO: 83;
(iv) a heavy chain variable domain comprising the CDR-H1 of SEQ ID NO: 4, the CDR-H2 of SEQ ID NO: 20, and the CDR-H3 of SEQ ID NO: 36, and a light chain variable domain comprising the CDR-L1 of SEQ ID NO: 52, the CDR-L2 of SEQ ID NO: 68, and the CDR-L3 of SEQ ID NO: 84;
(v) a heavy chain variable domain comprising the CDR-H1 of SEQ ID NO: 5, the CDR-H2 of SEQ ID NO: 21, and the CDR-H3 of SEQ ID NO: 37, and a light chain variable domain comprising the CDR-L1 of SEQ ID NO: 53, the CDR-L2 of SEQ ID NO: 69, and the CDR-L3 of SEQ ID NO: 85;
(vi) a heavy chain variable domain comprising the CDR-H1 of SEQ ID NO: 6, the CDR-H2 of SEQ ID NO: 22, and the CDR-H3 of SEQ ID NO: 38, and a light chain variable domain comprising the CDR-L1 of SEQ ID NO: 54, the CDR-L2 of SEQ ID NO: 70, and the CDR-L3 of SEQ ID NO: 86;
(vii) a heavy chain variable domain comprising the CDR-H1 of SEQ ID NO: 7, the CDR-H2 of SEQ ID NO: 23, and the CDR-H3 of SEQ ID NO: 39, and a light chain variable domain comprising the CDR-L1 of SEQ ID NO: 55, the CDR-L2 of SEQ ID NO:71, and the CDR-L3 of SEQ ID NO: 87;
(viii) a heavy chain variable domain comprising the CDR-H1 of SEQ ID NO: 8, the CDR-H2 of SEQ ID NO: 24, and the CDR-H3 of SEQ ID NO: 40, and a light chain variable domain comprising the CDR-L1 of SEQ ID NO: 56, the CDR-L2 of SEQ ID NO: 72, and the CDR-L3 of SEQ ID NO: 88;
(ix) a heavy chain variable domain comprising the CDR-H1 of SEQ ID NO: 9, the CDR-H2 of SEQ ID NO: 25, and the CDR-H3 of SEQ ID NO: 41, and a light chain variable domain comprising the CDR-L1 of SEQ ID NO: 57, the CDR-L2 of SEQ ID NO: 73, and the CDR-L3 of SEQ ID NO: 89;
(x) a heavy chain variable domain comprising the CDR-H1 of SEQ ID NO: 10, the CDR-H2 of SEQ ID NO: 26, and the CDR-H3 of SEQ ID NO: 42, and a light chain variable domain comprising the CDR-L1 of SEQ ID NO: 58, the CDR-L2 of SEQ ID NO: 74, and the CDR-L3 of SEQ ID NO: 90;
(xi) a heavy chain variable domain comprising the CDR-H1 of SEQ ID NO: 11, the CDR-H2 of SEQ ID NO: 27, and the CDR-H3 of SEQ ID NO: 43, and a light chain variable domain comprising the CDR-L1 of SEQ ID NO: 59, the CDR-L2 of SEQ ID NO: 75, and the CDR-L3 of SEQ ID NO: 91;
(xii) a heavy chain variable domain comprising the CDR-H1 of SEQ ID NO: 12, the CDR-H2 of SEQ ID NO: 28, and the CDR-H3 of SEQ ID NO: 44, and a light chain variable domain comprising the CDR-L1 of SEQ ID NO: 60, the CDR-L2 of SEQ ID NO: 76, and the CDR-L3 of SEQ ID NO: 92;
(xiii) a heavy chain variable domain comprising the CDR-H1 of SEQ ID NO: 13, the CDR-H2 of SEQ ID NO: 29, and the CDR-H3 of SEQ ID NO: 45, and a light chain variable domain comprising the CDR-L1 of SEQ ID NO: 61, the CDR-L2 of SEQ ID NO: 77, and the CDR-L3 of SEQ ID NO: 93;
(xiv) a heavy chain variable domain comprising the CDR-H1 of SEQ ID NO: 14, the CDR-H2 of SEQ ID NO: 30, and the CDR-H3 of SEQ ID NO: 46, and a light chain variable domain comprising the CDR-L1 of SEQ ID NO: 62, the CDR-L2 of SEQ ID NO: 78, and the CDR-L3 of SEQ ID NO: 94;
(xv) a heavy chain variable domain comprising the CDR-H1 of SEQ ID NO: 15, the CDR-H2 of SEQ ID NO: 31, and the CDR-H3 of SEQ ID NO: 47, and a light chain variable domain comprising the CDR-L1 of SEQ ID NO: 63, the CDR-L2 of SEQ ID NO: 79, and the CDR-L3 of SEQ ID NO: 95;
(xvi) a heavy chain variable domain comprising the CDR-H1 of SEQ ID NO: 16, the CDR-H2 of SEQ ID NO: 32, and the CDR-H3 of SEQ ID NO: 48, and a light chain variable domain comprising the CDR-L1 of SEQ ID NO: 64, the CDR-L2 of SEQ ID NO: 80, and the CDR-L3 of SEQ ID NO: 96;
(xvii) a heavy chain variable domain comprising the CDR-H1 of SEQ ID NO: 135, the CDR-H2 of SEQ ID NO: 137, and the CDR-H3 of SEQ ID NO: 139, and a light chain variable domain comprising the CDR-L1 of SEQ ID NO: 141, the CDR-L2 of SEQ ID NO: 143, and the CDR-L3 of SEQ ID NO: 145; and
(xviii) a heavy chain variable domain comprising the CDR-H1 of SEQ ID NO: 136, the CDR-H2 of SEQ ID NO: 138, and the CDR-H3 of SEQ ID NO: 140, and a light chain variable domain comprising the CDR-L1 of SEQ ID NO: 142, the CDR-L2 of SEQ ID NO: 144, and the CDR-L3 of SEQ ID NO: 146.

In an embodiment, the MuSK antibody-based molecule comprises:
(ii.a) a heavy chain variable domain comprising the CDR-H1 of SEQ ID NO: 2, the CDR-H2 of SEQ ID NO: 18, and the CDR-H3 of SEQ ID NO: 240, and a light chain variable domain comprising the CDR-L1 of SEQ ID NO: 50, the CDR-L2 of SEQ ID NO: 66, and the CDR-L3 of SEQ ID NO: 82 (X2m1);
(ii.b) a heavy chain variable domain comprising the CDR-H1 of SEQ ID NO: 2, the CDR-H2 of SEQ ID NO: 18, and the CDR-H3 of SEQ ID NO: 241, and a light chain variable domain comprising the CDR-L1 of SEQ ID NO: 50, the CDR-L2 of SEQ ID NO: 66, and the CDR-L3 of SEQ ID NO: 82 (X2m2);
(ii.c) a heavy chain variable domain comprising the CDR-H1 of SEQ ID NO: 2, the CDR-H2 of SEQ ID NO: 18, and the CDR-H3 of SEQ ID NO: 242, and a light chain variable domain comprising the CDR-L1 of SEQ ID NO: 50, the CDR-L2 of SEQ ID NO: 66, and the CDR-L3 of SEQ ID NO: 82 (X2m3);
(ii.d) a heavy chain variable domain comprising the CDR-H1 of SEQ ID NO: 2, the CDR-H2 of SEQ ID NO: 18, and the CDR-H3 of SEQ ID NO: 243, and a light chain variable domain comprising the CDR-L1 of SEQ ID NO: 50, the CDR-L2 of SEQ ID NO: 66, and the CDR-L3 of SEQ ID NO: 82 (X2m4);
(ii.e) a heavy chain variable domain comprising the CDR-H1 of SEQ ID NO: 2, the CDR-H2 of SEQ ID NO: 18, and the CDR-H3 of SEQ ID NO: 244, and a light chain variable domain comprising the CDR-L1 of SEQ ID NO: 50, the CDR-L2 of SEQ ID NO: 66, and the CDR-L3 of SEQ ID NO: 82 (X2m5);
(ii.f) a heavy chain variable domain comprising the CDR-H1 of SEQ ID NO: 2, the CDR-H2 of SEQ ID NO: 18, and the CDR-H3 of SEQ ID NO: 245, and a light chain variable domain comprising the CDR-L1 of SEQ ID NO: 50, the CDR-L2 of SEQ ID NO: 66, and the CDR-L3 of SEQ ID NO: 82 (X2m6);
(ii.g) a heavy chain variable domain comprising the CDR-H1 of SEQ ID NO: 2, the CDR-H2 of SEQ ID NO: 18, and the CDR-H3 of SEQ ID NO: 246, and a light chain variable domain comprising the CDR-L1 of SEQ ID NO: 50, the CDR-L2 of SEQ ID NO: 66, and the CDR-L3 of SEQ ID NO: 82 (X2m7);
(ii.f) a heavy chain variable domain comprising the CDR-H1 of SEQ ID NO: 2, the CDR-H2 of SEQ ID NO: 18, and the CDR-H3 of SEQ ID NO: 247, and a light chain variable domain comprising the CDR-L1 of SEQ ID NO: 50, the CDR-L2 of SEQ ID NO: 66, and the CDR-L3 of SEQ ID NO: 82 (X2m8).

In an embodiment, the MuSK antibody-based molecule comprises:
(xvii.a) a heavy chain variable domain comprising the CDR-H1 of SEQ ID NO: 135, the CDR-H2 of SEQ ID NO: 137, and the CDR-H3 of SEQ ID NO: 248, and a light chain variable domain comprising the CDR-L1 of SEQ ID NO: 141, the CDR-L2 of SEQ ID NO: 143, and the CDR-L3 of SEQ ID NO: 145 (X17m1);
(xvii.b) a heavy chain variable domain comprising the CDR-H1 of SEQ ID NO: 135, the CDR-H2 of SEQ ID NO: 137, and the CDR-H3 of SEQ ID NO: 249, and a light chain variable domain comprising the CDR-L1 of SEQ ID NO: 141, the CDR-L2 of SEQ ID NO: 143, and the CDR-L3 of SEQ ID NO: 145 (X17m2);
(xvii.c) a heavy chain variable domain comprising the CDR-H1 of SEQ ID NO: 135, the CDR-H2 of SEQ ID NO: 137, and the CDR-H3 of SEQ ID NO: 250, and a light chain variable domain comprising the CDR-L1 of SEQ ID NO: 141, the CDR-L2 of SEQ ID NO: 143, and the CDR-L3 of SEQ ID NO: 145 (X17m3);
(xvii.d) a heavy chain variable domain comprising the CDR-H1 of SEQ ID NO: 135, the CDR-H2 of SEQ ID NO: 137, and the CDR-H3 of SEQ ID NO: 251, and a light chain variable domain comprising the CDR-L1 of SEQ ID NO: 141, the CDR-L2 of SEQ ID NO: 143, and the CDR-L3 of SEQ ID NO: 145 (X17m6).

In an embodiment, the MuSK antibody-based molecule comprises:
(i) a heavy chain variable domain comprising the CDR-H1 of SEQ ID NO: 147, the CDR-H2 of SEQ ID NO: 150, and the CDR-H3 of SEQ ID NO: 156, and the light chain variable domain comprising the CDR-L1 of SEQ ID NO: 159, the CDR-L2 of SEQ ID NO: 170, and the CDR-L3 of SEQ ID NO: 180 (14D10);
(ii) a heavy chain variable domain comprising the CDR-H1 of SEQ ID NO: 147, the CDR-H2 of SEQ ID NO: 150, and the CDR-H3 of SEQ ID NO: 156, and the light chain variable domain comprising the CDR-L1 of SEQ ID NO: 159, the CDR-L2 of SEQ ID NO: 171, and the CDR-L3 of SEQ ID NO: 181 (7G4);
(iii) a heavy chain variable domain comprising the CDR-H1 of SEQ ID NO: 147, the CDR-H2 of SEQ ID NO: 150, and the CDR-H3 of SEQ ID NO: 156, and the light chain variable domain comprising the CDR-L1 of SEQ ID NO: 160, the CDR-L2 of SEQ ID NO: 172, and the CDR-L3 of SEQ ID NO: 182 (3C4);
(iv) a heavy chain variable domain comprising the CDR-H1 of SEQ ID NO: 147, the CDR-H2 of SEQ ID NO: 150, and the CDR-H3 of SEQ ID NO: 156, and the light chain variable domain comprising the CDR-L1 of SEQ ID NO: 159, the CDR-L2 of SEQ ID NO: 172, and the CDR-L3 of SEQ ID NO: 183 (3B2);
(v) a heavy chain variable domain comprising the CDR-H1 of SEQ ID NO: 147, the CDR-H2 of SEQ ID NO: 150, and the CDR-H3 of SEQ ID NO: 156, and the light chain variable domain comprising the CDR-L1 of SEQ ID NO: 159, the CDR-L2 of SEQ ID NO: 171, and the CDR-L3 of SEQ ID NO: 184 (3G3);
(vi) a heavy chain variable domain comprising the CDR-H1 of SEQ ID NO: 147, the CDR-H2 of SEQ ID NO: 150, and the CDR-H3 of SEQ ID NO: 156, and the light chain variable domain comprising the CDR-L1 of SEQ ID NO: 159, the CDR-L2 of SEQ ID NO: 173, and the CDR-L3 of SEQ ID NO: 185 (31G2);
(vii) a heavy chain variable domain comprising the CDR-H1 of SEQ ID NO: 147, the CDR-H2 of SEQ ID NO: 150, and the CDR-H3 of SEQ ID NO: 156, and the light chain variable domain comprising the CDR-L1 of SEQ ID NO: 159, the CDR-L2 of SEQ ID NO: 173, and the CDR-L3 of SEQ ID NO: 186 (31B7);
(viii) a heavy chain variable domain comprising the CDR-H1 of SEQ ID NO: 148, the CDR-H2 of SEQ ID NO: 151, and the CDR-H3 of SEQ ID NO: 157, and the light chain variable domain comprising the CDR-L1 of SEQ ID NO: 161, the CDR-L2 of SEQ ID NO: 174, and the CDR-L3 of SEQ ID NO: 187 (17H10);
(ix) a heavy chain variable domain comprising the CDR-H1 of SEQ ID NO: 148, the CDR-H2 of SEQ ID NO: 151, and the CDR-H3 of SEQ ID NO: 157, and the light chain variable domain comprising the CDR-L1 of SEQ ID NO: 162, the CDR-L2 of SEQ ID NO: 174, and the CDR-L3 of SEQ ID NO: 188 (2396);
(x) a heavy chain variable domain comprising the CDR-H1 of SEQ ID NO: 148, the CDR-H2 of SEQ ID NO: 151, and the CDR-H3 of SEQ ID NO: 157, and the light chain variable domain comprising the CDR-L1 of SEQ ID NO: 163, the CDR-L2 of SEQ ID NO: 174, and the CDR-L3 of SEQ ID NO: 188 (30E1);
(xi) a heavy chain variable domain comprising the CDR-H1 of SEQ ID NO: 148, the CDR-H2 of SEQ ID NO: 151, and the CDR-H3 of SEQ ID NO: 157, and the light chain variable domain comprising the CDR-L1 of SEQ ID NO: 164, the CDR-L2 of SEQ ID NO: 174, and the CDR-L3 of SEQ ID NO: 189 (30A11);
(xii) a heavy chain variable domain comprising the CDR-H1 of SEQ ID NO: 149, the CDR-H2 of SEQ ID NO: 152, and the CDR-H3 of SEQ ID NO: 158, and the light chain variable domain comprising the CDR-L1 of SEQ ID NO: 165, the CDR-L2 of SEQ ID NO: 175, and the CDR-L3 of SEQ ID NO: 190 (16F11);
(xiii) a heavy chain variable domain comprising the CDR-H1 of SEQ ID NO: 149, the CDR-H2 of SEQ ID NO: 152, and the CDR-H3 of SEQ ID NO: 158, and the light chain variable domain comprising the CDR-L1 of SEQ ID NO: 166, the CDR-L2 of SEQ ID NO: 176, and the CDR-L3 of SEQ ID NO: 191 (4C11);
(xiv) a heavy chain variable domain comprising the CDR-H1 of SEQ ID NO: 149, the CDR-H2 of SEQ ID NO: 152, and the CDR-H3 of SEQ ID NO: 158, and the light chain variable domain comprising the CDR-L1 of SEQ ID NO: 167, the CDR-L2 of SEQ ID NO: 177, and the CDR-L3 of SEQ ID NO: 192 (7A12);
(xv) a heavy chain variable domain comprising the CDR-H1 of SEQ ID NO: 149, the CDR-H2 of SEQ ID NO: 152, and the CDR-H3 of SEQ ID NO: 158, and the light chain variable domain comprising the CDR-L1 of SEQ ID NO: 168, the CDR-L2 of SEQ ID NO: 178, and the CDR-L3 of SEQ ID NO: 193 (7G12);
(xvi) a heavy chain variable domain comprising the CDR-H1 of SEQ ID NO: 149, the CDR-H2 of SEQ ID NO: 152, and the CDR-H3 of SEQ ID NO: 158, and the light chain variable domain comprising the CDR-L1 of SEQ ID NO: 169, the CDR-L2 of SEQ ID NO: 179, and the CDR-L3 of SEQ ID NO: 194 (7B8);
(xvii) a heavy chain variable domain comprising the CDR-H1 of SEQ ID NO: 147, the CDR-H2 of SEQ ID NO: 153, and the CDR-H3 of SEQ ID NO: 156, and the light chain variable domain comprising the CDR-L1 of SEQ ID NO: 159, the CDR-L2 of SEQ ID NO: 172, and the CDR-L3 of SEQ ID NO: 183 (3B2g1m1);
(xviii) a heavy chain variable domain comprising the CDR-H1 of SEQ ID NO: 147, the CDR-H2 of SEQ ID NO: 154, and the CDR-H3 of SEQ ID NO: 156, and the light chain variable domain comprising the CDR-L1 of SEQ ID NO: 159, the CDR-L2 of SEQ ID NO: 172, and the CDR-L3 of SEQ ID NO: 183 (3B2g1m2);
(xvix) a heavy chain variable domain comprising the CDR-H1 of SEQ ID NO: 147, the CDR-H2 of SEQ ID NO: 155, and the CDR-H3 of SEQ ID NO: 156, and the light chain variable domain comprising the CDR-L1 of SEQ ID NO: 159, the CDR-L2 of SEQ ID NO: 172, and the CDR-L3 of SEQ ID NO: 183 (3B2g1m4);
(xx) a heavy chain variable domain comprising the CDR-H1 of SEQ ID NO: 147, the CDR-H2 of SEQ ID NO: 153, and the CDR-H3 of SEQ ID NO: 156, and the light chain variable domain comprising the CDR-L1 of SEQ ID NO: 159, the CDR-L2 of SEQ ID NO: 172, and the CDR-L3 of SEQ ID NO: 195 (3B2g2m1);
(xxi) a heavy chain variable domain comprising the CDR-H1 of SEQ ID NO: 147, the CDR-H2 of SEQ ID NO: 154, and the CDR-H3 of SEQ ID NO: 156, and the light chain variable domain comprising the CDR-L1 of SEQ ID NO: 159, the CDR-L2 of SEQ ID NO: 172, and the CDR-L3 of SEQ ID NO: 195 (3B2g2m2); and
(xxii) a heavy chain variable domain comprising the CDR-H1 of SEQ ID NO: 147, the CDR-H2 of SEQ ID NO: 155, and the CDR-H3 of SEQ ID NO: 156, and the light chain variable domain comprising the CDR-L1 of SEQ ID NO: 159, the CDR-L2 of SEQ ID NO: 172, and the CDR-L3 of SEQ ID NO: 195 (3B2g2m4)

In an embodiment, the antibody-based molecule that binds to human muscle-specific tyrosine-protein kinase (MuSK) comprises a heavy chain variable domain and a light chain variable domain,
where the heavy chain variable domain comprises:
   a CDR-H1 amino acid sequence comprising SEQ ID NO: 147 or having 1, 2, 3, 4 or 5 amino acid alterations relative to SEQ ID NO: 147,
   a CDR-H2 amino acid sequence comprising SEQ ID NO: 153 or having 1, 2, 3, 4 or 5 amino acid alterations relative to SEQ ID NO: 153, and
   a CDR-H3 amino acid sequence comprising SEQ ID NO:156 or having 1, 2, 3, 4 or 5 amino acid alterations relative to SEQ ID NO:156 (3B2g2m1) and
where the light chain variable domain comprises:
   a CDR-L1 amino acid sequence comprising SEQ ID NO: 159 or having 1, 2, 3, 4 or 5 amino acid alterations relative to SEQ ID NO: 159,
   a CDR-L2 amino acid sequence comprising SEQ ID NO: 172 or 1, 2, 3, 4 or 5 amino acid alterations relative to SEQ ID NO: 172, and
   a CDR-L3 amino acid sequence comprising SEQ ID NO: 195 or having 1, 2, 3, 4 or 5 amino acid alterations relative to SEQ ID NO:195 (3B2g2m1).

In an embodiment, the CDR-H2 of the antibody comprises a proline (P) at position 3, a tryptophan (W) at position 4, and a serine (S) or asparagine (N) at position 5. In a preferred embodiment, the CDR-H2 of the antibody comprises a proline (P) at position 3, a tryptophan (W) at position 4, and a serine (S) at position 5.

In a more preferred embodiment, the antibody-based molecule that binds to human muscle-specific tyrosine-protein kinase (MuSK) comprises a heavy chain variable domain and a light chain variable domain,
where the heavy chain variable domain comprises:
   a CDR-H1 amino acid sequence comprising or consisting of SEQ ID NO: 147,
   a CDR-H2 amino acid sequence comprising or consisting of SEQ ID NO: 153, and
   a CDR-H3 amino acid sequence comprising or consisting of SEQ ID NO:156 (3B2g2m1) and
where the light chain variable domain comprises:
   a CDR-L1 amino acid sequence comprising or consisting of SEQ ID NO: 159,
   a CDR-L2 amino acid sequence comprising or consisting of SEQ ID NO: 172, and
   a CDR-L3 amino acid sequence comprising or consisting of SEQ ID NO:195 (3B2g2m1).

The MuSK antibody-based molecule as described herein may comprise a variable light (VL) chain, a variable heavy (VH) chain, or a combination of VL and VH chains. In some embodiments, the VH chain of the MuSK antibody-based molecule comprises any one of the VH amino acid sequences provided in Table 3 below, or an amino acid sequence that is at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, identical or similar to any one of the VH amino acid sequences listed in Table 3. In some embodiments, the VL chain of the MuSK antibody-based molecule comprises any one of the VL amino acid sequences provided in Table 3 below, or an amino acid sequence that is at least 60%, identical or similar to any one of the VL amino acid sequences listed in Table 3. In an embodiment, the identity or similarity is at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%.

| mAb/Fab | | | SEQ ID |
|---|---|---|---|
| name | Domain | Sequence | NO: |
| X1 | VH | EVQLVESGGGLVQPGGSLRLSCAASGFTFSSSSIHWVRQAPGKGLEWVA | 97 |
| | | SISSSSGSTSYADSVKGRFTISADTSKNTAYLQMNSLRAEDTAVYYCARKY | |
| | | WSQYYWAHYYGGLDYWGQGTLVTVSS | |
| | VL | DIQMTQSPSSLSASVGDRVTITCRASQSVSSAVAWYQQKPGKAPKLLIYSA | 98 |
| | | SSLYSGVPSRFSGSRSGTDFTLTISSLQPEDFATYYCQQSSSSLITFGQGT | |
| | | KVEIK | |
| X2 | VH | EVQLVESGGGLVQPGGSLRLSCAASGFTVSSSSIHWVRQAPGKGLEWVA | 99 |
| | | SISSSYGSTSYADSVKGRFTISADTSKNTAYLQMNSLRAEDTAVYYCARSE | |
| | | GDRYVSGYMGMDYWGQGTLVTVSS | |
| X2m1 | VH | EVQLVESGGGLVQPGGSLRLSCAASGFTVSSSSIHWVRQAPGKGLEWVA | 252 |
| | | SISSSYGSTSYADSVKGRFTISADTSKNTAYLQMNSLRAEDTAVYYCARSE | |
| | | GDRYVSGYFGFDYWGQGTLVTVSS | |
| X2m2 | VH | EVQLVESGGGLVQPGGSLRLSCAASGFTVSSSSIHWVRQAPGKGLEWVA | 253 |
| | | SISSSYGSTSYADSVKGRFTISADTSKNTAYLQMNSLRAEDTAVYYCARSE | |
| | | GDRYVSGYFGLDYWGQGTLVTVSS | |
| X2m3 | VH | EVQLVESGGGLVQPGGSLRLSCAASGFTVSSSSIHWVRQAPGKGLEWVA | 254 |
| | | SISSSYGSTSYADSVKGRFTISADTSKNTAYLQMNSLRAEDTAVYYCARSE | |
| | | GDRYVSGYSGFDYWGQGTLVTVSS | |
| X2m4 | VH | EVQLVESGGGLVQPGGSLRLSCAASGFTVSSSSIHWVRQAPGKGLEWVA | 255 |
| | | SISSSYGSTSYADSVKGRFTISADTSKNTAYLQMNSLRAEDTAVYYCARSE | |
| | | GDRYVSGYSGLDYWGQGTLVTVSS | |
| X2m5 | VH | EVQLVESGGGLVQPGGSLRLSCAASGFTVSSSSIHWVRQAPGKGLEWVA | 256 |
| | | SISSSYGSTSYADSVKGRFTISADTSKNTAYLQMNSLRAEDTAVYYCARSE | |
| | | GDRYVSGYFGMDYWGQGTLVTVSS | |
| X2m6 | VH | EVQLVESGGGLVQPGGSLRLSCAASGFTVSSSSIHWVRQAPGKGLEWVA | 257 |
| | | SISSSYGSTSYADSVKGRFTISADTSKNTAYLQMNSLRAEDTAVYYCARSE | |
| | | GDRYVSGYSGMDYWGQGTLVTVSS | |
| X2m7 | VH | EVQLVESGGGLVQPGGSLRLSCAASGFTVSSSSIHWVRQAPGKGLEWVA | 258 |
| | | SISSSYGSTSYADSVKGRFTISADTSKNTAYLQMNSLRAEDTAVYYCARSE | |
| | | GDRYVSGYMGFDYWGQGTLVTVSS | |
| X2m8 | VH | EVQLVESGGGLVQPGGSLRLSCAASGFTVSSSSIHWVRQAPGKGLEWVA | 259 |
| | | SISSSYGSTSYADSVKGRFTISADTSKNTAYLQMNSLRAEDTAVYYCARSE | |
| | | GDRYVSGYMGLDYWGQGTLVTVSS | |
| X2 | VL | DIQMTQSPSSLSASVGDRVTITCRASQSVSSAVAWYQQKPGKAPKLLIYSA | 100 |
| | | SSLYSGVPSRFSGSRSGTDFTLTISSLQPEDFATYYCQQSGVWLITFGQGT | |
| | | KVEIK | |
| X3 | VH | EVQLVESGGGLVQPGGSLRLSCAASGFTISSSSIHWVRQAPGKGLEWVAS | 101 |
| | | ISSSSGYTYYADSVKGRFTISADTSKNTAYLQMNSLRAEDTAVYYCARSWY | |
| | | EMWMSGYFGFDYWGQGTLVTVSS | |
| | VL | DIQMTQSPSSLSASVGDRVTITCRASQSVSSAVAWYQQKPGKAPKLLIYSA | 102 |
| | | SSLYSGVPSRFSGSRSGTDFTLTISSLQPEDFATYYCQQSSSSLITFGQGT | |
| | | KVEIK | |
| X4 | VH | EVQLVESGGGLVQPGGSLRLSCAASGFTVSSSSIHWVRQAPGKGLEWVA | 103 |
| | | SISSSSGSTYYADSVKGRFTISADTSKNTAYLQMNSLRAEDTAVYYCARGE | |
| | | HDYYVFGYLGMDYWGQGTLVTVSS | |
| | VL | DIQMTQSPSSLSASVGDRVTITCRASQSVSSAVAWYQQKPGKAPKLLIYSA | 104 |
| | | SSLYSGVPSRFSGSRSGTDFTLTISSLQPEDFATYYCQQSYKPGALITFGQ | |
| | | GTKVEIK | |
| X5 | VH | EVQLVESGGGLVQPGGSLRLSCAASGFTFYSSSIHWVRQAPGKGLEWVA | 105 |
| | | SISSSSGSTSYADSVKGRFTISADTSKNTAYLQMNSLRAEDTAVYYCARSY | |
| | | TMFYYGGWYGSGYFGMDYWGQGTLVTVSS | |
| | VL | DIQMTQSPSSLSASVGDRVTITCRASQSVSSAVAWYQQKPGKAPKLLIYSA | 106 |
| | | SSLYSGVPSRFSGSRSGTDFTLTISSLQPEDFATYYCQQSSSSLITFGQGT | |
| | | KVEIK | |
| X6 | VH | EVQLVESGGGLVQPGGSLRLSCAASGFTFSSSSIHWVRQAPGKGLEWVA | 107 |
| | | SISSYSGYTYYADSVKGRFTISADTSKNTAYLQMNSLRAEDTAVYYCARTY | |
| | | GSYYVSSYTGMDYWGQGTLVTVSS | |
| | VL | DIQMTQSPSSLSASVGDRVTITCRASQSVSSAVAWYQQKPGKAPKLLIYSA | 108 |
| | | SSLYSGVPSRFSGSRSGTDFTLTISSLQPEDFATYYCQQSSSSLITFGQGT | |
| | | KVEIK | |
| X7 | VH | EVQLVESGGGLVQPGGSLRLSCAASGFTLYSSSIHWVRQAPGKGLEWVA | 109 |
| | | SISSSYSSTYYADSVKGRFTISADTSKNTAYLQMNSLRAEDTAVYYCARLA | |
| | | GLYHYPGYLGLDYWGQGTLVTVSS | |
| | VL | DIQMTQSPSSLSASVGDRVTITCRASQSVSSAVAWYQQKPGKAPKLLIYSA | 110 |
| | | SSLYSGVPSRFSGSRSGTDFTLTISSLQPEDFATYYCQQSSRSSLLTFGQG | |
| | | TKVEIK | |
| X8 | VH | EVQLVESGGGLVQPGGSLRLSCAASGFTVSSSSIHWVRQAPGKGLEWVA | 111 |
| | | SISSSSGSTSYADSVKGRFTISADTSKNTAYLQMNSLRAEDTAVYYCARSW | |
| | | SYHPWYYHVGWYTGLDYWGQGTLVTVSS | |
| | VL | DIQMTQSPSSLSASVGDRVTITCRASQSVSSAVAWYQQKPGKAPKLLIYSA | 112 |
| | | SSLYSGVPSRFSGSRSGTDFTLTISSLQPEDFATYYCQQSSSSLITFGQGT | |
| | | KVEIK | |
| X9 | VH | EVQLVESGGGLVQPGGSLRLSCAASGFTFSSSSIHWVRQAPGKGLEWVA | 113 |
| | | SIYSSSGSTYYADSVKGRFTISADTSKNTAYLQMNSLRAEDTAVYYCARSG | |
| | | GEFYITSYYGMDYWGQGTLVTVSS | |
| | VL | DIQMTQSPSSLSASVGDRVTITCRASQSVSSAVAWYQQKPGKAPKLLIYSA | 114 |
| | | SSLYSGVPSRFSGSRSGTDFTLTISSLQPEDFATYYCQQSSSSLITFGQGT | |
| | | KVEIK | |
| X10 | VH | EVQLVESGGGLVQPGGSLRLSCAASGFTFSSSSIHWVRQAPGKGLEWVA | 115 |
| | | SISSSYSSTSYADSVKGRFTISADTSKNTAYLQMNSLRAEDTAVYYCARKY | |
| | | YRWRHNKYQGFDYWGQGTLVTVSS | |
| | VL | DIQMTQSPSSLSASVGDRVTITCRASQSVSSAVAWYQQKPGKAPKLLIYSA | 116 |
| | | SSLYSGVPSRFSGSRSGTDFTLTISSLQPEDFATYYCQQSLWYPVTFGQG | |
| | | TKVEIK | |
| X11 | VH | EVQLVESGGGLVQPGGSLRLSCAASGFTISSSSIHWVRQAPGKGLEWVAS | 117 |
| | | ISSYSGSTYYADSVKGRFTISADTSKNTAYLQMNSLRAEDTAVYYCARSWG | |
| | | SYYVSGFVGFDYWGQGTLVTVSS | |
| | VL | DIQMTQSPSSLSASVGDRVTITCRASQSVSSAVAWYQQKPGKAPKLLIYSA | 118 |
| | | SSLYSGVPSRFSGSRSGTDFTLTISSLQPEDFATYYCQQNSYYLITFGQGT | |
| | | KVEIK | |
| X12 | VH | EVQLVESGGGLVQPGGSLRLSCAASGFTISSSSIHWVRQAPGKGLEWVAY | 119 |
| | | ISPSSGYTSYADSVKGRFTISADTSKNTAYLQMNSLRAEDTAVYYCARQYW | |
| | | VPQWWVITQYFGMDYWGQGTLVTVSS | |
| | VL | DIQMTQSPSSLSASVGDRVTITCRASQSVSSAVAWYQQKPGKAPKLLIYSA | 120 |
| | | SSLYSGVPSRFSGSRSGTDFTLTISSLQPEDFATYYCQQSSSSLITFGQGT | |
| | | KVEIK | |
| X13 | VH | EVQLVESGGGLVQPGGSLRLSCAASGFTISSSSIHWVRQAPGKGLEWVAS | 121 |
| | | ISSSSGSTSYADSVKGRFTISADTSKNTAYLQMNSLRAEDTAVYYCARSSE | |
| | | HWYTIGYYGIDYWGQGTLVTVSS | |
| | VL | DIQMTQSPSSLSASVGDRVTITCRASQSVSSAVAWYQQKPGKAPKLLIYSA | 122 |
| | | SSLYSGVPSRFSGSRSGTDFTLTISSLQPEDFATYYCQQSYGSFSLITFGQ | |
| | | GTKVEIK | |
| X14 | VH | EVQLVESGGGLVQPGGSLRLSCAASGFTFSSSSIHWVRQAPGKGLEWVA | 123 |
| | | SISSSSGYTYYADSVKGRFTISADTSKNTAYLQMNSLRAEDTAVYYCARGS | |
| | | HHWFLWIYSGLDYWGQGTLVTVSS | |
| | VL | DIQMTQSPSSLSASVGDRVTITCRASQSVSSAVAWYQQKPGKAPKLLIYSA | 124 |
| | | SSLYSGVPSRFSGSRSGTDFTLTISSLQPEDFATYYCQQGSYHLITFGQGT | |
| | | KVEIK | |
| X15 | VH | EVQLVESGGGLVQPGGSLRLSCAASGFTVSSSSIHWVRQAPGKGLEWVA | 125 |
| | | SISSSYGSTSYADSVKGRFTISADTSKNTAYLQMNSLRAEDTAVYYCARSE | |
| | | GDRYVSGYMGMDYWGQGTLVTVSS | |
| | VL | DIQMTQSPSSLSASVGDRVTITCRASQSVSSAVAWYQQKPGKAPKLLIYSA | 126 |
| | | SSLYSGVPSRFSGSRSGTDFTLTISSLQPEDFATYYCQQSGVWLITFGQGT | |
| | | KVEIK | |
| X16 | VH | EVQLVESGGGLVQPGGSLRLSCAASGFTFSSSSIHWVRQAPGKGLEWVA | 127 |
| | | SIYSSYGYTSYADSVKGRFTISADTSKNTAYLQMNSLRAEDTAVYYCARNW | |
| | | GYYMYWGWYYALDYWGQGTLVTVSS | |
| | VL | DIQMTQSPSSLSASVGDRVTITCRASQSVSSAVAWYQQKPGKAPKLLIYSA | 128 |
| | | SSLYSGVPSRFSGSRSGTDFTLTISSLQPEDFATYYCQQWSSAQALITFGQ | |
| | | GTKVEIK | |
| X17 | VH | EVQLVESGGGLVQPGGSLRLSCAASGFTISYSSIHWVRQAPGKGLEWVAS | 131 |
| | | IYSSSGSTYYADSVKGRFTISADTSKNTAYLQMNSLRAEDTAVYYCARGDH | |
| | | GYYVFGYLGMDYWGQGTLVTVSS | |
| X17m1 | VH | EVQLVESGGGLVQPGGSLRLSCAASGFTISYSSIHWVRQAPGKGLEWVAS | 260 |
| | | IYSSSGSTYYADSVKGRFTISADTSKNTAYLQMNSLRAEDTAVYYCARGDH | |
| | | GYYVSGYLGMDYWGQGTLVTVSS | |
| X17m2 | VH | EVQLVESGGGLVQPGGSLRLSCAASGFTISYSSIHWRQAPGKGLEWVAS | 261 |
| | | IYSSSGSTYYADSVKGRFTISADTSKNTAYLQMNSLRAEDTAVYYCARGDH | |
| | | GYYVYGYLGMDYWGQGTLVTVSS | |
| X17m3 | VH | EVQLVESGGGLVQPGGSLRLSCAASGFTISYSSIHWRQAPGKGLEWVAS | 262 |
| | | IYSSSGSTYYADSVKGRFTISADTSKNTAYLQMNSLRAEDTAVYYCARGDH | |
| | | GYYVSGYLGFDYWGQGTLVTVSS | |
| X17m6 | VH | EVQLVESGGGLVQPGGSLRLSCAASGFTISYSSIHWVRQAPGKGLEWVAS | 263 |
| | | IYSSSGSTYYADSVKGRFTISADTSKNTAYLQMNSLRAEDTAVYYCARGEH | |
| | | GYYVSGYLGFDYWGQGTLVTVSS | |
| X17 | VL | DIQMTQSPSSLSASVGDRVTITCRASQSVSSAVAWYQQKPGKAPKLLIYSA | 132 |
| | | SSLYSGVPSRFSGSRSGTDFTLTISSLQPEDFATYYCQQSYKPGALITFGQ | |
| | | GTKVEIK | |
| X18 | VH | EVQLVESGGGLVQPGGSLRLSCAASGFTISSSSIHWVRQAPGKGLEWVAS | 133 |
| | | ISSSSGYTSYADSVKGRFTISADTSKNTAYLQMNSLRAEDTAVYYCARKYS | |
| | | KRAYPDYYWRGLDYWGQGTLVTVSS | |
| | VL | DIQMTQSPSSLSASVGDRVTITCRASQSVSSAVAWYQQKPGKAPKLLIYSA | 134 |
| | | SSLYSGVPSRFSGSRSGTDFTLTISSLQPEDFATYYCQQSYWWPITFGQGT | |
| | | KVEIK | |
| 14D10 | VH | ELQLVESGGGLVQPGGSLRLSCAASGFTFDDYGMSWVRQAPGKGLEWV | 196 |
| | | SAIPWNGGSTYYKESVKGRFTISRDNAKKTLYLQMNSLKSEDTAVYYCAKR | |
| | | SGRIAFGALDAWGQGTLVTVSS | |
| | VL | QAVVTQEPSLSVSPGGTVTLTCGLSSGSVTSSNYPDWYQQTPGQAPRTLI | 197 |
| | | YTTNSRHSGVPSRFSGSISGNKAALTITGAQPEDEADYYCALYMGGGSNV | |
| | | YVFGGGTKLTVL | |
| 7G4 | VH | ELQLVESGGGLVQPGGSLRLSCAASGFTFDDYGMSWVRQAPGKGLEWV | 198 |
| | | SAIPWNGGSTYYKESVKGRFTISRDNAKKTLYLQMNSLKSEDTAVYYCAKR | |
| | | SGRIAFGALDAWGQGTLVTVSS | |
| | VL | QAVVTQEPSLSVSPGGTVTLTCGLSSGSVTSSNYPDWYQQTPGQAPRALI | 199 |
| | | YSTNSRHSGVPSRFSGSISGNKAALTITGAQPEDEADYYCALYMGRGSNK | |
| | | DYVFGGGTKLTVL | |
| 3C4 | VH | ELQLVESGGGLVQPGGSLRLSCAASGFTFDDYGMSWVRQAPGKGLEWV | 200 |
| | | SAIPWNGGSTYYKESVKGRFTISRDNAKKTLYLQMNSLKSEDTAVYYCAKR | |
| | | SGRIAFGALDAWGQGTLVTVSS | |
| | VL | QAVVTQEPSLSVSPGGTVTLTCGLSSGSVTASNYPDWYQQTPGQAPRGLI | 201 |
| | | YSTDSRHSGVPSRFSGSISGNKAALTITGAQPEDEADYYCALYMYSDSKLY | |
| | | VFGGGTKLTVL | |
| 3B2 | VH | ELQLVESGGGLVQPGGSLRLSCAASGFTFDDYGMSWVRQAPGKGLEWV | 202 |
| | | SAIPWNGGSTYYKESVKGRFTISRDNAKKTLYLQMNSLKSEDTAVYYCAKR | |
| | | SGRIAFGALDAWGQGTLVTVSS | |
| | VL | QAVVTQEPSLSVSPGGTVTLTCGLSSGSVTSSNYPDWYQQTPGQAPRGLI | 203 |
| | | YSTDSRHSGVPSRFSGSISGNKAALTITGAQSEDEADYYCGLYMYSGSKN | |
| | | YVFGGGTKLTVL | |
| 3G3 | VH | ELQLVESGGGLVQPGGSLRLSCAASGFTFDDYGMSWVRQAPGKGLEWV | 204 |
| | | SAIPWNGGSTYYKESVKGRFTISRDNAKKTLYLQMNSLKSEDTAVYYCAKR | |
| | | SGRIAFGALDAWGQGTLVTVSS | |
| | VL | QTVVTQEPSLSVSPGGTVTLTCGLSSGSVTSSNYPDWYQQTPGQAPRALI | 205 |
| | | YSTNSRHSGVPSRFSGSTSGNKAALTITGAQPEDEADYYCALYMGSDIRN | |
| | | YVFGGGTKLTVL | |
| 31G2 | VH | ELQLVESGGGLVQPGGSLRLSCAASGFTFDDYGMSWVRQAPGKGLEWV | 206 |
| | | SAIPWNGGSTYYKESVKGRFTISRDNAKKTLYLQMNSLKSEDTAVYYCAKR | |
| | | SGRIAFGALDAWGQGTLVTVSS | |
| | VL | QAVVTQEPSLSVSPGGTVTLTCGLSSGSVTSSNYPDWYQQTPGQAPRALI | 207 |
| | | YSTNSRLSGVPSRFSGSFSGNKAALTITGAQPEDEADYYCALYMGSGSRN | |
| | | YVFGGGTKLTVL | |
| 31 B7 | VH | ELQLVESGGGLVQPGGSLRLSCAASGFTFDDYGMSWVRQAPGKGLEWV | 208 |
| | | SAIPWNGGSTYYKESVKGRFTISRDNAKKTLYLQMNSLKSEDTAVYYCAKR | |
| | | SGRIAFGALDAWGQGTLVTVSS | |
| | VL | QAVVTQEPSLSVSPGGTVTLTCGLSSGSVTSSNYPDWYQQTPGQAPRALI | 209 |
| | | YSTNSRLSGVPSRFSGSFSGNKAALTITGAQPEDEADYYCALYMGSESRN | |
| | | YVFGGGTKLTVL | |
| 17H10 | VH | QVQVQESGPGLVKPSQTLSLTCTVSGGSITARYYSWSWIRQPPGKGLEW | 210 |
| | | MGVIAYDGSTYYSPSLKSRTSISRDTSKNQFSLHLSSVTPDDTAVYYCARG | |
| | | SSRVAAAFDSWGQGTQVTVSS | |
| | VL | SYELTQSPSVSVALRQTAKITCGGNRIGGKSVQWYQQKPGQAPMLVIYAD | 211 |
| | | SRRPSGIPERFTGSNSGNTATLTITGAQAEDEADYYCHVWGSTASADFGG | |
| | | GTHLTVL | |
| 23B6 | VH | QVQVQESGPGLVKPSQTLSLTCTVSGGSITARYYSWSWIRQPPGKGLEW | 212 |
| | | MGVIAYDGSTYYSPSLKSRTSISRDTSKNQFSLHLSSVTPDDTAVYYCARG | |
| | | SSRVAAAFDSWGQGTQVTVSS | |
| | VL | SYELTQSPSVSVALRQTAKITCGGDNIGSKNAQWYQQKPGQAPVMVLYAD | 213 |
| | | SRRPSGIPERFSGSNSGNTATLTISGAQAEDEADYYCHVWDSSTNAWFGG | |
| | | GTHLTVL | |
| 30E1 | VH | QVQVQESGPGLVKPSQTLSLTCTVSGGSITARYYSWSWIRQPPGKGLEW | 214 |
| | | MGVIAYDGSTYYSPSLKSRTSISRDTSKNQFSLHLSSVTPDDTAVYYCARG | |
| | | SSRVAAAFDSWGQGTQVTVSS | |
| | VL | SYELTQSPSVSVALRRTAKITCGGDNIGSKNTQWYQQKPGQAPVLVIYADS | 215 |
| | | RRPSGIPERFSGSNSGNTATLTISGAQAEDEADYYCHVWDSSTNAWFGGG | |
| | | THLTVL | |
| 30A11 | VH | QVQVQESGPGLVKPSQTLSLTCTVSGGSITARYYSWSWIRQPPGKGLEW | 216 |
| | | MGVIAYDGSTYYSPSLKSRTSISRDTSKNQFSLHLSSVTPDDTAVYYCARG | |
| | | SSRVAAAFDSWGQGTQVTVSS | |
| | VL | SYELTQSPSVTVALRQTAKITCGGDNIASKNVQWYQQKPGQAPSLVIWAD | 217 |
| | | SRRPSGIPVRFSGSNFGNTATLTISGAQAEDEADYYCQVWDSSTNVAVFG | |
| | | GGTHLTVL | |
| 16F11 | VH | EVQLVESGGGLVQPGGSLSLSCVASGFTFSLYYMNWVRQAPGKGLEWLS | 218 |
| | | VIDTHSIAYYADSVKGRFTISRDNVKNTLYLQLNNLKPEDTALYYCVLGRTAL | |
| | | VRWGQGTQVTVSS | |
| | VL | DIVMTQSPSSVTASVGEKVTINCKSSQSVVFGSNQKSYLNWYQQRPGQSP | 219 |
| | | RLLIYYASTQESGIPDRFSGSGSTTDFTLTISSVQPEDAAVYYCQQAYSAPT | |
| | | FGSGTRLEIK | |
| 4C11 | VH | EVQLVESGGGLVQPGGSLSLSCVASGFTFSLYYMNWVRQAPGKGLEWLS | 220 |
| | | VIDTHSIAYYADSVKGRFTISRDNVKNTLYLQLNNLKPEDTALYYCVLGRTAL | |
| | | VRWGQGTQVTVSS | |
| | VL | DIVMTQSPSSVTASAGERVTINCRSSQSVLYSSNQKNYLNWYQQRLGQSP | 221 |
| | | RLLIYWASARESGVPDRFSGSGSTTNFTLTISSFQPEDAAVYYCQQSYKPP | |
| | | YGFGSGTRLEIK | |
| 7A12 | VH | EVQLVESGGGLVQPGGSLSLSCVASGFTFSLYYMNWVRQAPGKGLEWLS | 222 |
| | | VIDTHSIAYYADSVKGRFTISRDNVKNTLYLQLNNLKPEDTALYYCVLGRTAL | |
| | | VRWGQGTQVTVSS | |
| | VL | EIVLTQSPSSVTASIGEKVTINCESSQSVLYNQKNYLNWYQQRPGQSPRLLI | 223 |
| | | YWASTRQSGVPDRFSGSGSGSTTDFTLTISSFQPEDVAVYYCQQAYNAPL | |
| | | TFGPGTKVELK | |
| 7G12 | VH | EVQLVESGGGLVQPGGSLSLSCVASGFTFSLYYMNWVRQAPGKGLEWLS | 224 |
| | | VIDTHSIAYYADSVKGRFTISRDNVKNTLYLQLNNLKPEDTALYYCVLGRTAL | |
| | | VRWGQGTQVTVSS | |
| | VL | EIVLTQSPNSVTASVGEKVTINCKSSQRVQLGSNQKSYLNWYQQRPGQSP | 225 |
| | | RLLIYYASTQQSGIPDRFSGSGSATDFTLTINSVQPEDAAVYYCQQGYSAP | |
| | | FTFGQGTKVELK | |
| 7B8 | VH | EVQLVESGGGLVQPGGSLSLSCVASGFTFSLYYMNWVRQAPGKGLEWLS | 226 |
| | | VIDTHSIAYYADSVKGRFTISRDNVKNTLYLQLNNLKPEDTALYYCVLGRTAL | |
| | | VRWGQGTQVTVSS | |
| | VL | EIVLTQSPSSVTASAGEKVTINCKSSQSVLYNQKNYLAWYQQRPGQSPRLL | 227 |
| | | IYWASTRESGVPDRFSGSGSTTDFTLTISSFQPEDVAVYYCQQGYSVPYTF | |
| | | GSGTRLEIK | |
| 3B2g1m1 | VH | EVQLLESGGGLVQPGGSLRLSCAASGFTFSDYGMSWVRQAPGKGLEWVS | 228 |
| | | AIPWSGGSTYYKESVKGRFTISRDNSKNTLYLQMNSLRAEDTAVYYCAKRS | |
| | | GRIAFGALDAWGQGTLVTVSS | |
| | VL | QTVVTQEPSFSVSPGGTVTLTCGLSSGSVTSSNYPDWYQQTPGQAPRTLI | 229 |
| | | YSTDSRHSGVPDRFSGSILGNKAALTITGAQADDESDYYCGLYMYSGSKN | |
| | | YVFGGGTKLTVL | |
| 3B2g1m2 | VH | EVQLLESGGGLVQPGGSLRLSCAASGFTFSDYGMSWVRQAPGKGLEWVS | 230 |
| | | AIPGSGGSTYYKESVKGRFTISRDNSKNTLYLQMNSLRAEDTAVYYCAKRS | |
| | | GRIAFGALDAWGQGTLVTVSS | |
| | VL | QTVVTQEPSFSVSPGGTVTLTCGLSSGSVTSSNYPDWYQQTPGQAPRTLI | 231 |
| | | YSTDSRHSGVPDRFSGSILGNKAALTITGAQADDESDYYCGLYMYSGSKN | |
| | | YVFGGGTKLTVL | |
| 3B2g1m4 | VH | EVQLLESGGGLVQPGGSLRLSCAASGFTFSDYGMSWVRQAPGKGLEWVS | 232 |
| | | AIPWQGGSTYYKESVKGRFTISRDNSKNTLYLQMNSLRAEDTAVYYCAKR | |
| | | SGRIAFGALDAWGQGTLVTVSS | |
| | VL | QTVVTQEPSFSVSPGGTVTLTCGLSSGSVTSSNYPDWYQQTPGQAPRTLI | 233 |
| | | YSTDSRHSGVPDRFSGSILGNKAALTITGAQADDESDYYCGLYMYSGSKN | |
| | | YVFGGGTKLTVL | |
| 3B2g2m1 | VH | EVQLLESGGGLVQPGGSLRLSCAASGFTFSDYGMSWVRQAPGKGLEWVS | 234 |
| | | AIPWSGGSTYYKESVKGRFTISRDNSKNTLYLQMNSLRAEDTAVYYCAKRS | |
| | | GRIAFGALDAWGQGTLVTVSS | |
| | VL | QTVVTQEPSFSVSPGGTVTLTCGLSSGSVTSSNYPDWYQQTPGQAPRTLI | 235 |
| | | YSTDSRHSGVPDRFSGSILGNKAALTITGAQADDESDYYCGLYSYSGSKNY | |
| | | VFGGGTKLTVL | |
| 3B2g2m2 | VH | EVQLLESGGGLVQPGGSLRLSCAASGFTFSDYGMSWVRQAPGKGLEWVS | 236 |
| | | AIPGSGGSTYYKESVKGRFTISRDNSKNTLYLQMNSLRAEDTAVYYCAKRS | |
| | | GRIAFGALDAWGQGTLVTVSS | |
| | VL | QTVVTQEPSFSVSPGGTVTLTCGLSSGSVTSSNYPDWYQQTPGQAPRTLI | 237 |
| | | YSTDSRHSGVPDRFSGSILGNKAALTITGAQADDESDYYCGLYSYSGSKNY | |
| | | VFGGGTKLTVL | |
| 3B2g2m4 | VH | EVQLLESGGGLVQPGGSLRLSCAASGFTFSDYGMSWVRQAPGKGLEWVS | 238 |
| | | AIPWQGGSTYYKESVKGRFTISRDNSKNTLYLQMNSLRAEDTAVYYCAKR | |
| | | SGRIAFGALDAWGQGTLVTVSS | |
| | VL | QTVVTQEPSFSVSPGGTVTLTCGLSSGSVTSSNYPDWYQQTPGQAPRTLI | 239 |
| | | YSTDSRHSGVPDRFSGSILGNKAALTITGAQADDESDYYCGLYSYSGSKNY | |
| | | VFGGGTKLTVL | |

In an embodiment, the MuSK antibody-based molecule disclosed herein comprises:
a heavy chain variable domain comprising an amino acid sequence that is at least 80% identical to SEQ ID NO: 97 and a light chain variable domain comprising an amino acid sequence that is at least 80% identical to SEQ ID NO: 98; (ii) a heavy chain variable domain comprising an amino acid sequence that is at least 80% identical to any one of SEQ ID NOs: 99 and 252-259 and a light chain variable domain comprising an amino acid sequence that is at least 80% identical to SEQ ID NO: 100; (iii) a heavy chain variable domain comprising an amino acid sequence that is at least 80% identical to SEQ ID NO: 101 and a light chain variable domain comprising an amino acid sequence that is at least 80% identical to SEQ ID NO: 102; (iv) a heavy chain variable domain comprising an amino acid sequence that is at least 80% identical to SEQ ID NO: 103 and a light chain variable domain comprising an amino acid sequence that is at least 80% identical to SEQ ID NO: 104; (v) a heavy chain variable domain comprising an amino acid sequence that is at least 80% identical to SEQ ID NO: 105 and a light chain variable domain comprising an amino acid sequence that is at least 80% identical to SEQ ID NO: 106; (vi) a heavy chain variable domain comprising an amino acid sequence that is at least 80% identical to SEQ ID NO: 107 and a light chain variable domain comprising an amino acid sequence that is at least 80% identical to SEQ ID NO: 108; or (vii) a heavy chain variable domain comprising an amino acid sequence that is at least 80% identical to SEQ ID NO: 109 and a light chain variable domain comprising an amino acid sequence that is at least 80% identical to SEQ ID NO: 110; (viii) a heavy chain variable domain comprising an amino acid sequence that is at least 80% identical to SEQ ID NO: 111 and a light chain variable domain comprising an amino acid sequence that is at least 80% identical to SEQ ID NO: 112; (ix) a heavy chain variable domain comprising an amino acid sequence that is at least 80% identical to SEQ ID NO: 113 and a light chain variable domain comprising an amino acid sequence that is at least 80% identical to SEQ ID NO: 114; (x) a heavy chain variable domain comprising an amino acid sequence that is at least 80% identical to SEQ ID NO: 115 and a light chain variable domain comprising an amino acid sequence that is at least 80% identical to SEQ ID NO: 116; (xi) a heavy chain variable domain comprising an amino acid sequence that is at least 80% identical to SEQ ID NO: 117 and a light chain variable domain comprising an amino acid sequence that is at least 80% identical to SEQ ID NO: 118; (xii) a heavy chain variable domain comprising an amino acid sequence that is at least 80% identical to SEQ ID NO: 119 and a light chain variable domain comprising an amino acid sequence that is at least 80% identical to SEQ ID NO: 120; (xiii) a heavy chain variable domain comprising an amino acid sequence that is at least 80% identical to SEQ ID NO: 121 and a light chain variable domain comprising an amino acid sequence that is at least 80% identical to SEQ ID NO: 122; (xiv) a heavy chain variable domain comprising an amino acid sequence that is at least 80% identical to SEQ ID NO: 123 and a light chain variable domain comprising an amino acid sequence that is at least 80% identical to SEQ ID NO: 124; (xv) a heavy chain variable domain comprising an amino acid sequence that is at least 80% identical to SEQ ID NO: 125 and a light chain variable domain comprising an amino acid sequence that is at least 80% identical to SEQ ID NO: 126; (xvi) a heavy chain variable domain comprising an amino acid sequence that is at least 80% identical to SEQ ID NO: 127 and a light chain variable domain comprising an amino acid sequence that is at least 80% identical to SEQ ID NO: 128; (xvii) a heavy chain variable domain comprising an amino acid sequence that is at least 80% identical to any one of SEQ ID NOs: 131 and 260-263 and a light chain variable domain comprising an amino acid sequence that is at least 80% identical to SEQ ID NO: 132; and (xviii) a heavy chain variable domain comprising an amino acid sequence that is at least 80% identical to SEQ ID NO: 133 and a light chain variable domain comprising an amino acid sequence that is at least 80% identical to SEQ ID NO: 134.

In some embodiments, the MuSK antibody-based molecule disclosed herein comprises: (i) a heavy chain variable domain comprising an amino acid sequence that is at least 80% identical to SEQ ID NO: 196 and a light chain variable domain comprising an amino acid sequence that is at least 80% identical to SEQ ID NO: 197; (ii) a heavy chain variable domain comprising an amino acid sequence that is at least 80% identical to SEQ ID NO: 198 and a light chain variable domain comprising an amino acid sequence that is at least 80% identical to SEQ ID NO: 199; (iii) a heavy chain variable domain comprising an amino acid sequence that is at least 80% identical to SEQ ID NO: 200 and a light chain variable domain comprising an amino acid sequence that is at least 80% identical to SEQ ID NO: 201; (iv) a heavy chain variable domain comprising an amino acid sequence that is at least 80% identical to SEQ ID NO: 202 and a light chain variable domain comprising an amino acid sequence that is at least 80% identical to SEQ ID NO: 203; (v) a heavy chain variable domain comprising an amino acid sequence that is at least 80% identical to SEQ ID NO: 204 and a light chain variable domain comprising an amino acid sequence that is at least 80% identical to SEQ ID NO: 205; (vi) a heavy chain variable domain comprising an amino acid sequence that is at least 80% identical to SEQ ID NO: 206 and a light chain variable domain comprising an amino acid sequence that is at least 80% identical to SEQ ID NO: 207; (vii) a heavy chain variable domain comprising an amino acid sequence that is at least 80% identical to SEQ ID NO: 208 and a light chain variable domain comprising an amino acid sequence that is at least 80% identical to SEQ ID NO: 209; (viii) a heavy chain variable domain comprising an amino acid sequence that is at least 80% identical to SEQ ID NO: 210 and a light chain variable domain comprising an amino acid sequence that is at least 80% identical to SEQ ID NO: 211; (vix) a heavy chain variable domain comprising an amino acid sequence that is at least 80% identical to SEQ ID NO: 212 and a light chain variable domain comprising an amino acid sequence that is at least 80% identical to SEQ ID NO: 213; (x) a heavy chain variable domain comprising an amino acid sequence that is at least 80% identical to SEQ ID NO: 214 and a light chain variable domain comprising an amino acid sequence that is at least 80% identical to SEQ ID NO: 215; (xi) a heavy chain variable domain comprising an amino acid sequence that is at least 80% identical to SEQ ID NO: 216 and a light chain variable domain comprising an amino acid sequence that is at least 80% identical to SEQ ID NO: 217; (xii) a heavy chain variable domain comprising an amino acid sequence that is at least 80% identical to SEQ ID NO: 218 and a light chain variable domain comprising an amino acid sequence that is at least 80% identical to SEQ ID NO: 219; (xiii) a heavy chain variable domain comprising an amino acid sequence that is at least 80% identical to SEQ ID NO: 220 and a light chain variable domain comprising an amino acid sequence that is at least 80% identical to SEQ ID NO: 221; (xiv) a heavy chain variable domain comprising an amino acid sequence that is at least 80% identical to SEQ ID NO: 222 and a light chain variable domain comprising an amino acid sequence that is at least 80% identical to SEQ ID NO: 223; (xv) a heavy chain variable domain comprising an amino acid sequence that is at least 80% identical to SEQ ID NO: 224 and a light chain variable domain comprising an amino acid sequence that is at least 80% identical to SEQ ID NO: 225; (xvi) a heavy chain variable domain comprising an amino acid sequence that is at least 80% identical to SEQ ID NO: 226 and a light chain variable domain comprising an amino acid sequence that is at least 80% identical to SEQ ID NO: 227; (xvii) a heavy chain variable domain comprising an amino acid sequence that is at least 80% identical to SEQ ID NO: 228 and a light chain variable domain comprising an amino acid sequence that is at least 80% identical to SEQ ID NO: 229; (xviii) a heavy chain variable domain comprising an amino acid sequence that is at least 80% identical to SEQ ID NO: 230 and a light chain variable domain comprising an amino acid sequence that is at least 80% identical to SEQ ID NO: 231; (xix) a heavy chain variable domain comprising an amino acid sequence that is at least 80% identical to SEQ ID NO: 232 and a light chain variable domain comprising an amino acid sequence that is at least 80% identical to SEQ ID NO: 233; (xx) a heavy chain variable domain comprising an amino acid sequence that is at least 80% identical to SEQ ID NO: 234 and a light chain variable domain comprising an amino acid sequence that is at least 80% identical to SEQ ID NO: 235; (xxi) a heavy chain variable domain comprising an amino acid sequence that is at least 80% identical to SEQ ID NO: 236 and a light chain variable domain comprising an amino acid sequence that is at least 80% identical to SEQ ID NO: 237; (xxii) a heavy chain variable domain comprising an amino acid sequence that is at least 80% identical to SEQ ID NO: 238 and a light chain variable domain comprising an amino acid sequence that is at least 80% identical to SEQ ID NO: 239.

In a preferred embodiment, the MuSK antibody-based molecule (or the anti-MuSK antibody or antigen binding fragment thereof) disclosed herein comprises a heavy chain variable domain (VH) comprising an amino acid sequence that is at least 80% identical or similar to SEQ ID NO: 234 and a light chain variable domain (VL) comprising an amino acid sequence that is at least 80% identical or similar to SEQ ID NO: 235. In an embodiment, the identity or similarity is at least 81%, 82%, 83%, 84%, 85%, 86%, 87%, 88%, 89%, 90%, 91%, 92%, 93%, 94%, 95%, 96%, 97%, 98%, 99%. In a preferred embodiment, the MuSK antibody-based molecule disclosed herein comprises a heavy chain variable domain comprising amino acid sequence SEQ ID NO: 234 and a light chain variable domain comprising amino acid sequence SEQ ID NO: 235.

In a preferred embodiment, the antibody-based molecule that binds to human muscle-specific tyrosine-protein kinase (MuSK) comprises a heavy chain variable domain and a light chain variable domain, wherein the heavy chain variable domain comprises an amino acid sequence that is at least 80% identical or similar to SEQ ID NO: 234 and the light chain variable domain comprises an amino acid sequence that is at least 80% identical or similar to SEQ ID NO: 235, and
where the heavy chain variable domain comprises:
   a CDR-H1 amino acid sequence comprising SEQ ID NO: 147 or having 1, 2, 3, 4 or 5 amino acid alterations relative to SEQ ID NO: 147,
   a CDR-H2 amino acid sequence comprising SEQ ID NO: 153 or having 1, 2, 3, 4 or 5 amino acid alterations relative to SEQ ID NO: 153, and
   a CDR-H3 amino acid sequence comprising SEQ ID NO: 156 or having 1, 2, 3, 4 or 5 amino acid alterations relative to SEQ ID NO:156 (3B2g2m1) and
where the light chain variable domain comprises:
   a CDR-L1 amino acid sequence comprising SEQ ID NO: 159 or having 1, 2, 3, 4 or 5 amino acid alterations relative to SEQ ID NO: 159,
   a CDR-L2 amino acid sequence comprising SEQ ID NO: 172 or having 1, 2, 3, 4 or 5 amino acid alterations relative to SEQ ID NO: 172, and
   a CDR-L3 amino acid sequence comprising SEQ ID NO: 195 or having 1, 2, 3, 4 or 5 amino acid alterations relative to SEQ ID NO:195 (3B2g2m1).

In an embodiment, the identity or similarity is at least 81%, 82%, 83%, 84%, 85%, 86%, 87%, 88%, 89%, 90%, 91%, 92%, 93%, 94%, 95%, 96%, 97%, 98%, 99%.

In a more preferred embodiment, the antibody-based molecule that binds to human muscle-specific tyrosine-protein kinase (MuSK) comprises a heavy chain variable domain and a light chain variable domain, wherein the heavy chain variable domain comprises an amino acid sequence that is at least 80% identical or similar to SEQ ID NO: 234 and the light chain variable domain comprises an amino acid sequence that is at least 80% identical or similar to SEQ ID NO: 235, and
where the heavy chain variable domain comprises:
   a CDR-H1 amino acid sequence comprising or consisting of SEQ ID NO: 147,
   a CDR-H2 amino acid sequence comprising or consisting of SEQ ID NO: 153, and
   a CDR-H3 amino acid sequence comprising or consisting of SEQ ID NO:156 (3B2g2m1) and
where the light chain variable domain comprises:
   a CDR-L1 amino acid sequence comprising or consisting of SEQ ID NO: 159,
   a CDR-L2 amino acid sequence comprising or consisting of SEQ ID NO: 172, and
   a CDR-L3 amino acid sequence comprising or consisting of SEQ ID NO:195 (3B2g2m1).

In an embodiment, the identity or similarity is at least 81%, 82%, 83%, 84%, 85%, 86%, 87%, 88%, 89%, 90%, 91%, 92%, 93%, 94%, 95%, 96%, 97%, 98%, 99%.

In an embodiment, the antibody-based molecule that binds to human muscle-specific tyrosine-protein kinase (MuSK) comprises wild-type human IgG constant Fc region, a heavy chain variable domain and a light chain variable domain, where the wild-type human IgG constant Fc region comprising at least 80% sequence identity to SEQ ID NO: 266 or 267, where the heavy chain variable domain comprises an amino acid sequence that is at least 80% identical or similar to SEQ ID NO: 234 and the light chain variable domain comprises an amino acid sequence that is at least 80% identical or similar to SEQ ID NO: 235, and
where the heavy chain variable domain comprises:
   a CDR-H1 amino acid sequence comprising SEQ ID NO: 147 or having 1, 2, 3, 4 or 5 amino acid alterations relative to SEQ ID NO: 147,
   a CDR-H2 amino acid sequence comprising SEQ ID NO: 153 or having 1, 2, 3, 4 or 5 amino acid alterations relative to SEQ ID NO: 153, and
   a CDR-H3 amino acid sequence comprising SEQ ID NO: 156 or having 1, 2, 3, 4 or 5 amino acid alterations relative to SEQ ID NO:156 (3B2g2m1) and
where the light chain variable domain comprises:
   a CDR-L1 amino acid sequence comprising SEQ ID NO: 159 or having 1, 2, 3, 4 or 5 amino acid alterations relative to SEQ ID NO: 159,
   a CDR-L2 amino acid sequence comprising SEQ ID NO: 172 or having 1, 2, 3, 4 or 5 amino acid alterations relative to SEQ ID NO: 172, and
   a CDR-L3 amino acid sequence comprising SEQ ID NO: 195 or having 1, 2, 3, 4 or 5 amino acid alterations relative to SEQ ID NO:195 (3B2g2m1).

In an embodiment, the identity or similarity is at least 81%, 82%, 83%, 84%, 85%, 86%, 87%, 88%, 89%, 90%, 91%, 92%, 93%, 94%, 95%, 96%, 97%, 98%, 99%.

In an embodiment, the CDR-H2 of the antibody comprises a proline (P) at position 3, a tryptophan (W) at position 4, and a serine (S) or asparagine (N) at position 5. In a preferred embodiment, the CDR-H2 of the antibody comprises a proline (P) at position 3, a tryptophan (W) at position 4, and a serine (S) at position 5.

In an embodiment, the antibody-based molecule that binds to human muscle-specific tyrosine-protein kinase (MuSK) comprises wild-type human IgG constant Fc region, a heavy chain variable domain and a light chain variable domain, where the wild-type human IgG constant Fc region comprising SEQ ID NO: 266 or 267, where the heavy chain variable domain comprises an amino acid sequence that is at least 80% identical or similar to SEQ ID NO: 234 and the light chain variable domain comprises an amino acid sequence that is at least 80% identical or similar to SEQ ID NO: 235, and
where the heavy chain variable domain comprises:
   a CDR-H1 amino acid sequence comprising SEQ ID NO: 147 or having 1, 2, 3, 4 or 5 amino acid alterations relative to SEQ ID NO: 147,
   a CDR-H2 amino acid sequence comprising SEQ ID NO: 153 or having 1, 2, 3, 4 or 5 amino acid alterations relative to SEQ ID NO: 153, and
   a CDR-H3 amino acid sequence comprising SEQ ID NO: 156 or having 1, 2, 3, 4 or 5 amino acid alterations relative to SEQ ID NO:156 (3B2g2m1) and
where the light chain variable domain comprises:
   a CDR-L1 amino acid sequence comprising SEQ ID NO: 159 or having 1, 2, 3, 4 or 5 amino acid alterations relative to SEQ ID NO: 159,
   a CDR-L2 amino acid sequence comprising SEQ ID NO: 172 or having 1, 2, 3, 4 or 5 amino acid alterations relative to SEQ ID NO: 172, and
   a CDR-L3 amino acid sequence comprising SEQ ID NO: 195 or having 1, 2, 3, 4 or 5 amino acid alterations relative to SEQ ID NO:195 (3B2g2m1).

In an embodiment, the identity or similarity is at least 81%, 82%, 83%, 84%, 85%, 86%, 87%, 88%, 89%, 90%, 91%, 92%, 93%, 94%, 95%, 96%, 97%, 98%, 99%.

In an embodiment, the CDR-H2 of the antibody comprises a proline (P) at position 3, a tryptophan (W) at position 4, and a serine (S) or asparagine (N) at position 5. In a preferred embodiment, the CDR-H2 of the antibody comprises a proline (P) at position 3, a tryptophan (W) at position 4, and a serine (S) at position 5.

In an embodiment, the antibody-based molecule that binds to human muscle-specific tyrosine-protein kinase (MuSK) comprises wild-type human IgG constant Fc region, a heavy chain variable domain and a light chain variable domain, where the wild-type human IgG constant Fc region comprising SEQ ID NO: 266 or 267, where the heavy chain variable domain comprising SEQ ID NO: 234 and the light chain variable domain comprising SEQ ID NO: 235, and
where the heavy chain variable domain comprises:
   a CDR-H1 amino acid sequence comprising SEQ ID NO: 147 or having 1, 2, 3, 4 or 5 amino acid alterations relative to SEQ ID NO: 147,
   a CDR-H2 amino acid sequence comprising SEQ ID NO: 153 or having 1, 2, 3, 4 or 5 amino acid alterations relative to SEQ ID NO: 153, and
   a CDR-H3 amino acid sequence comprising SEQ ID NO: 156 or having 1, 2, 3, 4 or 5 amino acid alterations relative to SEQ ID NO:156 (3B2g2m1) and
where the light chain variable domain comprises:
   a CDR-L1 amino acid sequence comprising SEQ ID NO: 159 or having 1, 2, 3, 4 or 5 amino acid alterations relative to SEQ ID NO: 159,
   a CDR-L2 amino acid sequence comprising SEQ ID NO: 172 or having 1, 2, 3, 4 or 5 amino acid alterations relative to SEQ ID NO: 172, and
   a CDR-L3 amino acid sequence comprising SEQ ID NO: 195 or having 1, 2, 3, 4 or 5 amino acid alterations relative to SEQ ID NO:195 (3B2g2m1).

In an embodiment, the CDR-H2 of the antibody comprises a proline (P) at position 3, a tryptophan (W) at position 4, and a serine (S) or asparagine (N) at position 5. In a preferred embodiment, the CDR-H2 of the antibody comprises a proline (P) at position 3, a tryptophan (W) at position 4, and a serine (S) at position 5.

In an embodiment, the antibody-based molecule that binds to human muscle-specific tyrosine-protein kinase (MuSK) comprises wild-type human IgG constant Fc region, a heavy chain variable domain and a light chain variable domain, where the wild-type human IgG constant Fc region comprising SEQ ID NO: 266 or 267, where the heavy chain variable domain comprising SEQ ID NO: 234 and the light chain variable domain comprising SEQ ID NO: 235, and
where the heavy chain variable domain comprises:
   a CDR-H1 amino acid sequence comprising or consisting of SEQ ID NO: 147,
   a CDR-H2 amino acid sequence comprising or consisting of SEQ ID NO: 153, and
   a CDR-H3 amino acid sequence comprising or consisting of SEQ ID NO:156 (3B2g2m1) and
where the light chain variable domain comprises:
   a CDR-L1 amino acid sequence comprising or consisting of SEQ ID NO: 159,
   a CDR-L2 amino acid sequence comprising or consisting of SEQ ID NO: 172, and
   a CDR-L3 amino acid sequence comprising or consisting of SEQ ID NO:195 (3B2g2m1).
   a CDR-L3 amino acid sequence comprising SEQ ID NO: 195 or having 1, 2, 3, 4 or 5 amino acid alterations relative to SEQ ID NO:195 (3B2g2m1).

In an embodiment, the antibody-based molecule that binds to human muscle-specific tyrosine-protein kinase (MuSK) comprises wild-type human IgG constant Fc region, a heavy chain variable domain and a light chain variable domain,
where the wild-type human IgG constant Fc region comprising at least 80% sequence identity to SEQ ID NO: 266 or 267, wherein one or more of the following mutations (all numbered according to the EU numbering system) have been introduced: an N297A substitution; an N297Q substitution; an L234A substitution; an L234D substitution; an L234E substitution; an L234G substitution; an L234H substitution; an L234F substitution; an L234K substitution; an L234Q substitution; an L234R substitution; an L234S substitution; an L234T substitution; an L235A substitution; an L235D substitution; an L235E substitution; an L235F substitution; an L235G substitution; an L235V substitution; an L235H substitution; an L2351 substitution; an L235K substitution; an L235R substitution; an L235S substitution; L235T substitution; an L235Q substitution; an L237A substitution; an S239D substitution; an E233P substitution; an L234V substitution; a C236 deletion; a G236E substitution; a G236R substitution; a G236K substitution; a G237A substitution; a P238A substitution; an F243L substitution; a D265A substitution; an S267E substitution; an H268A substitution; an R292P substitution; a Y300L substitution; a K322A substitution; a K322Q substitution; an A327Q substitution; an L328F substitution; an L328R substitution; a P329A substitution; a P329G substitution; an A330L substitution; an A330S substitution; a P331S substitution; an 1332E substitution; or a P396L substitution; or each of the combinations of mutations described earlier in the fourth embodiment of this application,
where the heavy chain variable domain comprises an amino acid sequence that is at least 80% identical or similar to SEQ ID NO: 234 and the light chain variable domain comprises an amino acid sequence that is at least 80% identical or similar to SEQ ID NO: 235, and
where the heavy chain variable domain comprises:
   a CDR-H1 amino acid sequence comprising SEQ ID NO: 147 or having 1, 2, 3, 4 or 5 amino acid alterations relative to SEQ ID NO: 147,
   a CDR-H2 amino acid sequence comprising SEQ ID NO: 153 or having 1, 2, 3, 4 or 5 amino acid alterations relative to SEQ ID NO: 153, and
   a CDR-H3 amino acid sequence comprising SEQ ID NO: 156 or having 1, 2, 3, 4 or 5 amino acid alterations relative to SEQ ID NO:156 (3B2g2m1) and
where the light chain variable domain comprises:
   a CDR-L1 amino acid sequence comprising SEQ ID NO: 159 or having 1, 2, 3, 4 or 5 amino acid alterations relative to SEQ ID NO: 159,
   a CDR-L2 amino acid sequence comprising SEQ ID NO: 172 or having 1, 2, 3, 4 or 5 amino acid alterations relative to SEQ ID NO: 172, and
   a CDR-L3 amino acid sequence comprising SEQ ID NO: 195 or having 1, 2, 3, 4 or 5 amino acid alterations relative to SEQ ID NO:195 (3B2g2m1).

In an embodiment, the identity or similarity is at least 81%, 82%, 83%, 84%, 85%, 86%, 87%, 88%, 89%, 90%, 91%, 92%, 93%, 94%, 95%, 96%, 97%, 98%, 99%.

In an embodiment, the antibody-based molecule that binds to human muscle-specific tyrosine-protein kinase (MuSK) comprises wild-type human IgG constant Fc region, a heavy chain variable domain and a light chain variable domain,
where the wild-type human IgG constant Fc region comprising SEQ ID NO: 266 or 267, wherein L234A and/or L235A substitution(s) (numbered according to the EU numbering system) is(are) introduced into said Fc region, and
where the heavy chain variable domain comprises an amino acid sequence that is at least 80% identical or similar to SEQ ID NO: 234 and the light chain variable domain comprises an amino acid sequence that is at least 80% identical or similar to SEQ ID NO: 235, and
where the heavy chain variable domain comprises:
   a CDR-H1 amino acid sequence comprising SEQ ID NO: 147 or having 1, 2, 3, 4 or 5 amino acid alterations relative to SEQ ID NO: 147,
   a CDR-H2 amino acid sequence comprising SEQ ID NO: 153 or having 1, 2, 3, 4 or 5 amino acid alterations relative to SEQ ID NO: 153, and
   a CDR-H3 amino acid sequence comprising SEQ ID NO: 156 or having 1, 2, 3, 4 or 5 amino acid alterations relative to SEQ ID NO:156 (3B2g2m1) and
where the light chain variable domain comprises:
   a CDR-L1 amino acid sequence comprising SEQ ID NO: 159 or having 1, 2, 3, 4 or 5 amino acid alterations relative to SEQ ID NO: 159,
   a CDR-L2 amino acid sequence comprising SEQ ID NO: 172 or having 1, 2, 3, 4 or 5 amino acid alterations relative to SEQ ID NO: 172, and
   a CDR-L3 amino acid sequence comprising SEQ ID NO: 195 or having 1, 2, 3, 4 or 5 amino acid alterations relative to SEQ ID NO:195 (3B2g2m1).

In an embodiment, the identity or similarity is at least 81%, 82%, 83%, 84%, 85%, 86%, 87%, 88%, 89%, 90%, 91%, 92%, 93%, 94%, 95%, 96%, 97%, 98%, 99%.

In an embodiment, the antibody-based molecule that binds to human muscle-specific tyrosine-protein kinase (MuSK) comprises wild-type human IgG constant Fc region, a heavy chain variable domain and a light chain variable domain, where the wild-type human IgG constant Fc region comprising SEQ ID NO: 266 or 267, wherein L234A and L235A substitutions (numbered according to the EU numbering system) are introduced into said Fc region, and where the heavy chain variable domain comprising SEQ ID NO: 234 and the light chain variable domain comprising SEQ ID NO: 235, and
where the heavy chain variable domain comprises:
   a CDR-H1 amino acid sequence comprising or consisting of SEQ ID NO: 147,
   a CDR-H2 amino acid sequence comprising or consisting of SEQ ID NO: 153, and
   a CDR-H3 amino acid sequence comprising or consisting of SEQ ID NO:156 (3B2g2m1) and
where the light chain variable domain comprises:
   a CDR-L1 amino acid sequence comprising or consisting of SEQ ID NO: 159,
   a CDR-L2 amino acid sequence comprising or consisting of SEQ ID NO: 172, and
   a CDR-L3 amino acid sequence comprising or consisting of SEQ ID NO:195 (3B2g2m1).
   a CDR-L3 amino acid sequence comprising SEQ ID NO: 195 or having 1, 2, 3, 4 or 5 amino acid alterations relative to SEQ ID NO:195 (3B2g2m1).

In an embodiment, the anti-MuSK antibody or antigen binding fragment thereof, comprises:
a) A full length heavy chain comprising an amino acid sequence that is at least 80% identical or similar to SEQ ID NO: 268 and
b) A full length light chain comprising an amino acid sequence that is at least 80% identical or similar to SEQ ID NO: 269, and
c) Wherein one or more of the following mutations (all numbered according to the EU numbering system) have been introduced into the full length heavy chain: an N297A substitution; an N297Q substitution; an L234A substitution; an L234D substitution; an L234E substitution; an L234G substitution; an L234H substitution; an L234F substitution; an L234K substitution; an L234Q substitution; an L234R substitution; an L234S substitution; an L234T substitution; an L235A substitution; an L235D substitution; an L235E substitution; an L235F substitution; an L235G substitution; an L235V substitution; an L235H substitution; an L2351 substitution; an L235K substitution; an L235R substitution; an L235S substitution; L235T substitution; an L235Q substitution; an L237A substitution; an S239D substitution; an E233P substitution; an L234V substitution; a C236 deletion; a G236E substitution; a G236R substitution; a G236K substitution; a G237A substitution; a P238A substitution; an F243L substitution; a D265A substitution; an S267E substitution; an H268A substitution; an R292P substitution; a Y300L substitution; a K322A substitution; a K322Q substitution; an A327Q substitution; an L328F substitution; an L328R substitution; a P329A substitution; a P329G substitution; an A330L substitution; an A330S substitution; a P331S substitution; an 1332E substitution; a P396L substitution; or each of the combinations of mutations described earlier in the fourth embodiment of this application, preferably the mutations is L234A or L235A, more preferably the mutations are L234A and L235A In an embodiment, the identity or similarity is at least 81%, 82%, 83%, 84%, 85%, 86%, 87%, 88%, 89%, 90%, 91%, 92%, 93%, 94%, 95%, 96%, 97%, 98%, 99%.

In an embodiment, the anti-MuSK antibody or antigen binding fragment thereof, comprises:
a) A full length heavy chain comprising SEQ ID NO: 268 and
b) A full length light chain comprising SEQ ID NO: 269, and
c) Wherein the full length heavy chain comprises L234A and L235A mutations numbered according to the EU numbering system.

In an embodiment, the anti-MuSK antibody or antigen binding fragment thereof, comprises:
a) A full length heavy chain comprising an amino acid sequence that is at least 80% identical or similar to SEQ ID NO: 270 and
b) A full length light chain comprising an amino acid sequence that is at least 80% identical or similar to SEQ ID NO: 271, and
c) Wherein one or more of the following mutations (all numbered according to the EU numbering system) have been introduced into the full length heavy chain: an N297A substitution; an N297Q substitution; an L234A substitution; an L234D substitution; an L234E substitution; an L234G substitution; an L234H substitution; an L234F substitution; an L234K substitution; an L234Q substitution; an L234R substitution; an L234S substitution; an L234T substitution; an L235A substitution; an L235D substitution; an L235E substitution; an L235F substitution; an L235G substitution; an L235V substitution; an L235H substitution; an L2351 substitution; an L235K substitution; an L235R substitution; an L235S substitution; L235T substitution; an L235Q substitution; an L237A substitution; an S239D substitution; an E233P substitution; an L234V substitution; a C236 deletion; a G236E substitution; a G236R substitution; a G236K substitution; a G237A substitution; a P238A substitution; an F243L substitution; a D265A substitution; an S267E substitution; an H268A substitution; an R292P substitution; a Y300L substitution; a K322A substitution; a K322Q substitution; an A327Q substitution; an L328F substitution; an L328R substitution; a P329A substitution; a P329G substitution; an A330L substitution; an A330S substitution; a P331S substitution; an 1332E substitution; a P396L substitution; or each of the combinations of mutations described earlier in the fourth embodiment of this application, preferably the mutations is L234A or L235A, more preferably the mutations are L234A and L235A.

In an embodiment, the identity or similarity is at least 81%, 82%, 83%, 84%, 85%, 86%, 87%, 88%, 89%, 90%, 91%, 92%, 93%, 94%, 95%, 96%, 97%, 98%, 99%.

In an embodiment, the anti-MuSK antibody or antigen binding fragment thereof, comprises:
a) A full length heavy chain comprising SEQ ID NO: 270 and
b) A full length light chain comprising SEQ ID NO: 271, and
c) Wherein the full length heavy chain comprises L234A and L235A mutations numbered according the EU numbering system.

In a preferred embodiment, the MuSK antibody-based molecule (or the anti-MuSK antibody or antigen binding fragment thereof) disclosed herein comprises a heavy chain variable domain (VH) and a light chain variable domain (VL),
where the heavy chain variable domain comprises:
   a CDR-H1 amino acid sequence comprising SEQ ID NO: 147 or having 1, 2, 3, 4 or 5 amino acid alterations relative to SEQ ID NO: 147,
   a CDR-H2 amino acid sequence comprising SEQ ID NO: 150 or having 1, 2, 3, 4 or 5 amino acid alterations relative to SEQ ID NO: 150, and
   a CDR-H3 amino acid sequence comprising SEQ ID NO: 156 or having 1, 2, 3, 4 or 5 amino acid alterations relative to SEQ ID NO:156 and
where the light chain variable domain comprises:
   a CDR-L1 amino acid sequence comprising SEQ ID NO: 159 or having 1, 2, 3, 4 or 5 amino acid alterations relative to SEQ ID NO: 159,
   a CDR-L2 amino acid sequence comprising SEQ ID NO: 172 or having 1, 2, 3, 4 or 5 amino acid alterations relative to SEQ ID NO: 172, and
   a CDR-L3 amino acid sequence comprising SEQ ID NO: 183 or having 1, 2, 3, 4 or 5 amino acid alterations relative to SEQ ID NO:183.

In a preferred embodiment, the MuSK antibody-based molecule (or the anti-MuSK antibody or antigen binding fragment thereof) disclosed herein comprises a heavy chain variable domain (VH) comprising an amino acid sequence that is at least 80% identical or similar to SEQ ID NO: 202 and a light chain variable domain (VL) comprising an amino acid sequence that is at least 80% identical or similar to SEQ ID NO: 203. In an embodiment, the identity or similarity is at least 81%, 82%, 83%, 84%, 85%, 86%, 87%, 88%, 89%, 90%, 91%, 92%, 93%, 94%, 95%, 96%, 97%, 98%, 99%. In a preferred embodiment, the MuSK antibody-based molecule disclosed herein comprises a heavy chain variable domain comprising amino acid sequence SEQ ID NO: 202 and a light chain variable domain comprising amino acid sequence SEQ ID NO: 203.

In a preferred embodiment, the antibody-based molecule that binds to human muscle-specific tyrosine-protein kinase (MuSK) comprises a heavy chain variable domain and a light chain variable domain, wherein the heavy chain variable domain comprises an amino acid sequence that is at least 80% identical or similar to SEQ ID NO: 202 and the light chain variable domain comprises an amino acid sequence that is at least 80% identical or similar to SEQ ID NO: 203, and
where the heavy chain variable domain comprises:
   a CDR-H1 amino acid sequence comprising SEQ ID NO: 147 or having 1, 2, 3, 4 or 5 amino acid alterations relative to SEQ ID NO: 147,
   a CDR-H2 amino acid sequence comprising SEQ ID NO: 150 or having 1, 2, 3, 4 or 5 amino acid alterations relative to SEQ ID NO: 150, and
   a CDR-H3 amino acid sequence comprising SEQ ID NO: 156 or having 1, 2, 3, 4 or 5 amino acid alterations relative to SEQ ID NO:156 (3B2) and
where the light chain variable domain comprises:
   a CDR-L1 amino acid sequence comprising SEQ ID NO: 159 or having 1, 2, 3, 4 or 5 amino acid alterations relative to SEQ ID NO: 159,
   a CDR-L2 amino acid sequence comprising SEQ ID NO: 172 or having 1, 2, 3, 4 or 5 amino acid alterations relative to SEQ ID NO: 172, and
   a CDR-L3 amino acid sequence comprising SEQ ID NO: 183 or having 1, 2, 3, 4 or 5 amino acid alterations relative to SEQ ID NO:183 (3B2).

In an embodiment, the identity or similarity is at least 81%, 82%, 83%, 84%, 85%, 86%, 87%, 88%, 89%, 90%, 91%, 92%, 93%, 94%, 95%, 96%, 97%, 98%, 99%.

In a more preferred embodiment, the antibody-based molecule that binds to human muscle-specific tyrosine-protein kinase (MuSK) comprises a heavy chain variable domain and a light chain variable domain, wherein the heavy chain variable domain comprises an amino acid sequence that is at least 80% identical or similar to SEQ ID NO: 202 and a light chain variable domain (VL) comprising an amino acid sequence that is at least 80% identical or similar to SEQ ID NO: 203, and
where the heavy chain variable domain comprises:
   a CDR-H1 amino acid sequence comprising or consisting of SEQ ID NO: 147,
   a CDR-H2 amino acid sequence comprising or consisting of SEQ ID NO: 150, and
   a CDR-H3 amino acid sequence comprising or consisting of SEQ ID NO:156 (382) and
where the light chain variable domain comprises:
   a CDR-L1 amino acid sequence comprising or consisting of SEQ ID NO: 159,
   a CDR-L2 amino acid sequence comprising or consisting of SEQ ID NO: 172, and
   a CDR-L3 amino acid sequence comprising or consisting of SEQ ID NO:183 (3B2).

In an embodiment, the identity or similarity is at least 81%, 82%, 83%, 84%, 85%, 86%, 87%, 88%, 89%, 90%, 91%, 92%, 93%, 94%, 95%, 96%, 97%, 98%, 99%.

In an embodiment, the antibody-based molecule that binds to human muscle-specific tyrosine-protein kinase (MuSK) comprises a heavy chain constant domain and a light chain constant domain (or human lambda constant domain), a heavy chain variable domain and a light chain variable domain, where the heavy chain constant domain comprising at least 80% sequence identity to SEQ ID NO: 280 and the light chain constant domain comprising at least 80% sequence identity to SEQ ID NO: 281, where the heavy chain variable domain comprises an amino acid sequence that is at least 80% identical or similar to SEQ ID NO: 202 and the light chain variable domain comprises an amino acid sequence that is at least 80% identical or similar to SEQ ID NO: 203, and
where the heavy chain variable domain comprises:
   a CDR-H1 amino acid sequence comprising SEQ ID NO: 147 or having 1, 2, 3, 4 or 5 amino acid alterations relative to SEQ ID NO: 147,
   a CDR-H2 amino acid sequence comprising SEQ ID NO: 150 or having 1, 2, 3, 4 or 5 amino acid alterations relative to SEQ ID NO: 150, and
   a CDR-H3 amino acid sequence comprising SEQ ID NO: 156 or having 1, 2, 3, 4 or 5 amino acid alterations relative to SEQ ID NO:156 (3B2) and
where the light chain variable domain comprises:
   a CDR-L1 amino acid sequence comprising SEQ ID NO: 159 or having 1, 2, 3, 4 or 5 amino acid alterations relative to SEQ ID NO: 159,
   a CDR-L2 amino acid sequence comprising SEQ ID NO: 172 or having 1, 2, 3, 4 or 5 amino acid alterations relative to SEQ ID NO: 172, and
   a CDR-L3 amino acid sequence comprising SEQ ID NO: 183 or having 1, 2, 3, 4 or 5 amino acid alterations relative to SEQ ID NO:183 (3B2).

In an embodiment, the identity or similarity is at least 81%, 82%, 83%, 84%, 85%, 86%, 87%, 88%, 89%, 90%, 91%, 92%, 93%, 94%, 95%, 96%, 97%, 98%, 99%.

In an embodiment, the CDR-H2 of the antibody comprises a proline (P) at position 3, a tryptophan (W) at position 4, and a serine (S) or asparagine (N) at position 5. In a preferred embodiment, the CDR-H2 of the antibody comprises a proline (P) at position 3, a tryptophan (W) at position 4, and an asparagine (N) at position 5.

In an embodiment, the antibody-based molecule that binds to human muscle-specific tyrosine-protein kinase (MuSK) comprises a heavy chain constant domain and light chain a constant domain (or human lambda constant domain), a heavy chain variable domain and a light chain variable domain, where the heavy chain constant domain comprising at least 80% sequence identity to SEQ ID NO: 280 and the light chain constant domain comprising at least 80% sequence identity to SEQ ID NO: 281, where the heavy chain variable domain comprises an amino acid sequence that is at least 80% identical or similar to SEQ ID NO: 202 and the light chain variable domain comprises an amino acid sequence that is at least 80% identical or similar to SEQ ID NO: 203, and
where the heavy chain variable domain comprises:
   a CDR-H1 amino acid sequence comprising SEQ ID NO: 147 or having 1, 2, 3, 4 or 5 amino acid alterations relative to SEQ ID NO: 147,
   a CDR-H2 amino acid sequence comprising SEQ ID NO: 150 or having 1, 2, 3, 4 or 5 amino acid alterations relative to SEQ ID NO: 150, and
   a CDR-H3 amino acid sequence comprising SEQ ID NO: 156 or having 1, 2, 3, 4 or 5 amino acid alterations relative to SEQ ID NO:156 (3B2) and
where the light chain variable domain comprises:
   a CDR-L1 amino acid sequence comprising SEQ ID NO: 159 or having 1, 2, 3, 4 or 5 amino acid alterations relative to SEQ ID NO: 159,
   a CDR-L2 amino acid sequence comprising SEQ ID NO: 172 or having 1, 2, 3, 4 or 5 amino acid alterations relative to SEQ ID NO: 172, and
   a CDR-L3 amino acid sequence comprising SEQ ID NO: 183 or having 1, 2, 3, 4 or 5 amino acid alterations relative to SEQ ID NO:183 (3B2).

In an embodiment, the identity or similarity is at least 81%, 82%, 83%, 84%, 85%, 86%, 87%, 88%, 89%, 90%, 91%, 92%, 93%, 94%, 95%, 96%, 97%, 98%, 99%.

In an embodiment, the antibody-based molecule that binds to human muscle-specific tyrosine-protein kinase (MuSK) comprises a heavy chain constant domain and a light chain constant domain (or human lambda constant domain), a heavy chain variable domain and a light chain variable domain, where the heavy chain constant domain comprising at least 80% sequence identity to SEQ ID NO: 280 and the light chain constant domain comprising at least 80% sequence identity to SEQ ID NO: 281, where the heavy chain variable domain comprising SEQ ID NO: 202 and the light chain variable domain comprising SEQ ID NO: 203, and
where the heavy chain variable domain comprises:
   a CDR-H1 amino acid sequence comprising SEQ ID NO: 147 or having 1, 2, 3, 4 or 5 amino acid alterations relative to SEQ ID NO: 147,
   a CDR-H2 amino acid sequence comprising SEQ ID NO: 150 or having 1, 2, 3, 4 or 5 amino acid alterations relative to SEQ ID NO: 150, and
   a CDR-H3 amino acid sequence comprising SEQ ID NO: 156 or having 1, 2, 3, 4 or 5 amino acid alterations relative to SEQ ID NO:156 (3B2) and
where the light chain variable domain comprises:
   a CDR-L1 amino acid sequence comprising SEQ ID NO: 159 or having 1, 2, 3, 4 or 5 amino acid alterations relative to SEQ ID NO: 159,
   a CDR-L2 amino acid sequence comprising SEQ ID NO: 172 or having 1, 2, 3, 4 or 5 amino acid alterations relative to SEQ ID NO: 172, and
   a CDR-L3 amino acid sequence comprising SEQ ID NO: 183 or having 1, 2, 3, 4 or 5 amino acid alterations relative to SEQ ID NO:183 (3B2).

In an embodiment, the CDR-H2 of the antibody comprises a proline (P) at position 3, a tryptophan (W) at position 4, and a serine (S) or asparagine (N) at position 5. In a preferred embodiment, the CDR-H2 of the antibody comprises a proline (P) at position 3, a tryptophan (W) at position 4, and an asparagine (N) at position 5.

In an embodiment, the antibody-based molecule that binds to human muscle-specific tyrosine-protein kinase (MuSK) comprises a heavy chain constant domain and a light chain constant domain (or human lambda constant domain), a heavy chain variable domain and a light chain variable domain, where the heavy chain constant domain comprising at least 80% sequence identity to SEQ ID NO: 280 and the light chain constant domain comprising at least 80% sequence identity to SEQ ID NO: 281, where the heavy chain variable domain comprising SEQ ID NO: 202 and the light chain variable domain comprising SEQ ID NO: 203, and
where the heavy chain variable domain comprises:
   a CDR-H1 amino acid sequence comprising or consisting of SEQ ID NO: 147,
   a CDR-H2 amino acid sequence comprising or consisting of SEQ ID NO: 150, and
   a CDR-H3 amino acid sequence comprising or consisting of SEQ ID NO:156 (382) and
where the light chain variable domain comprises:
   a CDR-L1 amino acid sequence comprising or consisting of SEQ ID NO: 159,
   a CDR-L2 amino acid sequence comprising or consisting of SEQ ID NO: 172, and
   a CDR-L3 amino acid sequence comprising or consisting of SEQ ID NO:183 (3B2).

In an embodiment, the antibody-based molecule that binds to human muscle-specific tyrosine-protein kinase (MuSK) comprises a heavy chain constant domain and a light chain constant domain (or human lambda constant domain), a heavy chain variable domain and a light chain variable domain, where the heavy chain constant domain comprising at least 80% sequence identity to SEQ ID NO: 280 and the light chain constant domain comprising at least 80% sequence identity to SEQ ID NO: 281, wherein one or more of the following mutations (all numbered according to the EU numbering system) have been introduced: an N297A substitution; an N297Q substitution; an L234A substitution; an L234D substitution; an L234E substitution; an L234G substitution; an L234H substitution; an L234F substitution; an L234K substitution; an L234Q substitution; an L234R substitution; an L234S substitution; an L234T substitution; an L235A substitution; an L235D substitution; an L235E substitution; an L235F substitution; an L235G substitution; an L235V substitution; an L235H substitution; an L2351 substitution; an L235K substitution; an L235R substitution; an L235S substitution; L235T substitution; an L235Q substitution; an L237A substitution; an S239D substitution; an E233P substitution; an L234V substitution; a C236 deletion; a G236E substitution; a G236R substitution; a G236K substitution; a G237A substitution; a P238A substitution; an F243L substitution; a D265A substitution; an S267E substitution; an H268A substitution; an R292P substitution; a Y300L substitution; a K322A substitution; a K322Q substitution; an A327Q substitution; an L328F substitution; an L328R substitution; a P329A substitution; a P329G substitution; an A330L substitution; an A330S substitution; a P331S substitution; an 1332E substitution; or a P396L substitution; or each of the combinations of mutations described earlier in the fourth embodiment of this application,
where the heavy chain variable domain comprises an amino acid sequence that is at least 80% identical or similar to SEQ ID NO: 202 and the light chain variable domain comprises an amino acid sequence that is at least 80% identical or similar to SEQ ID NO: 203, and
where the heavy chain variable domain comprises:
   a CDR-H1 amino acid sequence comprising SEQ ID NO: 147 or having 1, 2, 3, 4 or 5 amino acid alterations relative to SEQ ID NO: 147,
   a CDR-H2 amino acid sequence comprising SEQ ID NO: 150 or having 1, 2, 3, 4 or 5 amino acid alterations relative to SEQ ID NO: 150, and
   a CDR-H3 amino acid sequence comprising SEQ ID NO: 156 or having 1, 2, 3, 4 or 5 amino acid alterations relative to SEQ ID NO:156 (3B2) and
where the light chain variable domain comprises:
   a CDR-L1 amino acid sequence comprising SEQ ID NO: 159 or having 1, 2, 3, 4 or 5 amino acid alterations relative to SEQ ID NO: 159,
   a CDR-L2 amino acid sequence comprising SEQ ID NO: 172 or having 1, 2, 3, 4 or 5 amino acid alterations relative to SEQ ID NO: 172, and
   a CDR-L3 amino acid sequence comprising SEQ ID NO: 183 or having 1, 2, 3, 4 or 5 amino acid alterations relative to SEQ ID NO:183 (3B2).

In an embodiment, the identity or similarity is at least 81%, 82%, 83%, 84%, 85%, 86%, 87%, 88%, 89%, 90%, 91%, 92%, 93%, 94%, 95%, 96%, 97%, 98%, 99%.

In an embodiment, the antibody-based molecule that binds to human muscle-specific tyrosine-protein kinase (MuSK) comprises a heavy chain constant domain and a light chain constant domain (or human lambda constant domain), a heavy chain variable domain and a light chain variable domain, where the heavy chain constant domain comprising at least 80% sequence identity to SEQ ID NO: 280 and the light chain constant domain comprising at least 80% sequence identity to SEQ ID NO: 281,
wherein L234A and/or L235A substitution(s) (numbered according to the EU numbering system) is(are) introduced into said Fc region, and
where the heavy chain variable domain comprises an amino acid sequence that is at least 80% identical or similar to SEQ ID NO: 202 and the light chain variable domain comprises an amino acid sequence that is at least 80% identical or similar to SEQ ID NO: 203, and
where the heavy chain variable domain comprises:
   a CDR-H1 amino acid sequence comprising SEQ ID NO: 147 or having 1, 2, 3, 4 or 5 amino acid alterations relative to SEQ ID NO: 147,
   a CDR-H2 amino acid sequence comprising SEQ ID NO: 150 or having 1, 2, 3, 4 or 5 amino acid alterations relative to SEQ ID NO: 150, and
   a CDR-H3 amino acid sequence comprising SEQ ID NO: 156 or having 1, 2, 3, 4 or 5 amino acid alterations relative to SEQ ID NO:156 (3B2) and
where the light chain variable domain comprises:
   a CDR-L1 amino acid sequence comprising SEQ ID NO: 159 or having 1, 2, 3, 4 or 5 amino acid alterations relative to SEQ ID NO: 159,
   a CDR-L2 amino acid sequence comprising SEQ ID NO: 172 or having 1, 2, 3, 4 or 5 amino acid alterations relative to SEQ ID NO: 172, and
   a CDR-L3 amino acid sequence comprising SEQ ID NO: 183 or having 1, 2, 3, 4 or 5 amino acid alterations relative to SEQ ID NO:183 (3B2).

In an embodiment, the identity or similarity is at least 81%, 82%, 83%, 84%, 85%, 86%, 87%, 88%, 89%, 90%, 91%, 92%, 93%, 94%, 95%, 96%, 97%, 98%, 99%.

In an embodiment, the antibody-based molecule that binds to human muscle-specific tyrosine-protein kinase (MuSK) comprises a heavy chain constant domain and a light chain constant domain (or human lambda constant domain), a heavy chain variable domain and a light chain variable domain, where the heavy chain constant domain comprising at least 80% sequence identity to SEQ ID NO: 280 and the light chain constant domain comprising at least 80% sequence identity to SEQ ID NO: 281, wherein L234A and L235A substitutions (numbered according to the EU numbering system) are introduced into said Fc region, and
where the heavy chain variable domain comprising SEQ ID NO: 202 and the light chain variable domain comprising SEQ ID NO: 203, and
where the heavy chain variable domain comprises:
   a CDR-H1 amino acid sequence comprising or consisting of SEQ ID NO: 147,
   a CDR-H2 amino acid sequence comprising or consisting of SEQ ID NO: 150, and
   a CDR-H3 amino acid sequence comprising or consisting of SEQ ID NO:156 (382) and
where the light chain variable domain comprises:
   a CDR-L1 amino acid sequence comprising or consisting of SEQ ID NO: 159,
   a CDR-L2 amino acid sequence comprising or consisting of SEQ ID NO: 172, and
   a CDR-L3 amino acid sequence comprising or consisting of SEQ ID NO:183 (3B2).

In an embodiment, the anti-MuSK antibody or antigen binding fragment thereof, comprises:
a) a full length heavy chain comprising an amino acid sequence that is at least 80% identical or similar to SEQ ID NO: 282 and
b) a full length light chain comprising an amino acid sequence that is at least 80% identical or similar to SEQ ID NO: 283, and
c) wherein one or more of the following mutations (all numbered according to the EU numbering system) have been introduced into the full length heavy chain: an N297A substitution; an N297Q substitution; an L234A substitution; an L234D substitution; an L234E substitution; an L234G substitution; an L234H substitution; an L234F substitution; an L234K substitution; an L234Q substitution; an L234R substitution; an L234S substitution; an L234T substitution; an L235A substitution; an L235D substitution; an L235E substitution; an L235F substitution; an L235G substitution; an L235V substitution; an L235H substitution; an L2351 substitution; an L235K substitution; an L235R substitution; an L235S substitution; L235T substitution; an L235Q substitution; an L237A substitution; an S239D substitution; an E233P substitution; an L234V substitution; a C236 deletion; a G236E substitution; a G236R substitution; a G236K substitution; a G237A substitution; a P238A substitution; an F243L substitution; a D265A substitution; an S267E substitution; an H268A substitution; an R292P substitution; a Y300L substitution; a K322A substitution; a K322Q substitution; an A327Q substitution; an L328F substitution; an L328R substitution; a P329A substitution; a P329G substitution; an A330L substitution; an A330S substitution; a P331S substitution; an 1332E substitution; a P396L substitution; or each of the combinations of mutations described earlier in the fourth embodiment of this application, preferably the mutations is L234A or L235A, more preferably the mutations are L234A and L235A

In an embodiment, the identity or similarity is at least 81%, 82%, 83%, 84%, 85%, 86%, 87%, 88%, 89%, 90%, 91%, 92%, 93%, 94%, 95%, 96%, 97%, 98%, 99%.

In an embodiment, the anti-MuSK antibody or antigen binding fragment thereof, comprises:
d) A full length heavy chain comprising SEQ ID NO: 282 and
e) A full length light chain comprising SEQ ID NO: 283, and
f) Wherein the full length heavy chain comprises L234A and L235A mutations numbered according to the EU numbering system.

In an embodiment, the anti-MuSK antibody or antigen binding fragment thereof, comprises:
a) a full length heavy chain comprising an amino acid sequence that is at least 80% identical or similar to SEQ ID NO: 282 and
b) a full length light chain comprising an amino acid sequence that is at least 80% identical or similar to SEQ ID NO: 283, and
c) wherein one or more of the following mutations (all numbered according to the EU numbering system) have been introduced into the full length heavy chain: an N297A substitution; an N297Q substitution; an L234A substitution; an L234D substitution; an L234E substitution; an L234G substitution; an L234H substitution; an L234F substitution; an L234K substitution; an L234Q substitution; an L234R substitution; an L234S substitution; an L234T substitution; an L235A substitution; an L235D substitution; an L235E substitution; an L235F substitution; an L235G substitution; an L235V substitution; an L235H substitution; an L2351 substitution; an L235K substitution; an L235R substitution; an L235S substitution; L235T substitution; an L235Q substitution; an L237A substitution; an S239D substitution; an E233P substitution; an L234V substitution; a C236 deletion; a G236E substitution; a G236R substitution; a G236K substitution; a G237A substitution; a P238A substitution; an F243L substitution; a D265A substitution; an S267E substitution; an H268A substitution; an R292P substitution; a Y300L substitution; a K322A substitution; a K322Q substitution; an A327Q substitution; an L328F substitution; an L328R substitution; a P329A substitution; a P329G substitution; an A330L substitution; an A330S substitution; a P331S substitution; an 1332E substitution; a P396L substitution; or each of the combinations of mutations described earlier in the fourth embodiment of this application, preferably the mutations is L234A or L235A, more preferably the mutations are L234A and L235A In an embodiment, the identity or similarity is at least 81%, 82%, 83%, 84%, 85%, 86%, 87%, 88%, 89%, 90%, 91%, 92%, 93%, 94%, 95%, 96%, 97%, 98%, 99%.

In an embodiment, the anti-MuSK antibody or antigen binding fragment thereof, comprises:
a) a full length heavy chain comprising SEQ ID NO: 282 and
b) a full length light chain comprising SEQ ID NO: 283, and
c) wherein the full length heavy chain comprises L234A and L235A mutations numbered according the EU numbering system.

In the context of the application, the heavy chain constant domain described herein may contain additional lysine (or K) residue (s) at the C-terminal.

The properties of the anti-MuSK antibody described herein may be measured in accordance with the assays described herein. An activating activity of a MuSK agonist antibody may be measured relative to a control, for example a negative control antibody (such as an isotype control) that may not bind MuSK. A preferred control antibody not binding to MuSK is motavizumab which targets RSV (Review, MAbs, 1(5), 439-442, September-Octo 2009, DOI: 10.4161/mabs.1.5.9496). A preferred positive control agonist MuSK antibody is mAb #13 from Genentech. Another preferred positive control molecule for evidencing an activating MuSK activity is agrin (rat agrin from R&D systems, 550-AG).

By binding to an epitope of MuSK, the anti-MuSK antibody or antigen binding fragment of the invention are able to elicit an agonistic MuSK activity. Within the context of the application “elicit an agonistic MuSK activity” may be replaced by “activate MuSK”. An agonistic MuSK activity or an activation of MuSK may be triggered at the molecular and/or at the cellular level and/or in a more biological complex system as a NMJ, a synapse, a living organism. In the context of the application, an agonistic MuSK activity may be replaced by the triggering of a MuSK-induced signal or by the induction of MuSK activation in a muscle cell at the NMJ. A MuSK-induced signal (or MuSK activation or MuSK activity) may be at least one of the induction of MuSK dimerization, the induction of MuSK tyrosine phosphorylation, the induction or increase of induction of AChRs clustering at the NMJ (or clustering in vitro in myotubes AChR patches), the increase of the number or percentage of fully innervated NMJ, the decrease of the number or percentage of fully denervated NMJ, maintenance of the number or percentage of fully innervated NMJ (disease stabilization/disease progression stabilization), an improvement of the reliability of synaptic transmission, an improvement of motor performance, a prevention/stabilization or even a reduction/decrease of motor neuron death, an extension of the lifespan of a treated subject.

A MuSK-induced signal by the anti-MuSK antibody of the invention may be the induction of MuSK dimerization, which may be assessed by western blotting. In the context of the invention, an agonistic activity of MuSK may have been assessed when the induction of MuSK dimerization is increased of at least 10%, 20%, 30%, 40%, 50%, 60%, 70%, 80%, 90%, or 100% or more in an experiment using the antibody of the invention by comparison with the same experimental setting without any antibody or with a negative control or with a negative control antibody. Alternatively, in the context of the invention, an agonistic activity of MuSK antibody may have been assessed when the induction of MuSK dimerization is the same or about the same (20% less, 10% less or the same or 10% more or 20% more) in an experiment using the antibody of the invention by comparison with the same experimental setting without a positive control antibody. Such a MuSK dimerization may be assessed without agrin. A positive control in the assessment of MuSK dimerization is agrin.

A MuSK-induced signal by the anti-MuSK antibody of the invention may be the induction of MuSK tyrosine phosphorylation and such phosphorylation may be assessed by western blotting using an antibody specific for tyrosine phosphorylation. In the context of the invention, an agonistic activity of MuSK may have been assessed when the induction of MuSK tyrosine phosphorylation is increased of at least 10%, 20%, 30%, 40%, 50%, 60%, 70%, 80%, 90%, 100%, 120%, 150%, 180%, 200% or more in an experiment using the antibody of the invention by comparison with the same experimental setting without any antibody. Alternatively, in the context of the invention, an agonistic activity of MuSK may have been assessed when the induction of MuSK tyrosine phosphorylation is the same or about the same (20% less, 10% less or the same or 10% more or 20% more) in an experiment using the antibody of the invention by comparison with the same experimental setting without a positive control antibody. Such a MuSK tyrosine phosphorylation may be assessed without agrin. A positive control in the assessment of MuSK tyrosine phosphorylation is agrin.

A MuSK-induced signal by the anti-MuSK antibody of the invention may be the induction of acetylcholine receptor (AChR) clustering at the NMJ and such clustering may be assessed by staining of AChR using an antibody specifically binding to AChR and visualising such staining in fluorescent microscopy using techniques known to the skilled person. Alternatively, the clustering may be assessed in vitro in myotubes AChR patches. A preferred antibody used to visualise AChR clustering is an antibody specific for AChR. More preferred antibody is AlexaFluor488 conjugated a-bungarotoxin (B13422, ThermoFisher). Usually the region to be analysed in fixed in paraformaldehyde and incubated at room temperature with the relevant antibody of the invention or with a positive or negative control and subsequently each region is washed with PBS and observed under an epi-fluorescent microscopy. In the context of the invention, an agonistic activity of MuSK may have been assessed when the induction of AChR clustering at the NMJ is the same or about the same (i.e. 20% less, 10% less or the same or 10% more or 20% more) or is increased of at least 10%, 20%, 30%, 40%, 50%, 60%, 70%, 80%, 90%, or 100% in an experiment using the antibody of the invention by comparison with the same experimental setting without any antibody. Such a AchR clustering may be assessed without agrin. A positive control in the assessment of AchR clustering is agrin.

In a preferred embodiment, the anti-MuSK antibody of the invention exhibits an induction or increase of induction of acetylcholine receptor clustering at the NMJ and such clustering may be assessed by visualizing a staining or an increased staining for AchRs at the NMJ of diaphragms of mice compared to the staining obtained without MuSK agonist antibody. In an embodiment, this induction or increase of clustering of AchRs at the NMJ results in a more normal/physiological NMJ morphology maintaining synaptic innervation and/or pre- and post-synaptic alignment.

A MuSK-induced signal by the anti-MuSK antibody of the invention in a muscle cell at the NMJ may be the increase of the number or percentage of fully innervated NMJ, the decrease of the number or percentage of fully denervated NMJ, maintenance of the number or percentage of fully innervated NMJ (disease stabilization/disease progression stabilization), an improvement of the reliability of synaptic transmission, a prevention/stabilization or even a reduction/decrease of motor neuron death. Each of these features could be assessed using techniques known to the skilled person such as staining of AchR using the a-bungarotoxin antibody as earlier defined herein, presynaptic labelling and quantifying innervation by fluorescent confocal microscopy, EMG single fibre EMG, electrophysiology of single synapses, staining of motor neuron cell bodies in bone marrow specific regions. All these assays have been described in Cantor S et al 2018 (Elife, 2018; 7:e34375).

NMJ repair may be the induction or increase of nerve sprouting and/or the increase of the innervation status of the NMJ. Each of these effects may be assessed using techniques known to the skilled person.

### Polynucleotides

Another aspect of the present invention is directed to a polynucleotide for use in the treatment of a disease or condition resulting from a genetic defect in a fetus wherein the polynucleotide is administered to the mother of said fetus, said polynucleotide comprising a nucleotide sequence which encodes the antibody-based molecule of any preceding claims, or a VH, VL or CDR thereof.

The polynucleotide compositions can result in the generation of the antibody-based molecule in the subject within at least about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 20 hours, 25 hours, 30 hours, 35 hours, 40 hours, 45 hours, 50 hours, or 60 hours of administration of the composition to the subject. The composition can result in generation of the antibody-based molecule in the subject within at least about 1 day, 2 days, 3 days, 4 days, 5 days, 6 days, 7 days, 8 days, 9 days, or 10 days of administration of the composition to the subject The composition can result in generation of the antibody-based molecule in the subject within about 1 hour to about 6 days, about 1 hour to about 5 days, about 1 hour to about 4 days, about 1 hour to about 3 days, about 1 hour to about 2 days, about 1 hour to about 1 day, about 1 hour to about 72 hours, about 1 hour to about 60 hours, about 1 hour to about 48 hours, about 1 hour to about 36 hours, about 1 hour to about 24 hours, about 1 hour to about 12 hours, or about 1 hour to about 6 hours of administration of the composition to the subject.

The polynucleotide composition, when administered to the subject in need thereof, can result in the persistent generation of the antibody-based molecule in the subject. The composition can result in the generation of the antibody-based molecule in the subject for at least about 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, 42 days, 43 days, 44 days, 45 days, 46 days, 47 days, 48 days, 49 days, 50 days, 51 days, 52 days, 53 days, 54 days, 55 days, 56 days, 57 days, 58 days, 59 days, or 60 days.

In one embodiment, the polynucleotide encoding the antibody-based molecule of the present invention comprises a nucleotide sequence encoding any one, any two, any three, any four, any five, or any six of the CDRs described supra, including the heavy chain CDRs of SEQ ID NOs: 1-48, 135-140, 147-158, 240-251 and the light chain CDRs of SEQ ID NOs: 49-96, 141-146, and 159-195.

In an embodiment, the polynucleotide comprises a nucleotide sequence encoding a VH domain, where the VH domain comprises (i) a heavy chain variable domain comprising the CDR-H1 of SEQ ID NO: 1, the CDR-H2 of SEQ ID NO: 17, and the CDR-H3 of SEQ ID NO: 33; (ii) a heavy chain variable domain comprising the CDR-H1 of SEQ ID NO: 2, the CDR-H2 of SEQ ID NO: 18, and the CDR-H3 of SEQ ID NO: 34; (iii) a heavy chain variable domain comprising the CDR-H1 of SEQ ID NO: 3, the CDR-H2 of SEQ ID NO: 19, and the CDR-H3 of SEQ ID NO: 35; (iv) a heavy chain variable domain comprising the CDR-H1 of SEQ ID NO: 4, the CDR-H2 of SEQ ID NO: 20, and the CDR-H3 of SEQ ID NO: 36; (v) a heavy chain variable domain comprising the CDR-H1 of SEQ ID NO: 5, the CDR-H2 of SEQ ID NO: 21, and the CDR-H3 of SEQ ID NO: 37; (vi) a heavy chain variable domain comprising the CDR-H1 of SEQ ID NO: 6, the CDR-H2 of SEQ ID NO: 22, and the CDR-H3 of SEQ ID NO: 38; (vii) a heavy chain variable domain comprising the CDR-H1 of SEQ ID NO: 7, the CDR-H2 of SEQ ID NO: 23, and the CDR-H3 of SEQ ID NO: 39; (viii) a heavy chain variable domain comprising the CDR-H1 of SEQ ID NO: 8, the CDR-H2 of SEQ ID NO: 24, and the CDR-H3 of SEQ ID NO: 40; (ix) a heavy chain variable domain comprising the CDR-H1 of SEQ ID NO: 9, the CDR-H2 of SEQ ID NO: 25, and the CDR-H3 of SEQ ID NO: 41; (x) a heavy chain variable domain comprising the CDR-H1 of SEQ ID NO: 10, the CDR-H2 of SEQ ID NO: 26, and the CDR-H3 of SEQ ID NO: 42; (xi) a heavy chain variable domain comprising the CDR-H1 of SEQ ID NO: 11, the CDR-H2 of SEQ ID NO: 27, and the CDR-H3 of SEQ ID NO: 43; (xii) a heavy chain variable domain comprising the CDR-H1 of SEQ ID NO: 12, the CDR-H2 of SEQ ID NO: 28, and the CDR-H3 of SEQ ID NO: 44; (xiii) a heavy chain variable domain comprising the CDR-H1 of SEQ ID NO: 13, the CDR-H2 of SEQ ID NO: 29, and the CDR-H3 of SEQ ID NO: 45; (xiv) a heavy chain variable domain comprising the CDR-H1 of SEQ ID NO: 14, the CDR-H2 of SEQ ID NO: 30, and the CDR-H3 of SEQ ID NO: 46; (xv) a heavy chain variable domain comprising the CDR-H1 of SEQ ID NO: 15, the CDR-H2 of SEQ ID NO: 31, and the CDR-H3 of SEQ ID NO: 47; (xvi) a heavy chain variable domain comprising the CDR-H1 of SEQ ID NO: 16, the CDR-H2 of SEQ ID NO: 32, and the CDR-H3 of SEQ ID NO: 48; (xvii) a heavy chain variable domain comprising the CDR-H1 of SEQ ID NO: 135, the CDR-H2 of SEQ ID NO: 137, and the CDR-H3 of SEQ ID NO: 139; and (xviii) a heavy chain variable domain comprising the CDR-H1 of SEQ ID NO: 136, the CDR-H2 of SEQ ID NO: 138, and the CDR-H3 of SEQ ID NO: 140.

In some embodiments, the polynucleotide comprises a nucleotide sequence encoding a VH domain, where the VH domain comprises (ii.a) a heavy chain variable domain comprising the CDR-H1 of SEQ ID NO: 2, the CDR-H2 of SEQ ID NO: 18, and the CDR-H3 of SEQ ID NO: 240 (X2m1); (ii.b) a heavy chain variable domain comprising the CDR-H1 of SEQ ID NO: 2, the CDR-H2 of SEQ ID NO: 18, and the CDR-H3 of SEQ ID NO: 241 (X2m2); (ii.c) a heavy chain variable domain comprising the CDR-H1 of SEQ ID NO: 2, the CDR-H2 of SEQ ID NO: 18, and the CDR-H3 of SEQ ID NO: 242 (X2m3); (ii.d) a heavy chain variable domain comprising the CDR-H1 of SEQ ID NO: 2, the CDR-H2 of SEQ ID NO: 18, and the CDR-H3 of SEQ ID NO: 243 (X2m4); (ii.e) a heavy chain variable domain comprising the CDR-H1 of SEQ ID NO: 2, the CDR-H2 of SEQ ID NO: 18, and the CDR-H3 of SEQ ID NO: 244 (X2m5); (ii.f) a heavy chain variable domain comprising the CDR-H1 of SEQ ID NO: 2, the CDR-H2 of SEQ ID NO: 18, and the CDR-H3 of SEQ ID NO: 245 (X2m6); (ii.g) a heavy chain variable domain comprising the CDR-H1 of SEQ ID NO: 2, the CDR-H2 of SEQ ID NO: 18, and the CDR-H3 of SEQ ID NO: 246 (X2m7); (ii.h) a heavy chain variable domain comprising the CDR-H1 of SEQ ID NO: 2, the CDR-H2 of SEQ ID NO: 18, and the CDR-H3 of SEQ ID NO: 247 (X2m8).

In some embodiments, the polynucleotide comprises a nucleotide sequence encoding a VH domain, where the VH domain comprises (xvii.a) a heavy chain variable domain comprising the CDR-H1 of SEQ ID NO: 135, the CDR-H2 of SEQ ID NO: 137, and the CDR-H3 of SEQ ID NO: 248 (X17m1); (xvii.b) a heavy chain variable domain comprising the CDR-H1 of SEQ ID NO: 135, the CDR-H2 of SEQ ID NO: 137, and the CDR-H3 of SEQ ID NO: 249 (X17m2); (xvii.c) a heavy chain variable domain comprising the CDR-H1 of SEQ ID NO: 135, the CDR-H2 of SEQ ID NO: 137, and the CDR-H3 of SEQ ID NO: 250 (X17m3); (xvii.d) a heavy chain variable domain comprising the CDR-H1 of SEQ ID NO: 135, the CDR-H2 of SEQ ID NO: 137, and the CDR-H3 of SEQ ID NO: 251 (X17m6).

In an embodiment, the polynucleotide comprises a nucleotide sequence encoding a VH domain, where the VH domain comprises: (xix) a heavy chain variable domain comprising the CDR-H1 of SEQ ID NO: 147, the CDR-H2 of SEQ ID NO: 150, and the CDR-H3 of SEQ ID NO: 156; (xx) a heavy chain variable domain comprising the CDR-H1 of SEQ ID NO: 148, the CDR-H2 of SEQ ID NO: 151, and the CDR-H3 of SEQ ID NO: 157; (xxi) a heavy chain variable domain comprising the CDR-H1 of SEQ ID NO: 149, the CDR-H2 of SEQ ID NO: 152, and the CDR-H3 of SEQ ID NO: 158.

In an embodiment, the polynucleotide comprises a nucleotide sequence encoding a VH domain, where the VH domain comprises: (xxii) a heavy chain variable domain comprising the CDR-H1 of SEQ ID NO: 147, the CDR-H2 of SEQ ID NO: 153, and the CDR-H3 of SEQ ID NO:156; (xxiii) a heavy chain variable domain comprising the CDR-H1 of SEQ ID NO: 147, the CDR-H2 of SEQ ID NO: 154, and the CDR-H3 of SEQ ID NO: 156; (xxiv) a heavy chain variable domain comprising the CDR-H1 of SEQ ID NO: 147, the CDR-H2 of SEQ ID NO: 155, and the CDR-H3 of SEQ ID NO: 156.

In an embodiment, the polynucleotide comprises a nucleotide sequence encoding a VL domain, where the VL domain comprises (i) a light chain variable domain comprising the CDR-L1 of SEQ ID NO: 49, the CDR-L2 of SEQ ID NO: 65, and the CDR-L3 of SEQ ID NO: 81; (ii) a light chain variable domain comprising the CDR-L1 of SEQ ID NO: 50, the CDR-L2 of SEQ ID NO: 66, and the CDR-L3 of SEQ ID NO: 82; (iii) a light chain variable domain comprising the CDR-L1 of SEQ ID NO: 51, the CDR-L2 of SEQ ID NO: 67, and the CDR-L3 of SEQ ID NO: 83; (iv) a light chain variable domain comprising the CDR-L1 of SEQ ID NO: 52, the CDR-L2 of SEQ ID NO: 68, and the CDR-L3 of SEQ ID NO: 84; (v) a light chain variable domain comprising the CDR-L1 of SEQ ID NO: 53, the CDR-L2 of SEQ ID NO: 69, and the CDR-L3 of SEQ ID NO: 85; (vi) a light chain variable domain comprising the CDR-L1 of SEQ ID NO: 54, the CDR-L2 of SEQ ID NO: 70, and the CDR-L3 of SEQ ID NO: 86; (vii) a light chain variable domain comprising the CDR-L1 of SEQ ID NO: 55, the CDR-L2 of SEQ ID NO:71, and the CDR-L3 of SEQ ID NO: 87; (viii) a light chain variable domain comprising the CDR-L1 of SEQ ID NO: 56, the CDR-L2 of SEQ ID NO: 72, and the CDR-L3 of SEQ ID NO: 88; (ix) a light chain variable domain comprising the CDR-L1 of SEQ ID NO: 57, the CDR-L2 of SEQ ID NO: 73, and the CDR-L3 of SEQ ID NO: 89; (x) a light chain variable domain comprising the CDR-L1 of SEQ ID NO: 58, the CDR-L2 of SEQ ID NO: 74, and the CDR-L3 of SEQ ID NO: 90; (xi) a light chain variable domain comprising the CDR-L1 of SEQ ID NO: 59, the CDR-L2 of SEQ ID NO: 75, and the CDR-L3 of SEQ ID NO: 91; (xii) a light chain variable domain comprising the CDR-L1 of SEQ ID NO: 60, the CDR-L2 of SEQ ID NO: 76, and the CDR-L3 of SEQ ID NO: 92; (xiii) a light chain variable domain comprising the CDR-L1 of SEQ ID NO: 61, the CDR-L2 of SEQ ID NO: 77, and the CDR-L3 of SEQ ID NO: 93; (xiv) a light chain variable domain comprising the CDR-L1 of SEQ ID NO: 62, the CDR-L2 of SEQ ID NO: 78, and the CDR-L3 of SEQ ID NO: 94; (xv) a light chain variable domain comprising the CDR-L1 of SEQ ID NO: 63, the CDR-L2 of SEQ ID NO: 79, and the CDR-L3 of SEQ ID NO: 95; (xvi) a light chain variable domain comprising the CDR-L1 of SEQ ID NO: 64, the CDR-L2 of SEQ ID NO: 80, and the CDR-L3 of SEQ ID NO: 96; (xvii) a light chain variable domain comprising the CDR-L1 of SEQ ID NO: 141, the CDR-L2 of SEQ ID NO: 143, and the CDR-L3 of SEQ ID NO: 145; and (xviii) a light chain variable domain comprising the CDR-L1 of SEQ ID NO: 142, the CDR-L2 of SEQ ID NO: 144, and the CDR-L3 of SEQ ID NO: 146.

In an embodiment, the polynucleotide comprises a nucleotide sequence encoding a VL domain, where the VL domain comprises (xix) a light chain variable domain comprising the CDR-L1 of SEQ ID NO: 159, the CDR-L2 of SEQ ID NO: 170, and the CDR-L3 of SEQ ID NO: 180; (xx) a light chain variable domain comprising the CDR-L1 of SEQ ID NO: 159, the CDR-L2 of SEQ ID NO: 171, and the CDR-L3 of SEQ ID NO: 181; (xxi) a light chain variable domain comprising the CDR-L1 of SEQ ID NO: 160, the CDR-L2 of SEQ ID NO: 172, and the CDR-L3 of SEQ ID NO: 182; (xxii) a light chain variable domain comprising the CDR-L1 of SEQ ID NO: 159, the CDR-L2 of SEQ ID NO: 172, and the CDR-L3 of SEQ ID NO: 183; (xxiii) a light chain variable domain comprising the CDR-L1 of SEQ ID NO: 159, the CDR-L2 of SEQ ID NO: 171, and the CDR-L3 of SEQ ID NO: 184; (xxiv) a light chain variable domain comprising the CDR-L1 of SEQ ID NO: 159, the CDR-L2 of SEQ ID NO: 173, and the CDR-L3 of SEQ ID NO: 185; (xxv) a light chain variable domain comprising the CDR-L1 of SEQ ID NO: 159, the CDR-L2 of SEQ ID NO: 173, and the CDR-L3 of SEQ ID NO: 186; (xxvi) a light chain variable domain comprising the CDR-L1 of SEQ ID NO: 161, the CDR-L2 of SEQ ID NO: 174, and the CDR-L3 of SEQ ID NO: 187; (xxvii) a light chain variable domain comprising the CDR-L1 of SEQ ID NO: 162, the CDR-L2 of SEQ ID NO: 174, and the CDR-L3 of SEQ ID NO: 188; (xxviii) a light chain variable domain comprising the CDR-L1 of SEQ ID NO: 163, the CDR-L2 of SEQ ID NO: 174, and the CDR-L3 of SEQ ID NO: 188; (xxix) a light chain variable domain comprising the CDR-L1 of SEQ ID NO: 164, the CDR-L2 of SEQ ID NO: 174, and the CDR-L3 of SEQ ID NO: 189; (xxx) a light chain variable domain comprising the CDR-L1 of SEQ ID NO: 165, the CDR-L2 of SEQ ID NO: 175, and the CDR-L3 of SEQ ID NO: 190; (xxxi) a light chain variable domain comprising the CDR-L1 of SEQ ID NO: 166, the CDR-L2 of SEQ ID NO: 176, and the CDR-L3 of SEQ ID NO: 191; (xxxi) a light chain variable domain comprising the CDR-L1 of SEQ ID NO: 167, the CDR-L2 of SEQ ID NO: 177, and the CDR-L3 of SEQ ID NO: 192; (xxxii) a light chain variable domain comprising the CDR-L1 of SEQ ID NO: 168, the CDR-L2 of SEQ ID NO: 178, and the CDR-L3 of SEQ ID NO: 193; (xxxiii) a light chain variable domain comprising the CDR-L1 of SEQ ID NO: 169, the CDR-L2 of SEQ ID NO: 179, and the CDR-L3 of SEQ ID NO: 194.

In an embodiment, the polynucleotide comprises a nucleotide sequence encoding a VL domain, where the VL domain comprises (xxxiv) a light chain variable domain comprising the CDR-L1 of SEQ ID NO: 159, the CDR-L2 of SEQ ID NO: 172, and the CDR-L3 of SEQ ID NO: 183; (xxxv) a light chain variable domain comprising the CDR-L1 of SEQ ID NO: 159, the CDR-L2 of SEQ ID NO: 172, and the CDR-L3 of SEQ ID NO: 195.

In an embodiment, the isolated polynucleotide encoding the MuSK antibody-based molecule encodes any one of the VH and/or VL domain sequences as provided in Table 3. The nucleic acid molecules described herein include isolated polynucleotides, portions of expression vectors or portions of linear DNA sequences, including linear DNA sequences used for in vitro transcription/translation, and vectors compatible with prokaryotic, eukaryotic or filamentous phage expression, secretion, and/or display of the antibodies or binding fragments thereof described herein.

In a preferred embodiment, the polynucleotide comprises a nucleotide sequence encoding a VH that comprises an amino acid sequence that is at least 80% identical or similar to SEQ ID NO: 234. In another preferred embodiment, the polynucleotide comprises a nucleotide sequence encoding a VL that comprises an amino acid sequence that is at least 80% identical or similar to SEQ ID NO: 235

In a preferred embodiment, the polynucleotide comprises a nucleotide sequence encoding an antibody-based molecule that binds to human muscle-specific tyrosine-protein kinase (MuSK), said molecule comprising a heavy chain variable domain and a light chain variable domain, wherein the heavy chain variable domain comprises an amino acid sequence that is at least 80% identical or similar to SEQ ID NO: 234 and the light chain variable domain comprises an amino acid sequence that is at least 80% identical or similar to SEQ ID NO: 235, and
where the heavy chain variable domain comprises:
   a CDR-H1 amino acid sequence comprising SEQ ID NO: 147 or having 1, 2, 3, 4 or 5 amino acid alterations relative to SEQ ID NO: 147,
   a CDR-H2 amino acid sequence comprising SEQ ID NO: 153 or having 1, 2, 3, 4 or 5 amino acid alterations relative to SEQ ID NO: 153, and
   a CDR-H3 amino acid sequence comprising SEQ ID NO: 156 or having 1, 2, 3, 4 or 5 amino acid alterations relative to SEQ ID NO:156 (3B2g2m1) and
where the light chain variable domain comprises:
   a CDR-L1 amino acid sequence comprising SEQ ID NO: 159 or having 1, 2, 3, 4 or 5 amino acid alterations relative to SEQ ID NO: 159,
   a CDR-L2 amino acid sequence comprising SEQ ID NO: 172 or having 1, 2, 3, 4 or 5 amino acid alterations relative to SEQ ID NO: 172, and
   a CDR-L3 amino acid sequence comprising SEQ ID NO: 195 or having 1, 2, 3, 4 or 5 amino acid alterations relative to SEQ ID NO:195 (3B2g2m1).

In an embodiment, the identity or similarity is at least 81%, 82%, 83%, 84%, 85%, 86%, 87%, 88%, 89%, 90%, 91%, 92%, 93%, 94%, 95%, 96%, 97%, 98%, 99%.

In a more preferred embodiment, the polynucleotide comprises a nucleotide sequence encoding an antibody-based molecule that binds to human muscle-specific tyrosine-protein kinase (MuSK), that comprises a heavy chain variable domain and a light chain variable domain, wherein the heavy chain variable domain comprises an amino acid sequence that is at least 80% identical or similar to SEQ ID NO: 234 and the light chain variable domain comprises an amino acid sequence that is at least 80% identical or similar to SEQ ID NO: 235, and
where the heavy chain variable domain comprises:
   a CDR-H1 amino acid sequence comprising or consisting of SEQ ID NO: 147,
   a CDR-H2 amino acid sequence comprising or consisting of SEQ ID NO: 153, and
   a CDR-H3 amino acid sequence comprising or consisting of SEQ ID NO:156 (3B2g2m1) and
where the light chain variable domain comprises:
   a CDR-L1 amino acid sequence comprising or consisting of SEQ ID NO: 159,
   a CDR-L2 amino acid sequence comprising or consisting of SEQ ID NO: 172, and
   a CDR-L3 amino acid sequence comprising or consisting of SEQ ID NO:195 (3B2g2m1).

In an embodiment, the identity or similarity is at least 81%, 82%, 83%, 84%, 85%, 86%, 87%, 88%, 89%, 90%, 91%, 92%, 93%, 94%, 95%, 96%, 97%, 98%, 99%. In a more preferred embodiment, the polynucleotide comprises or consists of a nucleotide sequence that is at least 80% 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO: 264. In an even more preferred embodiment, the polynucleotide comprises or consists of SEQ ID NO:264. In another more preferred embodiment, the polynucleotide comprises or consists of a nucleotide sequence that is at least 80% 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO: 265. In an even more preferred embodiment, the polynucleotide comprises or consists of SEQ ID NO:265.

In a more preferred embodiment, the polynucleotide comprises a nucleotide sequence encoding an antibody-based molecule that binds to human muscle-specific tyrosine-protein kinase (MuSK), that comprises a heavy chain variable domain and a light chain variable domain, wherein the heavy chain variable domain is encoded by a nucleotide sequence that is at least 80% identical to SEQ ID NO: 264 and the light chain variable domain is encoded by a nucleotide sequence that is at least 80% identical to SEQ ID NO: 265. In an embodiment, the identity is at least 81%, 82%, 83%, 84%, 85%, 86%, 87%, 88%, 89%, 90%, 91%, 92%, 93%, 94%, 95%, 96%, 97%, 98%, 99%.

In an embodiment, the polynucleotide comprises:
a) a nucleotide sequence that is at least 80% identical to SEQ ID NO:276 encoding the full length heavy chain, and
b) a nucleotide sequence that is at least 80% identical to SEQ ID NO:278 encoding the full length light chain.

In an embodiment, the identity is at least 81%, 82%, 83%, 84%, 85%, 86%, 87%, 88%, 89%, 90%, 91%, 92%, 93%, 94%, 95%, 96%, 97%, 98%, 99%.

In an embodiment, the polynucleotide comprises:
a) a nucleotide sequence that is at least 80% identical to SEQ ID NO:277 encoding the heavy chain variable domain, and
b) a nucleotide sequence that is at least 80% identical to SEQ ID NO:279 encoding the light chain variable domain.

In an embodiment, the identity is at least 81%, 82%, 83%, 84%, 85%, 86%, 87%, 88%, 89%, 90%, 91%, 92%, 93%, 94%, 95%, 96%, 97%, 98%, 99%.

In an embodiment, the polynucleotide comprises:
a) a nucleotide sequence that is at least 80% identical to SEQ ID NO:276 encoding the full length heavy chain, wherein said nucleotide sequence comprising a nucleotide sequence that is at least 80% identical to SEQ ID NO: 277 encoding the heavy chain variable domain, and
b) a nucleotide sequence that is at least 80% identical to SEQ ID NO:278 encoding the full length light chain, wherein said nucleotide sequence comprising a nucleotide sequence that is at least 80% identical to SEQ ID NO:279 encoding the light chain variable domain.

In an embodiment, the identity is at least 81%, 82%, 83%, 84%, 85%, 86%, 87%, 88%, 89%, 90%, 91%, 92%, 93%, 94%, 95%, 96%, 97%, 98%, 99%.

In an embodiment, the polynucleotide comprises:
a) a nucleotide sequence SEQ ID NO:276 and
b) a nucleotide sequence SEQ ID NO:278.

The polynucleotides of the invention may be produced by chemical synthesis such as solid phase polynucleotide synthesis on an automated polynucleotide synthesizer and assembled into complete single or double stranded molecules. Alternatively, the polynucleotides may be produced by other techniques such a PCR followed by routine cloning. Techniques for producing or obtaining polynucleotides of a given sequence are well known in the art.

The polynucleotides may comprise at least one non-coding sequence, such as a promoter or enhancer sequence, intron, polyadenylation signal, a cis sequence facilitating RepA binding, and the like. The polynucleotide sequences may also comprise additional sequences encoding for example a linker sequence, a marker or a tag sequence, such as a histidine tag or an HA tag to facilitate purification or detection of the protein, a signal sequence, a fusion protein partner such as RepA, Fc portion, or bacteriophage coat protein such as pIX or pIII.

In a preferred embodiment, the polynucleotide comprises a nucleotide sequence encoding a VH that comprises an amino acid sequence that is at least 80% identical or similar to SEQ ID NO: 202. In another preferred embodiment, the polynucleotide comprises a nucleotide sequence encoding a VL that comprises an amino acid sequence that is at least 80% identical or similar to SEQ ID NO: 203

In a preferred embodiment, the polynucleotide comprises a nucleotide sequence encoding an antibody-based molecule that binds to human muscle-specific tyrosine-protein kinase (MuSK), said molecule comprising a heavy chain variable domain and a light chain variable domain, wherein the heavy chain variable domain comprises an amino acid sequence that is at least 80% identical or similar to SEQ ID NO: 202 and the light chain variable domain comprises an amino acid sequence that is at least 80% identical or similar to SEQ ID NO: 203, and
where the heavy chain variable domain comprises:
   a CDR-H1 amino acid sequence comprising SEQ ID NO: 147 or having 1, 2, 3, 4 or 5 amino acid alterations relative to SEQ ID NO: 147,
   a CDR-H2 amino acid sequence comprising SEQ ID NO: 150 or having 1, 2, 3, 4 or 5 amino acid alterations relative to SEQ ID NO: 150, and
   a CDR-H3 amino acid sequence comprising SEQ ID NO: 156 or having 1, 2, 3, 4 or 5 amino acid alterations relative to SEQ ID NO:156 (3B2) and
where the light chain variable domain comprises:
   a CDR-L1 amino acid sequence comprising SEQ ID NO: 159 or having 1, 2, 3, 4 or 5 amino acid alterations relative to SEQ ID NO: 159,
   a CDR-L2 amino acid sequence comprising SEQ ID NO: 172 or having 1, 2, 3, 4 or 5 amino acid alterations relative to SEQ ID NO: 172, and
   a CDR-L3 amino acid sequence comprising SEQ ID NO: 183 or having 1, 2, 3, 4 or 5 amino acid alterations relative to SEQ ID NO:183 (3B2).

In an embodiment, the identity or similarity is at least 81%, 82%, 83%, 84%, 85%, 86%, 87%, 88%, 89%, 90%, 91%, 92%, 93%, 94%, 95%, 96%, 97%, 98%, 99%.

In a more preferred embodiment, the polynucleotide comprises a nucleotide sequence encoding an antibody-based molecule that binds to human muscle-specific tyrosine-protein kinase (MuSK), that comprises a heavy chain variable domain and a light chain variable domain, wherein the heavy chain variable domain comprises an amino acid sequence that is at least 80% identical or similar to SEQ ID NO: 202 and the light chain variable domain comprises an amino acid sequence that is at least 80% identical or similar to SEQ ID NO: 203, and
where the heavy chain variable domain comprises:
   a CDR-H1 amino acid sequence comprising or consisting of SEQ ID NO: 147,
   a CDR-H2 amino acid sequence comprising or consisting of SEQ ID NO: 150, and
   a CDR-H3 amino acid sequence comprising or consisting of SEQ ID NO:156 (382) and
where the light chain variable domain comprises:
   a CDR-L1 amino acid sequence comprising or consisting of SEQ ID NO: 159,
   a CDR-L2 amino acid sequence comprising or consisting of SEQ ID NO: 172, and
   a CDR-L3 amino acid sequence comprising or consisting of SEQ ID NO:183 (3B2).

In an embodiment, the identity or similarity is at least 81%, 82%, 83%, 84%, 85%, 86%, 87%, 88%, 89%, 90%, 91%, 92%, 93%, 94%, 95%, 96%, 97%, 98%, 99%.

In a more preferred embodiment, the polynucleotide comprises a nucleotide sequence encoding an antibody-based molecule that binds to human muscle-specific tyrosine-protein kinase (MuSK), that comprises a heavy chain variable domain and a light chain variable domain, wherein the heavy chain variable domain is encoded by a nucleotide sequence that is at least 80% identical to SEQ ID NO: 284 and the light chain variable domain is encoded by a nucleotide sequence that is at least 80% identical to SEQ ID NO: 285. In an embodiment, the identity is at least 81%, 82%, 83%, 84%, 85%, 86%, 87%, 88%, 89%, 90%, 91%, 92%, 93%, 94%, 95%, 96%, 97%, 98%, 99%.

In an embodiment, the polynucleotide comprises:
a) a nucleotide sequence that is at least 80% identical to SEQ ID NO:288 encoding the full length heavy chain, and
b) a nucleotide sequence that is at least 80% identical to SEQ ID NO:289 encoding the full length light chain.

In an embodiment, the identity is at least 81%, 82%, 83%, 84%, 85%, 86%, 87%, 88%, 89%, 90%, 91%, 92%, 93%, 94%, 95%, 96%, 97%, 98%, 99%.

In an embodiment, the polynucleotide comprises:
a) a nucleotide sequence that is at least 80% identical to SEQ ID NO:284 encoding the heavy chain variable domain, and
b) a nucleotide sequence that is at least 80% identical to SEQ ID NO:285 encoding the light chain variable domain.

In an embodiment, the identity is at least 81%, 82%, 83%, 84%, 85%, 86%, 87%, 88%, 89%, 90%, 91%, 92%, 93%, 94%, 95%, 96%, 97%, 98%, 99%.

In an embodiment, the polynucleotide comprises:
a) a nucleotide sequence that is at least 80% identical to SEQ ID NO:288 encoding the full length heavy chain, wherein said nucleotide sequence comprising a nucleotide sequence that is at least 80% identical to SEQ ID NO: 284 encoding the heavy chain variable domain, and
b) a nucleotide sequence that is at least 80% identical to SEQ ID NO:289 encoding the full length light chain, wherein said nucleotide sequence comprising a nucleotide sequence that is at least 80% identical to SEQ ID NO:285 encoding the light chain variable domain.

In an embodiment, the identity is at least 81%, 82%, 83%, 84%, 85%, 86%, 87%, 88%, 89%, 90%, 91%, 92%, 93%, 94%, 95%, 96%, 97%, 98%, 99%.

In an embodiment, the polynucleotide comprises:
a) a nucleotide sequence SEQ ID NO:288 and
b) a nucleotide sequence SEQ ID NO:289.

### Vector

In another aspect, there is provided an expression vector for use in the treatment of a disease or condition resulting from a genetic defect in a fetus wherein the expression vector is administered to the mother of said fetus, comprising the polynucleotide encoding the antibody-based molecule as described herein, preferably operably linked to a regulatory region which allows expression of the antibody-based molecule or VH, VL or CDR thereof in a host cell or cell-free expression system.

Such vectors include, without limitation, plasmid vectors, viral vectors, including without limitation, vaccina vector, lentiviral vector, adenoviral vector, adeno-associated viral vector, vectors for baculovirus expression, transposon based vectors or any other vector suitable for introduction of the polynucleotides described herein into a given organism or genetic background by any means to facilitate expression of the encoded antibody polypeptide. In one embodiment, the polynucleotide encoding the VH, alone or together with the polynucleotide encoding the VL as described herein, are combined with sequences of a promoter, a translation initiation segment (e.g., a ribosomal binding sequence and start codon), a 3′ untranslated region, polyadenylation signal, a termination codon, and transcription termination to form one or more expression vector constructs.

In one embodiment, the vector is an adenoviral-associated viral (AAV) vector. A number of therapeutic AAV vectors suitable for delivery of the polynucleotides encoding antibodies described herein to the central nervous system are known in the art. See e.g., Deverman et al., “Gene Therapy for Neurological Disorders: Progress and Prospects,” Nature Rev. 17:641-659 (2018), which in hereby incorporated by reference in its entirety. Suitable AAV vectors include serotypes AAV1, AAV2, AAV3, AAV4, AAV5, AAV6, AAV7, AAV8, AAV9, AAV10, or AAV11 in their native form or engineered for enhanced tropism.

AAV vectors known to have tropism for the CNS that are particularly suited for therapeutic expression of the MuSK antibodies described herein include, AAV1, AAV2, AAV4, AAV5, AAV8 and AAV9 in their native form or engineered for enhanced tropism. In one embodiment, the AAV vector is an AAV2 vector.

In another embodiment, the AAV vector is an AAV5 vector (Vitale et al., “Anti-tau Conformational scFv MC1 Antibody Efficiently Reduces Pathological Tau Species in Adult JNPL3 Mice,” Acta Neuropathol. Commun. 6:82 (2018), which is hereby incorporate by reference in its entirety). In another embodiment, the AAV vector is an AAV9 vector (Haiyan et al., “Targeting Root Cause by Systemic scAAV9-hIDS Gene Delivery: Functional Correction and Reversal of Severe MPSII in Mice,” Mol. Ther. Methods Clin. Dev. 10:327-340 (2018), which is hereby incorporated by reference in its entirety). In another embodiment, the AAV vector is an AAVrh10 vector (Liu et al., “Vectored Intracerebral Immunizations with the Anti-Tau Monoclonal Antibody PHF1 Markedly Reduces Tau Pathology in Mutant Transgenic Mice,” J. Neurosci. 36(49): 12425-35 (2016), which is hereby incorporated by reference in its entirety).

In another embodiment the AAV vector is a hybrid vector comprising the genome of one serotype, e.g., AAV2, and the capsid protein of another serotype, e.g., AAV1 or AAV3-9 to control tropism. See e.g., Broekman et al., “Adeno-associated Virus Vectors Serotyped with AAV8 Capsid are More Efficient than AAV-1 or -2 Serotypes for Widespread Gene Delivery to the Neonatal Mouse Brain,” Neuroscience 138:501-510 (2006), which is hereby incorporated by reference in its entirety. In one embodiment, the AAV vector is an AAV2/8 hybrid vector (Ising et al., “AAV-mediated Expression of Anti-Tau ScFv Decreases Tau Accumulation in a Mouse Model of Tauopathy,” J. Exp. Med. 214(5):1227 (2017), which is hereby incorporated by reference in its entirety). In another embodiment the AAV vector is an AAV2/9 hybrid vector (Simon et al., “A Rapid Gene Delivery-Based Mouse Model for Early-Stage Alzheimer Disease-Type Tauopathy,” J. Neuropath. Exp. Neurol. 72(11): 1062-71 (2013), which is hereby incorporated by reference in its entirety).

In another embodiment, the AAV vector is one that has been engineered or selected for its enhanced CNS transduction after intraparenchymal administration, e.g., AAV-DJ (Grimm et al., J. Viol. 82:5887-5911 (2008), which is hereby incorporated by reference in its entirety); increased transduction of neural stem and progenitor cells, e.g., SCH9 and AAV4.18 (Murlidharan et al., J. Virol. 89: 3976-3987 (2015) and Ojala et al., Mol. Ther. 26:304-319 (2018), which are hereby incorporated by reference in their entirety); enhanced retrograde transduction, e.g., rAAV2-retro (Muller et al., Nat. Biotechnol. 21:1040-1046 (2003), which is hereby incorporated by reference in its entirety); selective transduction into brain endothelial cells, e.g., AAV-BRI (Korbelin et al., EMBO Mol. Med. 8: 609-625 (2016), which is hereby incorporated by reference in its entirety); or enhanced transduction of the adult CNS after IV administration, e.g., AAV-PHP.B and AAVPHP.eB (Deverman et al., Nat. Biotechnol. 34:204-209 (2016) and Chan et al., Nat. Neurosci. 20: 1172-1179 (2017), which are hereby incorporated by reference in their entirety.

In accordance with this embodiment, the expression vector construct encoding the antibody-based molecule includes the polynucleotide encoding the heavy chain polypeptide, a functional fragment thereof, a variant thereof, or combinations thereof. The expression construct can alternatively include a nucleic acid sequence encoding the light chain polypeptide, a functional fragment thereof, a variant thereof, or combinations thereof. In an embodiment, the expression vector construct includes a nucleic acid sequence encoding the heavy chain polypeptide, a functional fragment thereof, or a variant thereof, and the light chain polypeptide, a functional fragment thereof, or a variant thereof.

In an embodiment, the expression construct further comprises a promoter sequence suitable for driving expression of the antibody-based molecule. Suitable promoter sequences include, without limitation, the elongation factor 1-alpha promoter (EF1a) promoter, a phosphoglycerate kinase-1 promoter (PGK) promoter, a cytomegalovirus immediate early gene promoter (CMV), a chimeric liver-specific promoter (LSP), a cytomegalovirus enhancer/chicken beta-actin promoter (CAG), a tetracycline responsive promoter (TRE), a transthyretin promoter (TTR), a simian virus 40 promoter (SV40) and a CK6 promoter. Other promoters suitable for driving gene expression in mammalian cells that are known in the art are also suitable for incorporation into the expression constructs disclosed herein.

In an embodiment, the expression construct (or expression vector) further encodes a linker sequence. The linker sequence can encode an amino acid sequence that spatially separates and/or links the one or more components of the expression construct (heavy chain and light chain components of the encoded antibody).

In a preferred embodiment, the expression vector comprises a polynucleotide that encodes an antibody-based molecule that binds to human muscle-specific tyrosine-protein kinase (MuSK) and that comprises a heavy chain variable domain and a light chain variable domain, wherein the heavy chain variable domain comprises an amino acid sequence that is at least 80% identical or similar to SEQ ID NO: 234 and the light chain variable domain comprises an amino acid sequence that is at least 80% identical or similar to SEQ ID NO: 235, and
where the heavy chain variable domain comprises:
   a CDR-H1 amino acid sequence comprising SEQ ID NO: 147 or having 1, 2, 3, 4 or 5 amino acid alterations relative to SEQ ID NO: 147,
   a CDR-H2 amino acid sequence comprising SEQ ID NO: 153 or having 1, 2, 3, 4 or 5 amino acid alterations relative to SEQ ID NO: 153, and
   a CDR-H3 amino acid sequence comprising SEQ ID NO: 156 or having 1, 2, 3, 4 or 5 amino acid alterations relative to SEQ ID NO:156 (3B2g2m1) and
where the light chain variable domain comprises:
   a CDR-L1 amino acid sequence comprising SEQ ID NO: 159 or having 1, 2, 3, 4 or 5 amino acid alterations relative to SEQ ID NO: 159,
   a CDR-L2 amino acid sequence comprising SEQ ID NO: 172 or having 1, 2, 3, 4 or 5 amino acid alterations relative to SEQ ID NO: 172, and
   a CDR-L3 amino acid sequence comprising SEQ ID NO: 195 or having 1, 2, 3, 4 or 5 amino acid alterations relative to SEQ ID NO:195 (3B2g2m1).

In an embodiment, the identity or similarity is at least 81%, 82%, 83%, 84%, 85%, 86%, 87%, 88%, 89%, 90%, 91%, 92%, 93%, 94%, 95%, 96%, 97%, 98%, 99%.

In a more preferred embodiment, the expression vector comprises a polynucleotide encoding an antibody-based molecule that binds to human muscle-specific tyrosine-protein kinase (MuSK) and comprising a heavy chain variable domain and a light chain variable domain, wherein the heavy chain variable domain comprises an amino acid sequence that is at least 80% identical or similar to SEQ ID NO: 234 and the light chain variable domain comprises an amino acid sequence that is at least 80% identical or similar to SEQ ID NO: 235, and
where the heavy chain variable domain comprises:
   a CDR-H1 amino acid sequence comprising or consisting of SEQ ID NO: 147,
   a CDR-H2 amino acid sequence comprising or consisting of SEQ ID NO: 153, and
   a CDR-H3 amino acid sequence comprising or consisting of SEQ ID NO:156 (3B2g2m1) and
where the light chain variable domain comprises:
   a CDR-L1 amino acid sequence comprising or consisting of SEQ ID NO: 159,
   a CDR-L2 amino acid sequence comprising or consisting of SEQ ID NO: 172, and
   a CDR-L3 amino acid sequence comprising or consisting of SEQ ID NO:195 (3B2g2m1).

In an embodiment, the identity or similarity is at least 81%, 82%, 83%, 84%, 85%, 86%, 87%, 88%, 89%, 90%, 91%, 92%, 93%, 94%, 95%, 96%, 97%, 98%, 99%.

In an embodiment, the expression vector comprises a polynucleotide encoding an antibody-based molecule that binds to human muscle-specific tyrosine-protein kinase (MuSK) and comprising:
a) a nucleotide sequence that is at least 80% identical to SEQ ID NO:276 encoding the full length heavy chain, and
b) a nucleotide sequence that is at least 80% identical to SEQ ID NO:278 encoding the full length light chain.

In an embodiment, the identity is at least 81%, 82%, 83%, 84%, 85%, 86%, 87%, 88%, 89%, 90%, 91%, 92%, 93%, 94%, 95%, 96%, 97%, 98%, 99%.

In an embodiment, the expression vector comprises a polynucleotide encoding an antibody-based molecule that binds to human muscle-specific tyrosine-protein kinase (MuSK) and comprising:
a) a nucleotide sequence that is at least 80% identical to SEQ ID NO:277 encoding the heavy chain variable domain, and
b) a nucleotide sequence that is at least 80% identical to SEQ ID NO:279 encoding the light chain variable domain.

In an embodiment, the identity is at least 81%, 82%, 83%, 84%, 85%, 86%, 87%, 88%, 89%, 90%, 91%, 92%, 93%, 94%, 95%, 96%, 97%, 98%, 99%.

In an embodiment, the expression vector comprises a polynucleotide encoding an antibody-based molecule that binds to human muscle-specific tyrosine-protein kinase (MuSK) and comprising:
c) a nucleotide sequence that is at least 80% identical to SEQ ID NO:276 encoding the full length heavy chain, wherein said nucleotide sequence comprising a nucleotide sequence that is at least 80% identical to SEQ ID NO: 277 encoding the heavy chain variable domain, and
d) a nucleotide sequence that is at least 80% identical to SEQ ID NO:278 encoding the full length light chain, wherein said nucleotide sequence comprising a nucleotide sequence that is at least 80% identical to SEQ ID NO:279 encoding the light chain variable domain.

In an embodiment, the identity is at least 81%, 82%, 83%, 84%, 85%, 86%, 87%, 88%, 89%, 90%, 91%, 92%, 93%, 94%, 95%, 96%, 97%, 98%, 99%.

In an embodiment, the expression vector comprises a polynucleotide encoding an antibody-based molecule that binds to human muscle-specific tyrosine-protein kinase (MuSK) and comprising:
a) a nucleotide sequence SEQ ID NO:276 and
b) a nucleotide sequence SEQ ID NO:278.

In a preferred embodiment, the expression vector comprises a polynucleotide that encodes an antibody-based molecule that binds to human muscle-specific tyrosine-protein kinase (MuSK) and that comprises a heavy chain variable domain and a light chain variable domain, wherein the heavy chain variable domain comprises an amino acid sequence that is at least 80% identical or similar to SEQ ID NO: 202 and the light chain variable domain comprises an amino acid sequence that is at least 80% identical or similar to SEQ ID NO: 203, and
where the heavy chain variable domain comprises:
   a CDR-H1 amino acid sequence comprising SEQ ID NO: 147 or having 1, 2, 3, 4 or 5 amino acid alterations relative to SEQ ID NO: 147,
   a CDR-H2 amino acid sequence comprising SEQ ID NO: 150 or having 1, 2, 3, 4 or 5 amino acid alterations relative to SEQ ID NO: 150, and
   a CDR-H3 amino acid sequence comprising SEQ ID NO: 156 or having 1, 2, 3, 4 or 5 amino acid alterations relative to SEQ ID NO:156 (3B2) and
where the light chain variable domain comprises:
   a CDR-L1 amino acid sequence comprising SEQ ID NO: 159 or having 1, 2, 3, 4 or 5 amino acid alterations relative to SEQ ID NO: 159,
   a CDR-L2 amino acid sequence comprising SEQ ID NO: 172 or having 1, 2, 3, 4 or 5 amino acid alterations relative to SEQ ID NO: 172, and
   a CDR-L3 amino acid sequence comprising SEQ ID NO: 183 or having 1, 2, 3, 4 or 5 amino acid alterations relative to SEQ ID NO:183 (3B2).

In an embodiment, the identity or similarity is at least 81%, 82%, 83%, 84%, 85%, 86%, 87%, 88%, 89%, 90%, 91%, 92%, 93%, 94%, 95%, 96%, 97%, 98%, 99%.

In a more preferred embodiment, the expression vector comprises a polynucleotide encoding an antibody-based molecule that binds to human muscle-specific tyrosine-protein kinase (MuSK) and comprising a heavy chain variable domain and a light chain variable domain, wherein the heavy chain variable domain comprises an amino acid sequence that is at least 80% identical or similar to SEQ ID NO: 202 and the light chain variable domain comprises an amino acid sequence that is at least 80% identical or similar to SEQ ID NO: 203, and
where the heavy chain variable domain comprises:
   a CDR-H1 amino acid sequence comprising or consisting of SEQ ID NO: 147,
   a CDR-H2 amino acid sequence comprising or consisting of SEQ ID NO: 150, and
   a CDR-H3 amino acid sequence comprising or consisting of SEQ ID NO:156 (382) and
where the light chain variable domain comprises:
   a CDR-L1 amino acid sequence comprising or consisting of SEQ ID NO: 159,
   a CDR-L2 amino acid sequence comprising or consisting of SEQ ID NO: 172, and
   a CDR-L3 amino acid sequence comprising or consisting of SEQ ID NO:183 (3B2).

In an embodiment, the identity or similarity is at least 81%, 82%, 83%, 84%, 85%, 86%, 87%, 88%, 89%, 90%, 91%, 92%, 93%, 94%, 95%, 96%, 97%, 98%, 99%.

In an embodiment, the expression vector comprises a polynucleotide encoding an antibody-based molecule that binds to human muscle-specific tyrosine-protein kinase (MuSK) and comprising:
a) a nucleotide sequence that is at least 80% identical to SEQ ID NO:288 encoding the full length heavy chain, and
b) a nucleotide sequence that is at least 80% identical to SEQ ID NO:289 encoding the full length light chain.

In an embodiment, the identity is at least 81%, 82%, 83%, 84%, 85%, 86%, 87%, 88%, 89%, 90%, 91%, 92%, 93%, 94%, 95%, 96%, 97%, 98%, 99%.

In an embodiment, the expression vector comprises a polynucleotide encoding an antibody-based molecule that binds to human muscle-specific tyrosine-protein kinase (MuSK) and comprising:
a) a nucleotide sequence that is at least 80% identical to SEQ ID NO:284 encoding the heavy chain variable domain, and
b) a nucleotide sequence that is at least 80% identical to SEQ ID NO:285 encoding the light chain variable domain.

In an embodiment, the identity is at least 81%, 82%, 83%, 84%, 85%, 86%, 87%, 88%, 89%, 90%, 91%, 92%, 93%, 94%, 95%, 96%, 97%, 98%, 99%.

In an embodiment, the expression vector comprises a polynucleotide encoding an antibody-based molecule that binds to human muscle-specific tyrosine-protein kinase (MuSK) and comprising:
a) a nucleotide sequence that is at least 80% identical to SEQ ID NO:288 encoding the full length heavy chain, wherein said nucleotide sequence comprising a nucleotide sequence that is at least 80% identical to SEQ ID NO: 284 encoding the heavy chain variable domain, and
b) a nucleotide sequence that is at least 80% identical to SEQ ID NO:289 encoding the full length light chain, wherein said nucleotide sequence comprising a nucleotide sequence that is at least 80% identical to SEQ ID NO:285 encoding the light chain variable domain.

In an embodiment, the identity is at least 81%, 82%, 83%, 84%, 85%, 86%, 87%, 88%, 89%, 90%, 91%, 92%, 93%, 94%, 95%, 96%, 97%, 98%, 99%.

In an embodiment, the expression vector comprises a polynucleotide encoding an antibody-based molecule that binds to human muscle-specific tyrosine-protein kinase (MuSK) and comprising:
a) a nucleotide sequence SEQ ID NO:288 and
b) a nucleotide sequence SEQ ID NO:289.

### Host Cell

Another aspect of the present invention is a host cell or cell-free expression system for use in the treatment of a disease or condition resulting from a genetic defect in a fetus wherein the host cell or cell-free expression system is administered to the mother of said fetus, wherein the cell contains the expression vector encoding the antibody-based molecules and optionally producing said antibodies-based molecules as described herein.

The antibody-based molecules described herein can optionally be produced by a cell line, a mixed cell line, an immortalized cell or clonal population of immortalized cells, as well known in the art (see e.g., Ausubel et al., ed., Current Protocols in Molecular Biology, John Wiley & Sons, Inc., NY, N.Y. (1987-2001); Sambrook et al., Molecular Cloning: A Laboratory Manual, 2nd Edition, Cold Spring Harbor, N.Y. (1989); Harlow and Lane, Antibodies, a Laboratory Manual, Cold Spring Harbor, N.Y. (1989); Colligan et al., eds., Current Protocols in Immunology, John Wiley & Sons, Inc., NY (1994-2001); Colligan et al., Current Protocols in Protein Science, John Wiley & Sons, NY, N.Y., (1997-2001), which are hereby incorporated by reference in their entirety).

In some embodiments, the host cell chosen for expression may be of mammalian origin. Suitable mammalian host cells include, without limitation, COS-1 cells, COS-7 cells, HEK293 cells, BHK21 cells, CHO cells, BSC-1 cells, HeG2 cells, SP210 cells, HeLa cells, mammalian myeloma cells, mammalian lymphoma cells, or any derivative, immortalized or transformed cell thereof. Other suitable host cells include, without limitation, yeast cells, insect cells, and plant cells. Alternatively, the host cell may be selected from a species or organism incapable of glycosylating polypeptides, e.g., a prokaryotic cell or organism, such as BL21, BL21(DE3), BL21-GOLD(DE3), XL1-Blue, JM109, HMS174, HMS174 (DE3), and any of the natural or engineered E. coli spp, Klebsiella spp., or Pseudomonas spp strains.

The antibody-based molecules described herein can be prepared by any of a variety of techniques using the isolated polynucleotides, vectors, and host cells described supra. In general, antibodies can be produced by cell culture techniques, including the generation of monoclonal antibodies via conventional techniques, or via transfection of antibody genes, heavy chains and/or light chains into suitable bacterial or mammalian cell hosts, in order to allow for the production of antibodies, wherein the antibodies may be recombinant.

In an embodiment, the antibody-based molecule described herein is a monoclonal antibody or functional binding fragment thereof. Standard molecular biology techniques are used to prepare the recombinant expression vector, transfect the host cells, select for transformants, culture the host cells and recover the antibody from the culture medium. Transfecting the host cell can be carried out using a variety of techniques commonly used for the introduction of exogenous DNA into a prokaryotic or eukaryotic host cell, e.g., by electroporation, calcium-phosphate precipitation, DEAE-dextran transfection and the like. Although it is possible to express the antibodies described herein in either prokaryotic or eukaryotic host cells, expression of antibodies in eukaryotic cells, in particular mammalian cells is sometimes preferable, because such eukaryotic cells (and in particular mammalian cells) are more likely than prokaryotic cells to assemble and secrete a property folded and immunologically active antibody.

As noted above, exemplary mammalian host cells for expressing the recombinant antibodies of the invention include Chinese Hamster Ovary (CHO cells) (including dhfr-CHO cells, described in Urlaub and Chasin, Proc. Natl. Acad. Sci. USA, 77: 4216-4220 (1980), which is hereby incorporated by reference in its entirety). Other suitable mammalian host cells include, without limitation, NS0 myeloma cells, COS cells, and SP2 cells. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown.

Host cells can also be used to produce functional antibody fragments, such as Fab fragments or scFv molecules. It is understood that variations on the above procedure are within the scope of the present invention. For example, it may be desirable to transfect a host cell with DNA encoding functional fragments of either the light chain and/or the heavy chain of an antibody described herein. Recombinant DNA technology may also be used to remove some or all of the DNA encoding either or both of the light and heavy chains that is not necessary for binding to the antigens of interest. The molecules expressed from such truncated DNA molecules are also encompassed by the antibodies described herein.

The antibodies and antibody binding fragments are recovered and purified from recombinant cell cultures by known methods including, but not limited to, protein A purification, ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. High performance liquid chromatography (“HPLC”) can also be used for purification.

In a preferred embodiment, the host cell expresses an antibody-based molecule that binds to human muscle-specific tyrosine-protein kinase (MuSK), that comprises a heavy chain variable domain and a light chain variable domain, wherein the heavy chain variable domain comprises an amino acid sequence that is at least 80% identical or similar to SEQ ID NO: 234 and the light chain variable domain comprises an amino acid sequence that is at least 80% identical or similar to SEQ ID NO: 235, and
where the heavy chain variable domain comprises:
   a CDR-H1 amino acid sequence comprising SEQ ID NO: 147 or having 1, 2, 3, 4 or 5 amino acid alterations relative to SEQ ID NO: 147,
   a CDR-H2 amino acid sequence comprising SEQ ID NO: 153 or having 1, 2, 3, 4 or 5 amino acid alterations relative to SEQ ID NO: 153, and
   a CDR-H3 amino acid sequence comprising SEQ ID NO: 156 or having 1, 2, 3, 4 or 5 amino acid alterations relative to SEQ ID NO:156 (3B2g2m1) and
where the light chain variable domain comprises:
   a CDR-L1 amino acid sequence comprising SEQ ID NO: 159 or having 1, 2, 3, 4 or 5 amino acid alterations relative to SEQ ID NO: 159,
   a CDR-L2 amino acid sequence comprising SEQ ID NO: 172 or having 1, 2, 3, 4 or 5 amino acid alterations relative to SEQ ID NO: 172, and
   a CDR-L3 amino acid sequence comprising SEQ ID NO: 195 or having 1, 2, 3, 4 or 5 amino acid alterations relative to SEQ ID NO:195 (3B2g2m1).

In an embodiment, the identity or similarity is at least 81%, 82%, 83%, 84%, 85%, 86%, 87%, 88%, 89%, 90%, 91%, 92%, 93%, 94%, 95%, 96%, 97%, 98%, 99%.

In a more preferred embodiment, the host cell expresses an antibody-based molecule that binds to human muscle-specific tyrosine-protein kinase (MuSK), that comprises a heavy chain variable domain and a light chain variable domain, wherein the heavy chain variable domain comprises an amino acid sequence that is at least 80% identical or similar to SEQ ID NO: 234 and the light chain variable domain comprises an amino acid sequence that is at least 80% identical or similar to SEQ ID NO: 235, and
where the heavy chain variable domain comprises:
   a CDR-H1 amino acid sequence comprising or consisting of SEQ ID NO: 147,
   a CDR-H2 amino acid sequence comprising or consisting of SEQ ID NO: 153, and
   a CDR-H3 amino acid sequence comprising or consisting of SEQ ID NO:156 (3B2g2m1) and
where the light chain variable domain comprises:
   a CDR-L1 amino acid sequence comprising or consisting of SEQ ID NO: 159,
   a CDR-L2 amino acid sequence comprising or consisting of SEQ ID NO: 172, and
   a CDR-L3 amino acid sequence comprising or consisting of SEQ ID NO:195 (3B2g2m1).

In an embodiment, the identity or similarity is at least 81%, 82%, 83%, 84%, 85%, 86%, 87%, 88%, 89%, 90%, 91%, 92%, 93%, 94%, 95%, 96%, 97%, 98%, 99%.

In an embodiment, the host cell expresses an antibody-based molecule that binds to human muscle-specific tyrosine-protein kinase (MuSK), that comprises:
a) a nucleotide sequence that is at least 80% identical to SEQ ID NO:276 encoding the full length heavy chain, and
b) a nucleotide sequence that is at least 80% identical to SEQ ID NO:278 encoding the full length light chain.

In an embodiment, the identity is at least 81%, 82%, 83%, 84%, 85%, 86%, 87%, 88%, 89%, 90%, 91%, 92%, 93%, 94%, 95%, 96%, 97%, 98%, 99%.

In an embodiment, the host cell expresses an antibody-based molecule that binds to human muscle-specific tyrosine-protein kinase (MuSK), that comprises:
a) a nucleotide sequence that is at least 80% identical to SEQ ID NO:277 encoding the heavy chain variable domain, and
b) a nucleotide sequence that is at least 80% identical to SEQ ID NO:279 encoding the light chain variable domain.

In an embodiment, the identity is at least 81%, 82%, 83%, 84%, 85%, 86%, 87%, 88%, 89%, 90%, 91%, 92%, 93%, 94%, 95%, 96%, 97%, 98%, 99%.

In an embodiment, the host cell expresses an antibody-based molecule that binds to human muscle-specific tyrosine-protein kinase (MuSK), that comprises:
a) a nucleotide sequence that is at least 80% identical to SEQ ID NO:276 encoding the full length heavy chain, wherein said nucleotide sequence comprising a nucleotide sequence that is at least 80% identical to SEQ ID NO: 277 encoding the heavy chain variable domain, and
b) a nucleotide sequence that is at least 80% identical to SEQ ID NO:278 encoding the full length light chain, wherein said nucleotide sequence comprising a nucleotide sequence that is at least 80% identical to SEQ ID NO:279 encoding the light chain variable domain.

In an embodiment, the identity is at least 81%, 82%, 83%, 84%, 85%, 86%, 87%, 88%, 89%, 90%, 91%, 92%, 93%, 94%, 95%, 96%, 97%, 98%, 99%.

In an embodiment, the host cell expresses an antibody-based molecule that binds to human muscle-specific tyrosine-protein kinase (MuSK), that comprises:
a) a nucleotide sequence SEQ ID NO:276 and
b) a nucleotide sequence SEQ ID NO:278.

In a preferred embodiment, the host cell expresses an antibody-based molecule that binds to human muscle-specific tyrosine-protein kinase (MuSK), that comprises a heavy chain variable domain and a light chain variable domain, wherein the heavy chain variable domain comprises an amino acid sequence that is at least 80% identical or similar to SEQ ID NO: 202 and the light chain variable domain comprises an amino acid sequence that is at least 80% identical or similar to SEQ ID NO: 203, and
where the heavy chain variable domain comprises:
   a CDR-H1 amino acid sequence comprising SEQ ID NO: 147 or having 1, 2, 3, 4 or 5 amino acid alterations relative to SEQ ID NO: 147,
   a CDR-H2 amino acid sequence comprising SEQ ID NO: 150 or having 1, 2, 3, 4 or 5 amino acid alterations relative to SEQ ID NO: 150, and
   a CDR-H3 amino acid sequence comprising SEQ ID NO: 156 or having 1, 2, 3, 4 or 5 amino acid alterations relative to SEQ ID NO:156 (3B2) and
where the light chain variable domain comprises:
   a CDR-L1 amino acid sequence comprising SEQ ID NO: 159 or having 1, 2, 3, 4 or 5 amino acid alterations relative to SEQ ID NO: 159,
   a CDR-L2 amino acid sequence comprising SEQ ID NO: 172 or having 1, 2, 3, 4 or 5 amino acid alterations relative to SEQ ID NO: 172, and
   a CDR-L3 amino acid sequence comprising SEQ ID NO: 183 or having 1, 2, 3, 4 or 5 amino acid alterations relative to SEQ ID NO:183 (3B2).

In an embodiment, the identity or similarity is at least 81%, 82%, 83%, 84%, 85%, 86%, 87%, 88%, 89%, 90%, 91%, 92%, 93%, 94%, 95%, 96%, 97%, 98%, 99%.

In a more preferred embodiment, the host cell expresses an antibody-based molecule that binds to human muscle-specific tyrosine-protein kinase (MuSK), that comprises a heavy chain variable domain and a light chain variable domain, wherein the heavy chain variable domain comprises an amino acid sequence that is at least 80% identical or similar to SEQ ID NO: 202 and the light chain variable domain comprises an amino acid sequence that is at least 80% identical or similar to SEQ ID NO: 203, and
where the heavy chain variable domain comprises:
   a CDR-H1 amino acid sequence comprising or consisting of SEQ ID NO: 147,
   a CDR-H2 amino acid sequence comprising or consisting of SEQ ID NO: 150, and
   a CDR-H3 amino acid sequence comprising or consisting of SEQ ID NO:156 (382) and
where the light chain variable domain comprises:
   a CDR-L1 amino acid sequence comprising or consisting of SEQ ID NO: 159,
   a CDR-L2 amino acid sequence comprising or consisting of SEQ ID NO: 172, and
   a CDR-L3 amino acid sequence comprising or consisting of SEQ ID NO:183 (3B2).

In an embodiment, the identity or similarity is at least 81%, 82%, 83%, 84%, 85%, 86%, 87%, 88%, 89%, 90%, 91%, 92%, 93%, 94%, 95%, 96%, 97%, 98%, 99%.

In an embodiment, the host cell expresses an antibody-based molecule that binds to human muscle-specific tyrosine-protein kinase (MuSK), that comprises:
a) a nucleotide sequence that is at least 80% identical to SEQ ID NO:288 encoding the full length heavy chain, and
b) a nucleotide sequence that is at least 80% identical to SEQ ID NO:289 encoding the full length light chain.

In an embodiment, the identity is at least 81%, 82%, 83%, 84%, 85%, 86%, 87%, 88%, 89%, 90%, 91%, 92%, 93%, 94%, 95%, 96%, 97%, 98%, 99%.

In an embodiment, the host cell expresses an antibody-based molecule that binds to human muscle-specific tyrosine-protein kinase (MuSK), that comprises:
a) a nucleotide sequence that is at least 80% identical to SEQ ID NO:284 encoding the heavy chain variable domain, and
b) a nucleotide sequence that is at least 80% identical to SEQ ID NO:285 encoding the light chain variable domain.

In an embodiment, the identity is at least 81%, 82%, 83%, 84%, 85%, 86%, 87%, 88%, 89%, 90%, 91%, 92%, 93%, 94%, 95%, 96%, 97%, 98%, 99%.

In an embodiment, the host cell expresses an antibody-based molecule that binds to human muscle-specific tyrosine-protein kinase (MuSK), that comprises:
a) a nucleotide sequence that is at least 80% identical to SEQ ID NO:288 encoding the full length heavy chain, wherein said nucleotide sequence comprising a nucleotide sequence that is at least 80% identical to SEQ ID NO: 284 encoding the heavy chain variable domain, and
b) a nucleotide sequence that is at least 80% identical to SEQ ID NO:289 encoding the full length light chain, wherein said nucleotide sequence comprising a nucleotide sequence that is at least 80% identical to SEQ ID NO:285 encoding the light chain variable domain.

In an embodiment, the identity is at least 81%, 82%, 83%, 84%, 85%, 86%, 87%, 88%, 89%, 90%, 91%, 92%, 93%, 94%, 95%, 96%, 97%, 98%, 99%.

In an embodiment, the host cell expresses an antibody-based molecule that binds to human muscle-specific tyrosine-protein kinase (MuSK), that comprises:
a) a nucleotide sequence SEQ ID NO:288 and
b) a nucleotide sequence SEQ ID NO:289.

### Compositions

The antibody-based molecules or polynucleotide encoding the antibody-based molecules of the present invention may be advantageously administered as compositions. Therefore in a further aspect, there is provided a composition for use in the treatment of a disease or condition resulting from a genetic defect in a fetus, wherein the composition is administered to the mother of said fetus, comprising an antibody-based molecule, a polynucleotide, an expression vector or a host cell or cell free expression system as defined herein. In an embodiment, said composition is a pharmaceutical composition comprising at least one pharmaceutically acceptable carrier or excipients.

In another embodiment, a pharmaceutical composition comprising a recombinant nucleic acid sequence encoding the antibody-based molecule for use in the treatment of a disease or condition resulting from a genetic defect in a fetus, wherein the pharmaceutical composition is administered to the mother of the fetus to facilitate in vivo expression and formation of the antibody-based molecule for the treatment of the fetus. Expression vector constructs suitable for use in this embodiment of the invention are described supra.

In an embodiment, such compositions are pharmaceutical compositions comprising an active therapeutic agent (i.e., the MuSK antibody) and one or more of a variety of other pharmaceutically acceptable components. See REMINGTON: THE SCIENCE AND PRACTICE OF PHARMACY (21st Edition) (2005) (Troy, D. B. et al. (Eds.) Lippincott Williams & Wilkins (Publs.), Baltimore MD), which is hereby incorporated by reference in its entirety. The preferred form depends on the intended mode of administration and therapeutic application. The compositions can also include, depending on the formulation desired, pharmaceutically acceptable, non-toxic carriers, excipients, diluents, fillers, salts, buffers, detergents (e.g., a non-ionic detergent, such as Tween-20 or Tween-80), stabilizers (e.g., sugars or protein-free amino acids), preservatives, tissue fixatives, solubilizers, and/or other materials suitable for inclusion in a pharmaceutical composition, and which are vehicles commonly used to formulate pharmaceutical compositions for animal or human administration. The diluent is selected to not affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, or non-toxic, nontherapeutic, non-immunogenic stabilizers and the like. Examples of suitable aqueous and non-aqueous carriers which may be employed in the pharmaceutical compositions of the present invention include water, saline, phosphate-buffered saline, ethanol, dextrose, polyols (such as glycerol, propylene glycol, polyethylene glycol, and the like), and suitable mixtures thereof, vegetable oils, such as olive oil, corn oil, peanut oil, cottonseed oil, and sesame oil, carboxymethyl cellulose colloidal solutions, tragacanth gum and injectable organic esters, such as ethyl oleate, and/or various buffers. Other carriers are well-known in the pharmaceutical arts.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the present invention is contemplated.

The compositions may also include large, slowly metabolized macromolecules, such as proteins, polysaccharides like chitosan, polylactic acids, polyglycolic acids and copolymers (e.g., latex functionalized sepharose, agarose, cellulose, and the like), polymeric amino acids, amino acid copolymers, and lipid aggregates (e.g., oil droplets or liposomes). Suitability for carriers and other components of pharmaceutical compositions is determined based on the lack of significant negative impact on the desired biological properties of the active antibody-based molecule of the present invention (e.g., less than a substantial impact (e.g., 10% or less relative inhibition, 5% or less relative inhibition, etc.) on antigen binding).

The pharmaceutical compositions of the present invention may also comprise pharmaceutically acceptable antioxidants for instance (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like. The pharmaceutical compositions of the present invention may also comprise isotonicity agents, such as sugars, polyalcohols, such as mannitol, sorbitol, glycerol or sodium chloride in the compositions.

The pharmaceutical compositions of the present invention may also contain one or more adjuvants appropriate for the chosen route of administration such as preservatives, wetting agents, emulsifying agents, dispersing agents, preservatives or buffers, which may enhance the shelf life or effectiveness of the pharmaceutical composition. The antibodies of the present invention may be prepared with carriers that will protect the antibodies against rapid release, such as a controlled release formulation, including implants, transdermal patches, and microencapsulated delivery systems. Such carriers may include gelatin, glyceryl monostearate, glyceryl distearate, biodegradable, biocompatible polymers such as ethylene vinyl acetate, polyanhydrides, polyglycolic acid, collagen, polyorthoesters, and polylactic acid alone or with a wax, or other materials well-known in the art. Methods for the preparation of such formulations are generally known to those skilled in the art. See, e.g., SUSTAINED AND CONTROLLED RELEASE DRUG DELIVERY SYSTEMS, J. R. Robinson, ed., Marcel Dekker, Inc., New York, 1978.

In one embodiment, the antibodies of the present invention may be formulated to ensure proper distribution in vivo. Pharmaceutically acceptable carriers for parenteral administration include sterile aqueous solutions or dispersions and sterile powders for the extemporaneous preparation of sterile injectable solutions or dispersion. The use of such media and agents for pharmaceutically active substances is known in the art.

Pharmaceutical compositions for injection must typically be sterile and stable under the conditions of manufacture and storage. The composition may be formulated as a solution, microemulsion, liposome, or other ordered structure suitable to achieve high drug concentration. The carrier may be an aqueous or non-aqueous solvent or dispersion medium containing for instance water, ethanol, polyols (such as glycerol, propylene glycol, polyethylene glycol, and the like), and suitable mixtures thereof, vegetable oils, such as olive oil, and injectable organic esters, such as ethyl oleate. The proper fluidity may be maintained, for example, by the use of a coating such as lecithin, by the maintenance of the required particle size in the case of dispersion and by the use of surfactants. In many cases, it will be preferable to include isotonic agents, for example, sugars, polyalcohols such as glycerol, mannitol, sorbitol, or sodium chloride in the composition. Prolonged absorption of the injectable compositions may be brought about by including in the composition an agent that delays absorption, for example, monostearate salts and gelatin. Sterile injectable solutions may be prepared by incorporating the active compound in the required amount in an appropriate solvent with one or a combination of ingredients e.g. as enumerated above, as required, followed by sterilization microfiltration. Generally, dispersions are prepared by incorporating the active compound into a sterile vehicle that contains a basic dispersion medium and the required other ingredients. In the case of sterile powders for the preparation of sterile injectable solutions, examples of methods of preparation are vacuum drying and freeze-drying (lyophilization) that yield a powder of the active ingredient plus any additional desired ingredient from a previously sterile-filtered solution thereof.

For parenteral administration, agents of the present invention are typically formulated as injectable dosages of a solution or suspension of the substance in a physiologically acceptable diluent with a pharmaceutical carrier that can be a sterile liquid such as water, oil, saline, glycerol, or ethanol. Additionally, auxiliary substances, such as wetting or emulsifying agents, surfactants, pH buffering substances and the like can be present in compositions. Other components of pharmaceutical compositions are those of petroleum, animal, vegetable, or synthetic origin. Peanut oil, soybean oil, and mineral oil are all examples of useful materials. In general, glycols, such as propylene glycol or polyethylene glycol, are preferred liquid carriers, particularly for injectable solutions. Agents of the invention can be administered in the form of a depot injection or implant preparation which can be formulated in such a manner as to permit a sustained release of the active ingredient.

Typically, compositions are prepared as injectables, either as liquid solutions or suspensions; solid forms suitable for solution in, or suspension in, liquid vehicles prior to injection can also be prepared. The preparation also can be emulsified or encapsulated in liposomes or micro particles, such as polylactide, polyglycolide, or copolymer, for enhanced adjuvant effect (Langer, et al., Science 249:1527 (1990); Hanes, et al., Advanced Drug Delivery Reviews 28:97-119 (1997), which are hereby incorporated by reference in their entirety). Additional formulations suitable for other modes of administration include oral, intranasal, and pulmonary formulations, suppositories, and transdermal applications.

In an embodiment, the composition comprises the anti-MuSK antibody (or antigen-binding fragment) which comprises a heavy chain variable domain comprising the CDR-H1 of SEQ ID NO: 147, the CDR-H2 of SEQ ID NO: 153, and the CDR-H3 of SEQ ID NO: 156, and a light chain variable domain comprising the CDR-L1 of SEQ ID NO: 159, the CDR-L2 of SEQ ID NO: 172, and the CDR-L3 of SEQ ID NO: 195 (3B2g2m1).

In an embodiment, the composition comprises the anti-MuSK antibody (or antigen-binding fragment) which comprises a heavy chain variable domain comprising the CDR-H1 of SEQ ID NO: 147, the CDR-H2 of SEQ ID NO: 153, and the CDR-H3 of SEQ ID NO: 156, and the light chain variable domain comprising the CDR-L1 of SEQ ID NO: 159, the CDR-L2 of SEQ ID NO: 172, and the CDR-L3 of SEQ ID NO: 183 (3B2g1m1).

In an embodiment, the anti-MuSK antibody (or antigen-binding fragment) which comprises a heavy chain variable domain comprising the CDR-H1 of SEQ ID NO: 147, the CDR-H2 of SEQ ID NO: 154, and the CDR-H3 of SEQ ID NO: 156, and the light chain variable domain comprising the CDR-L1 of SEQ ID NO: 159, the CDR-L2 of SEQ ID NO: 172, and the CDR-L3 of SEQ ID NO: 183 (3B2g1m2).

In an embodiment, the anti-MuSK antibody (or antigen-binding fragment) which comprises a heavy chain variable domain comprising the CDR-H1 of SEQ ID NO: 147, the CDR-H2 of SEQ ID NO: 154, and the CDR-H3 of SEQ ID NO: 156, and the light chain variable domain comprising the CDR-L1 of SEQ ID NO: 159, the CDR-L2 of SEQ ID NO: 172, and the CDR-L3 of SEQ ID NO: 195 (3B2g2m2).

In an embodiment, the anti-MuSK antibody (or antigen-binding fragment) which comprises a heavy chain variable domain comprising the CDR-H1 of SEQ ID NO: 147, the CDR-H2 of SEQ ID NO: 150, and the CDR-H3 of SEQ ID NO: 156, and the light chain variable domain comprising the CDR-L1 of SEQ ID NO: 159, the CDR-L2 of SEQ ID NO: 172, and the CDR-L3 of SEQ ID NO: 183 (3B2).

In a preferred embodiment, the composition comprises an antibody or antigen-binding fragment that binds to human muscle-specific tyrosine-protein kinase (MuSK), that comprises a heavy chain variable domain and a light chain variable domain, wherein the heavy chain variable domain comprises an amino acid sequence that is at least 80% identical or similar to SEQ ID NO: 234 and the light chain variable domain comprises an amino acid sequence that is at least 80% identical or similar to SEQ ID NO: 235, and
where the heavy chain variable domain comprises:
   a CDR-H1 amino acid sequence comprising SEQ ID NO: 147 or having 1, 2, 3, 4 or 5 amino acid alterations relative to SEQ ID NO: 147,
   a CDR-H2 amino acid sequence comprising SEQ ID NO: 153 or having 1, 2, 3, 4 or 5 amino acid alterations relative to SEQ ID NO: 153, and
   a CDR-H3 amino acid sequence comprising SEQ ID NO:156 or having 1, 2, 3, 4 or 5 amino acid alterations relative to SEQ ID NO:156 (3B2g2m1) and
where the light chain variable domain comprises:
   a CDR-L1 amino acid sequence comprises SEQ ID NO: 159 or having 1, 2, 3, 4 or 5 amino acid alterations relative to SEQ ID NO: 159,
   a CDR-L2 amino acid sequence comprising SEQ ID NO: 172 or having 1, 2, 3, 4 or 5 amino acid alterations relative to SEQ ID NO: 172, and
   a CDR-L3 amino acid sequence comprising SEQ ID NO: 195 or having 1, 2, 3, 4 or 5 amino acid alterations relative to SEQ ID NO:195 (3B2g2m1).

In an embodiment, the identity or similarity is at least 81%, 82%, 83%, 84%, 85%, 86%, 87%, 88%, 89%, 90%, 91%, 92%, 93%, 94%, 95%, 96%, 97%, 98%, 99%.

In a more preferred embodiment, the composition comprises an antibody or antigen-binding fragment that binds to human muscle-specific tyrosine-protein kinase (MuSK), that comprises a heavy chain variable domain and a light chain variable domain, wherein the heavy chain variable domain comprises an amino acid sequence that is at least 80% identical or similar to SEQ ID NO: 234 and the light chain variable domain comprises an amino acid sequence that is at least 80% identical or similar to SEQ ID NO: 235, and
where the heavy chain variable domain comprises:
   a CDR-H1 amino acid sequence comprising or consisting of SEQ ID NO: 147,
   a CDR-H2 amino acid sequence comprising or consisting of SEQ ID NO: 153, and
   a CDR-H3 amino acid sequence comprising or consisting of SEQ ID NO:156 (3B2g2m1) and
where the light chain variable domain comprises:
   a CDR-L1 amino acid sequence comprising or consisting of SEQ ID NO: 159,
   a CDR-L2 amino acid sequence comprising or consisting of SEQ ID NO: 172, and
   a CDR-L3 amino acid sequence comprising or consisting of SEQ ID NO:195 (3B2g2m1).

In an embodiment, the identity or similarity is at least 81%, 82%, 83%, 84%, 85%, 86%, 87%, 88%, 89%, 90%, 91%, 92%, 93%, 94%, 95%, 96%, 97%, 98%, 99%.

In a preferred embodiment, composition comprises an antibody or antigen-binding fragment that binds to human muscle-specific tyrosine-protein kinase (MuSK) that comprises wild-type human IgG constant Fc region, a heavy chain variable domain and a light chain variable domain, where the wild-type human IgG constant Fc region comprising SEQ ID NO: 266 or 267, a heavy chain variable domain and a light chain variable domain, wherein the heavy chain variable domain comprises an amino acid sequence that is at least 80% identical or similar to SEQ ID NO: 234 and the light chain variable domain comprises an amino acid sequence that is at least 80% identical or similar to SEQ ID NO: 235, and
where the heavy chain variable domain comprises:
   a CDR-H1 amino acid sequence comprising SEQ ID NO: 147 or having 1, 2, 3, 4 or 5 amino acid alterations relative to SEQ ID NO: 147,
   a CDR-H2 amino acid sequence comprising SEQ ID NO: 153 or having 1, 2, 3, 4 or 5 amino acid alterations relative to SEQ ID NO: 153, and
   a CDR-H3 amino acid sequence comprising SEQ ID NO: 156 or having 1, 2, 3, 4 or 5 amino acid alterations relative to SEQ ID NO:156 (3B2g2m1) and
where the light chain variable domain comprises:
   a CDR-L1 amino acid sequence comprising SEQ ID NO: 159 or having 1, 2, 3, 4 or 5 amino acid alterations relative to SEQ ID NO: 159,
   a CDR-L2 amino acid sequence comprising SEQ ID NO: 172 or having 1, 2, 3, 4 or 5 amino acid alterations relative to SEQ ID NO: 172, and
   a CDR-L3 amino acid sequence comprising SEQ ID NO: 195 or having 1, 2, 3, 4 or 5 amino acid alterations relative to SEQ ID NO:195 (3B2g2m1).

In an embodiment, the identity or similarity is at least 81%, 82%, 83%, 84%, 85%, 86%, 87%, 88%, 89%, 90%, 91%, 92%, 93%, 94%, 95%, 96%, 97%, 98%, 99%.

In a more preferred embodiment, the composition comprises an antibody or antigen-binding fragment that binds to human muscle-specific tyrosine-protein kinase (MuSK) that comprises wild-type human IgG constant Fc region, a heavy chain variable domain and a light chain variable domain, where the wild-type human IgG constant Fc region comprising SEQ ID NO: 266 or 267, a heavy chain variable domain and a light chain variable domain, wherein the heavy chain variable domain comprises an amino acid sequence that is at least 80% identical or similar to SEQ ID NO: 234 and the light chain variable domain comprises an amino acid sequence that is at least 80% identical or similar to SEQ ID NO: 235, and
where the heavy chain variable domain comprises:
   a CDR-H1 amino acid sequence comprising or consisting of SEQ ID NO: 147,
   a CDR-H2 amino acid sequence comprising or consisting of SEQ ID NO: 153, and
   a CDR-H3 amino acid sequence comprising or consisting of SEQ ID NO:156 (3B2g2m1) and
where the light chain variable domain comprises:
   a CDR-L1 amino acid sequence comprising or consisting of SEQ ID NO: 159,
   a CDR-L2 amino acid sequence comprising or consisting of SEQ ID NO: 172, and
   a CDR-L3 amino acid sequence comprising or consisting of SEQ ID NO:195 (3B2g2m1).

In an embodiment, the identity or similarity is at least 81%, 82%, 83%, 84%, 85%, 86%, 87%, 88%, 89%, 90%, 91%, 92%, 93%, 94%, 95%, 96%, 97%, 98%, 99%.

In a preferred embodiment, the composition comprises an antibody or antigen-binding fragment that binds to human muscle-specific tyrosine-protein kinase (MuSK) that comprises wild-type human IgG constant Fc region wherein L234A and/or L235A substitution(s) numbered according the EU numbering system is(are) introduced into said Fc region, a heavy chain variable domain and a light chain variable domain, where the wild-type human IgG constant Fc region comprising SEQ ID NO: 266 or 267, a heavy chain variable domain and a light chain variable domain, wherein the heavy chain variable domain comprises an amino acid sequence that is at least 80% identical or similar to SEQ ID NO: 234 and the light chain variable domain comprises an amino acid sequence that is at least 80% identical or similar to SEQ ID NO: 235, and
where the heavy chain variable domain comprises:
   a CDR-H1 amino acid sequence comprising SEQ ID NO: 147 or having 1, 2, 3, 4 or 5 amino acid alterations relative to SEQ ID NO: 147,
   a CDR-H2 amino acid sequence comprising SEQ ID NO: 153 or having 1, 2, 3, 4 or 5 amino acid alterations relative to SEQ ID NO: 153, and
   a CDR-H3 amino acid sequence comprising SEQ ID NO: 156 or having 1, 2, 3, 4 or 5 amino acid alterations relative to SEQ ID NO:156 (3B2g2m1) and
where the light chain variable domain comprises:
   a CDR-L1 amino acid sequence comprising SEQ ID NO: 159 or having 1, 2, 3, 4 or 5 amino acid alterations relative to SEQ ID NO: 159,
   a CDR-L2 amino acid sequence comprising SEQ ID NO: 172 or having 1, 2, 3, 4 or 5 amino acid alterations relative to SEQ ID NO: 172, and
   a CDR-L3 amino acid sequence comprising SEQ ID NO: 195 or having 1, 2, 3, 4 or 5 amino acid alterations relative to SEQ ID NO:195 (3B2g2m1).

In an embodiment, the identity or similarity is at least 81%, 82%, 83%, 84%, 85%, 86%, 87%, 88%, 89%, 90%, 91%, 92%, 93%, 94%, 95%, 96%, 97%, 98%, 99%.

In a more preferred embodiment, the composition comprises an antibody or antigen-binding fragment that binds to human muscle-specific tyrosine-protein kinase (MuSK) that comprises wild-type human IgG constant Fc region wherein L234A and/or L235A substitution(s) numbered according the EU numbering system is(are) introduced into said Fc region, a heavy chain variable domain and a light chain variable domain, where the wild-type human IgG constant Fc region comprising SEQ ID NO: 266 or 267, a heavy chain variable domain and a light chain variable domain, wherein the heavy chain variable domain comprises an amino acid sequence that is at least 80% identical or similar to SEQ ID NO: 234 and the light chain variable domain comprises an amino acid sequence that is at least 80% identical or similar to SEQ ID NO: 235, and
where the heavy chain variable domain comprises:
   a CDR-H1 amino acid sequence comprising or consisting of SEQ ID NO: 147,
   a CDR-H2 amino acid sequence comprising or consisting of SEQ ID NO: 153, and
   a CDR-H3 amino acid sequence comprising or consisting of SEQ ID NO:156 (3B2g2m1) and
where the light chain variable domain comprises:
   a CDR-L1 amino acid sequence comprising or consisting of SEQ ID NO: 159,
   a CDR-L2 amino acid sequence comprising or consisting of SEQ ID NO: 172, and
   a CDR-L3 amino acid sequence comprising or consisting of SEQ ID NO:195 (3B2g2m1).

In an embodiment, the identity or similarity is at least 81%, 82%, 83%, 84%, 85%, 86%, 87%, 88%, 89%, 90%, 91%, 92%, 93%, 94%, 95%, 96%, 97%, 98%, 99%.

In an embodiment, the composition comprises an antibody or antigen-binding fragment that binds to human muscle-specific tyrosine-protein kinase (MuSK) that comprises:
a) a full length heavy chain comprising an amino acid sequence that is at least 80% identical or similar to SEQ ID NO: 268 and
b) a full length light chain comprising an amino acid sequence that is at least 80% identical or similar to SEQ ID NO: 269, and
c) wherein one or more of the following mutations (all numbered according to the EU numbering system) have been introduced into the full length heavy chain: an N297A substitution; an N297Q substitution; an L234A substitution; an L234D substitution; an L234E substitution; an L234G substitution; an L234H substitution; an L234F substitution; an L234K substitution; an L234Q substitution; an L234R substitution; an L234S substitution; an L234T substitution; an L235A substitution; an L235D substitution; an L235E substitution; an L235F substitution; an L235G substitution; an L235V substitution; an L235H substitution; an L2351 substitution; an L235K substitution; an L235R substitution; an L235S substitution; L235T substitution; an L235Q substitution; an L237A substitution; an S239D substitution; an E233P substitution; an L234V substitution; a C236 deletion; a G236E substitution; a G236R substitution; a G236K substitution; a G237A substitution; a P238A substitution; an F243L substitution; a D265A substitution; an S267E substitution; an H268A substitution; an R292P substitution; a Y300L substitution; a K322A substitution; a K322Q substitution; an A327Q substitution; an L328F substitution; an L328R substitution; a P329A substitution; a P329G substitution; an A330L substitution; an A330S substitution; a P331S substitution; an 1332E substitution; a P396L substitution; or each of the combinations of mutations described earlier in the fourth embodiment of this application, preferably the mutations is L234A or L235A, more preferably the mutations are L234A and L235A

In an embodiment, the composition comprises an antibody or antigen-binding fragment that binds to human muscle-specific tyrosine-protein kinase (MuSK) that comprises:
a) A full length heavy chain comprising SEQ ID NO: 268 and
b) A full length light chain comprising SEQ ID NO: 269, and
c) Wherein the full length heavy chain comprises L234A and L235A mutations numbered according the EU numbering system.

In an embodiment, the binding to an effector ligand is reduced of at least 10%, 20%, 30%, 40%, 50%, 60%, 70%, 80%, 90%, or is no longer detectable compared to the binding to the same ligand by the antibody not having any amino acid substitutions into its human IgG constant Fc region.

In an embodiment, the composition comprises an antibody or antigen-binding fragment that binds to human muscle-specific tyrosine-protein kinase (MuSK) that comprises:
a) a full length heavy chain comprising an amino acid sequence that is at least 80% identical or similar to SEQ ID NO: 270 and
b) a full length light chain comprising an amino acid sequence that is at least 80% identical or similar to SEQ ID NO: 271, and
c) wherein one or more of the following mutations (all numbered according to the EU numbering system) have been introduced into the full length heavy chain: an N297A substitution; an N297Q substitution; an L234A substitution; an L234D substitution; an L234E substitution; an L234G substitution; an L234H substitution; an L234F substitution; an L234K substitution; an L234Q substitution; an L234R substitution; an L234S substitution; an L234T substitution; an L235A substitution; an L235D substitution; an L235E substitution; an L235F substitution; an L235G substitution; an L235V substitution; an L235H substitution; an L2351 substitution; an L235K substitution; an L235R substitution; an L235S substitution; L235T substitution; an L235Q substitution; an L237A substitution; an S239D substitution; an E233P substitution; an L234V substitution; a C236 deletion; a G236E substitution; a G236R substitution; a G236K substitution; a G237A substitution; a P238A substitution; an F243L substitution; a D265A substitution; an S267E substitution; an H268A substitution; an R292P substitution; a Y300L substitution; a K322A substitution; a K322Q substitution; an A327Q substitution; an L328F substitution; an L328R substitution; a P329A substitution; a P329G substitution; an A330L substitution; an A330S substitution; a P331S substitution; an 1332E substitution; a P396L substitution; or each of the combinations of mutations described earlier in the fourth embodiment of this application, preferably the mutations is L234A or L235A, more preferably the mutations are L234A and L235A.

In an embodiment, the composition comprises an antibody or antigen-binding fragment that binds to human muscle-specific tyrosine-protein kinase (MuSK) that comprises:
a) a full length heavy chain comprising SEQ ID NO: 270 and
b) a full length light chain comprising SEQ ID NO: 271, and
c) wherein the full length heavy chain comprises L234A and L235A mutations numbered according the EU numbering system.

In an embodiment, the binding to an effector ligand is reduced of at least 10%, 20%, 30%, 40%, 50%, 60%, 70%, 80%, 90%, or is no longer detectable compared to the binding to the same ligand by the antibody not having any amino acid substitutions into its human IgG constant Fc region.

In a preferred embodiment, the composition comprises an antibody or antigen-binding fragment that binds to human muscle-specific tyrosine-protein kinase (MuSK), that comprises a heavy chain variable domain and a light chain variable domain, wherein the heavy chain variable domain comprises an amino acid sequence that is at least 80% identical or similar to SEQ ID NO: 202 and the light chain variable domain comprises an amino acid sequence that is at least 80% identical or similar to SEQ ID NO: 203, and
where the heavy chain variable domain comprises:
   a CDR-H1 amino acid sequence comprising SEQ ID NO: 147 or having 1, 2, 3, 4 or 5 amino acid alterations relative to SEQ ID NO: 147,
   a CDR-H2 amino acid sequence comprising SEQ ID NO: 150 or having 1, 2, 3, 4 or 5 amino acid alterations relative to SEQ ID NO: 150, and
   a CDR-H3 amino acid sequence comprising SEQ ID NO: 156 or having 1, 2, 3, 4 or 5 amino acid alterations relative to SEQ ID NO:156 (3B2) and
where the light chain variable domain comprises:
   a CDR-L1 amino acid sequence comprising SEQ ID NO: 159 or having 1, 2, 3, 4 or 5 amino acid alterations relative to SEQ ID NO: 159,
   a CDR-L2 amino acid sequence comprising SEQ ID NO: 172 or having 1, 2, 3, 4 or 5 amino acid alterations relative to SEQ ID NO: 172, and
   a CDR-L3 amino acid sequence comprising SEQ ID NO: 183 or having 1, 2, 3, 4 or 5 amino acid alterations relative to SEQ ID NO:183 (3B2).

In an embodiment, the identity or similarity is at least 81%, 82%, 83%, 84%, 85%, 86%, 87%, 88%, 89%, 90%, 91%, 92%, 93%, 94%, 95%, 96%, 97%, 98%, 99%.

In a more preferred embodiment, the composition comprises an antibody or antigen-binding fragment that binds to human muscle-specific tyrosine-protein kinase (MuSK), that comprises a heavy chain variable domain and a light chain variable domain, wherein the heavy chain variable domain comprises an amino acid sequence that is at least 80% identical or similar to SEQ ID NO: 202 and the light chain variable domain comprises an amino acid sequence that is at least 80% identical or similar to SEQ ID NO: 203, and
where the heavy chain variable domain comprises:
   a CDR-H1 amino acid sequence comprising or consisting of SEQ ID NO: 147,
   a CDR-H2 amino acid sequence comprising or consisting of SEQ ID NO: 150, and
   a CDR-H3 amino acid sequence comprising or consisting of SEQ ID NO:156 (3B2) and
where the light chain variable domain comprises:
   a CDR-L1 amino acid sequence comprising or consisting of SEQ ID NO: 159,
   a CDR-L2 amino acid sequence comprising or consisting of SEQ ID NO: 172, and
   a CDR-L3 amino acid sequence comprising or consisting of SEQ ID NO:183 (3B2).

In an embodiment, the identity or similarity is at least 81%, 82%, 83%, 84%, 85%, 86%, 87%, 88%, 89%, 90%, 91%, 92%, 93%, 94%, 95%, 96%, 97%, 98%, 99%.

In a more preferred embodiment, the composition comprises an antibody or antigen-binding fragment that binds to human muscle-specific tyrosine-protein kinase (MuSK) that comprises a heavy chain constant domain and light chain a constant domain (or human lambda constant domain), a heavy chain variable domain and a light chain variable domain, where the heavy chain constant domain comprising at least 80% sequence identity to SEQ ID NO: 280 and the light chain constant domain comprising at least 80% sequence identity to SEQ ID NO: 281, where the heavy chain variable domain comprises an amino acid sequence that is at least 80% identical or similar to SEQ ID NO: 202 and the light chain variable domain comprises an amino acid sequence that is at least 80% identical or similar to SEQ ID NO: 203, and
where the heavy chain variable domain comprises:
   a CDR-H1 amino acid sequence comprising SEQ ID NO: 147 or having 1, 2, 3, 4 or 5 amino acid alterations relative to SEQ ID NO: 147,
   a CDR-H2 amino acid sequence comprising SEQ ID NO: 150 or having 1, 2, 3, 4 or 5 amino acid alterations relative to SEQ ID NO: 150, and
   a CDR-H3 amino acid sequence comprising SEQ ID NO: 156 or having 1, 2, 3, 4 or 5 amino acid alterations relative to SEQ ID NO:156 (3B2) and
where the light chain variable domain comprises:
   a CDR-L1 amino acid sequence comprising SEQ ID NO: 159 or having 1, 2, 3, 4 or 5 amino acid alterations relative to SEQ ID NO: 159,
   a CDR-L2 amino acid sequence comprising SEQ ID NO: 172 or having 1, 2, 3, 4 or 5 amino acid alterations relative to SEQ ID NO: 172, and
   a CDR-L3 amino acid sequence comprising SEQ ID NO: 183 or having 1, 2, 3, 4 or 5 amino acid alterations relative to SEQ ID NO:183 (3B2).

In an embodiment, the identity or similarity is at least 81%, 82%, 83%, 84%, 85%, 86%, 87%, 88%, 89%, 90%, 91%, 92%, 93%, 94%, 95%, 96%, 97%, 98%, 99%.

In a preferred embodiment, the composition comprises an antibody or antigen-binding fragment that binds to human muscle-specific tyrosine-protein kinase (MuSK) that comprises a heavy chain constant domain and a light chain constant domain (or human lambda constant domain), a heavy chain variable domain and a light chain variable domain, where the heavy chain constant domain comprising at least 80% sequence identity to SEQ ID NO: 280 and the light chain constant domain comprising at least 80% sequence identity to SEQ ID NO: 281, wherein L234A and/or L235A substitution(s) (numbered according to the EU numbering system) is(are) introduced into said Fc region, and where the heavy chain variable domain comprises an amino acid sequence that is at least 80% identical or similar to SEQ ID NO: 202 and the light chain variable domain comprises an amino acid sequence that is at least 80% identical or similar to SEQ ID NO: 203, and
where the heavy chain variable domain comprises:
   a CDR-H1 amino acid sequence comprising SEQ ID NO: 147 or having 1, 2, 3, 4 or 5 amino acid alterations relative to SEQ ID NO: 147,
   a CDR-H2 amino acid sequence comprising SEQ ID NO: 150 or having 1, 2, 3, 4 or 5 amino acid alterations relative to SEQ ID NO: 150, and
   a CDR-H3 amino acid sequence comprising SEQ ID NO: 156 or having 1, 2, 3, 4 or 5 amino acid alterations relative to SEQ ID NO:156 (3B2) and
where the light chain variable domain comprises:
   a CDR-L1 amino acid sequence comprising SEQ ID NO: 159 or having 1, 2, 3, 4 or 5 amino acid alterations relative to SEQ ID NO: 159,
   a CDR-L2 amino acid sequence comprising SEQ ID NO: 172 or having 1, 2, 3, 4 or 5 amino acid alterations relative to SEQ ID NO: 172, and
   a CDR-L3 amino acid sequence comprising SEQ ID NO: 183 or having 1, 2, 3, 4 or 5 amino acid alterations relative to SEQ ID NO:183 (3B2).

In an embodiment, the identity or similarity is at least 81%, 82%, 83%, 84%, 85%, 86%, 87%, 88%, 89%, 90%, 91%, 92%, 93%, 94%, 95%, 96%, 97%, 98%, 99%.

In a more preferred embodiment, the composition comprises an antibody or antigen-binding fragment that binds to human muscle-specific tyrosine-protein kinase (MuSK) that comprises a heavy chain constant domain and a light chain constant domain (or human lambda constant domain), a heavy chain variable domain and a light chain variable domain, where the heavy chain constant domain comprising SEQ ID NO: 280 and the light chain constant domain comprising SEQ ID NO: 281, wherein L234A and L235A substitutions (numbered according to the EU numbering system) are introduced into said Fc region, and
where the heavy chain variable domain comprising SEQ ID NO: 202 and the light chain variable domain comprising SEQ ID NO: 203, and
where the heavy chain variable domain comprises:
   a CDR-H1 amino acid sequence comprising or consisting of SEQ ID NO: 147,
   a CDR-H2 amino acid sequence comprising or consisting of SEQ ID NO: 150, and
   a CDR-H3 amino acid sequence comprising or consisting of SEQ ID NO:156 (382) and
where the light chain variable domain comprises:
   a CDR-L1 amino acid sequence comprising or consisting of SEQ ID NO: 159,
   a CDR-L2 amino acid sequence comprising or consisting of SEQ ID NO: 172, and
   a CDR-L3 amino acid sequence comprising or consisting of SEQ ID NO:183 (3B2).

In an embodiment, the composition comprises an antibody or antigen-binding fragment that binds to human muscle-specific tyrosine-protein kinase (MuSK) that comprises:
a) a full length heavy chain comprising an amino acid sequence that is at least 80% identical or similar to SEQ ID NO: 282 and
b) a full length light chain comprising an amino acid sequence that is at least 80% identical or similar to SEQ ID NO: 283, and
c) wherein one or more of the following mutations (all numbered according to the EU numbering system) have been introduced into the full length heavy chain: an N297A substitution; an N297Q substitution; an L234A substitution; an L234D substitution; an L234E substitution; an L234G substitution; an L234H substitution; an L234F substitution; an L234K substitution; an L234Q substitution; an L234R substitution; an L234S substitution; an L234T substitution; an L235A substitution; an L235D substitution; an L235E substitution; an L235F substitution; an L235G substitution; an L235V substitution; an L235H substitution; an L2351 substitution; an L235K substitution; an L235R substitution; an L235S substitution; L235T substitution; an L235Q substitution; an L237A substitution; an S239D substitution; an E233P substitution; an L234V substitution; a C236 deletion; a G236E substitution; a G236R substitution; a G236K substitution; a G237A substitution; a P238A substitution; an F243L substitution; a D265A substitution; an S267E substitution; an H268A substitution; an R292P substitution; a Y300L substitution; a K322A substitution; a K322Q substitution; an A327Q substitution; an L328F substitution; an L328R substitution; a P329A substitution; a P329G substitution; an A330L substitution; an A330S substitution; a P331S substitution; an 1332E substitution; a P396L substitution; or each of the combinations of mutations described earlier in the fourth embodiment of this application, preferably the mutations is L234A or L235A, more preferably the mutations are L234A and L235A In an embodiment, the composition comprises an antibody or antigen-binding fragment that binds to human muscle-specific tyrosine-protein kinase (MuSK) that comprises:
a) a full length heavy chain comprising SEQ ID NO: 282 and
b) a full length light chain comprising SEQ ID NO: 283, and
c) wherein the full length heavy chain comprises L234A and L235A mutations numbered according the EU numbering system.

In an embodiment, the binding to an effector ligand is reduced of at least 10%, 20%, 30%, 40%, 50%, 60%, 70%, 80%, 90%, or is no longer detectable compared to the binding to the same ligand by the antibody not having any amino acid substitutions into its human IgG constant Fc region.

In the context of the application, the heavy chain constant domain described herein may contain additional lysine (or K) residue (s) at the C-terminal.

Any of the antibody-based molecule, polynucleotide, expression vector or a host cell or cell free expression system as defined herein can be packaged as a kit and optionally include instructions for use. The antibody-based molecule, a polynucleotide, an expression vector, a host cell, a cell-free expression system or a composition of the present invention can be administered by parenteral, topical, oral or intranasal means for therapeutic treatment.

In the context of the invention, any of the antibody-based molecule, polynucleotide, expression vector or a host cell or cell free expression system as defined herein, wherein the polynucleotide, the expression vector or the host cell or cell-free expression system is administered to the fetus (or newborn) after birth.

### Method of Treatment

The present invention also provides methods of treating a disease or condition resulting from a genetic defect in a fetus wherein the antibody-based molecule, the polynucleotide, the expression vector, the host cell or cell-free expression system or the pharmaceutical composition defined above is administered to the mother of said fetus. In an embodiment, the method comprises administering to the mother a therapeutically effective amount of an antibody-based molecule, a polynucleotide, an expression vector, a host cell or cell-free expression system or a pharmaceutical composition. Each of an antibody-based molecule, a polynucleotide, an expression vector, a host cell or cell-free expression system and a pharmaceutical composition has been extensively defined in earlier sections of the description. In such methods of treatment, the antibody-based molecules are typically formulated as pharmaceutical compositions. In an embodiment, the polynucleotide, the expression vector or the host cell or cell-free expression system, or the composition is administered to the fetus (or newborn) after birth.

As used herein, the term “therapeutically effective amount” is intended to mean the quantity or dose of antibody-based molecule that is sufficient to trigger reversed, delayed and/or prevented developmental abnormalities, reversal of developmental abnormalities, fetal weight gain, postnatal survival, prolonged postnatal survival, improved postnatal development and/or postnatal weight gain, relative to a fetus whose mother is not administered with the antibody-based molecule, wherein the treated fetus is assessed by size estimation, growth curve, physical movement, heart rate monitoring, prenatal survival and/or postnatal survival.

In an embodiment, the disease or condition resulting from a genetic defect is a neurodegenerative disease. In an embodiment, the neuromuscular disease is caused by other factors except for genetic defects. In an embodiment, the antibody-based molecule is an anti-MuSK antibody or antigen binding fragment thereof. The anti-MuSK antibody or antigen binding fragment thereof is able to elicit an MuSK agonistic activity in the fetus. In an embodiment, such agonistic MuSK activity is able to eradicate or at least alleviate the symptoms associated with a disease or condition associated with an impaired neuromuscular transmission. An appropriate amount or dose can be determined by a physician, as appropriate.

As used herein, a method of “preventing” a disease or condition means preventing the onset of the disease, preventing the worsening of symptoms, preventing the progression of the disease or condition or reducing the risk of a subject developing the disease or condition. As used herein, a method of “treating” a disease or condition means curing a disease or condition and/or alleviating or eradicating the symptoms associated with the disease or condition such that the patient's suffering is reduced.

### Use

In a further aspect, the invention relates to the use of an antibody-based molecule, a polynucleotide, an expression vector, a host cell or cell-free expression system, a pharmaceutical composition for the manufacture of a medicament for the treatment of a disease or condition resulting from a genetic defect in a fetus, wherein the antibody-based molecule, the polynucleotide, the expression vector, the host cell or the cell-free expression system, or the pharmaceutical composition is administered to the mother of said fetus. Each of an antibody-based molecule, a polynucleotide, an expression vector, a host cell or cell-free expression system and a pharmaceutical composition has been extensively defined in earlier sections of the description. In an embodiment, the disease or condition resulting from a genetic defect is a neurodegenerative disease. In an embodiment, the neuromuscular disease is caused by other factors except for genetic defects. In an embodiment, the antibody-based molecule is anti-MuSK antibody or antigen binding fragment thereof.

All documents cited in the present specification are hereby incorporated by reference in their entirety. Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention. Each embodiment described herein may be combined together with any other embodiment described herein, unless otherwise indicated.

The present invention is further described by the following examples which should not be construed as limiting the scope of the invention.

### EXAMPLES

The invention will be further understood with reference to the following non-limiting examples.

### Example 1: Dok7 CM, in a Mixed CBA-C57BL/6 Background, Chronically Injected with ARGX-119 (Full Length Heavy Chain SEQ ID NO: 268 and Full Length Light Chain SEQ ID NO: 269), are Fertile and have a Viable Offspring

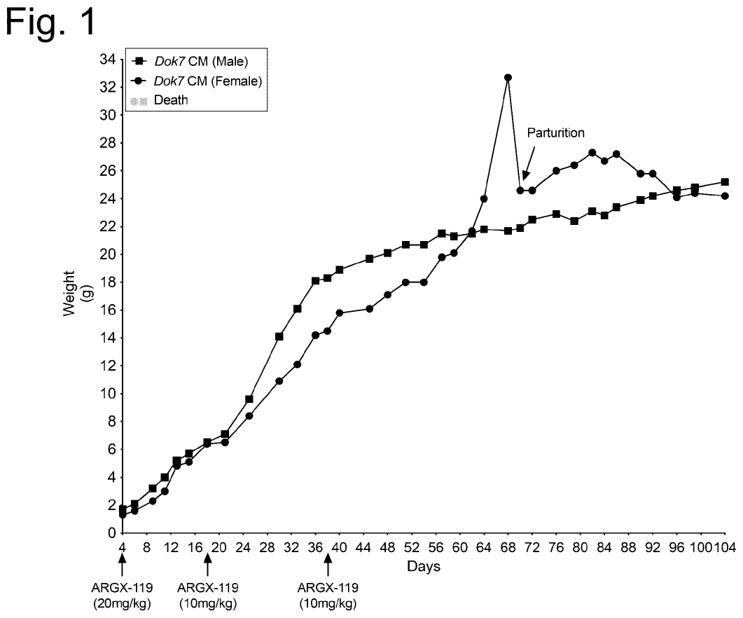

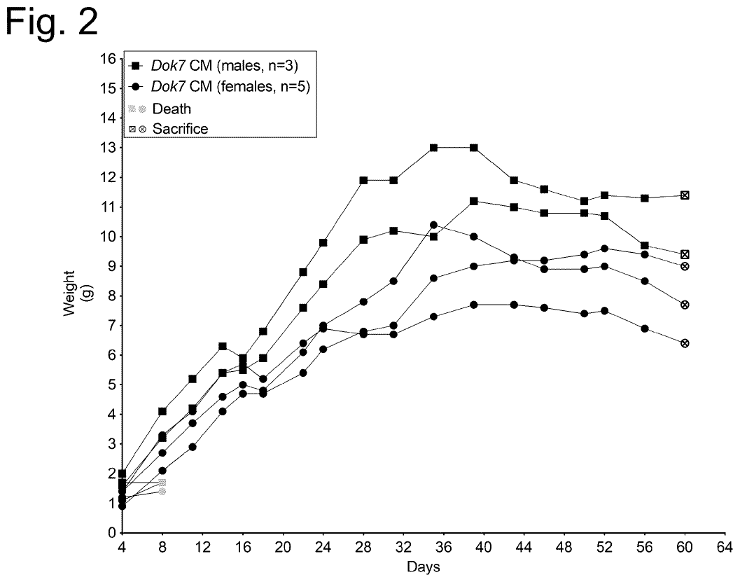

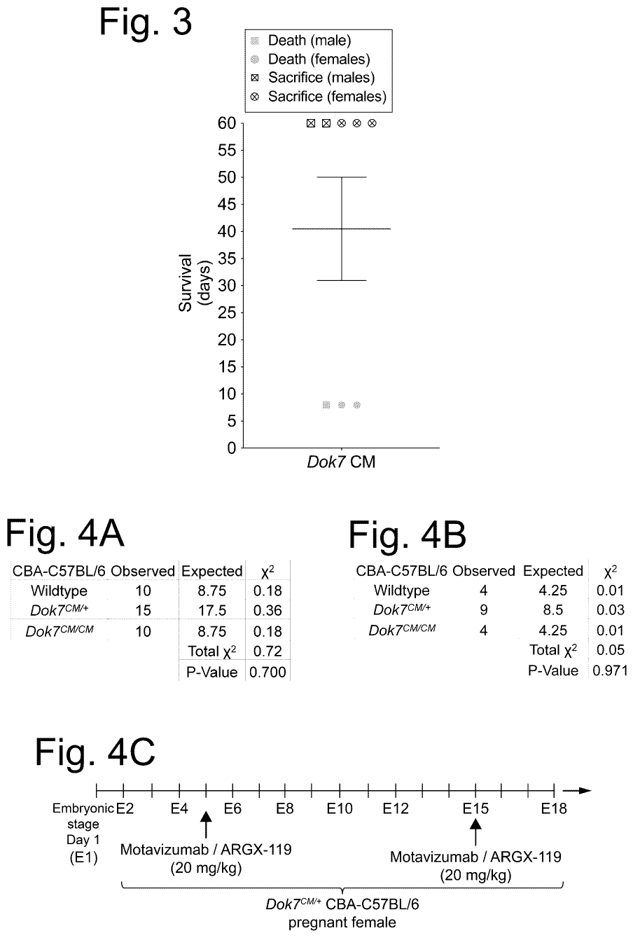

To determine whether Dok7 1124_1127dup (Dok7 CM) adult CBA-C57B16 mice injected with MuSK agonist antibody ARGX-119 can be fertile, Dok7 CM male and female mice were chronically injected with ARGX-119 at postnatal day 4 (P4) (20 mg/kg), postnatal day (P18) (10 mg/kg) and postnatal day (P38) (10 mg/kg) (FIG. 1). These male and female Dok7 CM mice are homozygous for DOK7 mutation (Dok7 1124_1127dup). They were rescued from early lethality and, once sexual maturity was reached, they were mated. The female mouse became pregnant and delivered eight Dok7 CM pups (FIG. 1). Five of eight Dok7 CM progeny, who were not themselves injected with ARGX-119, gained weight over the weeks after birth, and survived for two months, when the experiment was ended (FIGS. 2-3). Three of eight Dok7 CM progeny never gained weight, and died at P8 (FIGS. 2-3). Dok7 CM mice injected with ARGX-119 are fertile, ARGX-119 is transferred from Dok7 CM pregnant mothers, allowing the mutant Dok7 CM offspring to survive postnatally (FIGS. 1-3) (n=1 experiment).

### Example 2: Rescued Dok7 CM Progeny Receive ARGX-119 from the Blood (Placenta) of their Pregnant Mothers

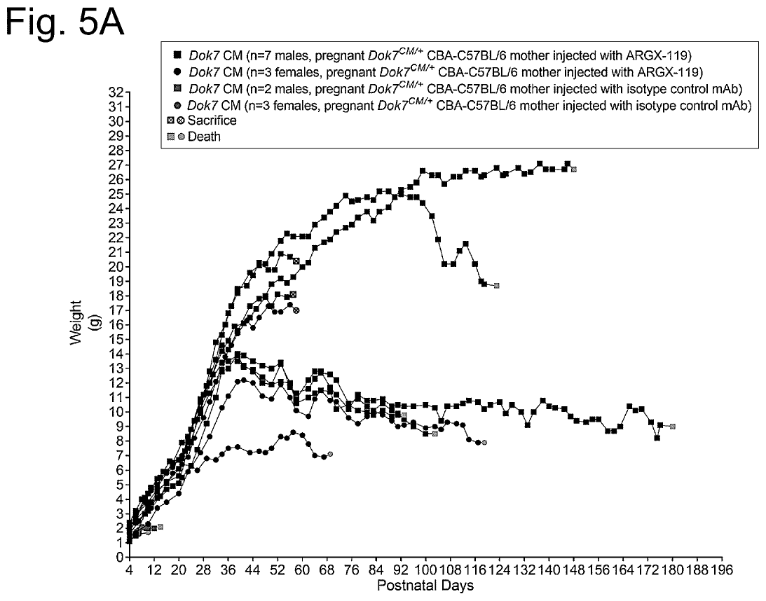

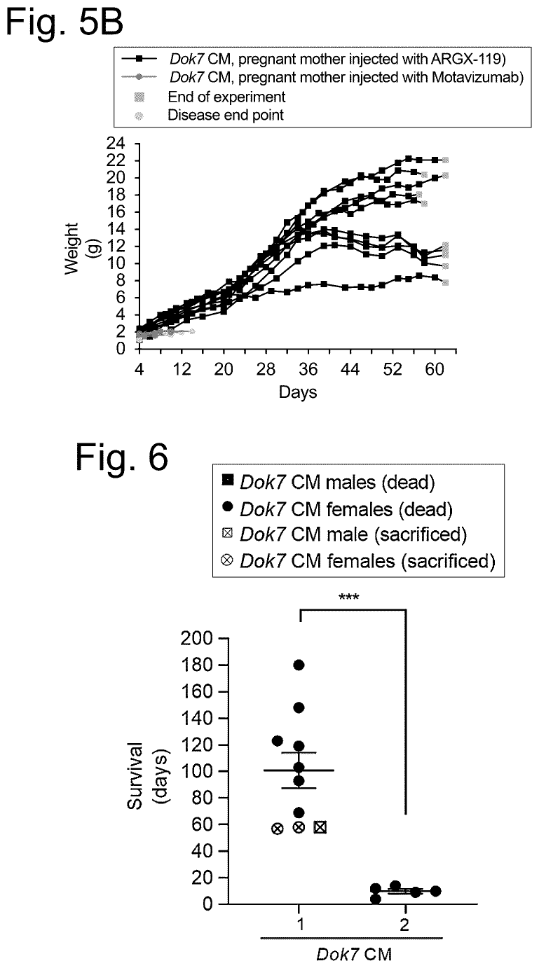

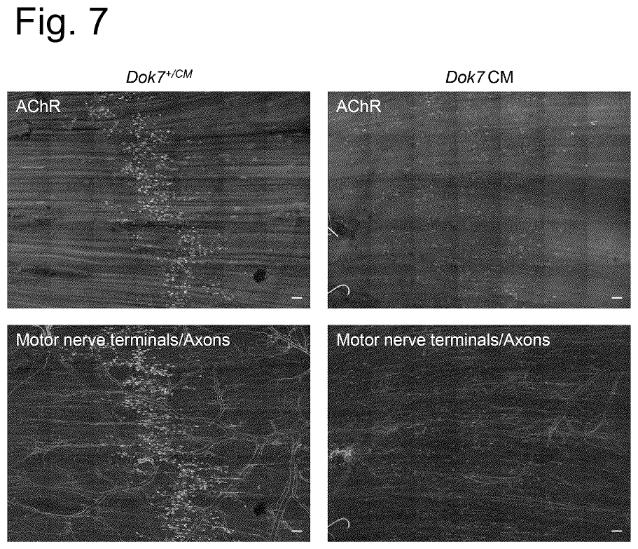

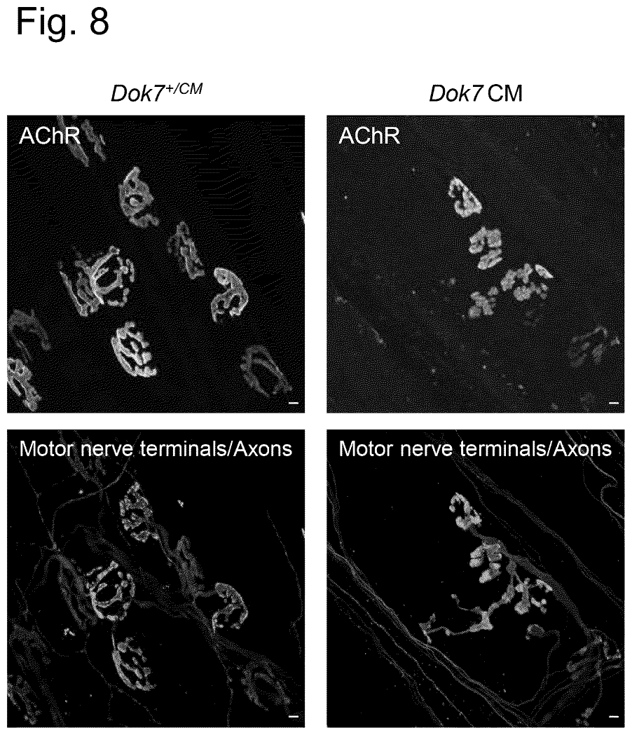

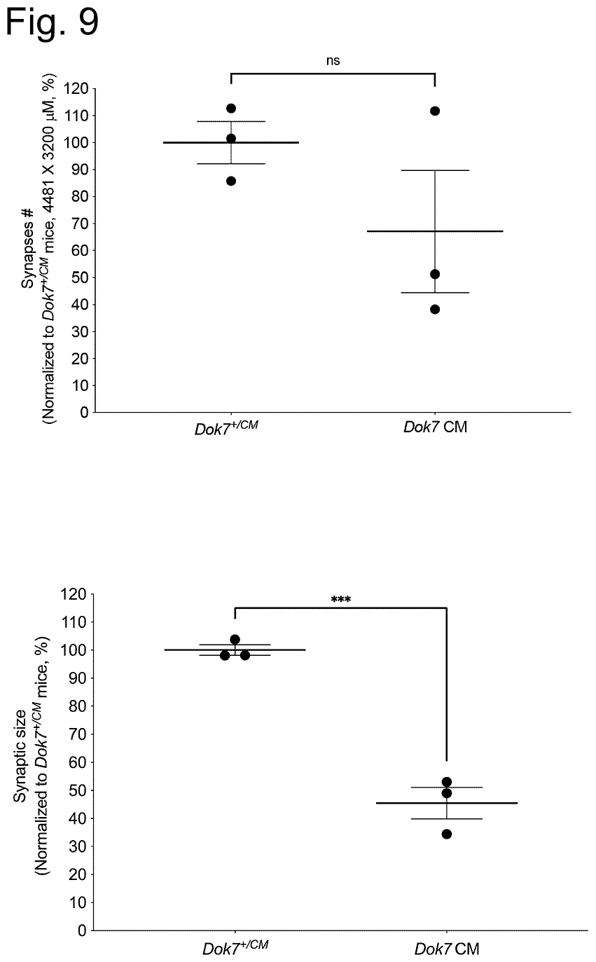

To determine if rescued progeny receive ARGX-119 from the blood (placenta) or milk of Dok7 CM (CBA-C57BL/6) female mice. Dok7^{CM/+} (CBA-C57BL/6) female mice were injected intraperitonially (IP), five, and fifteen days after fertilization with ARGX-119, or isotype control mAb (Mota-hIgG1LALAdelk) (20 mg/kg). Their homozygous Dok7^{CM/CM} progeny born from Dok7^{CM/+} mice injected with ARGX-119 or isotype control mAb were recovered postnatally at the expected Mendelian ratio. χ² analysis of the progeny shows that the occurrence of genotypes is unlikely to occur by chance (FIGS. 4A-4B). χ² analysis test is a statistical test used to compare observed results with expected results. The purpose of this test is to determine if a difference between observed data and expected data is due to chance, or if it is due to a relationship between the variables being studied. A p-value higher than 0.05 (>0.05) is not statistically significant and indicates strong evidence for the null hypothesis. FIGS. 4A-4B indicates that there is no significant difference between the two groups of 1) the observed progeny numbers of each genotype (WT, DOK7^{CM/+}, DOK7^{CM/CM}) and 2) the expected progeny numbers of each genotype (WT, DOK7^{CM/+}, DOK7^{CM/CM}), born from Dok7^{CM/+} mice. Newborn pups born from ARGX-119-injected mothers were transferred to Dok7^{CM/+} foster female mice that had received isotype control mAb. In addition, pups from isotype control mAb-injected mothers were transferred to Dok7^{CM/+} foster female mice that had been injected with ARGX-119. In neither case, were the progeny themselves injected with ARGX-119, or isotype control mAb. 10 of 10 Dok7 CM progeny, who were born from Dok7^{CM/+} female mice who had been injected with ARGX-119 during pregnancy and fostered after birth with isotype control mAb-injected Dok7^{CM/+} female mice, gained weight over the weeks after birth, and survived at least two months (FIGS. 5A and 5B, and 6). All 5 Dok7 CM progeny, who were born from isotype control mAb-injected Dok7^{CM/+} female mice and fostered with Dok7^{CM/+} female mice who had been injected with ARGX-119 during pregnancy, never gained weight and died during the first two weeks after birth (FIGS. 5A and 5B, and 6). This suggests that ARGX-119 is transferred from placenta of Dok7^{CM/+} pregnant mothers, allowing the offspring to survive postnatally for at least two months, and up to six months without being injected themselves with ARGX-119 (n=5 experiments), at which point we assessed the structure of their neuromuscular synapses (FIG. 7-8-9). ARGX-119 rescued synapse formation and maturation, as the neuromuscular synapses of these mice had developed the complex pretzel-like shape characteristic of fully mature mouse neuromuscular synapses (FIG. 7-8-9).

### Example 3: Dok7 CM Mice in a Full Inbred C57BL/6 Background Die at Birth, but Survive Postnatally if their Pregnant Mothers were Treated with ARGX-119

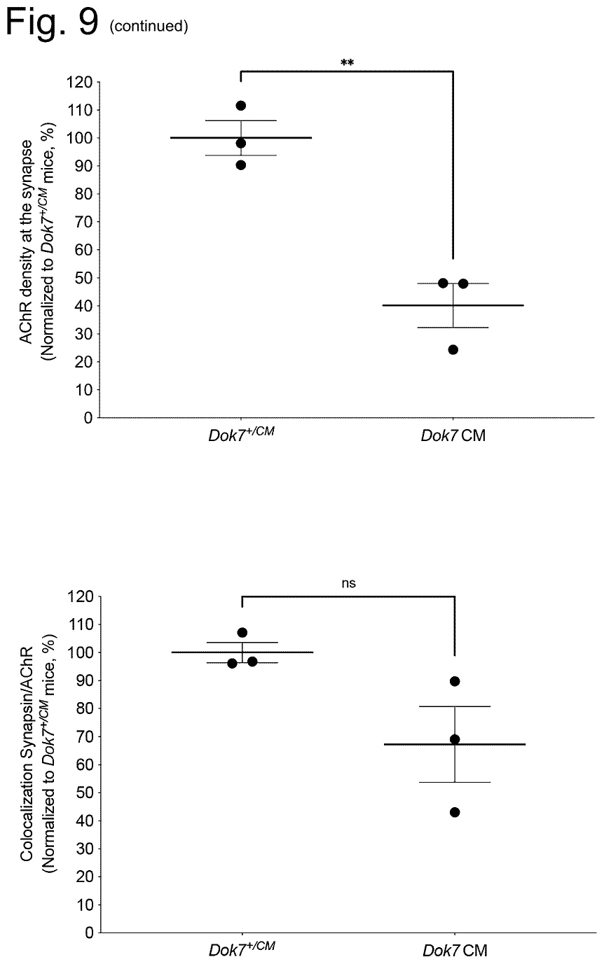

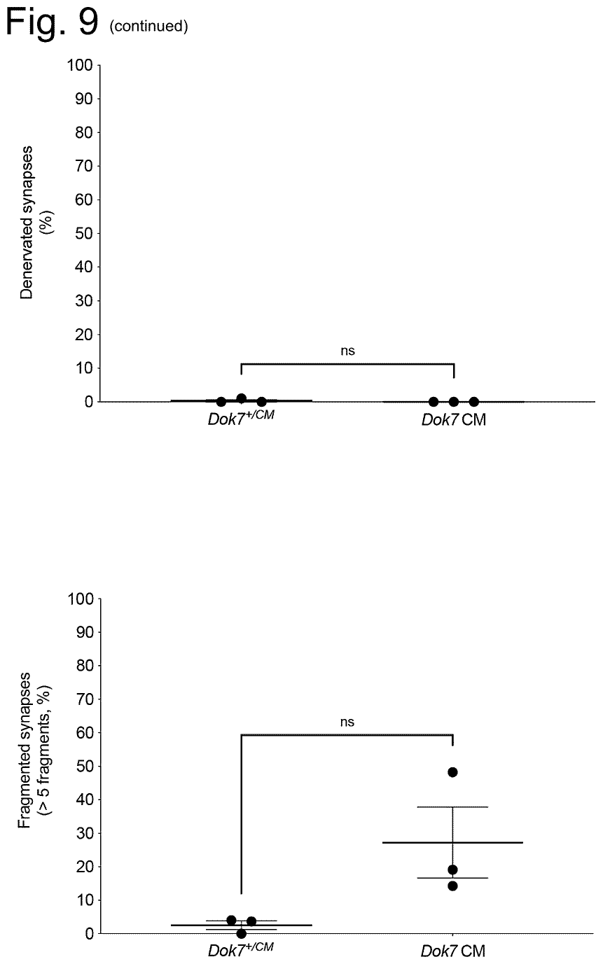

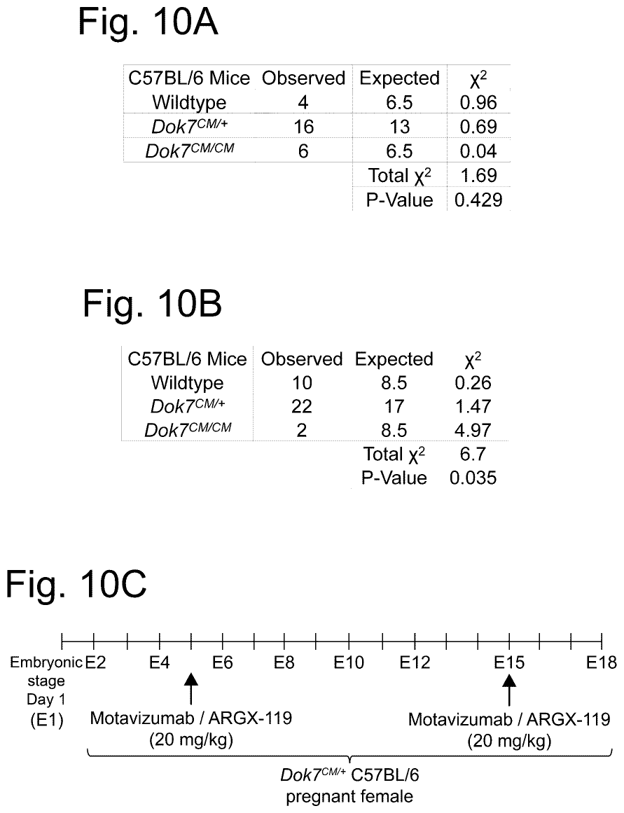

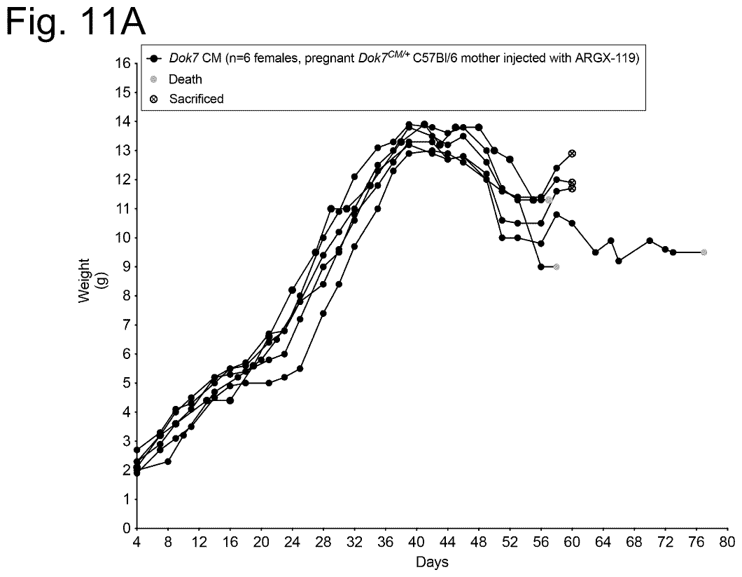

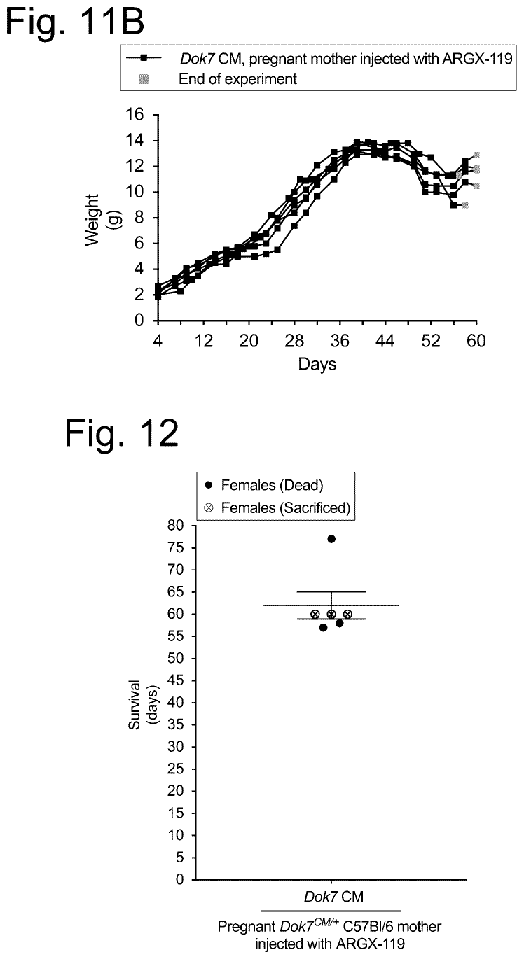

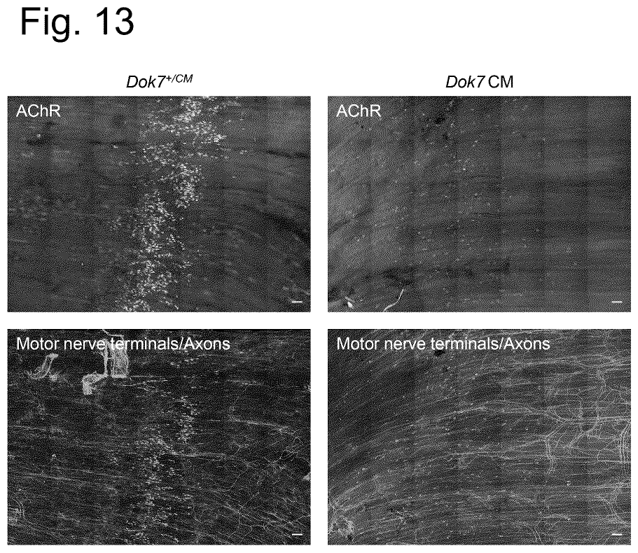

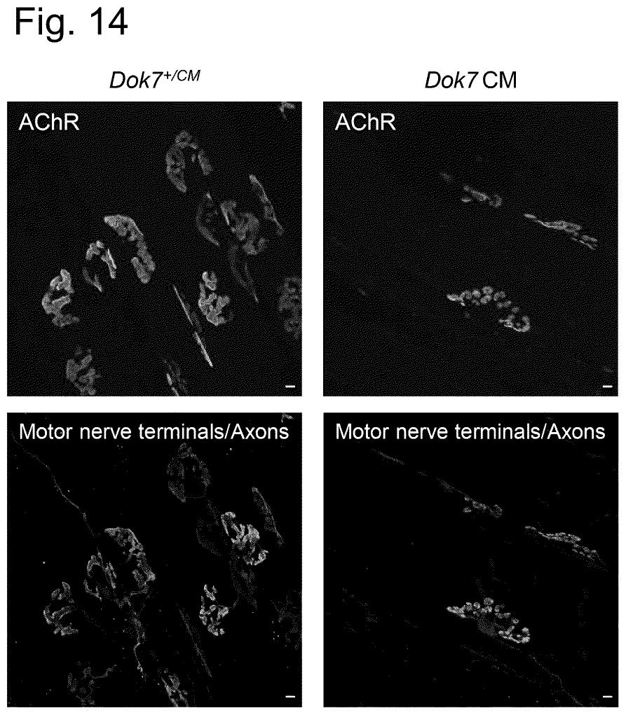

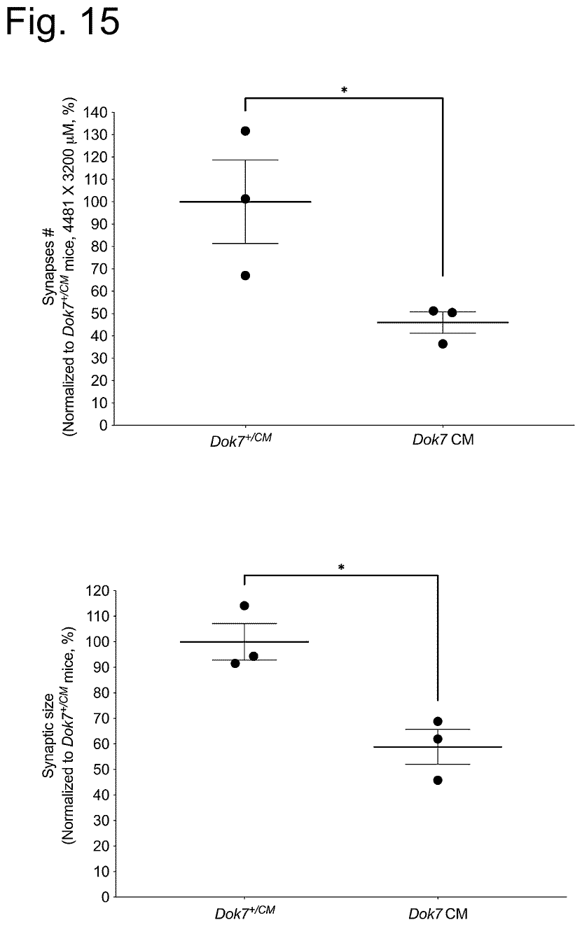

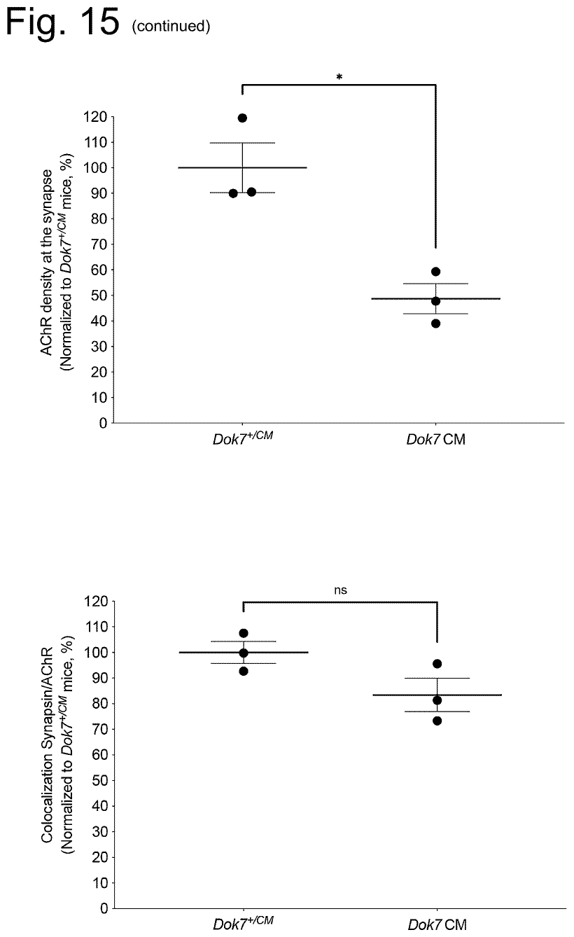

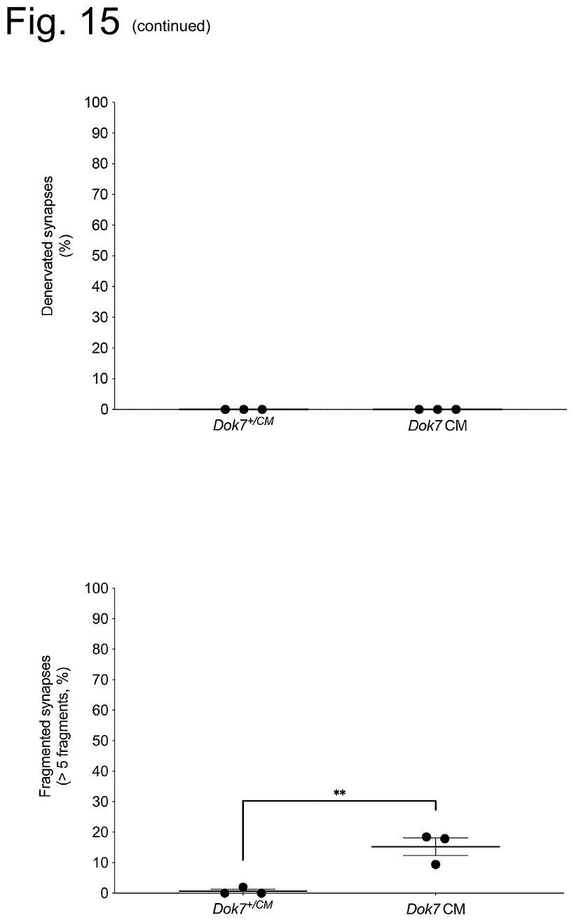

Dok7 CM mice in a full inbred C57BL/6 background die at birth (Oury, Julien et al. “Mechanism of Disease and Therapeutic Rescue of Dok7 Congenital Myasthenia.” Nature 595.7867 (2021): 404-408.). Inbred mouse strains are defined as colonies produced by a minimum of 20 generations of brother-sister mating, traceable to a single founding pair. To determine if ARGX-119 could rescue Dok7 CM mutant progeny in a full inbred background, Dok7^{CM/+} C57BL/6 female mice were injected intraperitonially (IP) five and fifteen days after fertilization either with ARGX-119 or isotype control mAb. The homozygous Dok7^{CM/CM} progeny born from Dok7^{CM/+} mice injected with ARGX-119 were recovered postnatally at the expected Mendelian ratio. χ² analysis of the progeny shows that the occurrence of genotypes is unlikely to occur by chance (FIG. 10A). Six of six Dok7 CM C57BL/6 progeny, born from Dok7 CM C57BL/6 female mice who had been injected with ARGX-119 during pregnancy, are rescued from neonatal lethality (FIG. 10A), gained weight (FIG. 11A) and, survived nearly two months without being injected themselves with ARGX-119 (FIG. 12). Dok7 CM C57BL/6 progeny, who were born from Dok7 CM C57BL/6 female mice who had been injected with isotype control mAb during pregnancy died at birth (FIGS. 10A-10B). These data suggest ARGX-119 can rescue full inbred Dok7 CM mice from neonatal lethality, and allows them to survive nearly two months without being injected themselves with ARGX-119 (n=4 experiments), at which point we assessed the structure of their neuromuscular synapses (FIG. 13-14-15). ARGX-119 rescued synapse formation and maturation, as the neuromuscular synapses of these mice had developed the complex pretzel-like shape characteristic of fully mature mouse neuromuscular synapses (FIG. 13-14-15).

### Example 4: MuSK Agonist Antibodies Rescue Agrin^{ΔZ/ΔZ} but not Rapsyn^{-/-} Mutant Mice from Neonatal Lethality

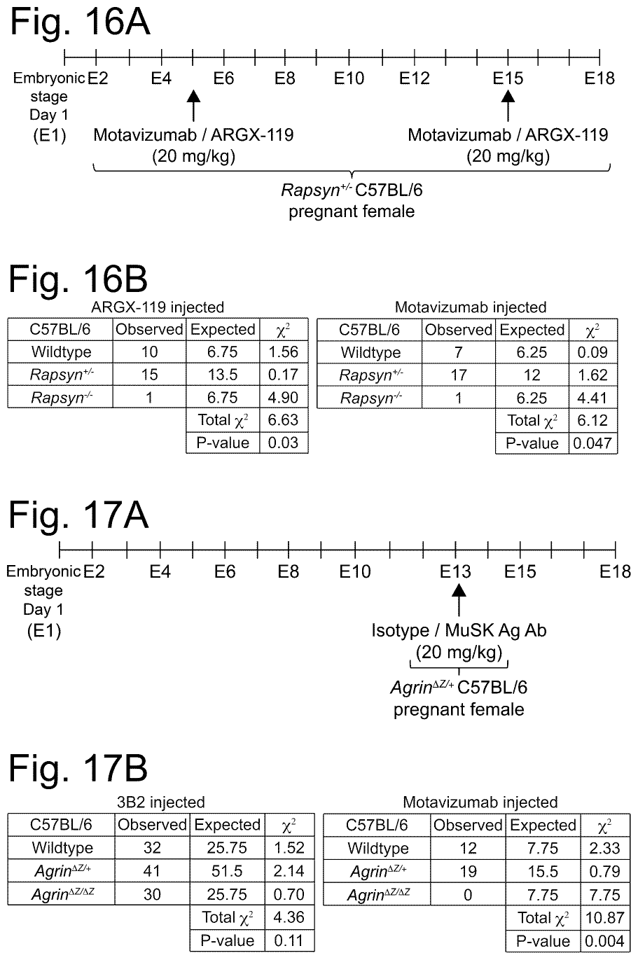

To determine if ARGX-119 could rescue Rapsyn^{-/-} mutant progeny, Rapsyn^{+/-} female mice in a C57BL/6 genetic background were injected twice at E5 and E15 with ARGX-119 (20 mg/kg) or isotype control motavizumab (20 mg/kg) after fertilization (FIG. 16A). Their progeny was not injected with ARGX-119 or motavizumab. X² analysis of the progeny shows that the occurrence of genotypes is unlikely to occur by chance, indicating that Rapsyn^{-/-} mice in a C57BL/6 genetic background born from Rapsyn^{+/-} mice injected with ARGX-119 don't survive postnatally (n=3 experiments) (FIG. 168).

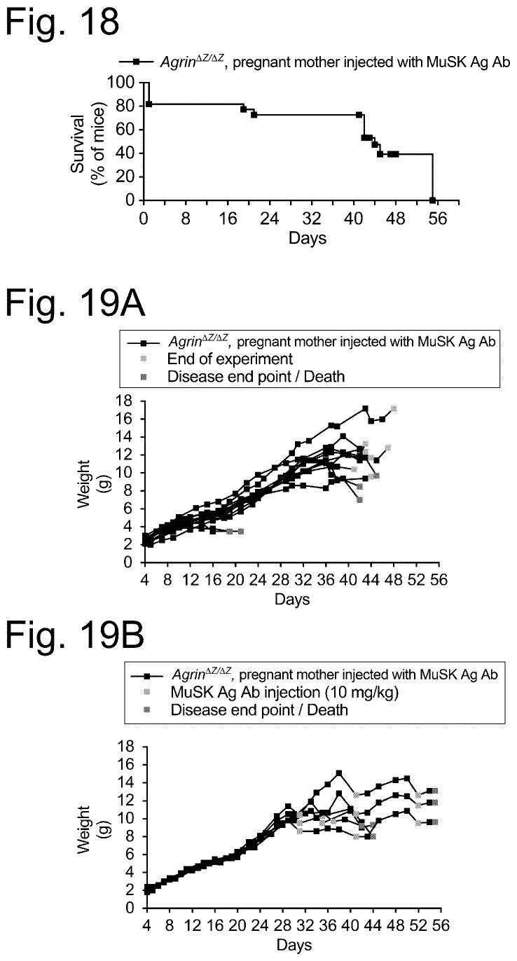

To determine if 382 (full length heavy chain SEQ ID NO: 282 and full length light chain SEQ ID NO: 283) could rescue Agrin^{ΔZ/ΔZ} mutant progeny, Agrin^{ΔZ/+} female mice in a C57BL/6 genetic background were injected twice at E13 with MuSK agonist antibodies 3B2 (20 mg/kg) or isotype control motavizumab (20 mg/kg). Their progeny (Agrin^{ΔZ/ΔZ} mutant progeny) was not injected with 3B2 or motavizumab (FIG. 17A). Progeny from Agrin^{ΔZ/+} C57BL/6 female, injected with 3B2 during pregnancy were recovered postnatally at the expected Mendelian ratio. X² analysis of the progeny shows that the occurrence of genotypes is unlikely to occur by chance, indicating that Agrin^{ΔZ/ΔZ} mice in a C57BL/6 genetic background born from Agrin^{ΔZ/+} mice injected with 382 are partially rescued by injecting their pregnant mothers with 3B2 (FIG. 17B). Agrin^{ΔZ/ΔZ} C57BL/6 progeny, who were born from an 3B2-injected Agrin^{ΔZ/+} C57BL/6 female mouse during pregnancy, can survive on average nearly 40 days, and up to 55 days, while Agrin^{ΔZ/ΔZ} C57B/L6 mice born from Agrin^{ΔZ/+} female injected with isotype control motavizumab die at birth (FIG. 18). Postnatal injection of MuSK agonist antibodies 3B2 shows a moderate effect on survival or weight gain of Agrin^{ΔZ/ΔZ} mice. Agrin^{ΔZ/ΔZ} C57BL/6 progeny, who were born from 3B2-injected Agrin^{ΔZ/+} C57BL/6 female mice during pregnancy ultimately shows disease relapse around P30. (FIGS. 19A and 19B)

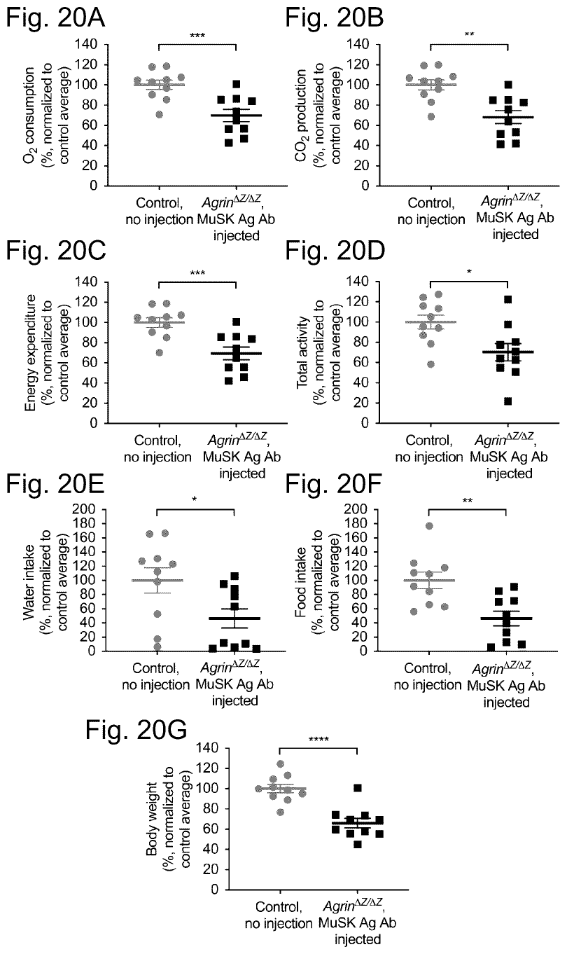

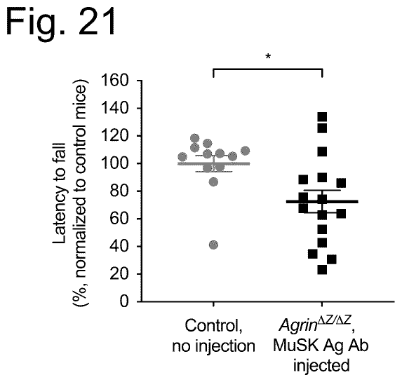

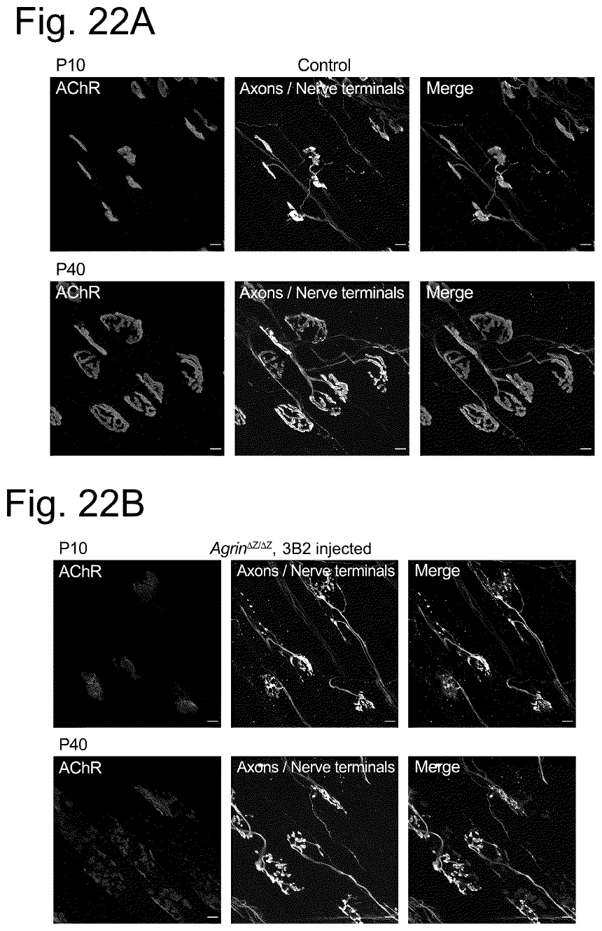

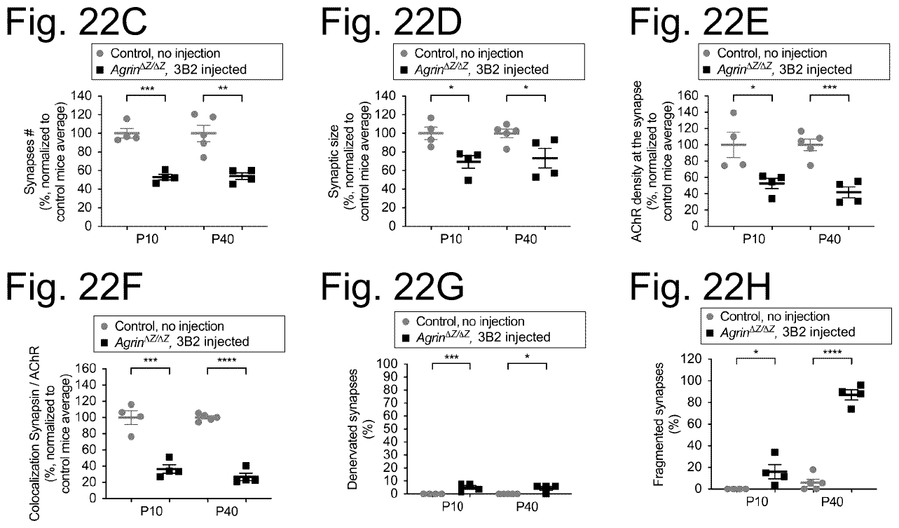

Agrin^{ΔZ/ΔZ} C57BL/6 progeny, who were born from an 3B2-injected Agrin^{ΔZ/+} C57BL/6 female mouse during pregnancy ultimately shows disease relapse around P30. When they lost weight (˜10% weight loss), they were re-injected with 382 (10 mg/kg), and monitored. Re-injection of 3B2 prolongs the survival and maintain body weight of the Agrin^{ΔZ/ΔZ} mice. These data suggest 3B2 can partially rescue Agrin^{ΔZ/ΔZ} mutant mice from neonatal lethality. P40 Agrin^{ΔZ/ΔZ} C57BL/6 mice, who were born from 3B2-injected Agrin^{ΔZ/+} C57BL/6 female mice during pregnancy, were placed in metabolic chambers and monitored 24 hours a day for 4 days. O₂ consumption, CO₂ production, energy expenditure, total activity, water intake, food intake, and body weight measurements were taken. The data shows that Agin^{ΔZ/ΔZ} mice display respiratory, energy expenditure, activity, water intake, food intake, and body weight deficits compared to wildtype mice when disease relapses (FIGS. 20A-20G). Agrin^{ΔZ/ΔZ} mice in a C57BL/6 background, who were born from 3B2-injected Agrin^{ΔZ/ΔZ} C57BL/6 female mice during pregnancy, display motor deficits at disease onset (10% weight loss) as assessed by the latency to fall from a rotating rotarod compared to non-injected wildtype mice. As shown in FIG. 21, Agrin^{ΔZ/ΔZ} mice display motor deficits when disease relapses. 3B2 partially restores synapse development in Agrin^{ΔZ/ΔZ} progeny born from Agrin^{ΔZ/+} C57BL/6 female, injected with 3B2 during pregnancy (FIGS. 22A-22F).

1. A method of treating a disease or condition resulting from a genetic defect in a fetus in a mother, comprising administering to the mother, a therapeutically effective amount of an antibody-based molecule.

2. The method of claim 1, wherein the disease or condition is:
a) a neuromuscular disease; or
b) not associated with the presence of a genetic defect.

3-4. (canceled)

5. The method of claim 1, wherein the fetus is characterized by abnormal development, abnormal growth, expected prenatal death and/or expected early postnatal death, absent the administration of the antibody-based molecule, abnormal neuromuscular junction formation, abnormal synapse development, or deficient motor function.

6. (canceled)

7. The method of claim 1, wherein the antibody-based molecule is administered to the mother intraperitonially, intravenously, subcutaneously in utero or intramuscularly.

8. The method of claim 1, wherein the mother is administered the antibody-based molecule as early as possible after the fetus is diagnosed with the genetic defect, or the mother is administered the antibody-based molecule shortly prior to planned or expected conception to enable the fetus to be treated as early as possible.

9-13. (canceled)

14. The method of claim 1, wherein the treatment further results in a rescue of abnormal synaptic development, abnormal synapse maturation or deficient motor function relative to a fetus whose mother is not administered with the antibody-based molecule or a fetus who is not administered with the combination, wherein the treated fetus is assessed by synapses counting, synapse size estimation, and/or acetylcholine receptor (achr) density estimation at the synapse.

15. The method of claim 1, wherein the antibody-based molecule is an anti-musk antibody or antigen binding fragment thereof.

16. The method of claim 1, wherein the antibody-based molecule is administered at the time at which NMJ are about to be formed.

17. The method of claim 1, wherein the antibody-based molecule:
a) binds the musk Frizzled (Fz)-like domain sequence of amino acid sequence SEQ ID NO: 129;
b) is an agonist musk antibody; or
c) has reduced or eliminated effector function.

18. The method of claim 1, wherein the antibody-based molecule comprises a heavy chain variable domain (VH) or a light chain variable domain (VL) comprising a complementarity determining region (CDR), wherein:
a) the VH or VL has an amino acid sequence that is at least 80% identical to the VH or VL as identified in table 3, and
b) the CDR has an amino acid sequence that is at least 80% identical to the CDR as identified in table 1 or 2.

19. The method of claim 15, wherein the anti-musk antibody or antigen binding fragment thereof comprises a VH and a VL,
wherein the VH comprises:
a) a CDR-H1 amino acid sequence which comprises the amino acid sequence of SEQ ID NO: 147 or has 1, 2, 3, 4 or 5 amino acid alterations relative to the amino acid sequence of SEQ ID NO: 147,
b) a CDR-H2 amino acid sequence which comprises the amino acid sequence of SEQ ID NO: 153 or has 1, 2, 3, 4 or 5 amino acid alterations relative to the amino acid sequence of SEQ ID NO: 153,
c) a CDR-H3 amino acid sequence which comprises the amino acid sequence of SEQ ID NO: 156 or has 1, 2, 3, 4 or 5 amino acid alterations relative to the amino acid sequence of SEQ ID NO: 156, and
wherein the VL comprises:
a) a CDR-L1 amino acid sequence which comprises the amino acid sequence of SEQ ID NO: 159 or has 1, 2, 3, 4 or 5 amino acid alterations relative to the amino acid sequence of SEQ ID NO: 159,
b) a CDR-L2 amino acid sequence which comprises the amino acid sequence of SEQ ID NO: 172 or has 1, 2, 3, 4 or 5 amino acid alterations relative to the amino acid sequence of SEQ ID NO: 172,
c) a CDR-L3 amino acid sequence which comprises the amino acid sequence of SEQ ID NO: 195 or has 1, 2, 3, 4 or 5 amino acid alterations relative to the amino acid sequence of SEQ ID NO: 195.

20. The method of claim 15, wherein the anti-musk antibody or antigen binding fragment thereof comprises:
a) a VH comprising an amino acid sequence that is at least 80% identical to the amino acid sequence of SEQ ID NO: 234, and
b) a VL comprising an amino acid sequence that is at least 80% identical to the amino acid sequence of SEQ ID NO: 235.

21. The method of claim 15, wherein the anti-musk antibody or antigen binding fragment thereof comprises:
1) a VH comprising an amino acid sequence that is at least 80% identical to the amino acid sequence of SEQ ID NO: 234, and a VL comprising an amino acid sequence that is at least 80% identical the amino acid sequence of SEQ ID NO: 235, and
2) wherein the VH comprises:
a) a CDR-H1 amino acid sequence which comprises the amino acid sequence of SEQ ID NO: 147 or has 1, 2, 3, 4 or 5 amino acid alterations relative to the amino acid sequence of SEQ ID NO: 147,
b) a CDR-H2 amino acid sequence which comprises the amino acid sequence of SEQ ID NO: 153 or has 1, 2, 3, 4 or 5 amino acid alterations relative to the amino acid sequence of SEQ ID NO: 153,
c) a CDR-H3 amino acid sequence which comprises the amino acid sequence of SEQ ID NO: 156 or has 1, 2, 3, 4 or 5 amino acid alterations relative to the amino acid sequence of SEQ ID NO: 156, and
3) wherein the VL comprises:
a) a CDR-L1 amino acid sequence which comprises the amino acid sequence of SEQ ID NO: 159 or has 1, 2, 3, 4 or 5 amino acid alterations relative to the amino acid sequence of SEQ ID NO: 159,
b) a CDR-L2 amino acid sequence which comprises the amino acid sequence of SEQ ID NO: 172 or has 1, 2, 3, 4 or 5 amino acid alterations relative to the amino acid sequence of SEQ ID NO: 172,
c) a CDR-L3 amino acid sequence which comprises the amino acid sequence of SEQ ID NO: 195 or has 1, 2, 3, 4 or 5 amino acid alterations relative to the amino acid sequence of SEQ ID NO: 195.

22. The method of claim 15, wherein the anti-musk antibody or antigen binding fragment thereof comprises:
a) a full length heavy chain comprising the amino acid sequence of SEQ ID NO: 268 and;
b) a full length light chain comprising the amino acid sequence of SEQ ID NO: 269,
c) wherein the full length heavy chain comprises L234A and L235A mutations numbered according the EU numbering system.

23. The method of claim 15, wherein the anti-musk antibody or antigen binding fragment thereof comprises:
a) a full length heavy chain comprising the amino acid sequence of SEQ ID NO: 270 and;
b) a full length light chain comprising the amino acid sequence of SEQ ID NO: 271,
wherein the full length heavy chain comprises L234A and L235A mutations numbered according the EU numbering system.

24. (canceled)

25. The method of claim 15, wherein the anti-musk antibody or antigen binding fragment thereof comprises:
a) a VH comprising an amino acid sequence that is at least 80% identical to the amino acid sequence of SEQ ID NO: 202, and
b) a VL comprising an amino acid sequence that is at least 80% identical to the amino acid sequence of SEQ ID NO: 203.

26. The method of claim 15, wherein the anti-musk antibody or antigen binding fragment thereof comprises:
1) a VH comprising an amino acid sequence that is at least 80% identical to the amino acid sequence of SEQ ID NO: 202, and a VL comprising an amino acid sequence that is at least 80% identical to the amino acid sequence of SEQ ID NO: 203, and
2) where the heavy chain variable domain comprises:
a) a CDR-H1 amino acid sequence comprising the amino acid sequence of SEQ ID NO: 147 or having 1, 2, 3, 4 or 5 amino acid alterations relative to the amino acid sequence of SEQ ID NO: 147,
b) a CDR-H2 amino acid sequence comprising the amino acid sequence of SEQ ID NO: 150 or having 1, 2, 3, 4 or 5 amino acid alterations relative to the amino acid sequence of SEQ ID NO: 150, and
c) a CDR-H3 amino acid sequence comprising the amino acid sequence of SEQ ID NO: 156 or having 1, 2, 3, 4 or 5 amino acid alterations relative to the amino acid sequence of SEQ ID NO:156 and
3) where the light chain variable domain comprises:
a) a CDR-L1 amino acid sequence comprising the amino acid sequence of SEQ ID NO: 159 or having 1, 2, 3, 4 or 5 amino acid alterations relative to the amino acid sequence of SEQ ID NO: 159,
b) a CDR-L2 amino acid sequence comprising the amino acid sequence of SEQ ID NO: 172 or having 1, 2, 3, 4 or 5 amino acid alterations relative to the amino acid sequence of SEQ ID NO: 172, and
c) a CDR-L3 amino acid sequence comprising the amino acid sequence of SEQ ID NO: 183 or having 1, 2, 3, 4 or 5 amino acid alterations relative to the amino acid sequence of SEQ ID NO:183.

27. The method of claim 15, wherein the anti-musk antibody or antigen binding fragment thereof comprises:
a) a full length heavy chain comprising the amino acid sequence of SEQ ID NO: 282 and
b) a full length light chain comprising the amino acid sequence of SEQ ID NO: 283,
c) wherein the full length heavy chain comprises L234A and L235A mutations numbered according the EU numbering system.

28. (canceled)

29. A polynucleotide comprising a nucleotide sequence which encodes the antibody-based molecule of claim 15, or a VH, VL or CDR thereof.

30. An expression vector comprising a polynucleotide operably linked to a regulatory region which allows expression of the antibody-based molecule of claim 1, or a VH, VL or CDR thereof, in a host cell or cell-free expression system.

31. A host cell or cell-free expression system comprising the expression vector of claim 30.

32. A pharmaceutical composition comprising the antibody-based molecule of claim 1.

33. The pharmaceutical composition of claim 32, further comprising at least one pharmaceutically acceptable carrier or excipient.

34-36. (canceled)


SEQUENCE LISTING (289 sequences)

SEQ ID NO: 1 - protein, 5 aa, synthetic construct
  REGION 1..5: note = X1 HCDR1
SSSIH

SEQ ID NO: 2 - protein, 5 aa, synthetic construct
  REGION 1..5: note = X2 HCDR1
SSSIH

SEQ ID NO: 3 - protein, 5 aa, synthetic construct
  REGION 1..5: note = X3 HCDR1
SSSIH

SEQ ID NO: 4 - protein, 5 aa, synthetic construct
  REGION 1..5: note = X4 HCDR1
SSSIH

SEQ ID NO: 5 - protein, 5 aa, synthetic construct
  REGION 1..5: note = X5 HCDR1
SSSIH

SEQ ID NO: 6 - protein, 5 aa, synthetic construct
  REGION 1..5: note = X6 HCDR1
SSSIH

SEQ ID NO: 7 - protein, 5 aa, synthetic construct
  REGION 1..5: note = X7 HCDR1
SSSIH

SEQ ID NO: 8 - protein, 5 aa, synthetic construct
  REGION 1..5: note = X8 HCDR1
SSSIH

SEQ ID NO: 9 - protein, 5 aa, synthetic construct
  REGION 1..5: note = X9 HCDR1
SSSIH

SEQ ID NO: 10 - protein, 5 aa, synthetic construct
  REGION 1..5: note = X10 HCDR1
SSSIH

SEQ ID NO: 11 - protein, 5 aa, synthetic construct
  REGION 1..5: note = X11 HCDR1
SSSIH

SEQ ID NO: 12 - protein, 5 aa, synthetic construct
  REGION 1..5: note = X12 HCDR1
SSSIH

SEQ ID NO: 13 - protein, 5 aa, synthetic construct
  REGION 1..5: note = X13 HCDR1
SSSIH

SEQ ID NO: 14 - protein, 5 aa, synthetic construct
  REGION 1..5: note = X14 HCDR1
SSSIH

SEQ ID NO: 15 - protein, 5 aa, synthetic construct
  REGION 1..5: note = X15 HCDR1
SSSIH

SEQ ID NO: 16 - protein, 5 aa, synthetic construct
  REGION 1..5: note = X16 HCDR1
SSSIH

SEQ ID NO: 17 - protein, 17 aa, synthetic construct
  REGION 1..17: note = X1 HCDR2
SISSSSGSTSYADSVKG

SEQ ID NO: 18 - protein, 17 aa, synthetic construct
  REGION 1..17: note = X2 HCDR2
SISSSYGSTSYADSVKG

SEQ ID NO: 19 - protein, 17 aa, synthetic construct
  REGION 1..17: note = X3 HCDR2
SISSSSGYTYYADSVKG

SEQ ID NO: 20 - protein, 17 aa, synthetic construct
  REGION 1..17: note = X4 HCDR2
SISSSSGSTYYADSVKG

SEQ ID NO: 21 - protein, 17 aa, synthetic construct
  REGION 1..17: note = X5 HCDR2
SISSSSGSTSYADSVKG

SEQ ID NO: 22 - protein, 17 aa, synthetic construct
  REGION 1..17: note = X6 HCDR2
SISSYSGYTYYADSVKG

SEQ ID NO: 23 - protein, 17 aa, synthetic construct
  REGION 1..17: note = X7 HCDR2
SISSSYSSTYYADSVKG

SEQ ID NO: 24 - protein, 17 aa, synthetic construct
  REGION 1..17: note = X8 HCDR2
SISSSSGSTSYADSVKG

SEQ ID NO: 25 - protein, 17 aa, synthetic construct
  REGION 1..17: note = X9 HCDR2
SIYSSSGSTYYADSVKG

SEQ ID NO: 26 - protein, 17 aa, synthetic construct
  REGION 1..17: note = X10 HCDR2
SISSSYSSTSYADSVKG

SEQ ID NO: 27 - protein, 17 aa, synthetic construct
  REGION 1..17: note = X11 HCDR2
SISSYSGSTYYADSVKG

SEQ ID NO: 28 - protein, 17 aa, synthetic construct
  REGION 1..17: note = X12 HCDR2
YISPSSGYTSYADSVKG

SEQ ID NO: 29 - protein, 17 aa, synthetic construct
  REGION 1..17: note = X13 HCDR2
SISSSSGSTSYADSVKG

SEQ ID NO: 30 - protein, 17 aa, synthetic construct
  REGION 1..17: note = X14 HCDR2
SISSSSGYTYYADSVKG

SEQ ID NO: 31 - protein, 17 aa, synthetic construct
  REGION 1..17: note = X15 HCDR2
SISSSYGSTSYADSVKG

SEQ ID NO: 32 - protein, 17 aa, synthetic construct
  REGION 1..17: note = X16 HCDR2
SIYSSYGYTSYADSVKG

SEQ ID NO: 33 - protein, 17 aa, synthetic construct
  REGION 1..17: note = X1 HCDR3
KYWSQYYWAHYYGGLDY

SEQ ID NO: 34 - protein, 15 aa, synthetic construct
  REGION 1..15: note = X2 HCDR3
SEGDRYVSGYMGMDY

SEQ ID NO: 35 - protein, 15 aa, synthetic construct
  REGION 1..15: note = X3 HCDR3
SWYEMWMSGYFGFDY

SEQ ID NO: 36 - protein, 15 aa, synthetic construct
  REGION 1..15: note = X4 HCDR3
GEHDYYVFGYLGMDY

SEQ ID NO: 37 - protein, 20 aa, synthetic construct
  REGION 1..20: note = X5 HCDR3
SYTMFYYGGWYGSGYFGMDY

SEQ ID NO: 38 - protein, 15 aa, synthetic construct
  REGION 1..15: note = X6 HCDR3
TYGSYYVSSYTGMDY

SEQ ID NO: 39 - protein, 15 aa, synthetic construct
  REGION 1..15: note = X7 HCDR3
LAGLYHYPGYLGLDY

SEQ ID NO: 40 - protein, 19 aa, synthetic construct
  REGION 1..19: note = X8 HCDR3
SWSYHPWYYHVGWYTGLDY

SEQ ID NO: 41 - protein, 15 aa, synthetic construct
  REGION 1..15: note = X9 HCDR3
SGGEFYITSYYGMDY

SEQ ID NO: 42 - protein, 15 aa, synthetic construct
  REGION 1..15: note = X10 HCDR3
KYYRWRHNKYQGFDY

SEQ ID NO: 43 - protein, 15 aa, synthetic construct
  REGION 1..15: note = X11 HCDR3
SWGSYYVSGFVGFDY

SEQ ID NO: 44 - protein, 17 aa, synthetic construct
  REGION 1..17: note = X12 HCDR3
QYWVPQWWITQYFGMDY

SEQ ID NO: 45 - protein, 15 aa, synthetic construct
  REGION 1..15: note = X13 HCDR3
SSEHWYTIGYYGIDY

SEQ ID NO: 46 - protein, 15 aa, synthetic construct
  REGION 1..15: note = X14 HCDR3
GSHHWFLWIYSGLDY

SEQ ID NO: 47 - protein, 15 aa, synthetic construct
  REGION 1..15: note = X15 HCDR3
SEGDRYVSGYMGMDY

SEQ ID NO: 48 - protein, 16 aa, synthetic construct
  REGION 1..16: note = X16 HCDR3
NWGYYMYWGWYYALDY

SEQ ID NO: 49 - protein, 11 aa, synthetic construct
  REGION 1..11: note = X1 LCDR1
RASQSVSSAVA

SEQ ID NO: 50 - protein, 11 aa, synthetic construct
  REGION 1..11: note = X2 LCDR1
RASQSVSSAVA

SEQ ID NO: 51 - protein, 11 aa, synthetic construct
  REGION 1..11: note = X3 LCDR1
RASQSVSSAVA

SEQ ID NO: 52 - protein, 11 aa, synthetic construct
  REGION 1..11: note = X4 LCDR1
RASQSVSSAVA

SEQ ID NO: 53 - protein, 11 aa, synthetic construct
  REGION 1..11: note = X5 LCDR1
RASQSVSSAVA

SEQ ID NO: 54 - protein, 11 aa, synthetic construct
  REGION 1..11: note = X6 LCDR1
RASQSVSSAVA

SEQ ID NO: 55 - protein, 11 aa, synthetic construct
  REGION 1..11: note = X7 LCDR1
RASQSVSSAVA

SEQ ID NO: 56 - protein, 11 aa, synthetic construct
  REGION 1..11: note = X8 LCDR1
RASQSVSSAVA

SEQ ID NO: 57 - protein, 11 aa, synthetic construct
  REGION 1..11: note = X9 LCDR1
RASQSVSSAVA

SEQ ID NO: 58 - protein, 11 aa, synthetic construct
  REGION 1..11: note = X10 LCDR1
RASQSVSSAVA

SEQ ID NO: 59 - protein, 11 aa, synthetic construct
  REGION 1..11: note = X11 LCDR1
RASQSVSSAVA

SEQ ID NO: 60 - protein, 11 aa, synthetic construct
  REGION 1..11: note = X12 LCDR1
RASQSVSSAVA

SEQ ID NO: 61 - protein, 11 aa, synthetic construct
  REGION 1..11: note = X13 LCDR1
RASQSVSSAVA

SEQ ID NO: 62 - protein, 11 aa, synthetic construct
  REGION 1..11: note = X14 LCDR1
RASQSVSSAVA

SEQ ID NO: 63 - protein, 11 aa, synthetic construct
  REGION 1..11: note = X15 LCDR1
RASQSVSSAVA

SEQ ID NO: 64 - protein, 11 aa, synthetic construct
  REGION 1..11: note = X16 LCDR1
RASQSVSSAVA

SEQ ID NO: 65 - protein, 7 aa, synthetic construct
  REGION 1..7: note = X1 LCDR2
SASSLYS

SEQ ID NO: 66 - protein, 7 aa, synthetic construct
  REGION 1..7: note = X2 LCDR2
SASSLYS

SEQ ID NO: 67 - protein, 7 aa, synthetic construct
  REGION 1..7: note = X3 LCDR2
SASSLYS

SEQ ID NO: 68 - protein, 7 aa, synthetic construct
  REGION 1..7: note = X4 LCDR2
SASSLYS

SEQ ID NO: 69 - protein, 7 aa, synthetic construct
  REGION 1..7: note = X5 LCDR2
SASSLYS

SEQ ID NO: 70 - protein, 7 aa, synthetic construct
  REGION 1..7: note = X6 LCDR2
SASSLYS

SEQ ID NO: 71 - protein, 7 aa, synthetic construct
  REGION 1..7: note = X7 LCDR2
SASSLYS

SEQ ID NO: 72 - protein, 7 aa, synthetic construct
  REGION 1..7: note = X8 LCDR2
SASSLYS

SEQ ID NO: 73 - protein, 7 aa, synthetic construct
  REGION 1..7: note = X9 LCDR2
SASSLYS

SEQ ID NO: 74 - protein, 7 aa, synthetic construct
  REGION 1..7: note = X10 LCDR2
SASSLYS

SEQ ID NO: 75 - protein, 7 aa, synthetic construct
  REGION 1..7: note = X11 LCDR2
SASSLYS

SEQ ID NO: 76 - protein, 7 aa, synthetic construct
  REGION 1..7: note = X12 LCDR2
SASSLYS

SEQ ID NO: 77 - protein, 7 aa, synthetic construct
  REGION 1..7: note = X13 LCDR2
SASSLYS

SEQ ID NO: 78 - protein, 7 aa, synthetic construct
  REGION 1..7: note = X14 LCDR2
SASSLYS

SEQ ID NO: 79 - protein, 7 aa, synthetic construct
  REGION 1..7: note = X15 LCDR2
SASSLYS

SEQ ID NO: 80 - protein, 7 aa, synthetic construct
  REGION 1..7: note = X16 LCDR2
SASSLYS

SEQ ID NO: 81 - protein, 9 aa, synthetic construct
  REGION 1..9: note = X1 LCDR3
QQSSSSLIT

SEQ ID NO: 82 - protein, 9 aa, synthetic construct
  REGION 1..9: note = X2 LCDR3
QQSGVWLIT

SEQ ID NO: 83 - protein, 9 aa, synthetic construct
  REGION 1..9: note = X3 LCDR3
QQSSSSLIT

SEQ ID NO: 84 - protein, 11 aa, synthetic construct
  REGION 1..11: note = X4 LCDR3
QQSYKPGALIT

SEQ ID NO: 85 - protein, 9 aa, synthetic construct
  REGION 1..9: note = X5 LCDR3
QQSSSSLIT

SEQ ID NO: 86 - protein, 9 aa, synthetic construct
  REGION 1..9: note = X6 LCDR3
QQSSSSLIT

SEQ ID NO: 87 - protein, 10 aa, synthetic construct
  REGION 1..10: note = X7 LCDR3
QQSSRSSLLT

SEQ ID NO: 88 - protein, 9 aa, synthetic construct
  REGION 1..9: note = X8 LCDR3
QQSSSSLIT

SEQ ID NO: 89 - protein, 9 aa, synthetic construct
  REGION 1..9: note = X9 LCDR3
QQSSSSLIT

SEQ ID NO: 90 - protein, 9 aa, synthetic construct
  REGION 1..9: note = X10 LCDR3
QQSLWYPVT

SEQ ID NO: 91 - protein, 9 aa, synthetic construct
  REGION 1..9: note = X11 LCDR3
QQNSYYLIT

SEQ ID NO: 92 - protein, 9 aa, synthetic construct
  REGION 1..9: note = X12 LCDR3
QQSSSSLIT

SEQ ID NO: 93 - protein, 11 aa, synthetic construct
  REGION 1..11: note = X13 LCDR3
QQSYGSFSLIT

SEQ ID NO: 94 - protein, 9 aa, synthetic construct
  REGION 1..9: note = X14 LCDR3
QQGSYHLIT

SEQ ID NO: 95 - protein, 9 aa, synthetic construct
  REGION 1..9: note = X15 LCDR3
QQSGVWLIT

SEQ ID NO: 96 - protein, 11 aa, synthetic construct
  REGION 1..11: note = X16 LCDR3
QQWSSAQALIT

SEQ ID NO: 97 - protein, 126 aa, synthetic construct
  REGION 1..126: note = X1 VH
EVQLVESGGGLVQPGGSLRLSCAASGFTFSSSSIHWVRQAPGKGLEWVASISSSSGSTSYADSVKGRFTISADTSKNTAYLQMNSLRAEDTAVYYCARKYWSQYYWAHYYGGLDYWGQGTLVTVSS

SEQ ID NO: 98 - protein, 107 aa, synthetic construct
  REGION 1..107: note = VL
DIQMTQSPSSLSASVGDRVTITCRASQSVSSAVAWYQQKPGKAPKLLIYSASSLYSGVPSRFSGSRSGTDFTLTISSLQPEDFATYYCQQSSSSLITFGQGTKVEIK

SEQ ID NO: 99 - protein, 124 aa, synthetic construct
  REGION 1..124: note = X2 VH
EVQLVESGGGLVQPGGSLRLSCAASGFTVSSSSIHWVRQAPGKGLEWVASISSSYGSTSYADSVKGRFTISADTSKNTAYLQMNSLRAEDTAVYYCARSEGDRYVSGYMGMDYWGQGTLVTVSS

SEQ ID NO: 100 - protein, 107 aa, synthetic construct
  REGION 1..107: note = X2 VL
DIQMTQSPSSLSASVGDRVTITCRASQSVSSAVAWYQQKPGKAPKLLIYSASSLYSGVPSRFSGSRSGTDFTLTISSLQPEDFATYYCQQSGVWLITFGQGTKVEIK

SEQ ID NO: 101 - protein, 124 aa, synthetic construct
  REGION 1..124: note = X3 VH
EVQLVESGGGLVQPGGSLRLSCAASGFTISSSSIHWVRQAPGKGLEWVASISSSSGYTYYADSVKGRFTISADTSKNTAYLQMNSLRAEDTAVYYCARSWYEMWMSGYFGFDYWGQGTLVTVSS

SEQ ID NO: 102 - protein, 107 aa, synthetic construct; identical to SEQ ID NO: 98
  REGION 1..107: note = VL

SEQ ID NO: 103 - protein, 124 aa, synthetic construct
  REGION 1..124: note = X4 VH
EVQLVESGGGLVQPGGSLRLSCAASGFTVSSSSIHWVRQAPGKGLEWVASISSSSGSTYYADSVKGRFTISADTSKNTAYLQMNSLRAEDTAVYYCARGEHDYYVFGYLGMDYWGQGTLVTVSS

SEQ ID NO: 104 - protein, 109 aa, synthetic construct
  REGION 1..109: note = VL
DIQMTQSPSSLSASVGDRVTITCRASQSVSSAVAWYQQKPGKAPKLLIYSASSLYSGVPSRFSGSRSGTDFTLTISSLQPEDFATYYCQQSYKPGALITFGQGTKVEIK

SEQ ID NO: 105 - protein, 129 aa, synthetic construct
  REGION 1..129: note = X5 VH
EVQLVESGGGLVQPGGSLRLSCAASGFTFYSSSIHWVRQAPGKGLEWVASISSSSGSTSYADSVKGRFTISADTSKNTAYLQMNSLRAEDTAVYYCARSYTMFYYGGWYGSGYFGMDYWGQGTLVTVSS

SEQ ID NO: 106 - protein, 107 aa, synthetic construct; identical to SEQ ID NO: 98
  REGION 1..107: note = VL

SEQ ID NO: 107 - protein, 124 aa, synthetic construct
  REGION 1..124: note = X6 VH
EVQLVESGGGLVQPGGSLRLSCAASGFTFSSSSIHWVRQAPGKGLEWVASISSYSGYTYYADSVKGRFTISADTSKNTAYLQMNSLRAEDTAVYYCARTYGSYYVSSYTGMDYWGQGTLVTVSS

SEQ ID NO: 108 - protein, 107 aa, synthetic construct; identical to SEQ ID NO: 98
  REGION 1..107: note = VL

SEQ ID NO: 109 - protein, 124 aa, synthetic construct
  REGION 1..124: note = X7 VH
EVQLVESGGGLVQPGGSLRLSCAASGFTLYSSSIHWVRQAPGKGLEWVASISSSYSSTYYADSVKGRFTISADTSKNTAYLQMNSLRAEDTAVYYCARLAGLYHYPGYLGLDYWGQGTLVTVSS

SEQ ID NO: 110 - protein, 108 aa, synthetic construct
  REGION 1..108: note = VL
DIQMTQSPSSLSASVGDRVTITCRASQSVSSAVAWYQQKPGKAPKLLIYSASSLYSGVPSRFSGSRSGTDFTLTISSLQPEDFATYYCQQSSRSSLLTFGQGTKVEIK

SEQ ID NO: 111 - protein, 128 aa, synthetic construct
  REGION 1..128: note = X8 VH
EVQLVESGGGLVQPGGSLRLSCAASGFTVSSSSIHWVRQAPGKGLEWVASISSSSGSTSYADSVKGRFTISADTSKNTAYLQMNSLRAEDTAVYYCARSWSYHPWYYHVGWYTGLDYWGQGTLVTVSS

SEQ ID NO: 112 - protein, 107 aa, synthetic construct; identical to SEQ ID NO: 98
  REGION 1..107: note = VL

SEQ ID NO: 113 - protein, 124 aa, synthetic construct
  REGION 1..124: note = X9 VH
EVQLVESGGGLVQPGGSLRLSCAASGFTFSSSSIHWVRQAPGKGLEWVASIYSSSGSTYYADSVKGRFTISADTSKNTAYLQMNSLRAEDTAVYYCARSGGEFYITSYYGMDYWGQGTLVTVSS

SEQ ID NO: 114 - protein, 107 aa, synthetic construct; identical to SEQ ID NO: 98
  REGION 1..107: note = VL

SEQ ID NO: 115 - protein, 124 aa, synthetic construct
  REGION 1..124: note = X10 VH
EVQLVESGGGLVQPGGSLRLSCAASGFTFSSSSIHWVRQAPGKGLEWVASISSSYSSTSYADSVKGRFTISADTSKNTAYLQMNSLRAEDTAVYYCARKYYRWRHNKYQGFDYWGQGTLVTVSS

SEQ ID NO: 116 - protein, 107 aa, synthetic construct
  REGION 1..107: note = VL
DIQMTQSPSSLSASVGDRVTITCRASQSVSSAVAWYQQKPGKAPKLLIYSASSLYSGVPSRFSGSRSGTDFTLTISSLQPEDFATYYCQQSLWYPVTFGQGTKVEIK

SEQ ID NO: 117 - protein, 124 aa, synthetic construct
  REGION 1..124: note = X11 VH
EVQLVESGGGLVQPGGSLRLSCAASGFTISSSSIHWVRQAPGKGLEWVASISSYSGSTYYADSVKGRFTISADTSKNTAYLQMNSLRAEDTAVYYCARSWGSYYVSGFVGFDYWGQGTLVTVSS

SEQ ID NO: 118 - protein, 107 aa, synthetic construct
  REGION 1..107: note = VL
DIQMTQSPSSLSASVGDRVTITCRASQSVSSAVAWYQQKPGKAPKLLIYSASSLYSGVPSRFSGSRSGTDFTLTISSLQPEDFATYYCQQNSYYLITFGQGTKVEIK

SEQ ID NO: 119 - protein, 126 aa, synthetic construct
  REGION 1..126: note = X12 VH
EVQLVESGGGLVQPGGSLRLSCAASGFTISSSSIHWVRQAPGKGLEWVAYISPSSGYTSYADSVKGRFTISADTSKNTAYLQMNSLRAEDTAVYYCARQYWVPQWWITQYFGMDYWGQGTLVTVSS

SEQ ID NO: 120 - protein, 107 aa, synthetic construct; identical to SEQ ID NO: 98
  REGION 1..107: note = VL

SEQ ID NO: 121 - protein, 124 aa, synthetic construct
  REGION 1..124: note = X13 VH
EVQLVESGGGLVQPGGSLRLSCAASGFTISSSSIHWVRQAPGKGLEWVASISSSSGSTSYADSVKGRFTISADTSKNTAYLQMNSLRAEDTAVYYCARSSEHWYTIGYYGIDYWGQGTLVTVSS

SEQ ID NO: 122 - protein, 109 aa, synthetic construct
  REGION 1..109: note = VL
DIQMTQSPSSLSASVGDRVTITCRASQSVSSAVAWYQQKPGKAPKLLIYSASSLYSGVPSRFSGSRSGTDFTLTISSLQPEDFATYYCQQSYGSFSLITFGQGTKVEIK

SEQ ID NO: 123 - protein, 124 aa, synthetic construct
  REGION 1..124: note = X14 VH
EVQLVESGGGLVQPGGSLRLSCAASGFTFSSSSIHWVRQAPGKGLEWVASISSSSGYTYYADSVKGRFTISADTSKNTAYLQMNSLRAEDTAVYYCARGSHHWFLWIYSGLDYWGQGTLVTVSS

SEQ ID NO: 124 - protein, 107 aa, synthetic construct
  REGION 1..107: note = VL
DIQMTQSPSSLSASVGDRVTITCRASQSVSSAVAWYQQKPGKAPKLLIYSASSLYSGVPSRFSGSRSGTDFTLTISSLQPEDFATYYCQQGSYHLITFGQGTKVEIK

SEQ ID NO: 125 - protein, 124 aa, synthetic construct; identical to SEQ ID NO: 99
  REGION 1..124: note = X15 VH

SEQ ID NO: 126 - protein, 107 aa, synthetic construct; identical to SEQ ID NO: 100
  REGION 1..107: note = VL

SEQ ID NO: 127 - protein, 125 aa, synthetic construct
  REGION 1..125: note = X16 VH
EVQLVESGGGLVQPGGSLRLSCAASGFTFSSSSIHWVRQAPGKGLEWVASIYSSYGYTSYADSVKGRFTISADTSKNTAYLQMNSLRAEDTAVYYCARNWGYYMYWGWYYALDYWGQGTLVTVSS

SEQ ID NO: 128 - protein, 109 aa, synthetic construct
  REGION 1..109: note = VL
DIQMTQSPSSLSASVGDRVTITCRASQSVSSAVAWYQQKPGKAPKLLIYSASSLYSGVPSRFSGSRSGTDFTLTISSLQPEDFATYYCQQWSSAQALITFGQGTKVEIK

SEQ ID NO: 129 - protein, 869 aa, Homo sapiens
MRELVNIPLVHILTLVAFSGTEKLPKAPVITTPLETVDALVEEVATFMCAVESYPQPEISWTRNKILIKLFDTRYSIRENGQLLTILSVEDSDDGIYCCTANNGVGGAVESCGALQVKMKPKITRPPINVKIIEGLKAVLPCTTMGNPKPSVSWIKGDSPLRENSRIAVLESGSLRIHNVQKEDAGQYRCVAKNSLGTAYSKVVKLEVEVFARILRAPESHNVTFGSFVTLHCTATGIPVPTITWIENGNAVSSGSIQESVKDRVIDSRLQLFITKPGLYTCIATNKHGEKFSTAKAAATISIAEWSKPQKDNKGYCAQYRGEVCNAVLAKDALVFLNTSYADPEEAQELLVHTAWNELKVVSPVCRPAAEALLCNHIFQECSPGVVPTPIPICREYCLAVKELFCAKEWLVMEEKTHRGLYRSEMHLLSVPECSKLPSMHWDPTACARLPHLDYNKENLKTFPPMTSSKPSVDIPNLPSSSSSSFSVSPTYSMTVIISIMSSFAIFVLLTITTLYCCRRRKQWKNKKRESAAVTLTTLPSELLLDRLHPNPMYQRMPLLLNPKLLSLEYPRNNIEYVRDIGEGAFGRVFQARAPGLLPYEPFTMVAVKMLKEEASADMQADFQREAALMAEFDNPNIVKLLGVCAVGKPMCLLFEYMAYGDLNEFLRSMSPHTVCSLSHSDLSMRAQVSSPGPPPLSCAEQLCIARQVAAGMAYLSERKFVHRDLATRNCLVGENMVVKIADFGLSRNIYSADYYKANENDAIPIRWMPPESIFYNRYTTESDVWAYGVVLWEIFSYGLQPYYGMAHEEVIYYVRDGNILSCPENCPVELYNLMRLCWSKLPADRPSFTSIHRILERMCERAEGTVSV

SEQ ID NO: 130 - protein, 139 aa, synthetic construct
  REGION 1..139: note = Fz-like domain of MuSK
DNKGYCAQYRGEVCNAVLAKDALVFLNTSYADPEEAQELLVHTAWNELKVVSPVCRPAAEALLCNHIFQECSPGVVPTPIPICREYCLAVKELFCAKEWLVMEEKTHRGLYRSEMHLLSVPECSKLPSMHWDPTACARL

SEQ ID NO: 131 - protein, 124 aa, synthetic construct
  REGION 1..124: note = X17 VH
EVQLVESGGGLVQPGGSLRLSCAASGFTISYSSIHWVRQAPGKGLEWVASIYSSSGSTYYADSVKGRFTISADTSKNTAYLQMNSLRAEDTAVYYCARGDHGYYVFGYLGMDYWGQGTLVTVSS

SEQ ID NO: 132 - protein, 109 aa, synthetic construct; identical to SEQ ID NO: 104
  REGION 1..109: note = X17 VL

SEQ ID NO: 133 - protein, 126 aa, synthetic construct
  REGION 1..126: note = X18 VH
EVQLVESGGGLVQPGGSLRLSCAASGFTISSSSIHWVRQAPGKGLEWVASISSSSGYTSYADSVKGRFTISADTSKNTAYLQMNSLRAEDTAVYYCARKYSKRAYPDYYWRGLDYWGQGTLVTVSS

SEQ ID NO: 134 - protein, 107 aa, synthetic construct
  REGION 1..107: note = VL
DIQMTQSPSSLSASVGDRVTITCRASQSVSSAVAWYQQKPGKAPKLLIYSASSLYSGVPSRFSGSRSGTDFTLTISSLQPEDFATYYCQQSYWWPITFGQGTKVEIK

SEQ ID NO: 135 - protein, 5 aa, synthetic construct
  REGION 1..5: note = X17 HCDR1
YSSIH

SEQ ID NO: 136 - protein, 5 aa, synthetic construct
  REGION 1..5: note = X18 HCDR1
SSSIH

SEQ ID NO: 137 - protein, 17 aa, synthetic construct
  REGION 1..17: note = X17 HCDR2
SIYSSSGSTYYADSVKG

SEQ ID NO: 138 - protein, 17 aa, synthetic construct
  REGION 1..17: note = X18 HCDR2
SISSSSGYTSYADSVKG

SEQ ID NO: 139 - protein, 15 aa, synthetic construct
  REGION 1..15: note = X17 HCDR3
GDHGYYVFGYLGMDY

SEQ ID NO: 140 - protein, 17 aa, synthetic construct
  REGION 1..17: note = X18 HCDR3
KYSKRAYPDYYWRGLDY

SEQ ID NO: 141 - protein, 11 aa, synthetic construct
  REGION 1..11: note = X17 LCDR1
RASQSVSSAVA

SEQ ID NO: 142 - protein, 11 aa, synthetic construct
  REGION 1..11: note = X18 LCDR1
RASQSVSSAVA

SEQ ID NO: 143 - protein, 7 aa, synthetic construct
  REGION 1..7: note = X17 LCDR2
SASSLYS

SEQ ID NO: 144 - protein, 7 aa, synthetic construct
  REGION 1..7: note = X18 LCDR2
SASSLYS

SEQ ID NO: 145 - protein, 11 aa, synthetic construct
  REGION 1..11: note = X17 LCDR3
QQSYKPGALIT

SEQ ID NO: 146 - protein, 9 aa, synthetic construct
  REGION 1..9: note = X18 LCDR3
QQSYWWPIT

SEQ ID NO: 147 - protein, 5 aa, synthetic construct
  REGION 1..5: note = 14D10 HCDR1
DYGMS

SEQ ID NO: 148 - protein, 7 aa, synthetic construct
  REGION 1..7: note = 17H10 HCDR1
ARYYSWS

SEQ ID NO: 149 - protein, 5 aa, synthetic construct
  REGION 1..5: note = 16F11 HCDR1
LYYMN

SEQ ID NO: 150 - protein, 17 aa, synthetic construct
  REGION 1..17: note = 14D10 HCDR2
AIPWNGGSTYYKESVKG

SEQ ID NO: 151 - protein, 16 aa, synthetic construct
  REGION 1..16: note = 17H10 HCDR2
VIAYDGSTYYSPSLKS

SEQ ID NO: 152 - protein, 16 aa, synthetic construct
  REGION 1..16: note = 16F11 HCDR2
VIDTHSIAYYADSVKG

SEQ ID NO: 153 - protein, 17 aa, synthetic construct
  REGION 1..17: note = 3B2g1m1 HCDR2
AIPWSGGSTYYKESVKG

SEQ ID NO: 154 - protein, 17 aa, synthetic construct
  REGION 1..17: note = 3B2g1m2 HCDR2
AIPGSGGSTYYKESVKG

SEQ ID NO: 155 - protein, 17 aa, synthetic construct
  REGION 1..17: note = 3B2g1m4 HCDR2
AIPWQGGSTYYKESVKG

SEQ ID NO: 156 - protein, 12 aa, synthetic construct
  REGION 1..12: note = 14D10 HCDR3
RSGRIAFGALDA

SEQ ID NO: 157 - protein, 11 aa, synthetic construct
  REGION 1..11: note = 17H10 HCDR3
GSSRVAAAFDS

SEQ ID NO: 158 - protein, 7 aa, synthetic construct
  REGION 1..7: note = 16F11 HCDR3
GRTALVR

SEQ ID NO: 159 - protein, 14 aa, synthetic construct
  REGION 1..14: note = 14D10 LCDR1
GLSSGSVTSSNYPD

SEQ ID NO: 160 - protein, 14 aa, synthetic construct
  REGION 1..14: note = 3C4 LCDR1
GLSSGSVTASNYPD

SEQ ID NO: 161 - protein, 11 aa, synthetic construct
  REGION 1..11: note = 17H10 LCDR1
GGNRIGGKSVQ

SEQ ID NO: 162 - protein, 11 aa, synthetic construct
  REGION 1..11: note = 23B6 LCDR1
GGDNIGSKNAQ

SEQ ID NO: 163 - protein, 11 aa, synthetic construct
  REGION 1..11: note = 30E1 LCDR1
GGDNIGSKNTQ

SEQ ID NO: 164 - protein, 11 aa, synthetic construct
  REGION 1..11: note = 30A11 LCDR1
GGDNIASKNVQ

SEQ ID NO: 165 - protein, 17 aa, synthetic construct
  REGION 1..17: note = 16F11 LCDR1
KSSQSVVFGSNQKSYLN

SEQ ID NO: 166 - protein, 17 aa, synthetic construct
  REGION 1..17: note = 4C11 LCDR1
RSSQSVLYSSNQKNYLN

SEQ ID NO: 167 - protein, 15 aa, synthetic construct
  REGION 1..15: note = 7A12 LCDR1
ESSQSVLYNQKNYLN

SEQ ID NO: 168 - protein, 17 aa, synthetic construct
  REGION 1..17: note = 7G12 LCDR1
KSSQRVQLGSNQKSYLN

SEQ ID NO: 169 - protein, 15 aa, synthetic construct
  REGION 1..15: note = 7B8 LCDR1
KSSQSVLYNQKNYLA

SEQ ID NO: 170 - protein, 7 aa, synthetic construct
  REGION 1..7: note = 14D10 LCDR2
TTNSRHS

SEQ ID NO: 171 - protein, 7 aa, synthetic construct
  REGION 1..7: note = 7G4 LCDR2
STNSRHS

SEQ ID NO: 172 - protein, 7 aa, synthetic construct
  REGION 1..7: note = 3C4 LCDR2
STDSRHS

SEQ ID NO: 173 - protein, 7 aa, synthetic construct
  REGION 1..7: note = 31G2 LCDR2
STNSRLS

SEQ ID NO: 174 - protein, 7 aa, synthetic construct
  REGION 1..7: note = 17H10 LCDR2
ADSRRPS

SEQ ID NO: 175 - protein, 7 aa, synthetic construct
  REGION 1..7: note = 16F11 LCDR2
YASTQES

SEQ ID NO: 176 - protein, 7 aa, synthetic construct
  REGION 1..7: note = 4C11 LCDR2
WASARES

SEQ ID NO: 177 - protein, 7 aa, synthetic construct
  REGION 1..7: note = 7A12 LCDR2
WASTRQS

SEQ ID NO: 178 - protein, 7 aa, synthetic construct
  REGION 1..7: note = 7G12 LCDR2
YASTQQS

SEQ ID NO: 179 - protein, 7 aa, synthetic construct
  REGION 1..7: note = 7B8 LCDR2
WASTRES

SEQ ID NO: 180 - protein, 12 aa, synthetic construct
  REGION 1..12: note = 14D10 LCDR3
ALYMGGGSNVYV

SEQ ID NO: 181 - protein, 13 aa, synthetic construct
  REGION 1..13: note = 7G4 LCDR3
ALYMGRGSNKDYV

SEQ ID NO: 182 - protein, 12 aa, synthetic construct
  REGION 1..12: note = 3C4 LCDR3
ALYMYSDSKLYV

SEQ ID NO: 183 - protein, 12 aa, synthetic construct
  REGION 1..12: note = 3B2 LCDR3
GLYMYSGSKNYV

SEQ ID NO: 184 - protein, 12 aa, synthetic construct
  REGION 1..12: note = 3G3 LCDR3
ALYMGSDIRNYV

SEQ ID NO: 185 - protein, 12 aa, synthetic construct
  REGION 1..12: note = 31G2 LCDR3
ALYMGSGSRNYV

SEQ ID NO: 186 - protein, 12 aa, synthetic construct
  REGION 1..12: note = 31B7 LCDR3
ALYMGSESRNYV

SEQ ID NO: 187 - protein, 10 aa, synthetic construct
  REGION 1..10: note = 17H10 LCDR3
HVWGSTASAD

SEQ ID NO: 188 - protein, 10 aa, synthetic construct
  REGION 1..10: note = 23B6 LCDR3
HVWDSSTNAW

SEQ ID NO: 189 - protein, 11 aa, synthetic construct
  REGION 1..11: note = 30A11 LCDR3
QVWDSSTNVAV

SEQ ID NO: 190 - protein, 8 aa, synthetic construct
  REGION 1..8: note = 16F11 LCDR3
QQAYSAPT

SEQ ID NO: 191 - protein, 9 aa, synthetic construct
  REGION 1..9: note = 4C11 LCDR3
QQSYKPPYG

SEQ ID NO: 192 - protein, 9 aa, synthetic construct
  REGION 1..9: note = 7A12 LCDR3
QQAYNAPLT

SEQ ID NO: 193 - protein, 9 aa, synthetic construct
  REGION 1..9: note = 7G12 LCDR3
QQGYSAPFT

SEQ ID NO: 194 - protein, 9 aa, synthetic construct
  REGION 1..9: note = 7B8 LCDR3
QQGYSVPYT

SEQ ID NO: 195 - protein, 12 aa, synthetic construct
  REGION 1..12: note = 3B2g2m1 LCDR3
GLYSYSGSKNYV

SEQ ID NO: 196 - protein, 121 aa, synthetic construct
  REGION 1..121: note = 14D10 VH
ELQLVESGGGLVQPGGSLRLSCAASGFTFDDYGMSWVRQAPGKGLEWVSAIPWNGGSTYYKESVKGRFTISRDNAKKTLYLQMNSLKSEDTAVYYCAKRSGRIAFGALDAWGQGTLVTVSS

SEQ ID NO: 197 - protein, 112 aa, synthetic construct
  REGION 1..112: note = VL
QAVVTQEPSLSVSPGGTVTLTCGLSSGSVTSSNYPDWYQQTPGQAPRTLIYTTNSRHSGVPSRFSGSISGNKAALTITGAQPEDEADYYCALYMGGGSNVYVFGGGTKLTVL

SEQ ID NO: 198 - protein, 121 aa, synthetic construct; identical to SEQ ID NO: 196
  REGION 1..121: note = 7G4 VH

SEQ ID NO: 199 - protein, 113 aa, synthetic construct
  REGION 1..113: note = VL
QAVVTQEPSLSVSPGGTVTLTCGLSSGSVTSSNYPDWYQQTPGQAPRALIYSTNSRHSGVPSRFSGSISGNKAALTITGAQPEDEADYYCALYMGRGSNKDYVFGGGTKLTVL

SEQ ID NO: 200 - protein, 121 aa, synthetic construct; identical to SEQ ID NO: 196
  REGION 1..121: note = 3C4 VH

SEQ ID NO: 201 - protein, 112 aa, synthetic construct
  REGION 1..112: note = VL
QAVVTQEPSLSVSPGGTVTLTCGLSSGSVTASNYPDWYQQTPGQAPRGLIYSTDSRHSGVPSRFSGSISGNKAALTITGAQPEDEADYYCALYMYSDSKLYVFGGGTKLTVL

SEQ ID NO: 202 - protein, 121 aa, synthetic construct; identical to SEQ ID NO: 196
  REGION 1..121: note = 3B2 VH

SEQ ID NO: 203 - protein, 112 aa, synthetic construct
  REGION 1..112: note = VL
QAVVTQEPSLSVSPGGTVTLTCGLSSGSVTSSNYPDWYQQTPGQAPRGLIYSTDSRHSGVPSRFSGSISGNKAALTITGAQSEDEADYYCGLYMYSGSKNYVFGGGTKLTVL

SEQ ID NO: 204 - protein, 121 aa, synthetic construct; identical to SEQ ID NO: 196
  REGION 1..121: note = 3G3 VH

SEQ ID NO: 205 - protein, 112 aa, synthetic construct
  REGION 1..112: note = VL
QTVVTQEPSLSVSPGGTVTLTCGLSSGSVTSSNYPDWYQQTPGQAPRALIYSTNSRHSGVPSRFSGSTSGNKAALTITGAQPEDEADYYCALYMGSDIRNYVFGGGTKLTVL

SEQ ID NO: 206 - protein, 121 aa, synthetic construct; identical to SEQ ID NO: 196
  REGION 1..121: note = 31G2 VH

SEQ ID NO: 207 - protein, 112 aa, synthetic construct
  REGION 1..112: note = VL
QAVVTQEPSLSVSPGGTVTLTCGLSSGSVTSSNYPDWYQQTPGQAPRALIYSTNSRLSGVPSRFSGSFSGNKAALTITGAQPEDEADYYCALYMGSGSRNYVFGGGTKLTVL

SEQ ID NO: 208 - protein, 121 aa, synthetic construct; identical to SEQ ID NO: 196
  REGION 1..121: note = 31B7 VH

SEQ ID NO: 209 - protein, 112 aa, synthetic construct
  REGION 1..112: note = VL
QAVVTQEPSLSVSPGGTVTLTCGLSSGSVTSSNYPDWYQQTPGQAPRALIYSTNSRLSGVPSRFSGSFSGNKAALTITGAQPEDEADYYCALYMGSESRNYVFGGGTKLTVL

SEQ ID NO: 210 - protein, 121 aa, synthetic construct
  REGION 1..121: note = 17H10 VH
QVQVQESGPGLVKPSQTLSLTCTVSGGSITARYYSWSWIRQPPGKGLEWMGVIAYDGSTYYSPSLKSRTSISRDTSKNQFSLHLSSVTPDDTAVYYCARGSSRVAAAFDSWGQGTQVTVSS

SEQ ID NO: 211 - protein, 107 aa, synthetic construct
  REGION 1..107: note = VL
SYELTQSPSVSVALRQTAKITCGGNRIGGKSVQWYQQKPGQAPMLVIYADSRRPSGIPERFTGSNSGNTATLTITGAQAEDEADYYCHVWGSTASADFGGGTHLTVL

SEQ ID NO: 212 - protein, 121 aa, synthetic construct; identical to SEQ ID NO: 210
  REGION 1..121: note = 23B6 VH

SEQ ID NO: 213 - protein, 107 aa, synthetic construct
  REGION 1..107: note = VL
SYELTQSPSVSVALRQTAKITCGGDNIGSKNAQWYQQKPGQAPVMVLYADSRRPSGIPERFSGSNSGNTATLTISGAQAEDEADYYCHVWDSSTNAWFGGGTHLTVL

SEQ ID NO: 214 - protein, 121 aa, synthetic construct; identical to SEQ ID NO: 210
  REGION 1..121: note = 30E1 VH

SEQ ID NO: 215 - protein, 107 aa, synthetic construct
  REGION 1..107: note = VL
SYELTQSPSVSVALRRTAKITCGGDNIGSKNTQWYQQKPGQAPVLVIYADSRRPSGIPERFSGSNSGNTATLTISGAQAEDEADYYCHVWDSSTNAWFGGGTHLTVL

SEQ ID NO: 216 - protein, 121 aa, synthetic construct; identical to SEQ ID NO: 210
  REGION 1..121: note = 30A11 VH

SEQ ID NO: 217 - protein, 108 aa, synthetic construct
  REGION 1..108: note = VL
SYELTQSPSVTVALRQTAKITCGGDNIASKNVQWYQQKPGQAPSLVIWADSRRPSGIPVRFSGSNFGNTATLTISGAQAEDEADYYCQVWDSSTNVAVFGGGTHLTVL

SEQ ID NO: 218 - protein, 115 aa, synthetic construct
  REGION 1..115: note = 16F11 VH
EVQLVESGGGLVQPGGSLSLSCVASGFTFSLYYMNWVRQAPGKGLEWLSVIDTHSIAYYADSVKGRFTISRDNVKNTLYLQLNNLKPEDTALYYCVLGRTALVRWGQGTQVTVSS

SEQ ID NO: 219 - protein, 112 aa, synthetic construct
  REGION 1..112: note = VL
DIVMTQSPSSVTASVGEKVTINCKSSQSVVFGSNQKSYLNWYQQRPGQSPRLLIYYASTQESGIPDRFSGSGSTTDFTLTISSVQPEDAAVYYCQQAYSAPTFGSGTRLEIK

SEQ ID NO: 220 - protein, 115 aa, synthetic construct; identical to SEQ ID NO: 218
  REGION 1..115: note = 4C11 VH

SEQ ID NO: 221 - protein, 113 aa, synthetic construct
  REGION 1..113: note = VL
DIVMTQSPSSVTASAGERVTINCRSSQSVLYSSNQKNYLNWYQQRLGQSPRLLIYWASARESGVPDRFSGSGSTTNFTLTISSFQPEDAAVYYCQQSYKPPYGFGSGTRLEIK

SEQ ID NO: 222 - protein, 115 aa, synthetic construct; identical to SEQ ID NO: 218
  REGION 1..115: note = 7A12 VH

SEQ ID NO: 223 - protein, 113 aa, synthetic construct
  REGION 1..113: note = VL
EIVLTQSPSSVTASIGEKVTINCESSQSVLYNQKNYLNWYQQRPGQSPRLLIYWASTRQSGVPDRFSGSGSGSTTDFTLTISSFQPEDVAVYYCQQAYNAPLTFGPGTKVELK

SEQ ID NO: 224 - protein, 115 aa, synthetic construct; identical to SEQ ID NO: 218
  REGION 1..115: note = 7G12 VH

SEQ ID NO: 225 - protein, 113 aa, synthetic construct
  REGION 1..113: note = VL
EIVLTQSPNSVTASVGEKVTINCKSSQRVQLGSNQKSYLNWYQQRPGQSPRLLIYYASTQQSGIPDRFSGSGSATDFTLTINSVQPEDAAVYYCQQGYSAPFTFGQGTKVELK

SEQ ID NO: 226 - protein, 115 aa, synthetic construct; identical to SEQ ID NO: 218
  REGION 1..115: note = 7B8 VH

SEQ ID NO: 227 - protein, 111 aa, synthetic construct
  REGION 1..111: note = VL
EIVLTQSPSSVTASAGEKVTINCKSSQSVLYNQKNYLAWYQQRPGQSPRLLIYWASTRESGVPDRFSGSGSTTDFTLTISSFQPEDVAVYYCQQGYSVPYTFGSGTRLEIK

SEQ ID NO: 228 - protein, 121 aa, synthetic construct
  REGION 1..121: note = 3B2g1m1 VH
EVQLLESGGGLVQPGGSLRLSCAASGFTFSDYGMSWVRQAPGKGLEWVSAIPWSGGSTYYKESVKGRFTISRDNSKNTLYLQMNSLRAEDTAVYYCAKRSGRIAFGALDAWGQGTLVTVSS

SEQ ID NO: 229 - protein, 112 aa, synthetic construct
  REGION 1..112: note = VL
QTVVTQEPSFSVSPGGTVTLTCGLSSGSVTSSNYPDWYQQTPGQAPRTLIYSTDSRHSGVPDRFSGSILGNKAALTITGAQADDESDYYCGLYMYSGSKNYVFGGGTKLTVL

SEQ ID NO: 230 - protein, 121 aa, synthetic construct
  REGION 1..121: note = 3B2g1m2 VH
EVQLLESGGGLVQPGGSLRLSCAASGFTFSDYGMSWVRQAPGKGLEWVSAIPGSGGSTYYKESVKGRFTISRDNSKNTLYLQMNSLRAEDTAVYYCAKRSGRIAFGALDAWGQGTLVTVSS

SEQ ID NO: 231 - protein, 112 aa, synthetic construct; identical to SEQ ID NO: 229
  REGION 1..112: note = VL

SEQ ID NO: 232 - protein, 121 aa, synthetic construct
  REGION 1..121: note = 3B2g1m4 VH
EVQLLESGGGLVQPGGSLRLSCAASGFTFSDYGMSWVRQAPGKGLEWVSAIPWQGGSTYYKESVKGRFTISRDNSKNTLYLQMNSLRAEDTAVYYCAKRSGRIAFGALDAWGQGTLVTVSS

SEQ ID NO: 233 - protein, 112 aa, synthetic construct; identical to SEQ ID NO: 229
  REGION 1..112: note = VL

SEQ ID NO: 234 - protein, 121 aa, synthetic construct; identical to SEQ ID NO: 228
  REGION 1..121: note = 3B2g2m1 VH

SEQ ID NO: 235 - protein, 112 aa, synthetic construct
  REGION 1..112: note = VL
QTVVTQEPSFSVSPGGTVTLTCGLSSGSVTSSNYPDWYQQTPGQAPRTLIYSTDSRHSGVPDRFSGSILGNKAALTITGAQADDESDYYCGLYSYSGSKNYVFGGGTKLTVL

SEQ ID NO: 236 - protein, 121 aa, synthetic construct; identical to SEQ ID NO: 230
  REGION 1..121: note = 3B2g2m2 VH

SEQ ID NO: 237 - protein, 112 aa, synthetic construct; identical to SEQ ID NO: 235
  REGION 1..112: note = VL

SEQ ID NO: 238 - protein, 121 aa, synthetic construct; identical to SEQ ID NO: 232
  REGION 1..121: note = 3B2g2m4 VH

SEQ ID NO: 239 - protein, 112 aa, synthetic construct; identical to SEQ ID NO: 235
  REGION 1..112: note = VL

SEQ ID NO: 240 - protein, 15 aa, synthetic construct
  REGION 1..15: note = X2m1 HCDR3
SEGDRYVSGYFGFDY

SEQ ID NO: 241 - protein, 15 aa, synthetic construct
  REGION 1..15: note = X2m2 HCDR3
SEGDRYVSGYFGLDY

SEQ ID NO: 242 - protein, 15 aa, synthetic construct
  REGION 1..15: note = X2m3 HCDR3
SEGDRYVSGYSGFDY

SEQ ID NO: 243 - protein, 15 aa, synthetic construct
  REGION 1..15: note = X2m4 HCDR3
SEGDRYVSGYSGLDY

SEQ ID NO: 244 - protein, 15 aa, synthetic construct
  REGION 1..15: note = X2m5 HCDR3
SEGDRYVSGYFGMDY

SEQ ID NO: 245 - protein, 15 aa, synthetic construct
  REGION 1..15: note = X2m6 HCDR3
SEGDRYVSGYSGMDY

SEQ ID NO: 246 - protein, 15 aa, synthetic construct
  REGION 1..15: note = X2m7 HCDR3
SEGDRYVSGYMGFDY

SEQ ID NO: 247 - protein, 15 aa, synthetic construct
  REGION 1..15: note = X2m8 HCDR3
SEGDRYVSGYMGLDY

SEQ ID NO: 248 - protein, 15 aa, synthetic construct
  REGION 1..15: note = X17m1 HCDR3
GDHGYYVSGYLGMDY

SEQ ID NO: 249 - protein, 15 aa, synthetic construct
  REGION 1..15: note = X17m2 HCDR3
GDHGYYVYGYLGMDY

SEQ ID NO: 250 - protein, 15 aa, synthetic construct
  REGION 1..15: note = X17m3 HCDR3
GDHGYYVSGYLGFDY

SEQ ID NO: 251 - protein, 15 aa, synthetic construct
  REGION 1..15: note = X17m6 HCDR3
GEHGYYVSGYLGFDY

SEQ ID NO: 252 - protein, 124 aa, synthetic construct
  REGION 1..124: note = X2m1 VH
EVQLVESGGGLVQPGGSLRLSCAASGFTVSSSSIHWVRQAPGKGLEWVASISSSYGSTSYADSVKGRFTISADTSKNTAYLQMNSLRAEDTAVYYCARSEGDRYVSGYFGFDYWGQGTLVTVSS

SEQ ID NO: 253 - protein, 124 aa, synthetic construct
  REGION 1..124: note = X2m2 VH
EVQLVESGGGLVQPGGSLRLSCAASGFTVSSSSIHWVRQAPGKGLEWVASISSSYGSTSYADSVKGRFTISADTSKNTAYLQMNSLRAEDTAVYYCARSEGDRYVSGYFGLDYWGQGTLVTVSS

SEQ ID NO: 254 - protein, 124 aa, synthetic construct
  REGION 1..124: note = X2m3 VH
EVQLVESGGGLVQPGGSLRLSCAASGFTVSSSSIHWVRQAPGKGLEWVASISSSYGSTSYADSVKGRFTISADTSKNTAYLQMNSLRAEDTAVYYCARSEGDRYVSGYSGFDYWGQGTLVTVSS

SEQ ID NO: 255 - protein, 124 aa, synthetic construct
  REGION 1..124: note = X2m4 VH
EVQLVESGGGLVQPGGSLRLSCAASGFTVSSSSIHWVRQAPGKGLEWVASISSSYGSTSYADSVKGRFTISADTSKNTAYLQMNSLRAEDTAVYYCARSEGDRYVSGYSGLDYWGQGTLVTVSS

SEQ ID NO: 256 - protein, 124 aa, synthetic construct
  REGION 1..124: note = X2m5 VH
EVQLVESGGGLVQPGGSLRLSCAASGFTVSSSSIHWVRQAPGKGLEWVASISSSYGSTSYADSVKGRFTISADTSKNTAYLQMNSLRAEDTAVYYCARSEGDRYVSGYFGMDYWGQGTLVTVSS

SEQ ID NO: 257 - protein, 124 aa, synthetic construct
  REGION 1..124: note = X2m6 VH
EVQLVESGGGLVQPGGSLRLSCAASGFTVSSSSIHWVRQAPGKGLEWVASISSSYGSTSYADSVKGRFTISADTSKNTAYLQMNSLRAEDTAVYYCARSEGDRYVSGYSGMDYWGQGTLVTVSS

SEQ ID NO: 258 - protein, 124 aa, synthetic construct
  REGION 1..124: note = X2m7 VH
EVQLVESGGGLVQPGGSLRLSCAASGFTVSSSSIHWVRQAPGKGLEWVASISSSYGSTSYADSVKGRFTISADTSKNTAYLQMNSLRAEDTAVYYCARSEGDRYVSGYMGFDYWGQGTLVTVSS

SEQ ID NO: 259 - protein, 124 aa, synthetic construct
  REGION 1..124: note = X2m8 VH
EVQLVESGGGLVQPGGSLRLSCAASGFTVSSSSIHWVRQAPGKGLEWVASISSSYGSTSYADSVKGRFTISADTSKNTAYLQMNSLRAEDTAVYYCARSEGDRYVSGYMGLDYWGQGTLVTVSS

SEQ ID NO: 260 - protein, 124 aa, synthetic construct
  REGION 1..124: note = X17m1 VH
EVQLVESGGGLVQPGGSLRLSCAASGFTISYSSIHWVRQAPGKGLEWVASIYSSSGSTYYADSVKGRFTISADTSKNTAYLQMNSLRAEDTAVYYCARGDHGYYVSGYLGMDYWGQGTLVTVSS

SEQ ID NO: 261 - protein, 124 aa, synthetic construct
  REGION 1..124: note = X17m2 VH
EVQLVESGGGLVQPGGSLRLSCAASGFTISYSSIHWVRQAPGKGLEWVASIYSSSGSTYYADSVKGRFTISADTSKNTAYLQMNSLRAEDTAVYYCARGDHGYYVYGYLGMDYWGQGTLVTVSS

SEQ ID NO: 262 - protein, 124 aa, synthetic construct
  REGION 1..124: note = X17m3 VH
EVQLVESGGGLVQPGGSLRLSCAASGFTISYSSIHWVRQAPGKGLEWVASIYSSSGSTYYADSVKGRFTISADTSKNTAYLQMNSLRAEDTAVYYCARGDHGYYVSGYLGFDYWGQGTLVTVSS

SEQ ID NO: 263 - protein, 124 aa, synthetic construct
  REGION 1..124: note = X17m6 VH
EVQLVESGGGLVQPGGSLRLSCAASGFTISYSSIHWVRQAPGKGLEWVASIYSSSGSTYYADSVKGRFTISADTSKNTAYLQMNSLRAEDTAVYYCARGEHGYYVSGYLGFDYWGQGTLVTVSS

SEQ ID NO: 264 - DNA, 1432 bp, synthetic construct
  misc_feature 1..1432: note = VH_3B2g2m1 coding sequence
  source 1..1432: mol_type = other DNA
aagcttgccgccaccatggaatggtcctgggtgttcctgttcttcctgtccgtgaccaccggcgtgcactctgaagttcagctgctggaatctggcggcggattggttcaacctggcggctctctgagactgtcttgtgccgcttctggcttcaccttctccgactacggcatgtcctgggtccgacaggctcctggcaaaggactggaatgggtgtccgccattccttggagcggcggctccacctactacaaagaatccgtgaagggccgctttaccatcagccgggacaactccaagaacaccctgtacctgcagatgaactccctgagagccgaggacaccgccgtgtactactgcgctaagagatccggcagaatcgccttcggcgctctggatgcttggggacagggaacactggtcaccgtgtcctccgcttctaccaagggaccctctgtgttccctctggctccttccagcaagtctacctctggcggaacagctgctctgggctgcctggtcaaggactactttcctgagcctgtgaccgtgtcttggaactctggcgctctgacatctggcgtgcacacctttccagctgtgctgcagtcctccggcctgtactctctgtcctctgtcgtgaccgtgccttccagctctctgggaacccagacctacatctgcaatgtgaaccacaagccttccaacaccaaggtggacaagaaggtggaacccaagtcctgcgacaagacccacacctgtcctccatgtcctgctccagaagctgctggcggcccatccgtgtttctgttccctccaaagcctaaggacaccctgatgatctctcggacccctgaagtgacctgcgtggtggtggatgtgtctcacgaggacccagaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctagggaaccccaggtttacaccttgcctccatctcgggacgagctgaccaagaaccaggtgtccctgacctgcctcgtgaagggattctacccctccgatatcgccgtggaatgggagtctaatggccagcctgagaacaactacaagacaacccctcctgtgctggactccgacggctcattctttctgtactccaagctgacagtggacaagtccagatggcagcagggcaacgtgttctcctgcagcgtgatgcacgaggccctgcacaatcactacacacagaagtccctgtctctgtcccctggctgatgaattc

SEQ ID NO: 265 - DNA, 739 bp, synthetic construct
  misc_feature 1..739: note = VL_3B2g2m1 coding sequence
  source 1..739: mol_type = other DNA
aagcttgccgccaccatgtctgtgcctacacaggttctgggactgctgctgctgtggctgaccgatgctagatgccagaccgtcgtgacccaagagccttccttctctgtgtctcctggcggcaccgtgacactgacatgtggactgtcctctggctccgtgacctccagcaactaccccgactggtatcagcagacccctggacaggctcctcggacactgatctactccaccgactccagacactctggcgtgcccgatagattctccggctctatcctgggcaacaaggccgctctgacaatcactggcgcccaggccgatgacgagtccgattactactgcggcctgtactcctactccggctccaagaactacgtgttcggcggaggcaccaagctgacagtgctgggacaacctaaggctgccccttccgtgactctgttccctccatcctctgaggaactgcaggccaacaaggctaccctcgtgtgcctgatctccgacttttaccctggcgctgtgaccgtggcctggaaggctgatagttctcctgtgaaggccggcgtggaaaccaccacaccttccaagcagtccaacaacaaatacgccgcctcctcctacctgtctctgacccctgaacagtggaagtcccaccggtcctacagctgccaagtgacccatgagggctccaccgtggaaaagaccgtggctcctaccgagtgctcctgatgaattc

SEQ ID NO: 266 - protein, 220 aa, synthetic construct
  REGION 1..220: note = Fcc with K (human domain)
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 267 - protein, 219 aa, synthetic construct
  REGION 1..219: note = Fcc without K (human domain)
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 268 - protein, 450 aa, synthetic construct
  REGION 1..450: note = ARGX-119 hIgG1, full length heavy chain, reduced effector function, delK
EVQLLESGGGLVQPGGSLRLSCAASGFTFSDYGMSWVRQAPGKGLEWVSAIPWSGGSTYYKESVKGRFTISRDNSKNTLYLQMNSLRAEDTAVYYCAKRSGRIAFGALDAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 269 - protein, 218 aa, synthetic construct
  REGION 1..218: note = ARGX-119 hIgG1, full length light chain, reduced effector function, delK
QTVVTQEPSFSVSPGGTVTLTCGLSSGSVTSSNYPDWYQQTPGQAPRTLIYSTDSRHSGVPDRFSGSILGNKAALTITGAQADDESDYYCGLYSYSGSKNYVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 270 - protein, 451 aa, synthetic construct
  REGION 1..451: note = ARGX-119 hIgG1, full length heavy chain, reduced effector function
EVQLLESGGGLVQPGGSLRLSCAASGFTFSDYGMSWVRQAPGKGLEWVSAIPWSGGSTYYKESVKGRFTISRDNSKNTLYLQMNSLRAEDTAVYYCAKRSGRIAFGALDAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 271 - protein, 218 aa, synthetic construct; identical to SEQ ID NO: 269
  REGION 1..218: note = ARGX-119 hIgG1, full length light chain, reduced effector function

SEQ ID NO: 272 - protein, 449 aa, synthetic construct
  REGION 1..449: note = Mota-hIgG1, full length heavy chain, reduced effector function, delK
QVTLRESGPALVKPTQTLTLTCTFSGFSLSTAGMSVGWIRQPPGKALEWLADIWWDDKKHYNPSLKDRLTISKDTSKNQVVLKVTNMDPADTATYYCARDMIFNFYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 273 - protein, 213 aa, synthetic construct
  REGION 1..213: note = Mota-hIgG1, full length light chain, reduced effector function, delK
DIQMTQSPSTLSASVGDRVTITCSASSRVGYMHWYQQKPGKAPKLLIYDTSKLASGVPSRFSGSGSGTEFTLTISSLQPDDFATYYCFQGSGYPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 274 - protein, 450 aa, synthetic construct
  REGION 1..450: note = Mota-hIgG1, full length heavy chain, reduced effector function
QVTLRESGPALVKPTQTLTLTCTFSGFSLSTAGMSVGWIRQPPGKALEWLADIWWDDKKHYNPSLKDRLTISKDTSKNQVVLKVTNMDPADTATYYCARDMIFNFYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 275 - protein, 213 aa, synthetic construct; identical to SEQ ID NO: 273
  REGION 1..213: note = Mota-hIgG1, full length light chain, reduced effector function

SEQ ID NO: 276 - DNA, 1350 bp, synthetic construct
  misc_feature 1..1350: note = coding DNA for HC_ARGX-119
  source 1..1350: mol_type = other DNA
gaagttcagctgctggaatctggcggcggattggttcaacctggcggctctctgagactgtcttgtgccgcttctggcttcaccttctccgactacggcatgtcctgggtccgacaggctcctggcaaaggactggaatgggtgtccgccattccttggagcggcggctccacctactacaaagaatccgtgaagggccgctttaccatcagccgggacaactccaagaacaccctgtacctgcagatgaactccctgagagccgaggacaccgccgtgtactactgcgctaagagatccggcagaatcgccttcggcgctctggatgcttggggacagggaacactggtcaccgtgtcctccgcttctaccaagggaccctctgtgttccctctggctccttccagcaagtctacctctggcggaacagctgctctgggctgcctggtcaaggactactttcctgagcctgtgaccgtgtcttggaactctggcgctctgacatctggcgtgcacacctttccagctgtgctgcagtcctccggcctgtactctctgtcctctgtcgtgaccgtgccttccagctctctgggaacccagacctacatctgcaatgtgaaccacaagccttccaacaccaaggtggacaagaaggtggaacccaagtcctgcgacaagacccacacctgtcctccatgtcctgctccagaagctgctggcggcccatccgtgtttctgttccctccaaagcctaaggacaccctgatgatctctcggacccctgaagtgacctgcgtggtggtggatgtgtctcacgaggacccagaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctagggaaccccaggtttacaccttgcctccatctcgggacgagctgaccaagaaccaggtgtccctgacctgcctcgtgaagggattctacccctccgatatcgccgtggaatgggagtctaatggccagcctgagaacaactacaagacaacccctcctgtgctggactccgacggctcattctttctgtactccaagctgacagtggacaagtccagatggcagcagggcaacgtgttctcctgcagcgtgatgcacgaggccctgcacaatcactacacacagaagtccctgtctctgtcccctggc

SEQ ID NO: 277 - DNA, 363 bp, synthetic construct
  misc_feature 1..363: note = coding DNA for VH_ARGX-119
  source 1..363: mol_type = other DNA
gaagttcagctgctggaatctggcggcggattggttcaacctggcggctctctgagactgtcttgtgccgcttctggcttcaccttctccgactacggcatgtcctgggtccgacaggctcctggcaaaggactggaatgggtgtccgccattccttggagcggcggctccacctactacaaagaatccgtgaagggccgctttaccatcagccgggacaactccaagaacaccctgtacctgcagatgaactccctgagagccgaggacaccgccgtgtactactgcgctaagagatccggcagaatcgccttcggcgctctggatgcttggggacagggaacactggtcaccgtgtcctcc

SEQ ID NO: 278 - DNA, 654 bp, synthetic construct
  misc_feature 1..654: note = coding DNA for LC_ARGX-119
  source 1..654: mol_type = other DNA
cagaccgtcgtgacccaagagccttccttctctgtgtctcctggcggcaccgtgacactgacatgtggactgtcctctggctccgtgacctccagcaactaccccgactggtatcagcagacccctggacaggctcctcggacactgatctactccaccgactccagacactctggcgtgcccgatagattctccggctctatcctgggcaacaaggccgctctgacaatcactggcgcccaggccgatgacgagtccgattactactgcggcctgtactcctactccggctccaagaactacgtgttcggcggaggcaccaagctgacagtgctgggacaacctaaggctgccccttccgtgactctgttccctccatcctctgaggaactgcaggccaacaaggctaccctcgtgtgcctgatctccgacttttaccctggcgctgtgaccgtggcctggaaggctgatagttctcctgtgaaggccggcgtggaaaccaccacaccttccaagcagtccaacaacaaatacgccgcctcctcctacctgtctctgacccctgaacagtggaagtcccaccggtcctacagctgccaagtgacccatgagggctccaccgtggaaaagaccgtggctcctaccgagtgctcc

SEQ ID NO: 279 - DNA, 336 bp, synthetic construct
  misc_feature 1..336: note = coding DNA for VL_ARGX-119
  source 1..336: mol_type = other DNA
cagaccgtcgtgacccaagagccttccttctctgtgtctcctggcggcaccgtgacactgacatgtggactgtcctctggctccgtgacctccagcaactaccccgactggtatcagcagacccctggacaggctcctcggacactgatctactccaccgactccagacactctggcgtgcccgatagattctccggctctatcctgggcaacaaggccgctctgacaatcactggcgcccaggccgatgacgagtccgattactactgcggcctgtactcctactccggctccaagaactacgtgttcggcggaggcaccaagctgacagtgctg

SEQ ID NO: 280 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 281 - protein, 106 aa, synthetic construct
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 282 - protein, 450 aa, synthetic construct
ELQLVESGGGLVQPGGSLRLSCAASGFTFDDYGMSWVRQAPGKGLEWVSAIPWNGGSTYYKESVKGRFTISRDNAKKTLYLQMNSLKSEDTAVYYCAKRSGRIAFGALDAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 283 - protein, 218 aa, synthetic construct
QAVVTQEPSLSVSPGGTVTLTCGLSSGSVTSSNYPDWYQQTPGQAPRGLIYSTDSRHSGVPSRFSGSISGNKAALTITGAQSEDEADYYCGLYMYSGSKNYVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 284 - DNA, 363 bp, synthetic construct
  source 1..363: mol_type = other DNA
gagttgcagctggtggagtctgggggaggcttggtgcagcctggggggtctctgagactctcctgtgcagcttctggattcacttttgatgattatggcatgagctgggtccgacaggctccagggaaggggctggaatgggtctcagctataccctggaatggtggtagcacatactataaagagtccgtgaagggccgattcaccatctccagagacaacgccaagaaaacgctgtatctgcaaatgaacagtctgaaatctgaggacacggccgtgtattactgtgcaaagcgatcaggccgaattgccttcggggctttggacgcatggggccaggggaccctggtcactgtctcctca

SEQ ID NO: 285 - DNA, 336 bp, synthetic construct
  source 1..336: mol_type = other DNA
caggctgtggtgacccaggagccgtccctgtcagtgtctccaggagggacggtcacactcacctgcggcctcagctctgggtctgtcacttccagtaactaccctgattggtaccagcagacaccaggccaggctccccgaggtcttatctacagcacagacagccgccactctggggtccccagtcgcttctccggatccatctctgggaacaaagccgccctcaccatcacgggggcccagtccgaggacgaggccgactattactgtggtctgtacatgtatagtggtagtaagaattatgtgttcggcggagggaccaagctgaccgtcctc

SEQ ID NO: 286 - DNA, 987 bp, synthetic construct
  source 1..987: mol_type = other DNA
gccagtacaaaaggtccaagtgtgttccctcttgctccctcatccaagagtaccagtggaggcaccgccgctcttggctgcttggttaaggattatttcccagagcctgtcactgtttcatggaactccggcgccttgacatctggtgtgcatacctttccagccgtgctgcagtcaagtggcctctacagcctcagtagcgtggtcactgtgcccagcagctctctcggcacacaaacttatatctgtaatgtgaatcataagccttcaaataccaaggtggataagaaagtggaaccaaaatcatgtgacaagacacacacctgccctccttgtccagcccccgaagcggccggtgggcccagcgtgttcctgtttcctcctaaacccaaagacactctgatgattagtaggaccccagaagtcacttgcgtggtggttgacgtgtcacatgaagatcccgaggtcaagttcaattggtatgttgacggggtcgaagttcacaacgctaaaactaaaccaagagaggaacagtataactctacctaccgggtggtgagtgttctgactgtcctccatcaagactggctgaatggcaaagaatacaagtgtaaggtgagcaacaaagccctgcccgctcctatagagaaaacaatatccaaagccaaaggtcaacctcgcgagccacaggtgtacaccctcccaccaagccgcgatgaacttactaagaaccaagtctctcttacttgcctggttaaggggttctatccatccgacattgcagtcgagtgggagtctaatggacagcctgagaacaactacaaaaccacccctcctgttctggattctgacggatctttcttcctttattctaaactcaccgtggataaaagcaggtggcagcagggcaacgtgttcagctgttccgttatgcatgaggccctgcataaccattatacccagaagtctttgtccctcagtccagga

SEQ ID NO: 287 - DNA, 318 bp, synthetic construct
  source 1..318: mol_type = other DNA
ggtcaacctaaagcagcaccttcagttactctgtttccacctagttcagaggaactgcaggccaataaagccacactcgtctgcctcatcagtgacttctacccaggagccgtgaccgtggcctggaaagccgacagtagccccgtgaaggccggggtggagacaacaactcctagtaaacagagtaataacaaatatgccgctagtagttatctctccctcactcccgagcagtggaagtctcacagaagttactcttgtcaggttactcacgagggttccacagtggaaaagactgtggcccctactgaatgtagt

SEQ ID NO: 288 - DNA, 1350 bp, synthetic construct
  source 1..1350: mol_type = other DNA
gagttgcagctggtggagtctgggggaggcttggtgcagcctggggggtctctgagactctcctgtgcagcttctggattcacttttgatgattatggcatgagctgggtccgacaggctccagggaaggggctggaatgggtctcagctataccctggaatggtggtagcacatactataaagagtccgtgaagggccgattcaccatctccagagacaacgccaagaaaacgctgtatctgcaaatgaacagtctgaaatctgaggacacggccgtgtattactgtgcaaagcgatcaggccgaattgccttcggggctttggacgcatggggccaggggaccctggtcactgtctcctcagccagtacaaaaggtccaagtgtgttccctcttgctccctcatccaagagtaccagtggaggcaccgccgctcttggctgcttggttaaggattatttcccagagcctgtcactgtttcatggaactccggcgccttgacatctggtgtgcatacctttccagccgtgctgcagtcaagtggcctctacagcctcagtagcgtggtcactgtgcccagcagctctctcggcacacaaacttatatctgtaatgtgaatcataagccttcaaataccaaggtggataagaaagtggaaccaaaatcatgtgacaagacacacacctgccctccttgtccagcccccgaagcggccggtgggcccagcgtgttcctgtttcctcctaaacccaaagacactctgatgattagtaggaccccagaagtcacttgcgtggtggttgacgtgtcacatgaagatcccgaggtcaagttcaattggtatgttgacggggtcgaagttcacaacgctaaaactaaaccaagagaggaacagtataactctacctaccgggtggtgagtgttctgactgtcctccatcaagactggctgaatggcaaagaatacaagtgtaaggtgagcaacaaagccctgcccgctcctatagagaaaacaatatccaaagccaaaggtcaacctcgcgagccacaggtgtacaccctcccaccaagccgcgatgaacttactaagaaccaagtctctcttacttgcctggttaaggggttctatccatccgacattgcagtcgagtgggagtctaatggacagcctgagaacaactacaaaaccacccctcctgttctggattctgacggatctttcttcctttattctaaactcaccgtggataaaagcaggtggcagcagggcaacgtgttcagctgttccgttatgcatgaggccctgcataaccattatacccagaagtctttgtccctcagtccagga

SEQ ID NO: 289 - DNA, 654 bp, synthetic construct
  source 1..654: mol_type = other DNA
caggctgtggtgacccaggagccgtccctgtcagtgtctccaggagggacggtcacactcacctgcggcctcagctctgggtctgtcacttccagtaactaccctgattggtaccagcagacaccaggccaggctccccgaggtcttatctacagcacagacagccgccactctggggtccccagtcgcttctccggatccatctctgggaacaaagccgccctcaccatcacgggggcccagtccgaggacgaggccgactattactgtggtctgtacatgtatagtggtagtaagaattatgtgttcggcggagggaccaagctgaccgtcctcggtcaacctaaagcagcaccttcagttactctgtttccacctagttcagaggaactgcaggccaataaagccacactcgtctgcctcatcagtgacttctacccaggagccgtgaccgtggcctggaaagccgacagtagccccgtgaaggccggggtggagacaacaactcctagtaaacagagtaataacaaatatgccgctagtagttatctctccctcactcccgagcagtggaagtctcacagaagttactcttgtcaggttactcacgagggttccacagtggaaaagactgtggcccctactgaatgtagt